SEQUENCE LISTING (556 sequences)

SEQ ID NO: 1 - protein, 5 aa, synthetic construct
  REGION 1..5: note = Sc02-004 HCDR1
GYLMS

SEQ ID NO: 2 - protein, 17 aa, synthetic construct
  REGION 1..17: note = Sc02-004 HCDR2
VISYDGSNKYYADSVKG

SEQ ID NO: 3 - protein, 10 aa, synthetic construct
  REGION 1..10: note = Sc02-004 HCDR3
ARRDTNLFDY

SEQ ID NO: 4 - protein, 11 aa, synthetic construct
  REGION 1..11: note = Sc02-004 LCDR1
RASQSISSYLN

SEQ ID NO: 5 - protein, 7 aa, synthetic construct
  REGION 1..7: note = Sc02-004 LCDR2
AASSLQS

SEQ ID NO: 6 - protein, 9 aa, synthetic construct
  REGION 1..9: note = Sc02-004 LCDR3
QQSYSTPPT

SEQ ID NO: 7 - protein, 5 aa, synthetic construct
  REGION 1..5: note = Sc02-025 HCDR1
SYYMH

SEQ ID NO: 8 - protein, 17 aa, synthetic construct
  REGION 1..17: note = Sc02-025 HCDR2
IINPSGGGTSYAQKFQG

SEQ ID NO: 9 - protein, 12 aa, synthetic construct
  REGION 1..12: note = Sc02-025 HCDR3
DYYVTYDSWFDS

SEQ ID NO: 10 - protein, 11 aa, synthetic construct
  REGION 1..11: note = Sc02-025 LCDR1
QGDSLRSYYAS

SEQ ID NO: 11 - protein, 7 aa, synthetic construct
  REGION 1..7: note = Sc02-025 LCDR2
GKNNRPS

SEQ ID NO: 12 - protein, 11 aa, synthetic construct
  REGION 1..11: note = Sc02-025 LCDR3
NSRDSSGNHVV

SEQ ID NO: 13 - protein, 114 aa, synthetic construct
  REGION 1..114: note = Sc02-004 VH
LVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTV

SEQ ID NO: 14 - protein, 104 aa, synthetic construct
  REGION 1..104: note = Sc02-004 VL
ELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEI

SEQ ID NO: 15 - protein, 117 aa, synthetic construct
  REGION 1..117: note = Sc02-025 VH
LVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVS

SEQ ID NO: 16 - protein, 98 aa, synthetic construct
  REGION 1..98: note = Sc02-025 VL
VSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTV

SEQ ID NO: 17 - protein, 244 aa, synthetic construct
  REGION 1..244: note = Sc02-004 scFv
AEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 18 - protein, 248 aa, synthetic construct
  REGION 1..248: note = Sc02-025 scFv
AQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAA

SEQ ID NO: 19 - protein, 15 aa, synthetic construct
  REGION 1..15: note = flexible linker
GGGGSGGGGSGGGGS

SEQ ID NO: 20 - protein, 15 aa, synthetic construct
  REGION 1..15: note = flexible linker
GTGGSGGTGSGTGTS

SEQ ID NO: 21 - protein, 12 aa, synthetic construct
  REGION 1..12: note = IgG4 hinge region (short)
ESKYGPPCPPCP

SEQ ID NO: 22 - protein, 45 aa, synthetic construct
  REGION 1..45: note = CD8a hinge region
TTTPAPRPPTPAPTIASQPLSLRPEACRPAAGGAVHTRGLDFACD

SEQ ID NO: 23 - protein, 39 aa, synthetic construct
  REGION 1..39: note = CD28 hinge region
IEVMYPPPYLDNEKSNGTIIHVKGKHLCPSPLFPGPSKP

SEQ ID NO: 24 - protein, 24 aa, synthetic construct
  REGION 1..24: note = CD8a transmembrane domain
IYIWAPLAGTCGVLLLSLVITLYC

SEQ ID NO: 25 - protein, 27 aa, synthetic construct
  REGION 1..27: note = CD28 transmembrane domain
FWVLVVVGGVLACYSLLVTVAFIIFWV

SEQ ID NO: 26 - protein, 113 aa, synthetic construct
  REGION 1..113: note = wildtype CD3z signaling domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 27 - protein, 112 aa, synthetic construct
  REGION 1..112: note = variant CD3z signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 41 aa, synthetic construct
  REGION 1..41: note = wildtype CD28 costimulatory signaling domain
RSKRSRLLHSDYMNMTPRRPGPTRKHYQPYAPPRDFAAYRS

SEQ ID NO: 29 - protein, 41 aa, synthetic construct
  REGION 1..41: note = variant CD28 costimulatory signaling domain with L186G/L187G substitutions with positions in reference to full length protein
RSKRSRGGHSDYMNMTPRRPGPTRKHYQPYAPPRDFAAYRS

SEQ ID NO: 30 - protein, 42 aa, synthetic construct
  REGION 1..42: note = 4-1BB costimulatory signaling domain
KRGRKKLLYIFKQPFMRPVQTTQEEDGCSCRFPEEEEGGCEL

SEQ ID NO: 31 - protein, 458 aa, synthetic construct
  REGION 1..458: note = pCTX206 CAR: GMSCFR signal peptide - sc02-004 scFv - IgG4 Hinge - CD28 TM - CD28 ICD - CD3z
  SIGNAL 1..21
MLLVTSLLLCELPHPAFLLIPAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 32 - protein, 462 aa, synthetic construct
  REGION 1..462: note = pCTX208 CAR: GMSCFR signal peptide - sc02-025 scFv - IgG4 Hinge - CD28 TM - CD28 ICD - CD3z
  SIGNAL 1..21
MLLVTSLLLCELPHPAFLLIPAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 33 - protein, 459 aa, synthetic construct
  REGION 1..459: note = CAR: GMSCFR signal peptide - sc02-004 scFv - IgG4 Hinge - CD28 TM - 4-1BB ICD - CD3z
  SIGNAL 1..21
MLLVTSLLLCELPHPAFLLIPAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 34 - protein, 463 aa, synthetic construct
  REGION 1..463: note = CAR: GMSCFR signal peptide - sc02-025 scFv - IgG4 Hinge - CD28 TM - 4-1BB ICD - CD3z
  SIGNAL 1..21
MLLVTSLLLCELPHPAFLLIPAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 35 - protein, 485 aa, synthetic construct
  REGION 1..485: note = CAR: GMSCFR signal peptide - sc02-004 scFv - CD28 Hinge - CD28 TM - CD28 ICD - CD3z
  SIGNAL 1..21
MLLVTSLLLCELPHPAFLLIPAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 36 - protein, 489 aa, synthetic construct
  REGION 1..489: note = CAR: GMSCFR signal peptide - sc02-025 scFv - CD28 Hinge - CD28 TM - CD28 ICD - CD3z
  SIGNAL 1..21
MLLVTSLLLCELPHPAFLLIPAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 37 - protein, 486 aa, synthetic construct
  REGION 1..486: note = CAR: GMSCFR signal peptide - sc02-004 scFv - CD28 Hinge - CD28 TM - 4-1BB ICD - CD3z
  SIGNAL 1..21
MLLVTSLLLCELPHPAFLLIPAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 38 - protein, 490 aa, synthetic construct
  REGION 1..490: note = CAR: GMSCFR signal peptide - sc02-025 scFv - CD28 Hinge - CD28 TM - 4-1BB ICD - CD3z
  SIGNAL 1..21
MLLVTSLLLCELPHPAFLLIPAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAIEVMYPPPYLDNEKSNGTIIHVKGKHLCPSPLFPGPSKPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 39 - protein, 455 aa, synthetic construct
  REGION 1..455: note = CAR: GMSCFR signal peptide - sc02-004 scFv - IgG4 Hinge - CD8a TM - CD28 ICD - CD3z
MLLVTSLLLCELPHPAFLLIPAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 40 - protein, 459 aa, synthetic construct
  REGION 1..459: note = CAR: GMSCFR signal peptide - sc02-025 scFv - IgG4 Hinge - CD8a TM - CD28 ICD - CD3z
MLLVTSLLLCELPHPAFLLIPAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 41 - protein, 488 aa, synthetic construct
  REGION 1..488: note = CAR: GMSCFR signal peptide - sc02-004 scFv - CD8a Hinge - CD8a TM - CD28 ICD - CD3z
  SIGNAL 1..21
MLLVTSLLLCELPHPAFLLIPAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - protein, 492 aa, synthetic construct
  REGION 1..492: note = CAR: GMSCFR signal peptide - sc02-025 scFv - CD8a Hinge - CD8a TM - CD28 ICD - CD3z
  SIGNAL 1..21
MLLVTSLLLCELPHPAFLLIPAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 43 - protein, 456 aa, synthetic construct
  REGION 1..456: note = CAR: GMSCFR signal peptide - sc02-004 scFv - IgG4 Hinge - CD8a TM - 4-1BB ICD - CD3z
  SIGNAL 1..21
MLLVTSLLLCELPHPAFLLIPAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 44 - protein, 460 aa, synthetic construct
  REGION 1..460: note = CAR: GMSCFR signal peptide - sc02-025 scFv - IgG4 Hinge - CD8a TM - 4-1BB ICD - CD3z
  SIGNAL 1..21
MLLVTSLLLCELPHPAFLLIPAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 45 - protein, 489 aa, synthetic construct
  REGION 1..489: note = CAR: GMSCFR signal peptide - sc02-004 scFv - CD8a Hinge - CD8a TM - 4-1BB ICD - CD3z
  SIGNAL 1..21
MLLVTSLLLCELPHPAFLLIPAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 493 aa, synthetic construct
  REGION 1..493: note = CAR: GMSCFR signal peptide - sc02-025 scFv - CD8a Hinge - CD8a TM - 4-1BB ICD - CD3z
  SIGNAL 1..21
MLLVTSLLLCELPHPAFLLIPAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 47 - protein, 21 aa, synthetic construct
  REGION 1..21: note = GMCSFR_Signal Peptide
MLLVTSLLLCELPHPAFLLIP

SEQ ID NO: 48 - protein, 359 aa, Homo sapiens
MAEAITYADLRFVKAPLKKSISSRLGQDPGADDDGEITYENVQVPAVLGVPSSLASSVLGDKAAVKSEQPTASWRAVTSPAVGRILPCRTTCLRYLLLGLLLTCLLLGVTAICLGVRYLQVSQQLQQTNRVLEVTNSSLRQQLRLKITQLGQSAEDLQGSRRELAQSQEALQVEQRAHQAAEGQLQACQADRQKTKETLQSEEQQRRALEQKLSNMENRLKPFFTCGSADTCCPSGWIMHQKSCFYISLTSKNWQESQKQCETLSSKLATFSEIYPQSHSYYFLNSLLPNGGSGNSYWTGLSSNKDWKLTDDTQRTRTYAQSSKCNKVHKTWSWWTLESESCRSSLPYICEMTAFRFPD

SEQ ID NO: 49 - protein, 378 aa, Homo sapiens
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 50 - protein, 314 aa, Homo sapiens
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQ

SEQ ID NO: 51 - protein, 279 aa, Mus musculus
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 52 - protein, 111 aa, Homo sapiens
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRAS

SEQ ID NO: 53 - protein, 179 aa, Homo sapiens
TTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQ

SEQ ID NO: 54 - protein, 24 aa, Homo sapiens
MSKEPLILWLMIEFWWLYLTPVTS

SEQ ID NO: 55 - protein, 22 aa, Mus musculus
MSKGLLLLWLVTELWWLYLTPA

SEQ ID NO: 56 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 57 - protein, 295 aa, Homo sapiens
  SIGNAL 1..20
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKG

SEQ ID NO: 58 - protein, 108 aa, Homo sapiens
SVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPK

SEQ ID NO: 59 - protein, 167 aa, Homo sapiens
VTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKG

SEQ ID NO: 60 - protein, 20 aa, Homo sapiens
MHPQVVILSLILHLADSVAG

SEQ ID NO: 61 - protein, 359 aa, Homo sapiens; identical to SEQ ID NO: 48

SEQ ID NO: 62 - protein, 292 aa, Homo sapiens
  SIGNAL 1..15
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFH

SEQ ID NO: 63 - protein, 290 aa, Homo sapiens
  SIGNAL 1..20
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKG

SEQ ID NO: 64 - protein, 314 aa, Homo sapiens; identical to SEQ ID NO: 50
  SIGNAL 1..24

SEQ ID NO: 65 - protein, 202 aa, Homo sapiens
  SIGNAL 1..21
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIG

SEQ ID NO: 66 - protein, 161 aa, Homo sapiens
LNGCANPLGLKNNSIPDKQITASSSYKTWGLHLFSWNPSYARLDKQGNFNAWVAGSYGNDQWLQVDLGSSKEVTGIITQGARNFGSVQFVASYKVAYSNDSANWTEYQDPRTGSSKIFPGNWDNHSHKKNLFETPILARYVRILPVAWHNRIALRLELLGC

SEQ ID NO: 67 - protein, 94 aa, Homo sapiens
  SIGNAL 1..30
MAPSLSPGPAALRRAPQLLLLLLAAECALAALLPAREATQFLRPRQRRAFQVFEEAKQGHLERECVEELCSREEAREVFENDPETDYFYPRYLD

SEQ ID NO: 68 - protein, 87 aa, Homo sapiens
  SIGNAL 1..24
MRVLGGRCGALLACLLLVLPVSEANFLSKQQASQVLVRKRRANSLLEETKQGNLERECIEELCNKEEAREVFENDPETDYFYPKYLV

SEQ ID NO: 69 - protein, 918 aa, Homo sapiens
AAGADAGPGPEPCATLVQGKFFGYFSAAAVFPANASRCSWTLRNPDPRRYTLYMKVAKAPVPCSGPGRVRTYQFDSFLESTRTYLGVESFDEVLRLCDPSAPLAFLQASKQFLQMRRQQPPQHDGLRPRAGPPGPTDDFSVEYLVVGNRNPSRAACQMLCRWLDACLAGSRSSHPCGIMQTPCACLGGEAGGPAAGPLAPRGDVCLRDAVAGGPENCLTSLTQDRGGHGATGGWKLWSLWGECTRDCGGGLQTRTRTCLPAPGVEGGGCEGVLEEGRQCNREACGPAGRTSSRSQSLRSTDARRREELGDELQQFGFPAPQTGDPAAEEWSPWSVCSSTCGEGWQTRTRFCVSSSYSTQCSGPLREQRLCNNSAVCPVHGAWDEWSPWSLCSSTCGRGFRDRTRTCRPPQFGGNPCEGPEKQTKFCNIALCPGRAVDGNWNEWSSWSACSASCSQGRQQRTRECNGPSYGGAECQGHWVETRDCFLQQCPVDGKWQAWASWGSCSVTCGAGSQRRERVCSGPFFGGAACQGPQDEYRQCGTQRCPEPHEICDEDNFGAVIWKETPAGEVAAVRCPRNATGLILRRCELDEEGIAYWEPPTYIRCVSIDYRNIQMMTREHLAKAQRGLPGEGVSEVIQTLVEISQDGTSYSGDLLSTIDVLRNMTEIFRRAYYSPTPGDVQNFVQILSNLLAEENRDKWEEAQLAGPNAKELFRLVEDFVDVIGFRMKDLRDAYQVTDNLVLSIHKLPASGATDISFPMKGWRATGDWAKVPEDRVTVSKSVFSTGLTEADEASVFVVGTVLYRNLGSFLALQRNTTVLNSKVISVTVKPPPRSLRTPLEIEFAHMYNGTTNQTCILWDETDVPSSSAPPQLGPWSWRGCRTVPLDALRTRCLCDRLSTFAILAQLSADANMEKATLPS

SEQ ID NO: 70 - protein, 13 aa, Homo sapiens
PVLSLNITCQMNK

SEQ ID NO: 71 - protein, 21 aa, Homo sapiens
LLIVTIVATMLVLAVTVTSLC

SEQ ID NO: 72 - protein, 21 aa, Homo sapiens
ALVSGMCCALFLLILLTGVLC

SEQ ID NO: 73 - protein, 21 aa, Homo sapiens
FFMINTSILLIFIFIVLLIHF

SEQ ID NO: 74 - protein, 21 aa, Homo sapiens
TIIGVSVLSVLVVSVVAVLVY

SEQ ID NO: 75 - protein, 21 aa, Homo sapiens
FSLFIVCTVTLTLFLMTILTV

SEQ ID NO: 76 - protein, 21 aa, Homo sapiens
ALVSGMCCALFLLILLTGVLC

SEQ ID NO: 77 - protein, 21 aa, Homo sapiens
LILFSLSISVSLFLMVMMTAS

SEQ ID NO: 78 - protein, 21 aa, Homo sapiens
AVILFFFTFFITTMVMLAALA

SEQ ID NO: 79 - protein, 21 aa, Homo sapiens
FALSLLAVALGLGVPMLHHLC

SEQ ID NO: 80 - protein, 21 aa, Homo sapiens
IYAGVCISVLVLLALLGVIIA

SEQ ID NO: 81 - protein, 21 aa, Homo sapiens
LLMIIAPSLGFVLFALFVAFL

SEQ ID NO: 82 - protein, 21 aa, Homo sapiens
VLFYLAVGIMFLVNTVLWVTI

SEQ ID NO: 83 - protein, 21 aa, Homo sapiens
LCYILDAILFLYGIVLTLLYC

SEQ ID NO: 84 - protein, 21 aa, Homo sapiens
IYIWAPLAGTCGVLLLSLVIT

SEQ ID NO: 85 - protein, 21 aa, Homo sapiens
FGCFCGFILIGLILYISLAIR

SEQ ID NO: 86 - protein, 21 aa, Homo sapiens
YVLLGAVVAAACVLILALFLV

SEQ ID NO: 87 - protein, 21 aa, Homo sapiens
VPVVLGVLTALVTAAALALIL

SEQ ID NO: 88 - protein, 27 aa, Homo sapiens
FWVLVVVGGVLACYSLLVTVAFIIFWV

SEQ ID NO: 89 - protein, 22 aa, Homo sapiens
MALIVLGGVAGLLLFIGLGIFF

SEQ ID NO: 90 - protein, 21 aa, Homo sapiens
GVLAGIVMGDLVLTVLIALAV

SEQ ID NO: 91 - protein, 21 aa, Homo sapiens
VTLIVGCGVSSLTLLMLVIIY

SEQ ID NO: 92 - protein, 21 aa, Homo sapiens
GVVIIVILLILTGAGLAAYFF

SEQ ID NO: 93 - protein, 42 aa, Homo sapiens
RLKIQVRKAAITSYEKSDGVYTGLSTRNQETYETLKHEKPPQ

SEQ ID NO: 94 - protein, 61 aa, Homo sapiens
RKELKRKKKWDLEISLDSGHEKKVISSLQEDRHLEEELKCQEQKEEQLQEGVHRKEPQGAT

SEQ ID NO: 95 - protein, 77 aa, Homo sapiens
CRKKRISANSTDPVKAAQFEPPGRQMIAIRKRQLEETNNDYETADGGYMTLNPRAPTDDDKNIYLTLPPNDHVNSNN

SEQ ID NO: 96 - protein, 77 aa, Homo sapiens
CRKKRISANSTDPVKAAQFEPPGRQMIAIRKRQPEETNNDYETADGGYMTLNPRAPTDDDKNIYLTLPPNDHVNSNN

SEQ ID NO: 97 - protein, 25 aa, Homo sapiens
KTNIRSSTRDWKDHKFKWRKDPQDK

SEQ ID NO: 98 - protein, 155 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 99 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 100 - protein, 261 aa, Homo sapiens
TLEDKELGSGNFGTVKKGYYQMKKVVKTVAVKILKNEANDPALKDELLAEANVMQQLDNPYIVRMIGICEAESWMLVMEMAELGPLNKYLQQNRHVKDKNIIELVHQVSMGMKYLEESNFVHRDLAARNVLLVTQHYAKISDFGLSKALRADENYYKAQTHGKWPVKWYAPECINYYKFSSKSDVWSFGVLMWEAFSYGQKPYRGMKGSEVTAMLEKGERMGCPAGCPREMYDLMNLCWTYDVENRPGFAAVELRLRNYYY

SEQ ID NO: 101 - protein, 263 aa, Homo sapiens
LIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSL

SEQ ID NO: 102 - protein, 85 aa, Homo sapiens
SWRRRQRRLRGASSAEAPDGDKDAPEPLDKVIILSPGISDATAPAWPPPGEDPGTTPPGHSVPVPATELGSTELVTTKTAGPEQQ

SEQ ID NO: 103 - protein, 52 aa, Homo sapiens
YFLGRLVPRGRGAAEAATRKQRITETESPYQELQGQRSDVYSDLNTQRPYYK

SEQ ID NO: 104 - protein, 86 aa, Homo sapiens
LWNKKRMRGPGKDPTRKCPDPRSASSPKQHPSESVYTALQRRETEVYACIENEDGSSPTAKQSPLSQERPHRFEDDGELNLVYENL

SEQ ID NO: 105 - protein, 29 aa, Homo sapiens
SSPKQHPSESVYTALQRRETEVYACIENE

SEQ ID NO: 106 - protein, 29 aa, Homo sapiens
DSKAGMEEDHTYEGLDIDQTATYEDIVTL

SEQ ID NO: 107 - protein, 303 aa, Homo sapiens
MAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 108 - protein, 274 aa, Homo sapiens
MESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKV

SEQ ID NO: 109 - protein, 45 aa, Homo sapiens
DSKAGMEEDHTYEGLDIDQTATYEDIVTLRTGEVKWSVGEHPGQE

SEQ ID NO: 110 - protein, 141 aa, Homo sapiens
HMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 111 - protein, 473 aa, Homo sapiens
KRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM

SEQ ID NO: 112 - protein, 185 aa, Homo sapiens
SYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 113 - protein, 175 aa, Homo sapiens
HRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 114 - protein, 179 aa, Homo sapiens
EGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 115 - protein, 187 aa, Homo sapiens
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 116 - protein, 198 aa, Homo sapiens
TKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 117 - protein, 189 aa, Homo sapiens
YLDLPWYLRMVCQWTQTRRRARNIPLEELQRNLQFHAFISYSEHDSAWVKSELVPYLEKEDIQICLHERNFVPGKSIVENIINCIEKSYKSIFVLSPNFVQSEWCHYELYFAHHNLFHEGSNNLILILLEPIPQNSIPNKYHKLKALMTQRTYLQWPKEKSKRGLFWANIRAAFNMKLTLVTENNDVKS

SEQ ID NO: 118 - protein, 189 aa, Homo sapiens
HLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 119 - protein, 193 aa, Homo sapiens
HHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 120 - protein, 193 aa, Homo sapiens
GWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 121 - protein, 522 aa, Homo sapiens
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 122 - protein, 274 aa, Homo sapiens
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 123 - protein, 303 aa, Homo sapiens; identical to SEQ ID NO: 107

SEQ ID NO: 124 - protein, 274 aa, Homo sapiens; identical to SEQ ID NO: 108

SEQ ID NO: 125 - protein, 46 aa, Homo sapiens
YKKRRVHLPQEGAFENTLYFNSQSSPGTSDMKDLVGNIEQNEHSVI

SEQ ID NO: 126 - protein, 57 aa, Homo sapiens
KLLVTIHDRREFAKFQSERSRARYEMASNPLYRKPISTHTVDFTFNKFNKSYNGTVD

SEQ ID NO: 127 - protein, 440 aa, Homo sapiens
LRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 128 - protein, 422 aa, Homo sapiens
HRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 129 - protein, 397 aa, Homo sapiens
RREVQDAVKCRVVDRQEEGNGDSGGSFQNGHAQLMTDFEKDVDLACRSVLNKDIAACRTATITGTLKRPSLPEEEKLKLAHAKGPPTNFNSLPANVSKLHLHGSPRYPGGPLPDFPNHSLTLKRDKAPKSSFVGDGDIFKKLDSELSRAQEKALDTSYVILPTATATLRPKPKEEPKYSIHIDQMPQTRLIHLSTAPEASLPARSPPSRQPPSGGPPEAPPAQPPPPPPPPPPPPQQPLPPPPNLEPAPPSLGDPGEPAAHPGPSTGPSTKNENVATLSVSSLERRKSRYAELDFEKIMHTRKRHQDMFQDLNRKLQHAAEKDKEVLGPDSKPEKQQTPNKRPWESLRKAHGTPTWVKKELEPLQPSPLELRSVEWERSGATIPLVGQDIIDLQTEV

SEQ ID NO: 130 - protein, 727 aa, Homo sapiens
MPPPADIVKVAIEWPGAYPKLMEIDQKKPLSAIIKEVCDGWSLANHEYFALQHADSSNFYITEKNRNEIKNGTILRLTTSPAQNAQQLHERIQSSSMDAKLEALKDLASLSRDVTFAQEFINLDGISLLTQMVESGTERYQKLQKIMKPCFGDMLSFTLTAFVELMDHGIVSWDTFSVAFIKKIASFVNKSAIDISILQRSLAILESMVLNSHDLYQKVAQEITIGQLIPHLQGSDQEIQTYTIAVINALFLKAPDERRQEMANILAQKQLRSIILTHVIRAQRAINNEMAHQLYVLQVLTFNLLEDRMMTKMDPQDQAQRDIIFELRRIAFDAESEPNNSSGSMEKRKSMYTRDYKKLGFINHVNPAMDFTQTPPGMLALDNMLYFAKHHQDAYIRIVLENSSREDKHECPFGRSSIELTKMLCEILKVGELPSETCNDFHPMFFTHDRSFEEFFCICIQLLNKTWKEMRATSEDFNKVMQVVKEQVMRALTTKPSSLDQFKSKLQNLSYTEILKIRQSERMNQEDFQSRPILELKEKIQPEILELIKQQRLNRLVEGTCFRKLNARRRQDKFWYCRLSPNHKVLHYGDLEESPQGEVPHDSLQDKLPVADIKAVVTGKDCPHMKEKGALKQNKEVLELAFSILYDSNCQLNFIAPDKHEYCIWTDGLNALLGKDMMSDLTRNDLDTLLSMEIKLRLLDLENIQIPDAPPPIPKEPSNYDFVYDCN

SEQ ID NO: 131 - protein, 776 aa, synthetic construct
  REGION 1..776: note = CER1
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 132 - protein, 487 aa, synthetic construct
  REGION 1..487: note = CER5
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 133 - protein, 487 aa, synthetic construct
  REGION 1..487: note = CER6
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 134 - protein, 500 aa, synthetic construct
  REGION 1..500: note = CER7
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 135 - protein, 740 aa, synthetic construct
  REGION 1..740: note = CER8
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLRKRRKETRFGQAFDSVMARGEPAVHFRAARSFNRERPERIEATLDSLGISDELKEKLEDVLIPEQQFTLGRMLGKGEFGSVREAQLKQEDGSFVKVAVKMLKADIIASSDIEEFLREAACMKEFDHPHVAKLVGVSLRSRAKGRLPIPMVILPFMKHGDLHAFLLASRIGENPFNLPLQTLIRFMVDIACGMEYLSSRNFIHRDLAARNCMLAEDMTVCVADFGLSRKIYSGDYYRQGCASKLPVKWLALESLADNLYTVQSDVWAFGVTMWEIMTRGQTPYAGIENAEIYNYLIGGNRLKQPPECMEDVYDLMYQCWSADPKQRPSFTCLRMELENILGQLSVLSASQDPLYINIERAEEPTAGGSLELPGRDQPYSGAGDGSGMGAVGGTPSDCRYILTPGGLAEQPGQAEHQPESPLNETQRLLLLQQGLLPHSSC

SEQ ID NO: 136 - protein, 352 aa, synthetic construct
  REGION 1..352: note = CER9
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLYFLGRLVPRGRGAAEAATRKQRITETESPYQELQGQRSDVYSDLNTQRPYYK

SEQ ID NO: 137 - protein, 352 aa, synthetic construct
  REGION 1..352: note = CER10
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTGVLAGIVMGDLVLTVLIALAVYFLGRLVPRGRGAAEAATRKQRITETESPYQELQGQRSDVYSDLNTQRPYYK

SEQ ID NO: 138 - protein, 722 aa, synthetic construct
  REGION 1..722: note = CER11
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 139 - protein, 342 aa, synthetic construct
  REGION 1..342: note = CER12
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLRLKIQVRKAAITSYEKSDGVYTGLSTRNQETYETLKHEKPPQ

SEQ ID NO: 140 - protein, 342 aa, synthetic construct
  REGION 1..342: note = CER13
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTLCYILDAILFLYGIVLTLLYCRLKIQVRKAAITSYEKSDGVYTGLSTRNQETYETLKHEKPPQ

SEQ ID NO: 141 - protein, 455 aa, synthetic construct
  REGION 1..455: note = CER15
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 142 - protein, 441 aa, synthetic construct
  REGION 1..441: note = CER16
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 143 - protein, 479 aa, synthetic construct
  REGION 1..479: note = CER17
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 144 - protein, 479 aa, synthetic construct
  REGION 1..479: note = CER18
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 145 - protein, 498 aa, synthetic construct
  REGION 1..498: note = CER19
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 146 - protein, 498 aa, synthetic construct
  REGION 1..498: note = CER20
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFSLFIVCTVTLTLFLMTILTVTKFRGFCFICYKTAQRLVFKDHPQGTEPDMYKYDAYLCFSSKDFTWVQNALLKHLDTQYSDQNRFNLCFEERDFVPGENRIANIQDAIWNSRKIVCLVSRHFLRDGWCLEAFSYAQGRCLSDLNSALIMVVVGSLSQYQLMKHQSIRGFVQKQQYLRWPEDFQDVGWFLHKLSQQILKKEKEKKKDNNIPLQTVATIS

SEQ ID NO: 147 - protein, 493 aa, synthetic construct
  REGION 1..493: note = CER21
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 148 - protein, 493 aa, synthetic construct
  REGION 1..493: note = CER22
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 149 - protein, 493 aa, synthetic construct
  REGION 1..493: note = CER23
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 150 - protein, 493 aa, synthetic construct
  REGION 1..493: note = CER24
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 151 - protein, 386 aa, synthetic construct
  REGION 1..386: note = CER25
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLWNKKRMRGPGKDPTRKCPDPRSASSPKQHPSESVYTALQRRETEVYACIENEDGSSPTAKQSPLSQERPHRFEDDGELNLVYENL

SEQ ID NO: 152 - protein, 485 aa, synthetic construct
  REGION 1..485: note = CER26
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKK

SEQ ID NO: 153 - protein, 475 aa, synthetic construct
  REGION 1..475: note = CER27
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 154 - protein, 489 aa, synthetic construct
  REGION 1..489: note = CER28
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 155 - protein, 574 aa, synthetic construct
  REGION 1..574: note = CER29
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 156 - protein, 603 aa, synthetic construct
  REGION 1..603: note = CER30
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 157 - protein, 574 aa, synthetic construct
  REGION 1..574: note = CER31
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMESSKKMDSPGALQTNPPLKLHTDRSAGTPVFVPEQGGYKEKFVKTVEDKYKCEKCHLVLCSPKQTECGHRFCESCMAALLSSSSPKCTACQESIVKDKVFKDNCCKREILALQIYCRNESRGCAEQLMLGHLLVHLKNDCHFEELPCVRPDCKEKVLRKDLRDHVEKACKYREATCSHCKSQVPMIALQKHEDTDCPCVVVSCPHKCSVQTLLRSELSAHLSECVNAPSTCSFKRYGCVFQGTNQQIKAHEASSAVQHVNLLKEWSNSLEKKV

SEQ ID NO: 158 - protein, 540 aa, synthetic construct
  REGION 1..540: note = CER85
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 159 - protein, 507 aa, synthetic construct
  REGION 1..507: note = CER86
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGYFLGRLVPRGRGAAEAATRKQRITETESPYQELQGQRSDVYSDLNTQRPYYK

SEQ ID NO: 160 - protein, 540 aa, synthetic construct
  REGION 1..540: note = CER87
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSWRRRQRRLRGASSAEAPDGDKDAPEPLDKVIILSPGISDATAPAWPPPGEDPGTTPPGHSVPVPATELGSTELVTTKTAGPEQQMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 161 - protein, 507 aa, synthetic construct
  REGION 1..507: note = CER88
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLYFLGRLVPRGRGAAEAATRKQRITETESPYQELQGQRSDVYSDLNTQRPYYKMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 162 - protein, 500 aa, synthetic construct
  REGION 1..500: note = CER89
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGDSKAGMEEDHTYEGLDIDQTATYEDIVTLRTGEVKWSVGEHPGQE

SEQ ID NO: 163 - protein, 541 aa, synthetic construct
  REGION 1..541: note = CER90
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGLWNKKRMRGPGKDPTRKCPDPRSASSPKQHPSESVYTALQRRETEVYACIENEDGSSPTAKQSPLSQERPHRFEDDGELNLVYENL

SEQ ID NO: 164 - protein, 692 aa, synthetic construct
  REGION 1..692: note = CER91
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGGSGATNFSLLKQAGDVEENPGPMASRGATRPNGPNTGNKICQFKLVLLGESAVGKSSLVLRFVKGQFHEFQESTIGAAFLTQTVCLDDTTVKFEIWDTAGQERYHSLAPMYYRGAQAAIVVYDITNEESFARAKNWVKELQRQASPNIVIALSGNKADLANKRAVDFQEAQSYADDNSLLFMETSAKTSMNVNEIFMAIAKKLPKNEPQNPGANSARGRGVDLTEPTQPTRNQCCSN

SEQ ID NO: 165 - protein, 931 aa, synthetic construct
  REGION 1..931: note = CER92
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMMAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLG

SEQ ID NO: 166 - protein, 861 aa, synthetic construct
  REGION 1..861: note = CER93
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 167 - protein, 828 aa, synthetic construct
  REGION 1..828: note = CER94
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMYFLGRLVPRGRGAAEAATRKQRITETESPYQELQGQRSDVYSDLNTQRPYYK

SEQ ID NO: 168 - protein, 821 aa, synthetic construct
  REGION 1..821: note = CER95
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMDSKAGMEEDHTYEGLDIDQTATYEDIVTLRTGEVKWSVGEHPGQE

SEQ ID NO: 169 - protein, 862 aa, synthetic construct
  REGION 1..862: note = CER96
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMLWNKKRMRGPGKDPTRKCPDPRSASSPKQHPSESVYTALQRRETEVYACIENEDGSSPTAKQSPLSQERPHRFEDDGELNLVYENL

SEQ ID NO: 170 - protein, 774 aa, synthetic construct
  REGION 1..774: note = CER97
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGAYFLGRLVPRGRGAAEAATRKQRITETESPYQELQGQRSDVYSDLNTQRPYYK

SEQ ID NO: 171 - protein, 767 aa, synthetic construct
  REGION 1..767: note = CER98
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGADSKAGMEEDHTYEGLDIDQTATYEDIVTLRTGEVKWSVGEHPGQE

SEQ ID NO: 172 - protein, 808 aa, synthetic construct
  REGION 1..808: note = CER99
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGALWNKKRMRGPGKDPTRKCPDPRSASSPKQHPSESVYTALQRRETEVYACIENEDGSSPTAKQSPLSQERPHRFEDDGELNLVYENL

SEQ ID NO: 173 - protein, 579 aa, synthetic construct
  REGION 1..579: note = CER102
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYLWNKKRMRGPGKDPTRKCPDPRSASSPKQHPSESVYTALQRRETEVYACIENEDGSSPTAKQSPLSQERPHRFEDDGELNLVYENL

SEQ ID NO: 174 - protein, 538 aa, synthetic construct
  REGION 1..538: note = CER103A
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYDSKAGMEEDHTYEGLDIDQTATYEDIVTLRTGEVKWSVGEHPGQE

SEQ ID NO: 175 - protein, 522 aa, synthetic construct
  REGION 1..522: note = CER103B
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYDSKAGMEEDHTYEGLDIDQTATYEDIVTL

SEQ ID NO: 176 - protein, 545 aa, synthetic construct
  REGION 1..545: note = CER104
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYYFLGRLVPRGRGAAEAATRKQRITETESPYQELQGQRSDVYSDLNTQRPYYK

SEQ ID NO: 177 - protein, 578 aa, synthetic construct
  REGION 1..578: note = CER105
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYSWRRRQRRLRGASSAEAPDGDKDAPEPLDKVIILSPGISDATAPAWPPPGEDPGTTPPGHSVPVPATELGSTELVTTKTAGPEQQ

SEQ ID NO: 178 - protein, 579 aa, synthetic construct
  REGION 1..579: note = CER106
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLWNKKRMRGPGKDPTRKCPDPRSASSPKQHPSESVYTALQRRETEVYACIENEDGSSPTAKQSPLSQERPHRFEDDGELNLVYENLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 179 - protein, 522 aa, synthetic construct
  REGION 1..522: note = CER107
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLDSKAGMEEDHTYEGLDIDQTATYEDIVTLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 180 - protein, 545 aa, synthetic construct
  REGION 1..545: note = CER108
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLYFLGRLVPRGRGAAEAATRKQRITETESPYQELQGQRSDVYSDLNTQRPYYKHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 181 - protein, 578 aa, synthetic construct
  REGION 1..578: note = CER109
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSWRRRQRRLRGASSAEAPDGDKDAPEPLDKVIILSPGISDATAPAWPPPGEDPGTTPPGHSVPVPATELGSTELVTTKTAGPEQQHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 182 - protein, 626 aa, synthetic construct
  REGION 1..626: note = CER110
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAYFLGRLVPRGRGAAEAATRKQRITETESPYQELQGQRSDVYSDLNTQRPYYK

SEQ ID NO: 183 - protein, 619 aa, synthetic construct
  REGION 1..619: note = CER111A
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLADSKAGMEEDHTYEGLDIDQTATYEDIVTLRTGEVKWSVGEHPGQE

SEQ ID NO: 184 - protein, 603 aa, synthetic construct
  REGION 1..603: note = CER111B
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLADSKAGMEEDHTYEGLDIDQTATYEDIVTL

SEQ ID NO: 185 - protein, 660 aa, synthetic construct
  REGION 1..660: note = CER112
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLALWNKKRMRGPGKDPTRKCPDPRSASSPKQHPSESVYTALQRRETEVYACIENEDGSSPTAKQSPLSQERPHRFEDDGELNLVYENL

SEQ ID NO: 186 - protein, 659 aa, synthetic construct
  REGION 1..659: note = CER113
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLASWRRRQRRLRGASSAEAPDGDKDAPEPLDKVIILSPGISDATAPAWPPPGEDPGTTPPGHSVPVPATELGSTELVTTKTAGPEQQ

SEQ ID NO: 187 - protein, 1050 aa, synthetic construct
  REGION 1..1050: note = CER114
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLM

SEQ ID NO: 188 - protein, 1050 aa, synthetic construct
  REGION 1..1050: note = CER115
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLALRRRVQETKFGGAFSEEDSQLVVNYRAKKSFCRRAIELTLQSLGVSEELQNKLEDVVIDRNLLVLGKVLGEGEFGSVMEGNLKQEDGTSQKVAVKTMKLDNFSQREIEEFLSEAACMKDFNHPNVIRLLGVCIELSSQGIPKPMVILPFMKYGDLHTFLLYSRLNTGPKYIHLQTLLKFMMDIAQGMEYLSNRNFLHRDLAARNCMLRDDMTVCVADFGLSKKIYSGDYYRQGRIAKMPVKWIAIESLADRVYTSKSDVWAFGVTMWEITTRGMTPYPGVQNHEMYDYLLHGHRLKQPEDCLDELYDIMYSCWSADPLDRPTFSVLRLQLEKLSESLPDAQDKESIIYINTQLLESCEGIANGPSLTGLDMNIDPDSIIASCTPGAAVSVVTAEVHENNLREERYILNGGNEEWEDVSSTPFAAVTPEKDGVLPEDRLTKNGVSWSHHSTLPLGSPSPDELLFVDDSLEDSEVLMMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 189 - protein, 767 aa, synthetic construct
  REGION 1..767: note = CER116
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 190 - protein, 767 aa, synthetic construct
  REGION 1..767: note = CER117
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 191 - protein, 571 aa, synthetic construct
  REGION 1..571: note = CER118
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKLWNKKRMRGPGKDPTRKCPDPRSASSPKQHPSESVYTALQRRETEVYACIENEDGSSPTAKQSPLSQERPHRFEDDGELNLVYENL

SEQ ID NO: 192 - protein, 530 aa, synthetic construct
  REGION 1..530: note = CER119A
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKDSKAGMEEDHTYEGLDIDQTATYEDIVTLRTGEVKWSVGEHPGQE

SEQ ID NO: 193 - protein, 514 aa, synthetic construct
  REGION 1..514: note = CER119
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKDSKAGMEEDHTYEGLDIDQTATYEDIVTL

SEQ ID NO: 194 - protein, 537 aa, synthetic construct
  REGION 1..537: note = CER120
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKYFLGRLVPRGRGAAEAATRKQRITETESPYQELQGQRSDVYSDLNTQRPYYK

SEQ ID NO: 195 - protein, 759 aa, synthetic construct
  REGION 1..759: note = CER121
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLSYLDLPWYLRMVCQWTQTRRRARNIPLEELQRNLQFHAFISYSGHDSFWVKNELLPNLEKEGMQICLHERNFVPGKSIVENIITCIEKSYKSIFVLSPNFVQSEWCHYELYFAHHNLFHEGSNSLILILLEPIPQYSIPSSYHKLKSLMARRTYLEWPKEKSKRGLFWANLRAAINIKLTEQAKKMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 196 - protein, 527 aa, synthetic construct
  REGION 1..527: note = CER122
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSYFLGRLVPRGRGAAEAATRKQRITETESPYQELQGQRSDVYSDLNTQRPYYK

SEQ ID NO: 197 - protein, 749 aa, synthetic construct
  REGION 1..749: note = CER123
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 198 - protein, 561 aa, synthetic construct
  REGION 1..561: note = CER124
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSLWNKKRMRGPGKDPTRKCPDPRSASSPKQHPSESVYTALQRRETEVYACIENEDGSSPTAKQSPLSQERPHRFEDDGELNLVYENL

SEQ ID NO: 199 - protein, 520 aa, synthetic construct
  REGION 1..520: note = CER125A
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSDSKAGMEEDHTYEGLDIDQTATYEDIVTLRTGEVKWSVGEHPGQE

SEQ ID NO: 200 - protein, 504 aa, synthetic construct
  REGION 1..504: note = CER125B
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSDSKAGMEEDHTYEGLDIDQTATYEDIVTL

SEQ ID NO: 201 - protein, 778 aa, synthetic construct
  REGION 1..778: note = CER126
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 202 - protein, 778 aa, synthetic construct
  REGION 1..778: note = CER127
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 203 - protein, 796 aa, synthetic construct
  REGION 1..796: note = CER128
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEACHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 204 - protein, 796 aa, synthetic construct
  REGION 1..796: note = CER129
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 205 - protein, 504 aa, synthetic construct
  REGION 1..504: note = hCER21
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 206 - protein, 585 aa, synthetic construct
  REGION 1..585: note = hCER29
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 207 - protein, 556 aa, synthetic construct
  REGION 1..556: note = hCER104
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYYFLGRLVPRGRGAAEAATRKQRITETESPYQELQGQRSDVYSDLNTQRPYYK

SEQ ID NO: 208 - protein, 778 aa, synthetic construct
  REGION 1..778: note = hCER116
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 209 - protein, 778 aa, synthetic construct
  REGION 1..778: note = hCER117
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 210 - protein, 538 aa, synthetic construct
  REGION 1..538: note = hCER122
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSYFLGRLVPRGRGAAEAATRKQRITETESPYQELQGQRSDVYSDLNTQRPYYK

SEQ ID NO: 211 - protein, 760 aa, synthetic construct
  REGION 1..760: note = hCER123
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 212 - protein, 789 aa, synthetic construct
  REGION 1..789: note = hCER126
ETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 213 - protein, 42 aa, Homo sapiens
ALYLLRRDQRLPPDAHKPPGGGSFRTPIQEEQADAHSTLAKI

SEQ ID NO: 214 - protein, 116 aa, Homo sapiens
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 215 - protein, 42 aa, Homo sapiens; identical to SEQ ID NO: 30

SEQ ID NO: 216 - protein, 48 aa, Homo sapiens
QRRKYRSNKGESPVEPAEPCHYSCPREEEGSTIPIQEDYRKPEPACSP

SEQ ID NO: 217 - protein, 29 aa, Homo sapiens
NRQRKIKKYRLQQAQKGTPMKPNTQATPP

SEQ ID NO: 218 - protein, 46 aa, Homo sapiens
KALIHLSDLREYRRFEKEKLKSQWNNDNPLFKSATTTVMNPKFAES

SEQ ID NO: 219 - protein, 38 aa, Homo sapiens
CWLTKKKYSSSVHDPNGEYMFMRAVNTAKKSRLTDVTL

SEQ ID NO: 220 - protein, 188 aa, Homo sapiens
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 221 - protein, 62 aa, Homo sapiens
KKVAKKPTNKAPHPKQEPQEINFPDDLPGSNTAAPVQETLHGCQPVTQEDGKESRISVQERQ

SEQ ID NO: 222 - protein, 97 aa, Homo sapiens
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 223 - protein, 39 aa, Homo sapiens
RTQIKKLCSWRDKNSAACVVYEDMSHSRCNTLSSPNQYQ

SEQ ID NO: 224 - protein, 37 aa, Homo sapiens
MEESVVRPSVFVVDGQTDIPFTRLGRSHRRQSCSVAR

SEQ ID NO: 225 - protein, 70 aa, Homo sapiens
MSKQRGTFSEVSLAQDPKRQQRKPKGNKSSISGTEQEIFQVELNLQNPSLNHQGIDKIYDCQGLLPPPEK

SEQ ID NO: 226 - protein, 47 aa, Homo sapiens
CWRKIKQSCEEENAGAEDQDGEGEGSKTALQPLKHSDSKEDDGQEIA

SEQ ID NO: 227 - protein, 45 aa, Homo sapiens
GQDGVRQSRASDKQTLLPNDQLYQPLKDREDDQYSHLQGNQLRRN

SEQ ID NO: 228 - protein, 45 aa, Homo sapiens
GHETGRLSGAADTQALLRNDQVYQPLRDRDDAQYSHLGGNWARNK

SEQ ID NO: 229 - protein, 55 aa, Homo sapiens
KNRKAKAKPVTRGAGAGGRQRGQNKERPPPVPNPDYEPIRKGQRDLYSGLNQRRI

SEQ ID NO: 230 - protein, 93 aa, Homo sapiens
KKLVKKFRQKKQRQWIGPTGMNQNMSFHRNHTATVRSHAENPTASHVDNEYSQPPRNSHLSAYPALEGALHRSSMQPDNSSDSDYDLHGAQRL

SEQ ID NO: 231 - protein, 141 aa, Homo sapiens
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 232 - protein, 61 aa, Homo sapiens
RKRWQNEKLGLDAGDEYEDENLYEGLNLDDCSMYEDISRGLQGTYQDVGSLNIGDVQLEKP

SEQ ID NO: 233 - protein, 24 aa, Homo sapiens
LCARPRRSPAQEDGKVYINMPGRG

SEQ ID NO: 234 - protein, 76 aa, Homo sapiens
AKTGRTSIQRDLKEQQPQALAPGRGPSHSSAFSMSPLSTAQAPLPNPRTAASIYEELLKHDTNIYCRMDHKAEVAS

SEQ ID NO: 235 - protein, 21 aa, Homo sapiens
LLMIIAPSLGFVLFALFVAFL

SEQ ID NO: 236 - protein, 27 aa, Homo sapiens
FWVLVVVGGVLACYSLLVTVAFIIFWV

SEQ ID NO: 237 - protein, 27 aa, Homo sapiens
IISFFLALTSTALLFLLFFLTLRFSVV

SEQ ID NO: 238 - protein, 21 aa, Homo sapiens
VAAILGLGLVLGLLGPLAILL

SEQ ID NO: 239 - protein, 21 aa, Homo sapiens
ILVIFSGMFLVFTLAGALFLH

SEQ ID NO: 240 - protein, 21 aa, Homo sapiens
FWLPIGCAAFVVVCILGCILI

SEQ ID NO: 241 - protein, 26 aa, Homo sapiens
IYLIIGICGGGSLLMVFVALLVFYIT

SEQ ID NO: 242 - protein, 23 aa, Homo sapiens
IAAIVGGTVAGIVLIGILLLVIW

SEQ ID NO: 243 - protein, 28 aa, Homo sapiens
PVLDAGPVLFWVILVLVVVVGSSAFLLC

SEQ ID NO: 244 - protein, 22 aa, Homo sapiens
ALVVIPIIFGILFAILLVLVFI

SEQ ID NO: 245 - protein, 21 aa, Homo sapiens
VGVVGGLLGSLVLLVWVLAVI

SEQ ID NO: 246 - protein, 21 aa, Homo sapiens
AALAVISFLLGLGLGVACVLA

SEQ ID NO: 247 - protein, 21 aa, Homo sapiens
VGLGLLLLLMGAGLAVQGWFL

SEQ ID NO: 248 - protein, 23 aa, Homo sapiens
LTAEVLGIICIVLMATVLKTIVL

SEQ ID NO: 249 - protein, 21 aa, Homo sapiens
LWVTVGLSVCLIALLVALAFV

SEQ ID NO: 250 - protein, 347 aa, synthetic construct
  REGION 1..347: note = Tim4 binding domain-CD28 transmembrane-CD28 costimulatory signaling domain
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTFWVLVVVGGVLACYSLLVTVAFIIFWVRSKRSRGGHSDYMNMTPRRPGPTRKHYQPYAPPRDFAAYRS

SEQ ID NO: 251 - protein, 341 aa, synthetic construct
  REGION 1..341: note = Tim4 binding domain-Tim4 transmembrane-CD28 costimulatory signaling domain
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLRSKRSRGGHSDYMNMTPRRPGPTRKHYQPYAPPRDFAAYRS

SEQ ID NO: 252 - protein, 454 aa, synthetic construct
  REGION 1..454: note = Tim4 binding domain-Tim4 transmembrane-4-1BB costimulatory signaling domain-CD3z ITAM containing activating domain
  SIGNAL 1..22
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 253 - protein, 496 aa, synthetic construct
  REGION 1..496: note = CER136 TIM4 binding domain-CD28 TM - CD28 costim- CD3z
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRSSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 254 - protein, 22 aa, Homo sapiens
MLLLVTSLLLCELPHPAFLLIP

SEQ ID NO: 255 - protein, 21 aa, Mus musculus
ILIIACCVGFVLMVLLFLAFL

SEQ ID NO: 256 - protein, 279 aa, Mus musculus; identical to SEQ ID NO: 51
  SIGNAL 1..22

SEQ ID NO: 257 - protein, 473 aa, synthetic construct
  REGION 1..473: note = Tim4 binding domain-CD28 hinge-CD28 transmembrane-CD28 costimulatory signaling domain-DAP12 signaling domain
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 258 - protein, 446 aa, synthetic construct
  REGION 1..446: note = Tim4 binding domain-modified IgG4 hinge-CD28 transmembrane-CD28 costimulatory signaling domain-DAP12 signaling domain
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQESKYGPPCPPCPFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 259 - protein, 434 aa, synthetic construct
  REGION 1..434: note = Tim4 binding domain-CD28 transmembrane-CD28 costimulatory signaling domain-DAP12 signaling domain
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 260 - protein, 108 aa, synthetic construct
  REGION 1..108: note = modified Tim1 IgV domain
SVKVGGEAGPSVTLPCHYSGAVTSMCWNRGSCSLFTCQNGIVWTNGTHVTYRKDTRYKLLGDLSRGDVSLTIENTAVSDSGVYCCRVEHRGWFNDMKITVSLEIVPPK

SEQ ID NO: 261 - protein, 48 aa, Homo sapiens
KKYFFKKEVQQLSVSFSSLQIKALQNAVEKEVQAEDNIYIENSLYATD

SEQ ID NO: 262 - protein, 43 aa, Homo sapiens
LRGKLMETYCSQKHTRLDYIGDSKNVLNDVQHGREDEDGLFTL

SEQ ID NO: 263 - protein, 303 aa, Homo sapiens; identical to SEQ ID NO: 107

SEQ ID NO: 264 - protein, 274 aa, Homo sapiens; identical to SEQ ID NO: 122

SEQ ID NO: 265 - protein, 193 aa, Homo sapiens; identical to SEQ ID NO: 119

SEQ ID NO: 266 - protein, 476 aa, synthetic construct
  REGION 1..476: note = Chimeric Tim - Tim1IgV - Tim1Mucin - Tim1TM - Tim1SD - CD3zSD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 267 - protein, 471 aa, synthetic construct
  REGION 1..471: note = Chimeric Tim - Tim1IgV - Tim1Mucin - Tim1TM - Tim4SD - CD3zSD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIALRGKLMETYCSQKHTRLDYIGDSKNVLNDVQHGREDEDGLFTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 268 - protein, 363 aa, synthetic construct
  REGION 1..363: note = Chimeric Tim - Tim1IgV - Tim1Mucin - CD28TM - CD28SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVRSKRSRLLHSDYMNMTPRRPGPTRKHYQPYAPPRDFAAYRS

SEQ ID NO: 269 - protein, 590 aa, synthetic construct
  REGION 1..590: note = Chimeric Tim - Tim1IgV - Tim1Mucin - Tim1TM - TRAF6SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 270 - protein, 596 aa, synthetic construct
  REGION 1..596: note = Chimeric Tim - Tim1IgV - Tim1Mucin - CD28TM - TRAF6SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 271 - protein, 619 aa, synthetic construct
  REGION 1..619: note = Chimeric Tim - Tim1IgV - Tim1Mucin - Tim1TM - TRAF2SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 272 - protein, 625 aa, synthetic construct
  REGION 1..625: note = Chimeric Tim - Tim1IgV - Tim1Mucin - Tim1TM - TRAF2SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 273 - protein, 621 aa, synthetic construct
  REGION 1..621: note = Chimeric Tim - Tim1IgV - Tim1Mucin - Tim1TM - TLR8SD - CD3zSD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 274 - protein, 415 aa, synthetic construct
  REGION 1..415: note = Chimeric Tim - Tim1IgV - Tim1Mucin - CD28TM - CD28SD - DAP12SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 275 - protein, 409 aa, synthetic construct
  REGION 1..409: note = Chimeric Tim - Tim1IgV - Tim1Mucin - Tim1TM - CD28SD - DAP12SD
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIARSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 276 - protein, 490 aa, synthetic construct
  REGION 1..490: note = Chimeric Tim - Tim4IgV - Tim4Mucin - Tim4TM - Tim4SD - CD3zSD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 277 - protein, 495 aa, synthetic construct
  REGION 1..495: note = Chimeric Tim - Tim4IgV - Tim4Mucin - Tim4TM - Tim1SD - CD3zSD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLKKYFFKKEVQQLSVSFSSLQIKALQNAVEKEVQAEDNIYIENSLYATDRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 278 - protein, 382 aa, synthetic construct
  REGION 1..382: note = Chimeric Tim - Tim4IgV - Tim4Mucin - CD28TM - CD28SD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRS

SEQ ID NO: 279 - protein, 609 aa, synthetic construct
  REGION 1..609: note = Chimeric Tim - Tim4IgV - Tim4Mucin - Tim4TM - TRAF6SD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 280 - protein, 615 aa, synthetic construct
  REGION 1..615: note = Chimeric Tim - Tim4IgV - Tim4Mucin - CD28TM - TRAF6SD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 281 - protein, 638 aa, synthetic construct
  REGION 1..638: note = Chimeric Tim - Tim4IgV - Tim4Mucin - Tim4TM - TRAF2SD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 282 - protein, 644 aa, synthetic construct
  REGION 1..644: note = Chimeric Tim - Tim4IgV - Tim4Mucin - CD28TM - TRAF2SD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVMAAASVTPPGSLELLQPGFSKTLLGTKLEAKYLCSACRNVLRRPFQAQCGHRYCSFCLASILSSGPQNCAACVHEGIYEEGISILESSSAFPDNAARREVESLPAVCPSDGCTWKGTLKEYESCHEGRCPLMLTECPACKGLVRLGEKERHLEHECPERSLSCRHCRAPCCGADVKAHHEVCPKFPLTCDGCGKKKIPREKFQDHVKTCGKCRVPCRFHAIGCLETVEGEKQQEHEVQWLREHLAMLLSSVLEAKPLLGDQSHAGSELLQRCESLEKKTATFENIVCVLNREVERVAMTAEAC

SEQ ID NO: 283 - protein, 640 aa, synthetic construct
  REGION 1..640: note = Chimeric Tim - Tim4IgV - Tim4Mucin - Tim1TM - TLR8SD - CD3zSD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQIYAGVCISVLVLLALLGVIIAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 284 - protein, 628 aa, synthetic construct
  REGION 1..628: note = Chimeric Tim - Tim4IgV - Tim1Mucin - Tim1TM - TLR8SD - CD3zSD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 285 - protein, 416 aa, synthetic construct
  REGION 1..416: note = Chimeric Tim - Tim4IgV - Tim1Mucin - Tim1TM - CD28SD - DAP12SD
  SIGNAL 01..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIARSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 286 - protein, 422 aa, synthetic construct
  REGION 1..422: note = Chimeric Tim - Tim4IgV - Tim1Mucin - CD28TM - CD28SD - DAP12SD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGFWVLVVVGGVLACYSLLVTVAFIIFWVRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 287 - protein, 18 aa, synthetic construct
  REGION 1..18: note = T2A self-cleaving peptide
EGRGSLLTCGDVEENPGP

SEQ ID NO: 288 - protein, 23 aa, synthetic construct
  REGION 1..23: note = T2A self-cleaving peptide variant
LEGGGEGRGSLLTCGDVEENPGP

SEQ ID NO: 289 - protein, 19 aa, synthetic construct
  REGION 1..19: note = T2A self-cleaving peptide variant
EGRGSLLTCGDVEENPGPR

SEQ ID NO: 290 - protein, 24 aa, synthetic construct
  REGION 1..24: note = T2A self-cleaving peptide variant
LEGGGEGRGSLLTCGDVEENPGPR

SEQ ID NO: 291 - protein, 19 aa, synthetic construct
  REGION 1..19: note = P2A self-cleaving peptide
ATNFSLLKQAGDVEENPGP

SEQ ID NO: 292 - protein, 27 aa, synthetic construct
  REGION 1..27: note = P2A self-cleaving peptide variant
RAKRSGSGATNFSLLKQAGDVEENPGP

SEQ ID NO: 293 - protein, 20 aa, synthetic construct
  REGION 1..20: note = E2A self-cleaving peptide
QCTNYALLKLAGDVESNPGP

SEQ ID NO: 294 - protein, 22 aa, synthetic construct
  REGION 1..22: note = F2A self-cleaving peptide
VKQTLNFDLLKLAGDVESNPGP

SEQ ID NO: 295 - protein, 285 aa, synthetic construct
  REGION 1..285: note = truncated EGFR
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCH

SEQ ID NO: 296 - protein, 192 aa, Homo sapiens
MQAIKCVVVGDGAVGKTCLLISYTTNAFPGEYIPTVFDNYSANVMVDGKPVNLGLWDTAGQEDYDRLRPLSYPQTDVFLICFSLVSPASFENVRAKWYPEVRHHCPNTPIILVGTKLDLRDDKDTIEKLKEKKLTPITYPQGLAMAKEIGAVKYLECSALTQRGLKTVFDEAIRAVLCPPPVKKRKRKCLLL

SEQ ID NO: 297 - protein, 215 aa, Homo sapiens
MASRGATRPNGPNTGNKICQFKLVLLGESAVGKSSLVLRFVKGQFHEFQESTIGAAFLTQTVCLDDTTVKFEIWDTAGQERYHSLAPMYYRGAQAAIVVYDITNEESFARAKNWVKELQRQASPNIVIALSGNKADLANKRAVDFQEAQSYADDNSLLFMETSAKTSMNVNEIFMAIAKKLPKNEPQNPGANSARGRGVDLTEPTQPTRNQCCSN

SEQ ID NO: 298 - protein, 207 aa, Homo sapiens
MTSRKKVLLKVIILGDSGVGKTSLMNQYVNKKFSNQYKATIGADFLTKEVMVDDRLVTMQIWDTAGQERFQSLGVAFYRGADCCVLVFDVTAPNTFKTLDSWRDEFLIQASPRDPENFPFVVLGNKIDLENRQVATKRAQAWCYSKNNIPYFETSAKEAINVEQAFQTIARNALKQETEVELYNEFPEPIKLDKNDRAKASAESCSC

SEQ ID NO: 299 - protein, 184 aa, Homo sapiens
MREYKLVVLGSGGVGKSALTVQFVQGIFVEKYDPTIEDSYRKQVEVDCQQCMLEILDTAGTEQFTAMRDLYMKNGQGFALVYSITAQSTFNDLQDLREQILRVKDTEDVPMILVGNKCDLEDERVVGKEQGQNLARQWCNCAFLESSAKSKINVNEIFYDLVRQINRKTPVEKKKPKKKSCLLL

SEQ ID NO: 300 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 301 - protein, 191 aa, Homo sapiens
MQTIKCVVVGDGAVGKTCLLISYTTNKFPSEYVPTVFDNYAVTVMIGGEPYTLGLFDTAGQEDYDRLRPLSYPQTDVFLVCFSVVSPSSFENVKEKWVPEITHHCPKTPFLLVGTQIDLRDDPSTIEKLAKNKQKPITPETAEKLARDLKAVKYVECSALTQKGLKNVFDEAILAALEPPEPKKSRRCVLL

SEQ ID NO: 302 - protein, 6 aa, synthetic construct
  REGION 1..6: note = Sc02-002 HCDR1
GFTFSD

SEQ ID NO: 303 - protein, 8 aa, synthetic construct
  REGION 1..8: note = Sc02-002 HCDR2
ISYDGSNK

SEQ ID NO: 304 - protein, 14 aa, synthetic construct
  REGION 1..14: note = Sc02-002 HCDR3
AKDRGSAQGYPLDY

SEQ ID NO: 305 - protein, 7 aa, synthetic construct
  REGION 1..7: note = Sc02-002 LCDR1
QSVSSTY

SEQ ID NO: 306 - skipped

SEQ ID NO: 307 - protein, 7 aa, synthetic construct
  REGION 1..7: note = Sc02-002 LCDR3
QQGSAFP

SEQ ID NO: 308 - protein, 8 aa, synthetic construct
  REGION 1..8: note = Sc02-003 HCDR1
GFTFSNYV

SEQ ID NO: 309 - protein, 8 aa, synthetic construct
  REGION 1..8: note = Sc02-003 HCDR2
ISYDGSNK

SEQ ID NO: 310 - protein, 11 aa, synthetic construct
  REGION 1..11: note = Sc02-003 HCDR3
AKSHSNMSFDY

SEQ ID NO: 311 - protein, 6 aa, synthetic construct
  REGION 1..6: note = Sc02-003 LCDR1
FTEHFQ

SEQ ID NO: 312 - skipped

SEQ ID NO: 313 - protein, 7 aa, synthetic construct
  REGION 1..7: note = Sc02-003 LCDR3
QQSYSTP

SEQ ID NO: 314 - protein, 8 aa, synthetic construct
  REGION 1..8: note = Sc02-004 HCDR1
GFTFSGYL

SEQ ID NO: 315 - protein, 8 aa, synthetic construct
  REGION 1..8: note = Sc02-004 HCDR2
ISYDGSNK

SEQ ID NO: 316 - protein, 12 aa, synthetic construct
  REGION 1..12: note = Sc02-004 HCDR3
ARARRDTNLFDY

SEQ ID NO: 317 - protein, 6 aa, synthetic construct
  REGION 1..6: note = Sc02-004 LCDR1
QSISSY

SEQ ID NO: 318 - skipped

SEQ ID NO: 319 - protein, 9 aa, synthetic construct
  REGION 1..9: note = Sc02-004 LCDR3
QQSYSTPPT

SEQ ID NO: 320 - protein, 8 aa, synthetic construct
  REGION 1..8: note = Sc02-024 HCDR1
GYTFTSYY

SEQ ID NO: 321 - protein, 8 aa, synthetic construct
  REGION 1..8: note = Sc02-024 HCDR2
INPSGGST

SEQ ID NO: 322 - protein, 13 aa, synthetic construct
  REGION 1..13: note = Sc02-024 HCDR3
ARDYVRTSHSGFD

SEQ ID NO: 323 - protein, 6 aa, synthetic construct
  REGION 1..6: note = Sc02-024 LCDR1
QSISSY

SEQ ID NO: 324 - skipped

SEQ ID NO: 325 - protein, 9 aa, synthetic construct
  REGION 1..9: note = Sc02-024 LCDR3
QQSYSTPPT

SEQ ID NO: 326 - protein, 8 aa, synthetic construct
  REGION 1..8: note = Sc02-025 HCDR1
GYTFTSYY

SEQ ID NO: 327 - protein, 8 aa, synthetic construct
  REGION 1..8: note = Sc02-025 HCDR2
INPSGGGT

SEQ ID NO: 328 - protein, 14 aa, synthetic construct
  REGION 1..14: note = Sc02-025 HCDR3
ARDYYVTYDSWFDS

SEQ ID NO: 329 - protein, 6 aa, synthetic construct
  REGION 1..6: note = Sc02-025 LCDR1
SLRSYY

SEQ ID NO: 330 - skipped

SEQ ID NO: 331 - protein, 11 aa, synthetic construct
  REGION 1..11: note = Sc02-025 LCDR3
NSRDSSGNHVV

SEQ ID NO: 332 - protein, 8 aa, synthetic construct
  REGION 1..8: note = Sc02-041 HCDR1
GFTFSNYV

SEQ ID NO: 333 - protein, 8 aa, synthetic construct
  REGION 1..8: note = Sc02-041 HCDR2
ISYDGSNK

SEQ ID NO: 334 - protein, 11 aa, synthetic construct
  REGION 1..11: note = Sc02-041 HCDR3
AKSHSNMAFDY

SEQ ID NO: 335 - protein, 6 aa, synthetic construct
  REGION 1..6: note = Sc02-041 LCDR1
QSISSY

SEQ ID NO: 336 - skipped

SEQ ID NO: 337 - protein, 9 aa, synthetic construct
  REGION 1..9: note = Sc02-041 LCDR3
QQSYSTPPT

SEQ ID NO: 338 - protein, 8 aa, synthetic construct
  REGION 1..8: note = Sc02-057 HCDR1
GYTFTSYY

SEQ ID NO: 339 - protein, 8 aa, synthetic construct
  REGION 1..8: note = Sc02-057 HCDR2
INPSGGST

SEQ ID NO: 340 - protein, 14 aa, synthetic construct
  REGION 1..14: note = Sc02-057 HCDR3
ARDYQLSMTSGFDY

SEQ ID NO: 341 - protein, 6 aa, synthetic construct
  REGION 1..6: note = Sc02-057 LCDR1
QSISSY

SEQ ID NO: 342 - skipped

SEQ ID NO: 343 - protein, 9 aa, synthetic construct
  REGION 1..9: note = Sc02-057 LCDR3
QQSYSTPPT

SEQ ID NO: 344 - protein, 8 aa, synthetic construct
  REGION 1..8: note = Sc02-132 HCDR1
GFTFSNYV

SEQ ID NO: 345 - protein, 8 aa, synthetic construct
  REGION 1..8: note = Sc02-132 HCDR2
ISYDGSNK

SEQ ID NO: 346 - protein, 11 aa, synthetic construct
  REGION 1..11: note = Sc02-132 HCDR3
VKSHSNMAFDY

SEQ ID NO: 347 - protein, 6 aa, synthetic construct
  REGION 1..6: note = Sc02-132 LCDR1
QSISSY

SEQ ID NO: 348 - skipped

SEQ ID NO: 349 - protein, 9 aa, synthetic construct
  REGION 1..9: note = Sc02-132 LCDR3
QQSYSTPPT

SEQ ID NO: 350 - protein, 120 aa, synthetic construct
  REGION 1..120: note = Sc02-002 VH
AEVQLVESGGGVVQPGRSLRLSCAASGFTFSDYTMSWVRQAPGKGLEWVAVISYDGSNKYYADSVKGRFTISRDNSKNTLYLQMNSLRAEDTAVYYCAKDRGSAQGYPLDYWGQGTLVTV

SEQ ID NO: 351 - protein, 115 aa, synthetic construct
  REGION 1..115: note = Sc02-002 VL
SELDIQMTQSPPTLSLSPGERATLSCRASQSVSSTYLTWYQQRPGQAPRLLIYGASSRATGIPDRFSGSGSGTDFTLTISRLEPEDVAVYYCQQGSAFPPTFGQGTKVEIKRAAA

SEQ ID NO: 352 - protein, 117 aa, synthetic construct
  REGION 1..117: note = Sc02-003 VH
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMSFDYWGQGTLVTV

SEQ ID NO: 353 - protein, 110 aa, synthetic construct
  REGION 1..110: note = Sc02-003 VL
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 354 - protein, 120 aa, synthetic construct
  REGION 1..120: note = Sc02-024 VH
AQVQLVQSGPEVKKPGASVKVSCKASGYTFTSYYMHWVRQAPGQGLEWMGIINPSGGSTSYAQKFQGRVTMTRDTSTSTVYMELSSLRSEDAAVYYCARDYVRTSHSGFDYWGQGTLVTV

SEQ ID NO: 355 - protein, 112 aa, synthetic construct
  REGION 1..112: note = Sc02-024 VL
SEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 356 - protein, 121 aa, synthetic construct
  REGION 1..121: note = Sc02-025 VH
AQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVS

SEQ ID NO: 357 - protein, 111 aa, synthetic construct
  REGION 1..111: note = Sc02-025 VL
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAA

SEQ ID NO: 358 - protein, 117 aa, synthetic construct
  REGION 1..117: note = Sc02-041 VH
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMAFDYWGQGTLVTV

SEQ ID NO: 359 - protein, 110 aa, synthetic construct; identical to SEQ ID NO: 353
  REGION 1..110: note = Sc02-041 VL

SEQ ID NO: 360 - protein, 120 aa, synthetic construct
  REGION 1..120: note = Sc02-057 VH
AQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYQLSMTSGFDYWGQGTLVTV

SEQ ID NO: 361 - protein, 112 aa, synthetic construct; identical to SEQ ID NO: 355
  REGION 1..112: note = Sc02-057 VL

SEQ ID NO: 362 - protein, 117 aa, synthetic construct
  REGION 1..117: note = Sc02-132 VH
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCVKSHSNMAFDYWGQGTLVTV

SEQ ID NO: 363 - protein, 110 aa, synthetic construct; identical to SEQ ID NO: 353
  REGION 1..110: note = Sc02-132 VL

SEQ ID NO: 364 - protein, 253 aa, synthetic construct
  REGION 1..253: note = sc02-002 VL-VH W linker scFv
SELDIQMTQSPPTLSLSPGERATLSCRASQSVSSTYLTWYQQRPGQAPRLLIYGASSRATGIPDRFSGSGSGTDFTLTISRLEPEDVAVYYCQQGSAFPPTFGQGTKVEIKRAAAGSTSGSGKPGSGEGSTKGAEVQLVESGGGVVQPGRSLRLSCAASGFTFSDYTMSWVRQAPGKGLEWVAVISYDGSNKYYADSVKGRFTISRDNSKNTLYLQMNSLRAEDTAVYYCAKDRGSAQGYPLDYWGQGTLVTV

SEQ ID NO: 365 - protein, 253 aa, synthetic construct
  REGION 1..253: note = sc02-002 VH-VL W linker scFv
AEVQLVESGGGVVQPGRSLRLSCAASGFTFSDYTMSWVRQAPGKGLEWVAVISYDGSNKYYADSVKGRFTISRDNSKNTLYLQMNSLRAEDTAVYYCAKDRGSAQGYPLDYWGQGTLVTVGSTSGSGKPGSGEGSTKGSELDIQMTQSPPTLSLSPGERATLSCRASQSVSSTYLTWYQQRPGQAPRLLIYGASSRATGIPDRFSGSGSGTDFTLTISRLEPEDVAVYYCQQGSAFPPTFGQGTKVEIKRAAA

SEQ ID NO: 366 - protein, 245 aa, synthetic construct
  REGION 1..245: note = sc02-003 VL-VH W linker scFv
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMSFDYWGQGTLVTV

SEQ ID NO: 367 - protein, 245 aa, synthetic construct
  REGION 1..245: note = sc02-003 VH-VL W linker scFv
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMSFDYWGQGTLVTVGSTSGSGKPGSGEGSTKGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 368 - protein, 246 aa, synthetic construct
  REGION 1..246: note = sc02-004 VL-VH W linker scFv
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTV

SEQ ID NO: 369 - protein, 246 aa, synthetic construct
  REGION 1..246: note = sc02-004 VH-VL W linker scFv
AEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVGSTSGSGKPGSGEGSTKGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 370 - protein, 250 aa, synthetic construct
  REGION 1..250: note = sc02-024 VL-VH W linker scFv
SEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAQVQLVQSGPEVKKPGASVKVSCKASGYTFTSYYMHWVRQAPGQGLEWMGIINPSGGSTSYAQKFQGRVTMTRDTSTSTVYMELSSLRSEDAAVYYCARDYVRTSHSGFDYWGQGTLVTV

SEQ ID NO: 371 - protein, 250 aa, synthetic construct
  REGION 1..250: note = sc02-024 VH-VL W linker scFv
AQVQLVQSGPEVKKPGASVKVSCKASGYTFTSYYMHWVRQAPGQGLEWMGIINPSGGSTSYAQKFQGRVTMTRDTSTSTVYMELSSLRSEDAAVYYCARDYVRTSHSGFDYWGQGTLVTVGSTSGSGKPGSGEGSTKGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 372 - protein, 250 aa, synthetic construct
  REGION 1..250: note = sc02-025 VL-VH W linker scFv
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAGSTSGSGKPGSGEGSTKGAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVS

SEQ ID NO: 373 - protein, 250 aa, synthetic construct
  REGION 1..250: note = sc02-025 VH-VL W linker scFv
AQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSGSTSGSGKPGSGEGSTKGSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAA

SEQ ID NO: 374 - protein, 245 aa, synthetic construct
  REGION 1..245: note = sc02-041 VL-VH W linker scFv
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMAFDYWGQGTLVTV

SEQ ID NO: 375 - protein, 245 aa, synthetic construct
  REGION 1..245: note = sc02-041 VH-VL W linker scFv
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMAFDYWGQGTLVTVGSTSGSGKPGSGEGSTKGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 376 - protein, 250 aa, synthetic construct
  REGION 1..250: note = sc02-057 VL-VH W linker scFv
SEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYQLSMTSGFDYWGQGTLVTV

SEQ ID NO: 377 - protein, 250 aa, synthetic construct
  REGION 1..250: note = sc02-057 VH-VL W linker scFv
AQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYQLSMTSGFDYWGQGTLVTVGSTSGSGKPGSGEGSTKGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 378 - protein, 245 aa, synthetic construct
  REGION 1..245: note = sc02-132 VL-VH W linker scFv
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCVKSHSNMAFDYWGQGTLVTV

SEQ ID NO: 379 - protein, 245 aa, synthetic construct
  REGION 1..245: note = sc02-132 VH-VL W linker scFv
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCVKSHSNMAFDYWGQGTLVTVGSTSGSGKPGSGEGSTKGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 380 - protein, 251 aa, synthetic construct
  REGION 1..251: note = sc02-002 VL-VH P 002 linker scFv
SELDIQMTQSPPTLSLSPGERATLSCRASQSVSSTYLTWYQQRPGQAPRLLIYGASSRATGIPDRFSGSGSGTDFTLTISRLEPEDVAVYYCQQGSAFPPTFGQGTKVEIKRAAALEGTGGSGGTGSGTGTAEVQLVESGGGVVQPGRSLRLSCAASGFTFSDYTMSWVRQAPGKGLEWVAVISYDGSNKYYADSVKGRFTISRDNSKNTLYLQMNSLRAEDTAVYYCAKDRGSAQGYPLDYWGQGTLVTV

SEQ ID NO: 381 - protein, 251 aa, synthetic construct
  REGION 1..251: note = sc02-002 VH-VL P 002 linker scFv
AEVQLVESGGGVVQPGRSLRLSCAASGFTFSDYTMSWVRQAPGKGLEWVAVISYDGSNKYYADSVKGRFTISRDNSKNTLYLQMNSLRAEDTAVYYCAKDRGSAQGYPLDYWGQGTLVTVLEGTGGSGGTGSGTGTSELDIQMTQSPPTLSLSPGERATLSCRASQSVSSTYLTWYQQRPGQAPRLLIYGASSRATGIPDRFSGSGSGTDFTLTISRLEPEDVAVYYCQQGSAFPPTFGQGTKVEIKRAAA

SEQ ID NO: 382 - protein, 243 aa, synthetic construct
  REGION 1..243: note = sc02-003 VL-VH P 002 linker scFv
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAALEGTGGSGGTGSGTGTAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMSFDYWGQGTLVTV

SEQ ID NO: 383 - protein, 243 aa, synthetic construct
  REGION 1..243: note = sc02-003 VH-VL P 002 linker scFv
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMSFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 384 - protein, 244 aa, synthetic construct
  REGION 1..244: note = sc02-004 VL-VH P 002 linker scFv
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAALEGTGGSGGTGSGTGTAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTV

SEQ ID NO: 385 - protein, 244 aa, synthetic construct; identical to SEQ ID NO: 17
  REGION 1..244: note = sc02-004 VH-VL P 002 linker scFv

SEQ ID NO: 386 - protein, 248 aa, synthetic construct
  REGION 1..248: note = sc02-024 VL-VH P 024 linker scFv
SEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAASSGGGGSGGGGSGGGGAQVQLVQSGPEVKKPGASVKVSCKASGYTFTSYYMHWVRQAPGQGLEWMGIINPSGGSTSYAQKFQGRVTMTRDTSTSTVYMELSSLRSEDAAVYYCARDYVRTSHSGFDYWGQGTLVTV

SEQ ID NO: 387 - protein, 248 aa, synthetic construct
  REGION 1..248: note = sc02-024 VH-VL P 024 linker scFv
AQVQLVQSGPEVKKPGASVKVSCKASGYTFTSYYMHWVRQAPGQGLEWMGIINPSGGSTSYAQKFQGRVTMTRDTSTSTVYMELSSLRSEDAAVYYCARDYVRTSHSGFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 388 - protein, 248 aa, synthetic construct
  REGION 1..248: note = sc02-025 VL-VH P 025 linker scFv
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAARGGGGSGGGGSGGGGSAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVS

SEQ ID NO: 389 - protein, 248 aa, synthetic construct; identical to SEQ ID NO: 18
  REGION 1..248: note = sc02-025 VH-VL P 025 linker scFv

SEQ ID NO: 390 - protein, 243 aa, synthetic construct
  REGION 1..243: note = sc02-041 VL-VH P 002 linker scFv
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAALEGTGGSGGTGSGTGTAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMAFDYWGQGTLVTV

SEQ ID NO: 391 - protein, 243 aa, synthetic construct
  REGION 1..243: note = sc02-041 VH-VL P 002 linker scFv
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMAFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 392 - protein, 248 aa, synthetic construct
  REGION 1..248: note = sc02-057 VL-VH P 024 linker scFv
SEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAASSGGGGSGGGGSGGGGAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYQLSMTSGFDYWGQGTLVTV

SEQ ID NO: 393 - protein, 248 aa, synthetic construct
  REGION 1..248: note = sc02-057 VH-VL P 024 linker scFv
AQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYQLSMTSGFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 394 - protein, 243 aa, synthetic construct
  REGION 1..243: note = sc02-132 VL-VH P 002 linker scFv
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAALEGTGGSGGTGSGTGTAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCVKSHSNMAFDYWGQGTLVTV

SEQ ID NO: 395 - protein, 243 aa, synthetic construct
  REGION 1..243: note = sc02-132 VH-VL P 002 linker scFv
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCVKSHSNMAFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAA

SEQ ID NO: 396 - protein, 18 aa, synthetic construct
  REGION 1..18: note = W scFv linker
GSTSGSGKPGSGEGSTKG

SEQ ID NO: 397 - protein, 16 aa, synthetic construct
  REGION 1..16: note = P 002 linker
LEGTGGSGGTGSGTGT

SEQ ID NO: 398 - protein, 16 aa, synthetic construct
  REGION 1..16: note = P 024 linker
SSGGGGSGGGGSGGGG

SEQ ID NO: 399 - protein, 16 aa, synthetic construct
  REGION 1..16: note = P 025 linker
RGGGGSGGGGSGGGGS

SEQ ID NO: 400 - protein, 474 aa, synthetic construct
  REGION 1..474: note = CAR: GMCSFR signal peptide (amino acids 1-21) - Myc tag (amino acids 22-32) - Sc02-025 scFv - IgG4 hinge - CD28 TM - 41BB - CD3z
  SIGNAL 1..21
MLLLVTSLLLCELPHPAFLLIPEQKLISEEDLAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 401 - protein, 472 aa, synthetic construct
  REGION 1..472: note = CAR: IgK signal peptide (amino acids 1-20) - Myc tag (amino acids 21-30) - Sc02-025 scFv - IgG4 hinge - CD28 TM - 41BB - CD3z
  SIGNAL 1..20
MVLQTQVFISLLLWISGAYGEQKLISEEDLAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 402 - protein, 470 aa, synthetic construct
  REGION 1..470: note = CAR: CD8a signal peptide (amino acids 1-18) - Myc tag (amino acids 19-28) - Sc02-025 scFv - IgG4 hinge - CD28 TM - 41BB - CD3z
  SIGNAL 1..18
MALPVTALLLPLALLLHAEQKLISEEDLAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 403 - protein, 472 aa, synthetic construct
  REGION 1..472: note = CAR: IL2 signal peptide (amino acids 1-20) - Myc tag (amino acids 21-30) - Sc02-025 scFv - IgG4 hinge - CD28 TM - 41BB - CD3z
  SIGNAL 1..20
MYRMQLLSCIALSLALVTNSEQKLISEEDLAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 404 - protein, 474 aa, synthetic construct; identical to SEQ ID NO: 400
  REGION 1..474: note = CAR: GMSCFR Signal peptide (amino acids 1-22) - Myc tag - Sc025 scFv - IgG4 hinge - CD28 TM - 41BB - CD3z
  SIGNAL 1..22

SEQ ID NO: 405 - protein, 447 aa, synthetic construct
  REGION 1..447: note = pCTX 831 CAR
SELDIQMTQSPPTLSLSPGERATLSCRASQSVSSTYLTWYQQRPGQAPRLLIYGASSRATGIPDRFSGSGSGTDFTLTISRLEPEDVAVYYCQQGSAFPPTFGQGTKVEIKRAAAGSTSGSGKPGSGEGSTKGAEVQLVESGGGVVQPGRSLRLSCAASGFTFSDYTMSWVRQAPGKGLEWVAVISYDGSNKYYADSVKGRFTISRDNSKNTLYLQMNSLRAEDTAVYYCAKDRGSAQGYPLDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 406 - protein, 447 aa, synthetic construct
  REGION 1..447: note = pCTX 832 CAR
AEVQLVESGGGVVQPGRSLRLSCAASGFTFSDYTMSWVRQAPGKGLEWVAVISYDGSNKYYADSVKGRFTISRDNSKNTLYLQMNSLRAEDTAVYYCAKDRGSAQGYPLDYWGQGTLVTVGSTSGSGKPGSGEGSTKGSELDIQMTQSPPTLSLSPGERATLSCRASQSVSSTYLTWYQQRPGQAPRLLIYGASSRATGIPDRFSGSGSGTDFTLTISRLEPEDVAVYYCQQGSAFPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 407 - protein, 439 aa, synthetic construct
  REGION 1..439: note = pCTX 833 CAR
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMSFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 408 - protein, 439 aa, synthetic construct
  REGION 1..439: note = pCTX 834 CAR
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMSFDYWGQGTLVTVGSTSGSGKPGSGEGSTKGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 409 - protein, 440 aa, synthetic construct
  REGION 1..440: note = pCTX 835 CAR
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 410 - protein, 440 aa, synthetic construct
  REGION 1..440: note = pCTX 836 CAR
AEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVGSTSGSGKPGSGEGSTKGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 411 - protein, 444 aa, synthetic construct
  REGION 1..444: note = pCTX 837 CAR
SEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAQVQLVQSGPEVKKPGASVKVSCKASGYTFTSYYMHWVRQAPGQGLEWMGIINPSGGSTSYAQKFQGRVTMTRDTSTSTVYMELSSLRSEDAAVYYCARDYVRTSHSGFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 412 - protein, 444 aa, synthetic construct
  REGION 1..444: note = pCTX 838 CAR
AQVQLVQSGPEVKKPGASVKVSCKASGYTFTSYYMHWVRQAPGQGLEWMGIINPSGGSTSYAQKFQGRVTMTRDTSTSTVYMELSSLRSEDAAVYYCARDYVRTSHSGFDYWGQGTLVTVGSTSGSGKPGSGEGSTKGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 413 - protein, 444 aa, synthetic construct
  REGION 1..444: note = pCTX 839 CAR
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAGSTSGSGKPGSGEGSTKGAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 414 - protein, 444 aa, synthetic construct
  REGION 1..444: note = pCTX 840 CAR
AQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSGSTSGSGKPGSGEGSTKGSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 415 - protein, 439 aa, synthetic construct
  REGION 1..439: note = pCTX 841 CAR
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMAFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 416 - protein, 439 aa, synthetic construct
  REGION 1..439: note = pCTX 842 CAR
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMAFDYWGQGTLVTVGSTSGSGKPGSGEGSTKGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 417 - protein, 444 aa, synthetic construct
  REGION 1..444: note = pCTX 843 CAR
SEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYQLSMTSGFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 418 - protein, 444 aa, synthetic construct
  REGION 1..444: note = pCTX 844 CAR
AQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYQLSMTSGFDYWGQGTLVTVGSTSGSGKPGSGEGSTKGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 419 - protein, 439 aa, synthetic construct
  REGION 1..439: note = pCTX 845 CAR
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAGSTSGSGKPGSGEGSTKGAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCVKSHSNMAFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 420 - protein, 439 aa, synthetic construct
  REGION 1..439: note = pCTX 846 CAR
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCVKSHSNMAFDYWGQGTLVTVGSTSGSGKPGSGEGSTKGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 421 - protein, 445 aa, synthetic construct
  REGION 1..445: note = pCTX 849 CAR
SELDIQMTQSPPTLSLSPGERATLSCRASQSVSSTYLTWYQQRPGQAPRLLIYGASSRATGIPDRFSGSGSGTDFTLTISRLEPEDVAVYYCQQGSAFPPTFGQGTKVEIKRAAALEGTGGSGGTGSGTGTAEVQLVESGGGVVQPGRSLRLSCAASGFTFSDYTMSWVRQAPGKGLEWVAVISYDGSNKYYADSVKGRFTISRDNSKNTLYLQMNSLRAEDTAVYYCAKDRGSAQGYPLDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 422 - protein, 445 aa, synthetic construct
  REGION 1..445: note = pCTX 850 CAR
AEVQLVESGGGVVQPGRSLRLSCAASGFTFSDYTMSWVRQAPGKGLEWVAVISYDGSNKYYADSVKGRFTISRDNSKNTLYLQMNSLRAEDTAVYYCAKDRGSAQGYPLDYWGQGTLVTVLEGTGGSGGTGSGTGTSELDIQMTQSPPTLSLSPGERATLSCRASQSVSSTYLTWYQQRPGQAPRLLIYGASSRATGIPDRFSGSGSGTDFTLTISRLEPEDVAVYYCQQGSAFPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 423 - protein, 437 aa, synthetic construct
  REGION 1..437: note = pCTX 851 CAR
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAALEGTGGSGGTGSGTGTAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMSFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 424 - protein, 437 aa, synthetic construct
  REGION 1..437: note = pCTX 852 CAR
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMSFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 425 - protein, 438 aa, synthetic construct
  REGION 1..438: note = pCTX 853 CAR
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAALEGTGGSGGTGSGTGTAEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 426 - protein, 438 aa, synthetic construct
  REGION 1..438: note = pCTX 854 CAR
AEVQLVESGGGLVQPGGSLRLSCAASGFTFSGYLMSWVRQAPGKGLEWVAVISYDGSNKYYADSVKGRFTISRDNSKNTLYLQMDSLRAEDTAVYYCARARRDTNLFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 427 - protein, 442 aa, synthetic construct
  REGION 1..442: note = pCTX 855 CAR
SEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAASSGGGGSGGGGSGGGGAQVQLVQSGPEVKKPGASVKVSCKASGYTFTSYYMHWVRQAPGQGLEWMGIINPSGGSTSYAQKFQGRVTMTRDTSTSTVYMELSSLRSEDAAVYYCARDYVRTSHSGFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 428 - protein, 442 aa, synthetic construct
  REGION 1..442: note = pCTX 856 CAR
AQVQLVQSGPEVKKPGASVKVSCKASGYTFTSYYMHWVRQAPGQGLEWMGIINPSGGSTSYAQKFQGRVTMTRDTSTSTVYMELSSLRSEDAAVYYCARDYVRTSHSGFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 429 - protein, 442 aa, synthetic construct
  REGION 1..442: note = pCTX 857 CAR
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAARGGGGSGGGGSGGGGSAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 430 - protein, 442 aa, synthetic construct
  REGION 1..442: note = pCTX 858 CAR
AQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 431 - protein, 437 aa, synthetic construct
  REGION 1..437: note = pCTX 859 CAR
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAALEGTGGSGGTGSGTGTAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMAFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 432 - protein, 437 aa, synthetic construct
  REGION 1..437: note = pCTX 860 CAR
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCAKSHSNMAFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 433 - protein, 442 aa, synthetic construct
  REGION 1..442: note = pCTX 861 CAR
SEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAASSGGGGSGGGGSGGGGAQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYQLSMTSGFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 434 - protein, 442 aa, synthetic construct
  REGION 1..442: note = pCTX 862 CAR
AQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDYQLSMTSGFDYWGQGTLVTVSSGGGGSGGGGSGGGGSEIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 435 - protein, 437 aa, synthetic construct
  REGION 1..437: note = pCTX 863 CAR
SELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAALEGTGGSGGTGSGTGTAEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCVKSHSNMAFDYWGQGTLVTVESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 436 - protein, 437 aa, synthetic construct
  REGION 1..437: note = pCTX 864 CAR
AEVQLVESGGGLVQPGGSLRLPCAASGFTFSNYVMHWVRQAPGKGLEWVAVISYDGSNKYYADSVKGRFTISRDNSKNTLYLQMNSLRAEDTAVYYCVKSHSNMAFDYWGQGTLVTVLEGTGGSGGTGSGTGTSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 437 - protein, 28 aa, synthetic construct
  REGION 1..28: note = CD28 transmembrane domain
MFWVLVVVGGVLACYSLLVTVAFIIFWV

SEQ ID NO: 438 - protein, 20 aa, synthetic construct
  REGION 1..20: note = IgK signal peptide
MVLQTQVFISLLLWISGAYG

SEQ ID NO: 439 - protein, 18 aa, synthetic construct
  REGION 1..18: note = CD8a signal peptide
MALPVTALLLPLALLLHA

SEQ ID NO: 440 - protein, 20 aa, synthetic construct
  REGION 1..20: note = IL-2 signal peptide
MYRMQLLSCIALSLALVTNS

SEQ ID NO: 441 - protein, 10 aa, synthetic construct
  REGION 1..10: note = Myc tag
EQKLISEEDL

SEQ ID NO: 442 - protein, 22 aa, synthetic construct
  REGION 1..22: note = GSG-p2A Peptide
GSGATNFSLLKQAGDVEENPGP

SEQ ID NO: 443 - protein, 357 aa, synthetic construct
  REGION 1..357: note = EGFRt Tag
MLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 444 - protein, 156 aa, synthetic construct
  REGION 1..156: note = RQR8 Tag
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPW

SEQ ID NO: 445 - protein, 1258 aa, synthetic construct
  REGION 1..1258: note = pCTX766: Sc025-BBz - p2A - TIM4-CD28 - p2A - EGFRt
MLLLVTSLLLCELPHPAFLLIPEQKLISEEDLAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQMFWVLVVVGGVLACYSLLVTVAFIIFWVRSKRSRLLHSDYMNMTPRRPGPTRKHYQPYAPPRDFAAYRSGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 446 - protein, 1258 aa, synthetic construct
  REGION 1..1258: note = pCTX768: TIM4-CD28 - p2A - Sc025-BBz - p2A - EGFRt
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQMFWVLVVVGGVLACYSLLVTVAFIIFWVRSKRSRLLHSDYMNMTPRRPGPTRKHYQPYAPPRDFAAYRSGSGATNFSLLKQAGDVEENPGPMLLLVTSLLLCELPHPAFLLIPEQKLISEEDLAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 447 - protein, 1031 aa, synthetic construct
  REGION 1..1031: note = pCTX770: Sc025-BBz - p2A - RQR8 - p2A - EGFRt
MLLLVTSLLLCELPHPAFLLIPEQKLISEEDLAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPWGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 448 - protein, 1031 aa, synthetic construct
  REGION 1..1031: note = pCTX771: RQR8 - p2A - Sc025-BBz - p2A - EGFRt
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPWGSGATNFSLLKQAGDVEENPGPMLLLVTSLLLCELPHPAFLLIPEQKLISEEDLAQVQLVQSGAEVKKPGASVKVSCKASGYTFTSYYMHWVRQAPGQGLEWMGIINPSGGGTSYAQKFQGRVTMTRDTSTSTVYMELSSLRSEDTAVYYCARDYYVTYDSWFDSWGQGTLVTVSRGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGAAAESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 449 - protein, 428 aa, synthetic construct
  REGION 1..428: note = Tim4 binding domain-Tim4 transmembrane domain-CD28 costimulatory signaling domain- DAP12 signaling domain (CER 256)
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRSKRSRLLHSDYMNMTPRRPGPTRKHYQPYAPPRDFAAYRSYFLGRLVPRGRGAAEAATRKQRITETESPYQELQGQRSDVYSDLNTQRPYYK

SEQ ID NO: 450 - protein, 510 aa, synthetic construct
  REGION 1..510: note = hTIM4-TIM4-TIM4tm-TLR2ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 451 - protein, 528 aa, synthetic construct
  REGION 1..528: note = hTIM4-TIM4-TIM4tm-TLR8ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 452-517 - skipped (66 entries)

SEQ ID NO: 518 - protein, 24 aa, synthetic construct
  REGION 1..24: note = human Tim4 signal peptide
MSKEPLILWLMIEFWWLYLTPVTS

SEQ ID NO: 519 - protein, 290 aa, synthetic construct [sequence omitted]

SEQ ID NO: 520 - protein, 27 aa, synthetic construct
  REGION 1..27: note = human CD28 transmembrane domain
FWVLVVVGGVLACYSLLVTVAFIIFWV

SEQ ID NO: 521 - protein, 21 aa, synthetic construct
  REGION 1..21: note = human Tim4 transmembrane domain
LLMIIAPSLGFVLFALFVAFL

SEQ ID NO: 522 - protein, 175 aa, synthetic construct; identical to SEQ ID NO: 113
  REGION 1..175: note = human TLR2 intracellular signaling domain

SEQ ID NO: 523 - protein, 193 aa, synthetic construct; identical to SEQ ID NO: 119
  REGION 1..193: note = human TLR8 intracellular signaling domain

SEQ ID NO: 524 - protein, 42 aa, synthetic construct
  REGION 1..42: note = human Tim4 intracellular signaling domain
RGKLMETYCSQKHTRLDYIGDSKNVLNDVQHGREDEDGLFTL

SEQ ID NO: 525 - protein, 43 aa, synthetic construct
  REGION 1..43: note = human Tim4 intracellular signaling domain
MRGKLMETYCSQKHTRLDYIGDSKNVLNDVQHGREDEDGLFTL

SEQ ID NO: 526 - skipped

SEQ ID NO: 527 - protein, 495 aa, synthetic construct
  REGION 1..495: note = hTIM4-TIM4-CD28tm-CD28icd-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 528 - protein, 670 aa, synthetic construct
  REGION 1..670: note = hTIM4-TIM4-CD28tm-CD28icd-TLR2ICD-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRSHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 529 - protein, 670 aa, synthetic construct
  REGION 1..670: note = hTIM4-TIM4-CD28tm-CD28icd-CD3zICD-TLR2ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 530 - protein, 688 aa, synthetic construct
  REGION 1..688: note = hTIM4-TIM4-CD28tm-CD28icd-TLR8ICD-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRSHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 531 - protein, 688 aa, synthetic construct
  REGION 1..688: note = hTIM4-TIM4-CD28tm-CD28icd-CD3zICD-TLR8ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 532 - protein, 629 aa, synthetic construct
  REGION 1..629: note = hTIM4-TIM4-CD28tm-TLR2ICD-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 533 - protein, 629 aa, synthetic construct
  REGION 1..629: note = hTIM4-TIM4-CD28tm-CD3zICD-TLR2ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 534 - protein, 647 aa, synthetic construct
  REGION 1..647: note = hTIM4-TIM4-CD28tm-TLR8ICD-CD3zICD
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 535 - protein, 647 aa, synthetic construct
  REGION 1..647: note = hTIM4-TIM4-CD28tm-CD3zICD-TLR8ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 536 - protein, 728 aa, synthetic construct
  REGION 1..728: note = hTIM4-TIM4-CD28tm-TRAF6ICD-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 537 - protein, 728 aa, synthetic construct
  REGION 1..728: note = hTIM4-TIM4-CD28tm-CD3zICD-TRAF6ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQFWVLVVVGGVLACYSLLVTVAFIIFWVRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 538 - protein, 489 aa, synthetic construct
  REGION 1..489: note = hTIM4-TIM4-TIM4tm-CD28icd-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 539 - protein, 664 aa, synthetic construct
  REGION 1..664: note = hTIM4-TIM4-TIM4tm-CD28icd-TLR2ICD-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRSKRSRLLHSDYMNMTPRRPGPTRKHYQPYAPPRDFAAYRSHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 540 - protein, 664 aa, synthetic construct
  REGION 1..664: note = hTIM4-TIM4-TIM4tm-CD28icd-CD3zICD-TLR2ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 541 - protein, 682 aa, synthetic construct
  REGION 1..682: note = hTIM4-TIM4-TIM4tm-CD28icd-TLR8ICD-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRSKRSRLLHSDYMNMTPRRPGPTRKHYQPYAPPRDFAAYRSHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 542 - protein, 682 aa, synthetic construct
  REGION 1..682: note = hTIM4-TIM4-TIM4tm-CD28icd-CD3zICD-TLR8ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 543 - protein, 623 aa, synthetic construct
  REGION 1..623: note = hTIM4-TIM4-TIM4tm-TLR2ICD-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 544 - protein, 623 aa, synthetic construct
  REGION 1..623: note = hTIM4-TIM4-TIM4tm-CD3zICD-TLR2ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 545 - protein, 641 aa, synthetic construct
  REGION 1..641: note = hTIM4-TIM4-TIM4tm-TLR8ICD-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 546 - protein, 641 aa, synthetic construct
  REGION 1..641: note = hTIM4-TIM4-TIM4tm-CD3zICD-TLR8ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 547 - protein, 722 aa, synthetic construct
  REGION 1..722: note = hTIM4-TIM4-TIM4tm-TRAF6ICD-CD3zICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLARVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 548 - protein, 722 aa, synthetic construct
  REGION 1..722: note = hTIM4-TIM4-TIM4tm-CD3zICD-TRAF6ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRMSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLA

SEQ ID NO: 549 - protein, 378 aa, synthetic construct
  REGION 1..378: note = hTIM4-TIM4-TIM4tm-TIM4ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLMRGKLMETYCSQKHTRLDYIGDSKNVLNDVQHGREDEDGLFTL

SEQ ID NO: 550 - protein, 665 aa, synthetic construct
  REGION 1..665: note = hTIM4-TIM4-TIM4tm-TIM4ICD-TLR2-CD3z
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRGKLMETYCSQKHTRLDYIGDSKNVLNDVQHGREDEDGLFTLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 551 - protein, 665 aa, synthetic construct
  REGION 1..665: note = hTIM4-TIM4-TIM4tm-TIM4ICD-CD3z-TLR2
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRGKLMETYCSQKHTRLDYIGDSKNVLNDVQHGREDEDGLFTLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 552 - protein, 683 aa, synthetic construct
  REGION 1..683: note = hTIM4-TIM4-TIM4tm-TIM4ICD-TLR8-CD3z
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRGKLMETYCSQKHTRLDYIGDSKNVLNDVQHGREDEDGLFTLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQYRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 553 - protein, 683 aa, synthetic construct
  REGION 1..683: note = hTIM4-TIM4-TIM4tm-TIM4ICD-CD3z-TLR8
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRGKLMETYCSQKHTRLDYIGDSKNVLNDVQHGREDEDGLFTLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 554 - protein, 552 aa, synthetic construct
  REGION 1..552: note = hTIM4-TIM4-TIM4tm-TLR2ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRGKLMETYCSQKHTRLDYIGDSKNVLNDVQHGREDEDGLFTLHRFHGLWYMKMMWAWLQAKRKPRKAPSRNICYDAFVSYSERDAYWVENLMVQELENFNPPFKLCLHKRDFIPGKWIIDNIIDSIEKSHKTVFVLSENFVKSEWCKYELDFSHFRLFDENNDAAILILLEPIEKKAIPQRFCKLRKIMNTKTYLEWPMDEAQREGFWVNLRAAIKS

SEQ ID NO: 555 - protein, 570 aa, synthetic construct
  REGION 1..570: note = hTIM4-TIM4-TIM4tm-TLR8ICD
  SIGNAL 1..24
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLRGKLMETYCSQKHTRLDYIGDSKNVLNDVQHGREDEDGLFTLHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 556 - protein, 598 aa, synthetic construct
  REGION 1..598: note = HPV16 E7 TCR? chain-P2A-TCR? chain; amino acids 309-335 are P2A sequence
MAPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSRAKRSGSGATNFSLLKQAGDVEENPGPMWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

(19) United States
(12) Patent Application Publication
(10) Pub. No.: US 2024/0285684 A1
(43) Pub. Date: Aug. 29, 2024

(54) ANTI-CD72 CHIMERIC RECEPTORS AND USES THEREOF

(71) Applicant: CERo Therapeutics, Inc., South San Francisco, CA (US)

(72) Inventors: Daniel Mark COREY, Menlo Park, CA (US); Nathan KIPNISS, San Francisco, CA (US); Yukiye KOIDE, San Francisco, CA (US); Brandon CIENIEWICZ, Sunnyvale, CA (US); Yan QU, Burlingame, CA (US); Sunil THOMAS, San Bruno, CA (US)

(21) Appl. No.: 18/418,120

(22) Filed: Jan. 19, 2024

Related U.S. Application Data
(63) Continuation of application No. 18/041,198, filed on Jan. 1, 0001, now abandoned.
(60) Provisional application No. 63/066,149, filed on Aug. 14, 2020.
(60) Provisional application No. 63/172,057, filed on Apr. 7, 2021.
(60) Provisional application No. 63/226,736, filed on Jul. 28, 2021.

(51) Int. Cl. A61K 35/17; A61K 39/00; A61P 35/00; C07K 14/725; C07K 16/28; C12N 15/63

(52) U.S. Cl. CPC A61K 35/17; A61K 39/001129; A61K 39/4611; A61K 39/4631; A61P 35/00; C07K 14/7051; C07K 16/2851; C12N 15/63; A61K 2239/13; A61K 2239/21; C07K 2317/565; C07K 2319/02; C07K 2319/03

(57) ABSTRACT

The present disclosure relates to compositions and methods comprising anti-CD72 chimeric antigen receptors, host cells modified to include anti-CD72 chimeric antigen receptor molecules. The provided compositions, methods, and uses include those for combination therapies involving administration of a chimeric engulfment receptor or chimeric Tim receptor, such as immune cells engineered to co-express both the anti-CD72 chimeric antigen receptor and the chimeric engulfment receptor or chimeric Tim receptor.

### REFERENCE TO AN ELECTRONIC SEQUENCE LISTING

The contents of the electronic sequence listing (413C1_SeqListing.xml; Size: 729,067 bytes; and Date of Creation: Jan. 18, 2024) is herein incorporated by reference in its entirety.

### BACKGROUND

Antigen escape or downregulation is one mechanism for relapse from chimeric antigen receptor (CAR) T-cell therapy. An approach to overcome this is to simultaneously target more than one antigen on cancer cells. CD72 is a C-type lectin receptor, with high restriction of expression to normal and pathologic B cells and subsets of myeloid blasts. Its expression is retained following CAR T-cell relapse, whereby CD72 marks residual leukemic/lymphoma blasts without detectable CD19 or CD22, due to downregulation or loss of antigen expression through immune pressure by CAR T cells. The high B cell restriction of CD72, its broad expression on malignant lymphoma and leukemia B cells and Acute Myelogenous Leukemia (AML) subsets, and its retained expression on relapsed CD19- and CD22-directed therapies, renders CD72 a suitable target for B cell lymphomas and leukemias and AML tumors.

### BRIEF DESCRIPTION OF THE SEVERAL VIEWS OF THE DRAWINGS

FIG. 1. Schematic showing CD72-directed CARs, which include sc02-025 or sc02-004 anti-CD72 binding domains, IgG4 hinge region, CD28-derived transmembrane and signaling domains, CD35-derived signaling domain, and T2A ribosome skip element. Truncated CD34 was included in pCTX206, pCTX207, or pCTX208 vectors to serve as a selection marker for successful transduction.

FIG. 2. Co-expression of CD72 CARs and tCD34 are shown. T cells were transduced with pCTX206 or pCTX208 and stained for cell surface expression of the CD72 CAR and a vector-encoded selection marker, truncated CD34 (tCD34). Values indicating co-expression of the CD72 CAR and tCD34 are indicated.

FIGS. 3A-3B. Transduction efficiency and fold expansion are shown for T cells transduced with pCTX206, pCTX207, or pCTX208. FIG. 3A. T cells were stimulated with TransACT in a 24 well gRex plate. After 24 h, cells were counted and transduced with 5 MOI of lentivirus encoding pCTX206, pCTX207, or pCTX208. After 6 d in culture, cells were resuspended, counted, and fold expansion was calculated. FIG. 3B. At 6 d post transduction, transduced T cells were resuspended and stained for the tCD34 marker gene encoded on pCTX206, pCTX207, and pCTX208. Cell staining was analyzed by flow cytometry. Results are for 1-3 independent transductions.

FIG. 4. Jeko-1 mantle cell lymphoma cells were evaluated for cell surface expression of CD72. Jurkat lymphoma cells (T cell lineage) and unstained cells were used as negative controls. Cells were stained with anti-CD72 antibodies and characterized by FACS.

FIG. 5. Cytotoxicity of T cells transduced with CD72-directed CAR vectors are shown. Jeko-1 cells were engineered to express mCherry; 25,000 mCherry+JeKo-1 cells were cultured at a 2:1, 1:1, or 0.5:1 effector cell:target cell ratio (E:T ratio) with CTX206, CTX207, or CTX208 CAR-T cells. Cultures were incubated at 37° C. for 4 d on a 96-well fibronectin-coated plate in an Incucyte cell culture and imaging device. Conditions were plated in duplicate, and error bars represent+/−SD.

FIGS. 6A-6C. Activation markers expressed in CAR-T cells upon CD72-target cell-specific activation are shown. FIG. 6A. CTX206 CAR-T cells were cultured with Jeko-1 cells for 96 hrs at 0.5:1, 1:1, and 2:1 E:T ratios followed by staining with CD19 to detect remaining Jeko-1 cells. Plots were obtained by excluding dead cells. Red squares indicate residual Jeko-1 cells left in culture. FIG. 6B. CTX206 and CTX208 CAR-T cells were assessed for expression and specific upregulation of indicated markers in response to Jeko-1 (CD72+) cells. Activation markers CD25 and CD137 were co-stained with CD4 and CD8 antibodies. T cells transduced with pCTX control vector served as a negative control. FIG. 6C. Upregulation of CD137 was measured in CTX206 or CTX208 CAR-T cells upon Jeko-1 target cell co-culture. Unstimulated CTX206 or CTX208 CAR-T cells were used as negative controls to assess constitutive or tonic activation signals in the absence of targets.

FIG. 7. Proliferation and CD137 expression in CTX206 or CTX208 CAR-T cells is shown 72 hours-post stimulation with Jeko-1 cells in the absence of cytokines. Cells used in the assay were first stimulated with TransAct (CD3/28) for 21 days. Cell Trace Violet dye dilution and anti-CD137 antibody staining were assessed by FACS. CD3/28 beads were used as positive controls.

FIG. 8. Proliferation and CD25 expression in CTX206 or CTX208 CAR-T cells is shown 72 hours-post stimulation with Jeko-1 cells in the absence of cytokines. Cells used in the assay were first stimulated with TransAct (CD3/28) for 21 days. Cell Trace Violet dye dilution and anti-CD137 antibody staining were assessed by FACS. CD3/28 beads were used as positive controls.

FIG. 9. Bulk cytokine secretion from CTX206, CTX207, and CTX208 CAR-T cells is shown. Multiplex cytokine analysis from bulk supernatants was obtained 48 hrs after co-culture with Jeko-1 targets.

FIGS. 10A-10F. Biodistribution and in vivo effects of CD72 CAR-T cells are shown. FIG. 10A. A murine model was used to evaluate the efficacy of CTX206 and CTX208 CAR-T cells. Cohorts were inoculated with 1e6 JeKo-1 ffluc cells via tail injection, and CTX206 CAR-T, CTX208 CAR-T, or control untransduced T cells. CAR T cells were administered 7 days after tumor inoculation. Peripheral blood was collected sixteens days post-CAR T cell infusion. FIGS. 10B-10C. Scrial bioluminescence imaging was performed to quantify tumor burden in mice following CAR-T infusion. FIG. 10B. Tumor burden, as measured by bioluminescence, is shown in mice receiving 0.5c6, 1c6, or 4c6 CTX206 CAR-T cells. FIG. 10C. Tumor burden, as measured by bioluminescence, is shown in mice receiving 2e6 CTX206 or 2e6 CTX208 CAR-T cells. FIG. 10D. Tumor-associated bioluminescence derived from JeKo-1 ffluc cells is shown in mice imaged at 11 days post-infusion with 2e6 CTX206 or 2c6 CTX208 CAR-T cells. FIG. 10E. Bone marrow was collected 7 days following infusion of 2e6 cells and the frequency of CD45+CD8+CD4+CD34+-T cells were quantified. FIG. 10F. The frequency of hCD45 cells in the bone marrow at day 7 post-CAR T infusion is shown.

FIG. 11. Schematic showing a CD72-directed CAR and a chimeric engulfment receptor (CER) or chimeric Tim1/4 receptor expressed in a single cell. FIGS. 12A-12C. Co-transduction of Chimeric Engulfment Receptors (CERs) with CTX206 and associated functional data is shown.

FIG. 12A. T cells were co-transduced with CTX206 and a CER, CTX137 or CTX140. Transduction with CTX137 is indicated by surface expression of EGFR and transduction with CTX206 is indicated by surface expression of tCD34. FIG. 12B. To evaluate cytotoxicity, T cells co-expressing both CTX206+CTX137 or CTX206+CTX140 were incubated at a 1:1 ratio with mCherry+JeKo-1 target cells for 2.5 d in an Incucyte live-cell imaging system. The number of mCherry+JeKo-1 cells is shown at each time point. FIG. 12C. CTX206 CAR-T cells were mixed with CER-transduced T cells (CER135, CER136, CER 137 (also referred to as CTX137), CER140 (also referred to as CTX140), CER141, CER142, CER143, CER144) in a 1:1 ratio and cultured with target Jekol mCherry T cells at a 0.25:1 effector:target ratio to evaluate cytotoxicity.

FIGS. 13A-13C. Co-transduction of Chimeric Engulfment Receptors (CERS) with CTX208 and associated functional data is shown. FIG. 13A. Representative plots of co-transduced cells are shown. Cells were transduced with either CTX137 alone, CTX208 alone, or equivalent amounts of CTX137 and CTX208. At 6 d post transduction, expression of the tEGFR (CTX137) and tCD34 (CTX208) tags were measured on transduced cells by flow cytometry. Gates were drawn using mock transduced cells (UNT). FIG. 13B-13C. Killing of Jeko-1 cells by T cells co-transduced with CTX137 and CTX208 is shown. T cells transduced with CTX208 or co-transduced with CTX137 and CTX208 were incubated at a 1:1 ratio (FIG. 13B.) or varying ratios (FIG. 13C.) with 25,000 Jeko-1 cells engineered to express mCherry. Cells were incubated for 4.5 d in an Incucyte live-cell imaging system. The number of mCherry+ target cells is shown at each time point. Wells were plated in duplicate, error bars indicate+/−SD.

FIG. 14. A murine model was used to evaluate in vivo tumor control by the co-infusion of CTX206 CAR-T cells and T cells expressing CERs CTX143 or CTX136. Cohorts were inoculated with 1e6 JeKo-1 ffluc via tail injection and 2.5e6 CTX206 CAR-T cells 7 days after tumor inoculation. After 72 hours, animals were infused with a second infusion of 2.5e6 T cells expressing CTX136 or CTX143. Serial bioluminescence imaging and quantification are illustrated through day +6 post CAR infusion (day +3 post CER infusion). Untransduced cells were infused as a control.

FIG. 15A-15B. Differentially expressed RNA transcripts from sorted chimeric TIM-4 receptor T cells (FIG. 15A.) and CAR-T cells (FIG. 15B.) after antigen encounter are shown. Gene ontology analysis was performed to assess the presence of divergent transcriptional programs. Cells were cultured similarly through day 8 post-activation and transduction. CAR and CER-T cells were sorted after 48 and 96 hrs after co-culture with Ag+ cells (JeKo-1). Heat maps show progression of transcriptional programs over time in the culture relative to baseline.

FIG. 16 show (left panel) PtdSer induction kinetics by a CD72-specific CAR and (right panel) CD72 specific CAR engagement primers target cells for CER-mediated clearance.

FIG. 17 shows screening of various CD72-specific scFvs for binding affinity.

FIG. 18 shows expression titers of various CD72-specific CAR T cells based on detection of co-expressed EGFRt tag using anti-EGFRt antibody staining.

FIG. 19 shows that vector copy number (VCN) titers correlate with CAR expression titers.

FIG. 20 shows CD72 specific scFv variable region orientation and linker selection for various CAR (IgG4 hinge, CD28 transmembrane domain, 4-1BB signaling domain, and CD3ζ signaling domain) constructs

FIG. 21 shows that evaluation of 4-1BB in various CD72 specific CAR T cells shows minimal auto-activation. Some variation observed between cell preparations. All CD72 specific scFvs have relatively low baseline activation (˜2fold over UNT).

FIG. 22 shows set up for serial stimulation assays. pCTX768 CER/CD72-CAR single cells elicit durable anti-lymphoma responses in vitro.

FIG. 23 is a graph showing that higher affinity scFvs show greater potency in initial stimulation rounds. Cytotoxicity evaluated by incucyte over time comparing CD72 CAR scFvs and linker orientations (VH-VL or VL-VH) across 32 constructs at MOIs 3 and 10. Shown are 0.25 Effector: Target ratios. Targets re-seeded every 72 hrs. Function observed throughout 4 rounds of recursive killing. High stringency applied to assay to differentiate CARs. Evaluated at low E:T (0.1, 0.25, and 1:1), normalized using % CAR+. Between rounds, 75% of T cells removed and reseeded with Jeko-1 targets.

FIG. 24 shows that high IFNγ secretion observed after co-culture with target cells in all CARs. CARs show some baseline secretion of IFN-γ (left bars). Upon stimulation with CD72+ Jeko-1 cells, all CARs have strong IFN-γ response (Right bars).

FIG. 25 shows that IFNγ secretion is similar between constructs and transduction rounds.

FIG. 26 shows that high TNFα was observed from CARs with high in vitro cytotoxicity. All CARs have negligible baseline secretion of TNF-α (left bars). Upon stimulation, variable TNF-α secretion is observed (right bars). CARs with best in vitro cytotoxicity have higher TNF-α

FIG. 27 shows that TNF-α secretion can be more variable, but high secretion may be correlated with cytotoxicity.

FIG. 28 shows that high IL-2 secretion was observed from CARs with high in vitro cytotoxicity. All CARs have negligible baseline IL-2 secretion (left bars). All CARs secrete IL-2 in response to targets. Magnitude of response correlates to strong in vitro function.

FIG. 29 shows that IL-2 secretion can be more variable, but high secretion may be correlated with cytotoxicity.

FIG. 30 shows EGFR: CAR surface expression (myc) ratio across constructs. CARs ranked from best to worst after each serial killing round. Highest % JeKo-1 elimination ranked #1. High concordance not necessarily associated with good function.

FIG. 31 shows that a subset of CARs improve over time in serial stimulation studies. Low E:T cytotoxicity predicts longer term function in vitro.

FIG. 32A shows evaluation of CD72 specific CARs (CTX836, CTX840, CTX842, CTX844, CTX845, and CTX850) in JeKo-1 Mantle cell lymphoma (MCL) model in NSG mice as measured by bioluminescence (BLI) imaging at day 9 post T-cell infusion. FIG. 32B shows bioluminescence measurement by individual CD72 specific CAR (CTX836, CTX840, CTX842, CTX844, CTX845, and CTX850). n=7-10 per group.

FIG. 33 shows exemplary schematic and design for dual targeting CER-CAR T cell.

FIG. 34 shows expression titers for tricistronic vectors pCTX768 and pCTX771 at 1× and 20× titers.

FIG. 35 shows PtdSer-CER (anti-Tim4) and CD72-CAR staining (anti-myc) of transduced T cells. Single cells express both a CAR and a CER. FACS plots shown are at day +10 post-transduction.

FIG. 36 shows that pCTX768 CER/CD72-CAR single cells elicit durable anti-lymphoma responses in vitro in serial stimulation assay.

FIG. 37 shows improvement of function by tailoring CER-T2A-CAR sequence order in a multi-cistronic vector. CTX768-CER-T2A-CAR shows superior cytotoxicity to CAR-alone in 4 rounds of serial killing. Order of placement within the multi-cistronic vector favors encoding the CER 1st, followed by the CAR.

FIG. 38 shows that dual specific CER/CD72 CAR T cell has sustained anti-tumor responses at low effector: target ratios. Cytotoxicity evaluated by incucyte over time at various effector:target ratios (right). 50% killing time (the amount of time it takes to kill 50% of target tumor cells) are calculated from the various conditions (left). CTX768-CER-T2A-CAR shows clearance of targets across a range of effector: target ratios in serial killing assays. 50% Killing Time (KT50) Rd 2 shows effect of T cell dilution.

FIG. 39 shows differential cytokine production observed between pCTX768 and pCTX771. CTX768-CER-T2A-CAR has equivalent IFN-γ secretion as CTX771 CAR alone, and lower TNF-α and IL-2 secretion despite superior killing.

FIG. 40 shows that CER co-expression with CAR does not alter antigen dependent activation. 4-1BB and CD25 are upregulated on T cells after activation. Both CTX768-CER-T2A-CAR and CTX771 CAR alone have comparable expression of activation markers, suggesting CER co-expression does not lead to overactivation

FIG. 41 shows dye dilution assay to monitor cell proliferation of pcTX768 and pCTX771 T cells. T cells labeled with CFSE dye before stimulation. After stimulation, dye dilutes with each cell division, leading to lower MFI. Extent of T cell proliferation in response to antigen is dependent on strength of signal. Enhanced proliferative capacity of CER-CAR (pCTX768) was observed compared to CAR-alone (pCTX771). Both CTX768-CER-T2A-CAR and CTX771 CAR alone proliferate in response to CD72+ target cells. CTX768-CER-T2A-CAR has slightly increased proliferation

FIG. 42 shows a schematic for assessing phagocytic and endocytic capabilities in engineered T cells.

FIG. 43 shows a chimeric Tim-4 protein in combination with a CD72 CAR (pCTX768) enhances tumor uptake in CD8+ and CD4+CD8+ T cells over CD72 CAR alone (pCTX771).

FIG. 44 shows that CER-CAR (pCTX768) T cells both engulf and kill JeKo-1 tumor targets. The frequency of pHrodo+/CT-Violet+ cells is shown across various T cell subsets after 14 hr of co-culture (left). Remaining JeKo-1-GFP+ cells quantified over time, 14 hr, 38 hr and 62 hr (right). CTX768 has capabilities to both engulf and kill JeKo-1 tumor targets.

FIG. 45 shows microscopy image of CER-CAR (pCTX768) engulfing pHrodo-labeled Jeko-1 targets.

FIG. 46 is schematic describing role of T cell maturation markers on function.

FIG. 47 shows dual-targeting CAR/CER CD8+ T cells have reduced effector memory and increased CCR7+ populations, including naïve and central memory phenotypes

FIG. 48 shows dual-targeting CAR/CER CD4+ T cells have less effector memory phenotype and increased CD45RA+CCR7+naïve T cells.

FIG. 49 shows tricistronic CER/CAR-EGFRt Constructs.

FIG. 50 shows for multiple CD72 CARs, high viability and fold expansion observed throughout cell production. CD4 and CD8 cells were mixed at a 1:1 ratio in the presence of TransACT. 24 h post stimulation, cells were transduced with a range of lentiviruses encoding CER constructs across different MOIs. Cells were expanded in the presence of IL-2, IL-7, and IL-15 in G-Rex vessels. Viabilities and fold expansion were quantified day +4 and day +6 post-transduction.

### DETAILED DESCRIPTION

The present disclosure provides CD72 specific chimeric antigen receptors (CARs) and method for use in cellular immunotherapy, for the treatment of diseases or conditions associated with CD72 expression, including cancers and tumors.

Antigen escape or downregulation is one mechanism for relapse from CAR T-cell therapy. An approach to overcome antigen escape or downregulation is to simultaneously target more than one antigen on cancer cells. CD72 is a C-type lectin receptor, with high restriction of expression to normal and pathologic B cells and subsets of myeloid blasts. Its expression is retained following CAR T-cell relapse, whereby CD72 marks residual leukemic/lymphoma blasts without detectable CD19 or CD22, due to downregulation or loss of antigen through immune pressure by CAR T cells. The high B cell restriction of CD72, its broad expression on malignant lymphoma and leukemia B cells and Acute Myelogenous Leukemia (AML) subsets, and its retained expression on relapsed CD19- and CD22-directed therapies, renders CD72 a suitable target for B cell lymphomas and leukemias and AML tumors by CD72 CARs of the present disclosure.

Compositions of the present disclosure are useful in methods of treating B-cell malignancies. In particular embodiments, the CD72 specific CARs of the present disclosure are useful in combination therapy strategies, e.g., with a chimeric engulfment receptor (CER), chimeric T-cell membrane protein (Tim) receptor, chemotherapy, radiation, molecularly targeted inhibitors, etc., or any combination thereof, for treating B-cell malignancies in a multi-pronged approach that improves CAR function, enhances small molecule therapy, limits tumor antigen escape, provides more durable, long term response to therapy. The CER or chimeric Tim receptor contains a binding domain directed against pro-engulfment target antigens (e.g., phosphatidylserine), linked to one or more effector domains which promote tumor uptake via endocytosis or phagocytosis. Target antigen engagement by the CAR, independently increases the pro-engulfment target antigen on target cells recognized by the CER or chimeric Tim receptor. Such increases in pro-engulfment target antigen density may amplify signaling from the CER or chimeric Tim receptor, resulting in enhanced function and limiting outgrowth of CD72-low variant clones. Concurrent cytotoxic therapy, such as molecularly targeted inhibitors, can also increase expression of pro-engulfment target antigen, resulting in enhanced CER or chimeric Tim receptor activation. In some embodiments, the CD72 specific CAR and CER or chimeric Tim receptor of the present disclosure are expressed within the same cell, such as immune cell, or more particularly a T cell, to produce a multi-specific and multifunctional engineered cell for the treatment of diseases associated with CD72 expression, such as hematological malignancies, including B-cell malignancies. In some embodiments, the CD72 specific CAR and CER or chimeric Tim receptor are encoded by the same vector or by separate vectors within the same engineered cell.

Prior to setting forth this disclosure in more detail, it may be helpful to an understanding thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

Terms understood by those in the art of antibody technology are each given the meaning acquired in the art, unless expressly defined differently herein. The term “antibody” is used in the broadest sense and includes polyclonal and monoclonal antibodies. An “antibody” may refer to an intact antibody comprising at least two heavy (H) chains and two light (L) chains inter-connected by disulfide bonds, as well as an antigen-binding portion (or antigen-binding domain) of an intact antibody that has or retains the capacity to bind a target molecule. An antibody may be naturally occurring, recombinantly produced, genetically engineered, or modified forms of immunoglobulins, for example intrabodies, peptibodies, nanobodies, single domain antibodies, SMIPs, multispecific antibodies (e.g., bispecific antibodies, diabodies, triabodies, tetrabodies, tandem di-scFV, tandem tri-scFv, ADAPTIR). A monoclonal antibody or antigen-binding portion thereof may be non-human, chimeric, humanized, or human, preferably humanized or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988). “Antigen-binding portion” or “antigen-binding domain” of an intact antibody is meant to encompass an “antibody fragment,” which indicates a portion of an intact antibody and refers to the antigenic determining variable regions or complementary determining regions of an intact antibody. Examples of antibody fragments include, but are not limited to, Fab, Fab′, F(ab′)2, and Fv fragments, Fab′-SH, F(ab′)2, diabodies, linear antibodies, scFv antibodies, VH, and multispecific antibodies formed from antibody fragments. A “Fab” (fragment antigen binding) is a portion of an antibody that binds to antigens and includes the variable region and CH1 of the heavy chain linked to the light chain via an inter-chain disulfide bond. An antibody may be of any class or subclass, including IgG and subclasses thereof (IgG1, IgG2, IgG3, IgG4), IgM, IgE, IgA, and IgD.

The term “variable region” or “variable domain” in the context of an antibody refers to the domain of an antibody heavy or light chain that is involved in binding of the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt ct al. Kuby Immunology, 6th ed., W.H. Freeman and Co., page 91 (2007)). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The terms “complementarity determining region” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (HCDR1, HCDR2, HCDR3) and three CDRs in each light chain variable region (LCDR1, LCDR2, LCDR3).

As used herein, the terms “binding domain”, “binding region”, and “binding moiety” refer to a molecule, such as a peptide, oligopeptide, polypeptide, or protein that possesses the ability to specifically and non-covalently bind, associate, unite, recognize, or combine with a target molecule (e.g., tumor antigen). A binding domain includes any naturally occurring, synthetic, semi-synthetic, or recombinantly produced binding partner for a biological molecule or other target of interest. In some embodiments, the binding domain is an antigen-binding domain, such as an antibody or functional binding domain or antigen-binding portion thereof. Exemplary binding domains include single chain antibody variable regions (e.g., domain antibodies, sFv, scFv, Fab), receptor cctodomains (e.g., TNF-α), ligands (e.g., cytokines, chemokines), or synthetic polypeptides selected for the specific ability to bind to a biological molecule.

“T cell receptor” (TCR) refers to a molecule found on the surface of T cells (also referred to as T lymphocytes) that is generally responsible for recognizing antigens bound to major histocompatibility complex (MHC) molecules. The TCR is generally composed of a disulfide-linked heterodimer of the highly variable α and β chains (also known as TCRα and TCRβ, respectively) in most T cells. In a small subset of T cells, the TCR is made up of a heterodimer of γ and δ chains (also known as TCRγ and TCRδ, respectively). Each chain of the TCR is a member of the immunoglobulin superfamily and possesses one N-terminal immunoglobulin variable domain, one immunoglobulin constant domain, a transmembrane region, and a short cytoplasmic tail at the C-terminal end (see Janeway et al., Immunobiology: The Immune System in Health and Disease, 3^{rd} Ed., Current Biology Publications, p. 4:33, 1997). TCRs of the present disclosure may be from various animal species, including human, mouse, rat, cat, dog, goat, horse, or other mammals. TCRs may be cell-bound (i.e., have a transmembrane region or domain) or in soluble form. TCRs include recombinantly produced, genetically engineered, fusion, or modified forms of TCRs, including for example, scTCRs, soluble TCRs, TCR fusion constructs (TRUC™; see, U.S. Patent Publication No. 2017/0166622).

The term “variable region” or “variable domain” of a TCR α-chain (Vα) and β-chain (Vβ), or Vγ and Vδ for γδ TCRs, are involved in binding of the TCR to antigen. The V_{α} and V_{β} of a native TCR generally have similar structures, with each variable domain comprising four conserved FRs and three CDRs. The V_{α} domain is encoded by two separate DNA segments, the variable gene segment (V gene) and the joining gene segment (J gene); the V_{β} domain is encoded by three separate DNA segments, the variable gene segment (V gene), the diversity gene segment (D gene), and the joining gene segment (J gene). A single V_{α} or V_{β} domain may be sufficient to confer antigen-binding specificity.

“Major histocompatibility complex molecule” (MHC molecule) refers to a glycoprotein that delivers a peptide antigen to a cell surface. MHC class I molecules are heterodimers composed of a membrane spanning a chain (with three α domains) and a non-covalently associated β2 microglobulin. MHC class II molecules are composed of two transmembrane glycoproteins, a and β, both of which span the membrane. Each chain has two domains. MHC class I molecules deliver peptides originating in the cytosol to the cell surface, where peptide:MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, or other mammals.

“Chimeric antigen receptor” (CAR) refers to a chimeric fusion protein comprising two or more distinct domains linked together in a way that does not occur naturally in a host cell and can function as a receptor when expressed on the surface of a cell. CARs are generally composed of an extracellular domain comprising a binding domain that binds a target antigen, an optional extracellular spacer domain, a transmembrane domain, and an intracellular signaling domain (e.g., comprising an immunoreceptor tyrosine-based activation motif (ITAM)), and optionally an intracellular costimulatory domain). In certain embodiments, an intracellular signaling domain of a CAR has an ITAM (e.g., CD3ζ) containing intracellular signaling domain and an intracellular costimulatory domain (e.g., CD28). In certain embodiments, a CAR is synthesized as a single polypeptide chain or is encoded by a nucleic acid molecule as a single chain polypeptide.

A variety of assays are known for identifying binding domains of the present disclosure that specifically bind a particular target, as well as determining binding domain affinities, such as Western blot, ELISA, analytical ultracentrifugation, spectroscopy, surface plasmon resonance (BIACORE®) analysis, and MHC tetramer analysis (see also, e.g., Scatchard et al., Ann. N.Y. Acad. Sci. 51:660, 1949; Wilson, Science 295:2103, 2002; Wolff et al., Cancer Res. 53:2560, 1993; Altman et al., Science 274:94-96, 1996; and U.S. Pat. Nos. 5,283,173, 5,468,614, or the equivalent). As used herein, “specifically binds” refers to an association or union of a binding domain, or a fusion protein thereof, to a target molecule with an affinity or Kₐ (i.e., an equilibrium association constant of a particular binding interaction with units of 1/M) equal to or greater than 10⁵ M⁻¹, while not significantly associating or uniting with any other molecules or components in a sample.

The terms “antigen” and “Ag” refer to a molecule that is capable of inducing an immune response. The immune response that is induced may involve antibody production, the activation of specific immunologically-competent cells, or both. Macromolecules, including proteins, glycoproteins, and glycolipids, can serve as an antigen. Antigens can be derived from recombinant or genomic DNA. As contemplated herein, an antigen need not be encoded (i) solely by a full length nucleotide sequence of a gene or (ii) by a “gene” at all. An antigen can be generated or synthesized, or an antigen can be derived from a biological sample. Such a biological sample can include, but is not limited, to a tissue sample, a tumor sample, a cell, or a biological fluid.

The term “epitope” or “antigenic epitope” includes any molecule, structure, amino acid sequence or protein determinant within an antigen that is specifically bound by a cognate immune binding molecule, such as an antibody or fragment thereof (e.g., scFv), T cell receptor (TCR), CAR, chimeric engulfment receptor, or other binding molecule, domain or protein. Epitopic determinants generally contain chemically active surface groupings of molecules, such as amino acids or sugar side chains, and can have specific three dimensional structural characteristics, as well as specific charge characteristics. An epitope may be a linear epitope or a conformational epitope.

As used herein, the term “Tim4” (T-cell immunoglobulin and mucin domain containing protein 4), also known as “TimD4”, refers to a phosphatidylserine receptor that is typically expressed on antigen presenting cells, such as macrophages and dendritic cells. Tim4 mediates the phagocytosis of apoptotic, necrotic, damaged, injured, or stressed cells, which present phosphatidylserine (PtdSer) on the exofacial (outer) leaflet of the cell membrane. Tim4 is also capable of binding to Tim1 expressed on the surface of T cells and inducing proliferation and survival. In certain embodiments, Tim4 refers to human Tim4. An exemplary human Tim4 protein comprises an amino acid sequence of SEQ ID NO:49.

As used herein, the term “Tim4 binding domain” refers to the N-terminal immunoglobulin-fold domain of Tim4 that possesses a metal ion-dependent pocket that selectively binds phosphatidylserine (PtdSer). An exemplary human Tim4 binding domain comprises an amino acid sequence of SEQ ID NO:50, and an exemplary mouse Tim4 binding domain comprises an amino acid sequence of SEQ ID NO:51. In certain embodiments, the Tim4 binding domain does not include a signal peptide.

A Tim4 binding domain includes a variable immunoglobulin (IgV) like domain (referred to herein as an “IgV domain”) and a Mucin like domain (“referred to herein as a “mucin domain”). An exemplary human Tim4 IgV domain comprises an amino acid sequence of SEQ ID NO:52, and an exemplary human Tim4 mucin domain comprises an amino acid sequence of SEQ ID NO:53. In certain embodiments, the Tim4 binding domain does not include a signal peptide. An exemplary human Tim4 signal peptide has the amino acid sequences of SEQ ID NO:54. An exemplary mouse Tim4 signal peptide has the amino acid sequences of SEQ ID NO:55.

As used herein, the term “Tim1” (T-cell immunoglobulin and mucin domain containing protein 1), refers to a phosphatidylserine receptor that is expressed on the surface of T cells. Tim1, as noted above is also capable of binding to Tim4 expressed on the surface of antigen presenting cells. In certain embodiments, Tim1 refers to human Tim1. An exemplary human Tim1 protein comprises an amino acid sequence of SEQ ID NO:56.

As used herein, the term “Tim1 binding domain” refers to the N-terminal immunoglobulin-fold domain of Tim1 that selectively binds PtdSer. An exemplary human Tim1 binding domain comprises an amino acid sequence of SEQ ID NO:57.

A Tim1 binding domain includes an IgV domain and a mucin domain. An exemplary human Tim1 IgV domain comprises an amino acid sequence of SEQ ID NO:58, and an exemplary human Tim1 mucin domain comprises an amino acid sequence of SEQ ID NO:59. In certain embodiments, the Tim1 binding domain does not include a signal peptide. An exemplary human Tim1 signal peptide has the amino acid sequences of SEQ ID NO:60.

As used herein, an “effector domain” is an intracellular portion of a fusion protein or chimeric receptor that can directly or indirectly promote a biological or physiological response in a cell expressing the effector domain when receiving the appropriate signal. In certain embodiments, an effector domain is part of a protein or protein complex that receives a signal when bound. In other embodiments, the effector domain is part of a protein or protein complex that binds directly to a target molecule, which triggers a signal from the effector domain. For example, in response to binding of the CER to a target molecule, the effector domain may transduce a signal to the interior of the host cell, eliciting an effector function, e.g., engulfment, phagolysosome maturation, or secretion of anti-inflammatory, and/or immunosuppressive cytokines. An effector domain may directly promote a cellular response when it contains one or more signaling domains or motifs. In other embodiments, an effector domain will indirectly promote a cellular response by associating with one or more other proteins that directly promote a cellular response.

An “engulfment signaling domain” refers to an intracellular effector domain, which, upon binding of the target molecule (e.g., phosphatidylserine) targeted by the extracellular domain of a CER expressed by a host cell, activates one or more signaling pathways in the host cell resulting in engulfment, including, in specific embodiments, cytoskeletal rearrangement of the host cell and internalization of the target cell or particle associated with the target antigen. In certain embodiments, an engulfment signaling domain activates one or more signaling pathways resulting in phagocytosis of the target cell or particle. In further embodiments, an engulfment signaling domain comprises a primary engulfment signaling domain and a secondary engulfment signaling domain.

“Junction amino acids” or “junction amino acid residues” refer to one or more (e.g., about 2-20) amino acid residues between two adjacent motifs, regions or domains of a polypeptide. Junction amino acids may result from the construct design of a chimeric protein (e.g., amino acid residues resulting from the use of a restriction enzyme site during the construction of a nucleic acid molecule encoding a fusion protein).

“CD72” or “cluster of differentiation 72” is a type II membrane protein containing a C-type lectin domain and is predominantly expressed on B-lineage cells as a homodimer. CD72 is expressed in all stages of B-cell development except plasma cells. CD72 is a co-receptor of B cells and regulates B cell activation. In some embodiments, CD72 refers to human CD72. An example of a human CD72 is set forth in UniProt P21854 or comprises the amino acid sequence set forth in SEQ ID NO:61.

A “disease” is a state of health of a subject wherein the subject cannot maintain homeostasis, and wherein, if the disease is not ameliorated, then the subject's health continues to deteriorate. In contrast, a “disorder” or “undesirable condition” in a subject is a state of health in which the subject is able to maintain homeostasis, but in which the subject's state of health is less favorable than it would be in the absence of the disorder or undesirable condition. Left untreated, a disorder or undesirable condition does not necessarily result in a further decrease in the subject's state of health.

“Nucleic acid molecule” and “polynucleotide” can be in the form of RNA or DNA, which includes cDNA, genomic DNA, and synthetic DNA. A nucleic acid molecule may be composed of naturally occurring nucleotides (such as deoxyribonucleotides and ribonucleotides), analogs of naturally occurring nucleotides (e.g., α-enantiomeric forms of naturally occurring nucleotides), or a combination of both. Modified nucleotides can have “modifications in or replacement of sugar moieties, or pyrimidine or purine base moieties. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. A nucleic acid molecule may be double stranded or single stranded, and if single stranded, may be the coding strand or non-coding (anti-sense strand). A coding molecule may have a coding sequence identical to a coding sequence known in the art or may have a different coding sequence, which, as the result of the redundancy or degeneracy of the genetic code, or by splicing, can encode the same polypeptide.

“Encoding” refers to the inherent property of specific polynucleotide sequences, such as DNA, cDNA, and mRNA sequences, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a polynucleotide encodes a protein if transcription and translation of mRNA corresponding to that polynucleotide produces the protein in a cell or other biological system. Both a coding strand and a non-coding strand can be referred to as encoding a protein or other product of the polynucleotide. Unless otherwise specified, a “nucleotide sequence encoding an amino acid sequence” includes all nucleotide sequences that are degenerate versions of each other and that encode the same amino acid sequence.

As used herein, the term “endogenous” or “native” refers to a gene, protein, compound, molecule or activity that is normally present in a host or host cell, including naturally occurring variants of the gene, protein, compound, molecule, or activity.

As used herein, “homologous” or “homolog” refers to a molecule or activity from a host cell that is related by ancestry to a second gene or activity, e.g., from the same host cell, from a different host cell, from a different organism, from a different strain, from a different species. For example, a heterologous molecule or heterologous gene encoding the molecule may be homologous to a native host cell molecule or gene that encodes the molecule, respectively, and may optionally have an altered structure, sequence, expression level or any combination thereof.

As used herein, “heterologous” nucleic acid molecule, construct or sequence refers to a nucleic acid molecule or portion of a nucleic acid molecule that is not native to a host cell, but can be homologous to a nucleic acid molecule or portion of a nucleic acid molecule from the host cell. The source of the heterologous nucleic acid molecule, construct or sequence can be from a different genus or species. In some embodiments, the heterologous nucleic acid molecules are not naturally occurring. In certain embodiments, a heterologous nucleic acid molecule is added (i.e., not endogenous or native) into a host cell or host genome by, for example, conjugation, transformation, transfection, transduction, electroporation, or the like, wherein the added molecule can integrate into the host cell genome or exist as extra-chromosomal genetic material (e.g., as a plasmid or other form of self-replicating vector), and can be present in multiple copies. In addition, “heterologous” refers to a non-native enzyme, protein or other activity encoded by a non-endogenous nucleic acid molecule introduced into the host cell, even if the host cell encodes a homologous protein or activity.

As used herein, the term “engineered,” “recombinant,” “modified” or “non-natural” refers to an organism, microorganism, cell, nucleic acid molecule, or vector that has been modified by introduction of a heterologous nucleic acid molecule, or refers to a cell or microorganism that has been genetically engineered by human intervention—that is, modified by introduction of a heterologous nucleic acid molecule, or refers to a cell or microorganism that has been altered such that expression of an endogenous nucleic acid molecule or gene is controlled, deregulated or constitutive, where such alterations or modifications can be introduced by genetic engineering. Human-generated genetic alterations can include, for example, modifications introducing nucleic acid molecules (which may include an expression control element, such as a promoter) encoding one or more proteins, chimeric receptors, or enzymes, or other nucleic acid molecule additions, deletions, substitutions, or other functional disruption of or addition to a cell's genetic material. Exemplary modifications include those in coding regions or functional fragments thereof heterologous or homologous polypeptides from a reference or parent molecule. Additional exemplary modifications include, for example, modifications in non-coding regulatory regions in which the modifications alter expression of a gene or operon.

As used here, the term “transgene” refers to a gene or polynucleotide encoding a protein of interest (e.g., CAR, CER, chimeric Tim receptor) whose expression is desired in a host cell and that has been transferred by genetic engineering techniques into a cell. A transgene may encode proteins of therapeutic interest as well as proteins that are reporters, tags, markers, suicide proteins, etc. A transgene may be from a natural source, modification of a natural gene, or a recombinant or synthetic molecule. In certain embodiments, a transgene is a component of a vector.

The term “overexpressed” or “overexpression” of an antigen refers to an abnormally high level of antigen expression in a cell. Overexpressed antigen or overexpression of antigen is often associated with a disease state, such as in hematological malignancies and cells forming a solid tumor within a specific tissue or organ of a subject. Solid tumors or hematological malignancies characterized by overexpression of a tumor antigen can be determined by standard assays known in the art.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “mature polypeptide” or “mature protein” refers to a protein or polypeptide that is secreted or localized in the cell membrane or inside certain cell organelles (e.g., the endoplasmic reticulum, golgi, or endosome) and does not include an N-terminal signal peptide.

A “signal peptide”, also referred to as “signal sequence”, “leader sequence”, “leader peptide”, “localization signal” or “localization sequence”, is a short peptide (usually 15-30 amino acids in length) present at the N-terminus of newly synthesized proteins that are destined for the secretory pathway. A signal peptide typically comprises a short stretch of hydrophilic, positively charged amino acids at the N-terminus, a central hydrophobic domain of 5-15 residues, and a C-terminal region with a cleavage site for a signal peptidase. In eukaryotes, a signal peptide prompts translocation of the newly synthesized protein to the endoplasmic reticulum where it is cleaved by the signal peptidase, creating a mature protein that then proceeds to its appropriate destination. For polypeptide sequences disclosures herein, where a signal peptides is noted, the polypeptide sequence absent the signal peptide is also contemplated.

The “percent identity” between two or more nucleic acid or amino acid sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, page 10, published Mar. 13, 1997; Lehninger, Biochemistry, Second Edition; Worth Publishers, Inc. NY:NY (1975), pp. 71-77; Lewin, Genes IV, Oxford University Press, NY and Cell Press, Cambridge, MA (1990), p. 8).

The term “chimeric” refers to any nucleic acid molecule or protein that is not endogenous and comprises a combination of sequences joined or linked together that are not naturally found joined or linked together in nature. For example, a chimeric nucleic acid molecule may comprise nucleic acids encoding various domains from multiple different genes. In another example, a chimeric nucleic acid molecule may comprise regulatory sequences and coding sequences that are derived from different sources, or regulatory sequences and coding sequences that are derived from the same source but arranged in a manner different than that found in nature.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifics a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The phrase “under transcriptional control” or “operatively linked” as used herein means that a promoter is in the correct location and orientation in relation to a polynucleotide to control the initiation of transcription by RNA polymerase and expression of the polynucleotide.

A “vector” is a nucleic acid molecule that is capable of transporting another nucleic acid. Vectors may be, for example, plasmids, cosmids, viruses, or phage. The term should also be construed to include non-plasmid and non-viral compounds which facilitate transfer of nucleic acid into cells. An “expression vector” is a vector that is capable of directing the expression of a protein encoded by one or more genes carried by the vector when it is present in the appropriate environment.

In certain embodiments, the vector is a viral vector. Examples of viral vectors include, but are not limited to, adenovirus vectors, adeno-associated virus vectors, retrovirus vectors, gammaretrovirus vectors, and lentivirus vectors. “Retroviruses” are viruses having an RNA genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses. “Lentivirus” refers to a genus of retroviruses that are capable of infecting dividing and non-dividing cells. Examples of lentiviruses include, but are not limited to HIV (human immunodeficiency virus, including HIV type 1 and HIV type 2, equine infectious anemia virus, feline immunodeficiency virus (FIV), bovine immune deficiency virus (BIV), and simian immunodeficiency virus (SIV).

In other embodiments, the vector is a non-viral vector. Examples of non-viral vectors include lipid-based DNA vectors, modified mRNA (modRNA), self-amplifying mRNA, closed-ended linear duplex (CELiD) DNA, and transposon-mediated gene transfer (PiggyBac, Sleeping Beauty). Where a non-viral delivery system is used, the delivery vehicle can be a liposome. Lipid formulations can be used to introduce nucleic acids into a host cell in vitro, ex vivo, or in vivo. The nucleic acid may be encapsulated in the interior of a liposome, interspersed within the lipid bilayer of a liposome, attached to a liposome via a linking molecule that is associated with both the liposome and the nucleic acid, contained or complexed with a micelle, or otherwise associated with a lipid.

As used herein, the term “expression cassette” refers to a distinct component of a vector nucleic acid comprising at least one transgene and regulatory sequences controlling its expression (e.g., promoter, 3′UTR) in a host cell. A tandem expression cassette refers to a component of a vector nucleic acid comprising at least two transgenes under the control of the same set of regulatory sequences for tandem expression of the at least two transgenes. In certain embodiments, the tandem expression cassette comprises at least two transgenes under the control of the same promoter. In certain embodiments, the first transgene and second transgene are separated by an internal ribosome entry site (IRES), furin cleavage site, or self-cleaving viral 2A peptide to allow for co-expression of two proteins from a single mRNA.

A “particle” refers to a fragment of a cell or a small object of at least 10 nm and up to 50 μm in diameter. A particle may be derived from a living cell or organism, the environment, or synthetic. A particle can be a viral particle, prion particle, protein particle, synthetic particle, small mineral particle, or cellular debris.

As used herein, the term “engulfment” refers to a receptor-mediated process wherein endogenous or exogenous cells or particles greater than 10 nm in diameter are internalized by a phagocyte or host cell of the present disclosure. Engulfment is typically composed of multiple steps: (1) tethering of the target cell or particle via binding of an engulfment receptor to a pro-engulfment marker or antigenic marker directly or indirectly (via a bridging molecule) on a target cell or particle; and (2) internalization or engulfment of the whole target cell or particle, or a portion thereof. In certain embodiments, internalization may occur via cytoskeletal rearrangement of a phagocyte or host cell to form a phagosome, a membrane-bound compartment containing the internalized target. Engulfment may further include maturation of the phagosome, wherein the phagosome becomes increasingly acidic and fuses with lysosomes (to form a phagolysosome), whereupon the engulfed target is degraded (e.g., “phagocytosis”). Alternatively, phagosome-lysosome fusion may not be observed in engulfment. In yet another embodiment, a phagosome may regurgitate or discharge its contents to the extracellular environment before complete degradation. In some embodiments, engulfment refers to phagocytosis. In some embodiments, engulfment includes tethering of the target cell or particle by the phagocyte of host cell of the present disclosure, but not internalization. In some embodiments, engulfment includes tethering of the target cell or particle by the phagocyte of host cell of the present disclosure and internalization of part of the target cell or particle.

As used herein, the term “phagocytosis” refers to an engulfment process of cells or large particles (≥0.5 μm) wherein tethering of a target cell or particle, engulfment of the target cell or particle, and degradation of the internalized target cell or particle occurs. In certain embodiments, phagocytosis comprises formation of a phagosome that encompasses the internalized target cell or particle and phagosome fusion with a lysosome to form a phagolysosome, wherein the contents therein are degraded. In certain embodiments, following binding of a CER expressed on a host cell of the present disclosure to a target antigen expressed by a target cell or particle, a phagocytic synapse is formed; an actin-rich phagocytic cup is generated at the phagocytic synapse; phagocytic arms are extended around the target cell or particle through cytoskeletal rearrangements; and ultimately, the target cell or particle is pulled into the phagocyte or host cell through force generated by motor proteins. As used herein, “phagocytosis” includes the process of “efferocytosis”, which specifically refers to the phagocytosis of apoptotic or necrotic cells in a non-inflammatory manner.

The term “immune system cell” or “immune cell” means any cell of the immune system that originates from a hematopoietic stem cell in the bone marrow. Hematopoietic stem cells give rise to two major lineages: myeloid progenitor cells (which give rise to myeloid cells such as monocytes, macrophages, dendritic cells, megakaryocytes and granulocytes) and lymphoid progenitor cells (which give rise to lymphoid cells such as T cells, B cells and natural killer (NK) cells). Exemplary immune system cells include a CD4+ T cell, a CD8+ T cell, a CD4-CD8-double negative T cell, a γδ T cell, a regulatory T cell, a natural killer cell, and a dendritic cell. Macrophages and dendritic cells may also be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

The term “T cells” refers to cells of T cell lineage. “Cells of T cell lineage” refer to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25+ immature and inactivated T cells; cells that have undergone CD4 or CD8 linage commitment; thymocyte progenitor cells that are CD4⁺CD8⁺ double positive; single positive CD4⁺ or CD8⁺; TCRαβ or TCR γδ; or mature and functional or activated T cells. The term “T cells” encompasses naïve T cells (CD45 RA+, CCR7+, CD62L+, CD27+, CD45RO−), central memory T cells (CD45RO⁺, CD62L⁺, CD8⁺), effector memory T cells (CD45RA+, CD45RO−, CCR7−, CD62L−, CD27−), mucosal-associated invariant T (MAIT) cells, Tregs, natural killer T cells, and tissue resident T cells.

The term “B cells” refers to cells of the B cell lineage. “Cells of B cell lineage” refers to cells that show at least one phenotypic characteristic of a B cell or a precursor or progenitor thereof that distinguishes the cells from other lymphoid cells, and cells of the erythroid or myeloid lineages. Such phenotypic characteristics can include expression of one or more proteins specific for B cells (e.g., CD19⁺, CD72+, CD24+, CD20⁺), or a physiological, morphological, functional, or immunological feature specific for a B cell. For example, cells of the B cell lineage may be progenitor or precursor cells committed to the B cell lineage (e.g., pre-pro-B cells, pro-B cells, and pre-B cells); immature and inactivated B cells or mature and functional or activated B cells. Thus, “B cells” encompass naïve B cells, plasma cells, regulatory B cells, marginal zone B cells, follicular B cells, lymphoplasmacytoid cells, plasmablast cells, and memory B cells (e.g., CD27⁺, IgD⁻).

The term “cytotoxic activity,” also referred to as “cytolytic activity,” with respect to a cell (e.g., T cell) that expresses an immune receptor (e.g., TCR) on its surface, means that upon antigen-specific signaling (e.g., via the TCR) the cell induces a target cell to undergo apoptosis. In some embodiments, a cytotoxic cell may induce apoptosis in a target cell via the release of cytotoxins, such as perforin, granzyme, and granulysin, from granules. Perforins insert into the target cell membrane and form pores that allow water and salts to rapidly enter the target cell. Granzymes are serine proteases that induce apoptosis in the target cell. Granulysin is also capable of forming pores in the target cell membrane and is a proinflammatory molecule. In some embodiments, a cytotoxic cell may induce apoptosis in a target cell via interaction of Fas ligand, which is upregulated on T cell following antigen-specific signaling, with Fas molecules expressed on the target cell. Fas is an apoptosis-signaling receptor molecule on the surface of a number of different cells.

A “disease” is a state of health of a subject wherein the subject cannot maintain homeostasis, and wherein, if the disease is not ameliorated, then the subject's health continues to deteriorate. In contrast, a “disorder” or “undesirable condition” in a subject is a state of health in which the subject is able to maintain homeostasis, but in which the subject's state of health is less favorable than it would be in the absence of the disorder or undesirable condition. Left untreated, a disorder or undesirable condition does not necessarily result in a further decrease in the subject's state of health.

The term “cancer” as used herein is defined as disease characterized by the rapid and uncontrolled growth of aberrant cells. The aberrant cells may form solid tumors or constitute a hematological malignancy. Cancer cells can spread locally or through the bloodstream and lymphatic system to other parts of the body. Examples of various cancers include, but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “subject.” “patient” and “individual” are used interchangeably herein and are intended to include living organisms in which an immune response can be elicited (e.g., mammals). Examples of subjects include humans, primates, cows, horses, goats, sheep, dogs, cats, mice, rats, rabbits, guinea pigs, pigs, and transgenic species thereof.

“Adoptive cellular immunotherapy” or “adoptive immunotherapy” refers to the administration of naturally occurring or genetically engineered, disease antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

“Autologous” refers to a graft (e.g., organ, tissue, cells) derived from the same subject to which it is later to be re-introduced.

“Allogeneic” refers to a graft derived from a different subject of the same species.

A “therapeutically effective amount” or “effective amount” of a chimeric protein or cell expressing a chimeric protein of this disclosure (e.g., a tandem expression cassette or a cell expressing a tandem expression cassette) refers to that amount of protein or cells sufficient to result in amelioration of one or more symptoms of the disease, disorder, or undesired condition being treated. When referring to an individual active ingredient or a cell expressing a single active ingredient, administered alone, a therapeutically effective dose refers to the effects of that ingredient or cell expressing that ingredient alone. When referring to a combination, a therapeutically effective dose refers to the combined amounts of active ingredients or combined adjunctive active ingredient with a cell expressing an active ingredient that results in a therapeutic effect, whether administered serially or simultaneously.

“Treat” or “treatment” or “ameliorate” refers to medical management of a disease, disorder, or undesired condition of a subject. In general, an appropriate dose or treatment regimen comprising a host cell expressing a chimeric protein of this disclosure is administered in an amount sufficient to elicit a therapeutic or prophylactic benefit. Therapeutic or prophylactic/preventive benefit includes improved clinical outcome; lessening or alleviation of symptoms associated with a disease, disorder, or undesired condition; decreased occurrence of symptoms; improved quality of life; longer disease-free status; diminishment of extent of disease, disorder, or undesired condition; stabilization of disease state; delay of disease progression; remission; survival; prolonged survival; or any combination thereof.

The term “anti-tumor effect” refers to a biological effect which can be manifested by a decrease in tumor volume, a decrease in the number of tumor cells, a decrease in the number of metastases, an increase in life expectancy, or amelioration of various physiological symptoms associated with a cancerous condition. An “anti-tumor effect” can also be manifested by prevention of a hematological malignancy or tumor formation.

Additional definitions are provided throughout the present disclosure.

### I. Anti-CD72 Chimeric Antigen Receptors

The present disclosure provides a chimeric antigen receptor comprising an extracellular domain comprising a binding domain that specifically binds to CD72; an intracellular signaling domain, wherein the intracellular signaling domain comprises an immunoreceptor tyrosine-based activation motif (ITAM); and a transmembrane domain connecting the extracellular domain and intracellular signaling domain.

Binding domains suitable for use in CARs of the present disclosure include any antigen-binding polypeptide. A binding domain may comprise an antibody or antigen binding fragment thereof, including for example, a full length heavy chain, Fab fragment, Fab′, F(ab′)2, sFv, VH domain, VL domain, dAb, VHH, CDR, and scFv. In certain embodiments, a CAR binding domain is murine, chimeric, human, or humanized.

In some embodiments, the binding domain comprises:
(i) a heavy chain variable (VH) region, wherein the VH region comprises a heavy chain complementarity determining region 1 (HCDR-1) comprising the amino acid sequence set forth in SEQ ID NO:1; a heavy chain complementarity determining region 2 (HCDR-2) comprising the amino acid sequence set forth in SEQ ID NO:2; and a heavy chain complementarity determining region 3 (HCDR-3) comprising the amino acid sequence set forth in SEQ ID NO:3; and a light chain variable (VL) region, wherein the VL region comprises a light chain complementarity determining region 1 (LCDR-1) comprising the amino acid sequence set forth in SEQ ID NO:4; a light chain complementarity determining region 2 (LCDR-2) comprising the amino acid sequence set forth in SEQ ID NO:5; and a light chain complementarity determining region 3 (LCDR-3) comprising the amino acid sequence set forth in SEQ ID NO:6;
(ii) a heavy chain variable (VH) region, wherein the VH region comprises a heavy chain complementarity determining region 1 (HCDR-1) comprising the amino acid sequence set forth in SEQ ID NO:7; a heavy chain complementarity determining region 2 (HCDR-2) comprising the amino acid sequence set forth in SEQ ID NO:8; and a heavy chain complementarity determining region 3 (HCDR-3) comprising the amino acid sequence set forth in SEQ ID NO:9; and a light chain variable (VL) region, wherein the VL region comprises a light chain complementarity determining region 1 (LCDR-1) comprising the amino acid sequence set forth in SEQ ID NO: 10; a light chain complementarity determining region 2 (LCDR-2) comprising the amino acid sequence set forth in SEQ ID NO:11; and a light chain complementarity determining region 3 (LCDR-3) comprising the amino acid sequence set forth in SEQ ID NO:12;
(iii) a heavy chain variable (VH) region, wherein the VH region comprises a heavy chain complementarity determining region 1 (HCDR-1) comprising the amino acid sequence set forth in SEQ ID NO:302; a heavy chain complementarity determining region 2 (HCDR-2) comprising the amino acid sequence set forth in SEQ ID NO:303; and a heavy chain complementarity determining region 3 (HCDR-3) comprising the amino acid sequence set forth in SEQ ID NO:304; and a light chain variable (VL) region, wherein the VL region comprises a light chain complementarity determining region 1 (LCDR-1) comprising the amino acid sequence set forth in SEQ ID NO:305; a light chain complementarity determining region 2 (LCDR-2) comprising the amino acid sequence set forth in SEQ ID NO:306; and a light chain complementarity determining region 3 (LCDR-3) comprising the amino acid sequence set forth in SEQ ID NO:307;
(iv) a heavy chain variable (VH) region, wherein the VH region comprises a heavy chain complementarity determining region 1 (HCDR-1) comprising the amino acid sequence set forth in SEQ ID NO:308; a heavy chain complementarity determining region 2 (HCDR-2) comprising the amino acid sequence set forth in SEQ ID NO:309; and a heavy chain complementarity determining region 3 (HCDR-3) comprising the amino acid sequence set forth in SEQ ID NO:310; and a light chain variable (VL) region, wherein the VL region comprises a light chain complementarity determining region 1 (LCDR-1) comprising the amino acid sequence set forth in SEQ ID NO:311; a light chain complementarity determining region 2 (LCDR-2) comprising the amino acid sequence set forth in SEQ ID NO:312; and a light chain complementarity determining region 3 (LCDR-3) comprising the amino acid sequence set forth in SEQ ID NO:313;
(v) a heavy chain variable (VH) region, wherein the VH region comprises a heavy chain complementarity determining region 1 (HCDR-1) comprising the amino acid sequence set forth in SEQ ID NO:314; a heavy chain complementarity determining region 2 (HCDR-2) comprising the amino acid sequence set forth in SEQ ID NO:315; and a heavy chain complementarity determining region 3 (HCDR-3) comprising the amino acid sequence set forth in SEQ ID NO:316; and a light chain variable (VL) region, wherein the VL region comprises a light chain complementarity determining region 1 (LCDR-1) comprising the amino acid sequence set forth in SEQ ID NO:317; a light chain complementarity determining region 2 (LCDR-2) comprising the amino acid sequence set forth in SEQ ID NO:318; and a light chain complementarity determining region 3 (LCDR-3) comprising the amino acid sequence set forth in SEQ ID NO:319;
(vi) a heavy chain variable (VH) region, wherein the VH region comprises a heavy chain complementarity determining region 1 (HCDR-1) comprising the amino acid sequence set forth in SEQ ID NO:320; a heavy chain complementarity determining region 2 (HCDR-2) comprising the amino acid sequence set forth in SEQ ID NO:321; and a heavy chain complementarity determining region 3 (HCDR-3) comprising the amino acid sequence set forth in SEQ ID NO:322; and a light chain variable (VL) region, wherein the VL region comprises a light chain complementarity determining region 1 (LCDR-1) comprising the amino acid sequence set forth in SEQ ID NO:323; a light chain complementarity determining region 2 (LCDR-2) comprising the amino acid sequence set forth in SEQ ID NO:324; and a light chain complementarity determining region 3 (LCDR-3) comprising the amino acid sequence set forth in SEQ ID NO:325;
(vii) a heavy chain variable (VH) region, wherein the VH region comprises a heavy chain complementarity determining region 1 (HCDR-1) comprising the amino acid sequence set forth in SEQ ID NO:326; a heavy chain complementarity determining region 2 (HCDR-2) comprising the amino acid sequence set forth in SEQ ID NO:327; and a heavy chain complementarity determining region 3 (HCDR-3) comprising the amino acid sequence set forth in SEQ ID NO:328; and a light chain variable (VL) region, wherein the VL region comprises a light chain complementarity determining region 1 (LCDR-1) comprising the amino acid sequence set forth in SEQ ID NO:329; a light chain complementarity determining region 2 (LCDR-2) comprising the amino acid sequence set forth in SEQ ID NO:330; and a light chain complementarity determining region 3 (LCDR-3) comprising the amino acid sequence set forth in SEQ ID NO:331;
(viii) a heavy chain variable (VH) region, wherein the VH region comprises a heavy chain complementarity determining region 1 (HCDR-1) comprising the amino acid sequence set forth in SEQ ID NO:332; a heavy chain complementarity determining region 2 (HCDR-2) comprising the amino acid sequence set forth in SEQ ID NO:333; and a heavy chain complementarity determining region 3 (HCDR-3) comprising the amino acid sequence set forth in SEQ ID NO:334; and a light chain variable (VL) region, wherein the VL region comprises a light chain complementarity determining region 1 (LCDR-1) comprising the amino acid sequence set forth in SEQ ID NO:335; a light chain complementarity determining region 2 (LCDR-2) comprising the amino acid sequence set forth in SEQ ID NO:336; and a light chain complementarity determining region 3 (LCDR-3) comprising the amino acid sequence set forth in SEQ ID NO:337;
(ix) a heavy chain variable (VH) region, wherein the VH region comprises a heavy chain complementarity determining region 1 (HCDR-1) comprising the amino acid sequence set forth in SEQ ID NO:338; a heavy chain complementarity determining region 2 (HCDR-2) comprising the amino acid sequence set forth in SEQ ID NO:339; and a heavy chain complementarity determining region 3 (HCDR-3) comprising the amino acid sequence set forth in SEQ ID NO:340; and a light chain variable (VL) region, wherein the VL region comprises a light chain complementarity determining region 1 (LCDR-1) comprising the amino acid sequence set forth in SEQ ID NO:341; a light chain complementarity determining region 2 (LCDR-2) comprising the amino acid sequence set forth in SEQ ID NO:342; and a light chain complementarity determining region 3 (LCDR-3) comprising the amino acid sequence set forth in SEQ ID NO:343; or
(x) a heavy chain variable (VH) region, wherein the VH region comprises a heavy chain complementarity determining region 1 (HCDR-1) comprising the amino acid sequence set forth in SEQ ID NO:344; a heavy chain complementarity determining region 2 (HCDR-2) comprising the amino acid sequence set forth in SEQ ID NO:345; and a heavy chain complementarity determining region 3 (HCDR-3) comprising the amino acid sequence set forth in SEQ ID NO:346; and a light chain variable (VL) region, wherein the VL region comprises a light chain complementarity determining region 1 (LCDR-1) comprising the amino acid sequence set forth in SEQ ID NO:347; a light chain complementarity determining region 2 (LCDR-2) comprising the amino acid sequence set forth in SEQ ID NO:348; and a light chain complementarity determining region 3 (LCDR-3) comprising the amino acid sequence set forth in SEQ ID NO:349.

In some embodiments, the binding domain of the CAR comprises:
(i) a VH region comprising the amino acid sequence set forth in SEQ ID NO: 13 or a sequence having at least 90%, 91%, 92%, 93%, 94%, 95%, 96%, 97%, 98%, or 99% sequence identity to SEQ ID NO: 13, and a VL region comprising the amino acid sequence set forth in SEQ ID NO:14 or a sequence having at least 90%, 91%, 92%, 93%, 94%, 95%, 96%, 97%, 98%, or 99% sequence identity to SEQ ID NO:14;
(ii) a VH region comprising the amino acid sequence set forth in SEQ ID NO: 15 or a sequence having at least 90%, 91%, 92%, 93%, 94%, 95%, 96%, 97%, 98%, or 99% sequence identity to SEQ ID NO: 15, and a VL region comprising the amino acid sequence set forth in SEQ ID NO: 16 or a sequence having at least 90%, 91%, 92%, 93%, 94%, 95%, 96%, 97%, 98%, or 99% sequence identity to SEQ ID NO:16;
(iii) a VH region comprising the amino acid sequence set forth in SEQ ID NO:350 or a sequence having at least 90%, 91%, 92%, 93%, 94%, 95%, 96%, 97%, 98%, or 99% sequence identity to SEQ ID NO:350, and a VL region comprising the amino acid sequence set forth in SEQ ID NO:351 or a sequence having at least 90%, 91%, 92%, 93%, 94%, 95%, 96%, 97%, 98%, or 99% sequence identity to SEQ ID NO:351;
(iv) a VH region comprising the amino acid sequence set forth in SEQ ID NO:352 or a sequence having at least 90%, 91%, 92%, 93%, 94%, 95%, 96%, 97%, 98%, or 99% sequence identity to SEQ ID NO:352, and a VL region comprising the amino acid sequence set forth in SEQ ID NO:353 or a sequence having at least 90%, 91%, 92%, 93%, 94%, 95%, 96%, 97%, 98%, or 99% sequence identity to SEQ ID NO:353;
(v) a VH region comprising the amino acid sequence set forth in SEQ ID NO:354 or a sequence having at least 90%, 91%, 92%, 93%, 94%, 95%, 96%, 97%, 98%, or 99% sequence identity to SEQ ID NO:354, and a VL region comprising the amino acid sequence set forth in SEQ ID NO:355 or a sequence having at least 90%, 91%, 92%, 93%, 94%, 95%, 96%, 97%, 98%, or 99% sequence identity to SEQ ID NO:355;
(vi) a VH region comprising the amino acid sequence set forth in SEQ ID NO:356 or a sequence having at least 90%, 91%, 92%, 93%, 94%, 95%, 96%, 97%, 98%, or 99% sequence identity to SEQ ID NO:356, and a VL region comprising the amino acid sequence set forth in SEQ ID NO:357 or a sequence having at least 90%, 91%, 92%, 93%, 94%, 95%, 96%, 97%, 98%, or 99% sequence identity to SEQ ID NO:357;
(vii) a VH region comprising the amino acid sequence set forth in SEQ ID NO:358 or a sequence having at least 90%, 91%, 92%, 93%, 94%, 95%, 96%, 97%, 98%, or 99% sequence identity to SEQ ID NO:358, and a VL region comprising the amino acid sequence set forth in SEQ ID NO:359 or a sequence having at least 90%, 91%, 92%, 93%, 94%, 95%, 96%, 97%, 98%, or 99% sequence identity to SEQ ID NO:359;
(viii) a VH region comprising the amino acid sequence set forth in SEQ ID NO:360 or a sequence having at least 90%, 91%, 92%, 93%, 94%, 95%, 96%, 97%, 98%, or 99% sequence identity to SEQ ID NO:360, and a VL region comprising the amino acid sequence set forth in SEQ ID NO:361 or a sequence having at least 90%, 91%, 92%, 93%, 94%, 95%, 96%, 97%, 98%, or 99% sequence identity to SEQ ID NO:361; or
(ix) a VH region comprising the amino acid sequence set forth in SEQ ID NO:362 or a sequence having at least 90%, 91%, 92%, 93%, 94%, 95%, 96%, 97%, 98%, or 99% sequence identity to SEQ ID NO:362, and a VL region comprising the amino acid sequence set forth in SEQ ID NO:363 or a sequence having at least 90%, 91%, 92%, 93%, 94%, 95%, 96%, 97%, 98%, or 99% sequence identity to SEQ ID NO:363.

In some embodiments, the VH region and VL region are joined by a flexible linker. In some embodiments, the binding domain comprises a scFv comprising a VH region, a VL region, and a flexible linker, which can be in a VH-linker-VL orientation or a VL-linker-VH orientation. In some embodiments, the flexible linker has from about 5 to about 50 amino acids in length and comprises a sequence rich in glycine, serine, and/or threonine. Exemplary linkers include linkers having (GGGGS)ₓ or (GGGS)ₓ, where x=2-5. In some embodiments, the flexible linker comprises the amino acid sequence set forth in SEQ ID NO: 19, SEQ ID NO:20, SEQ ID NO:396, SEQ ID NO:397, SEQ ID NO: 398, or SEQ ID NO:399.

In some embodiments, the binding domain comprises the amino acid sequence set forth in any one of SEQ ID NOS: 17, 18, and 364-395 or a sequence having at least 90%, 91%, 92%, 93%, 94%, 95%, 96%, 97%, 98%, or 99% sequence identity to the amino acid sequence set forth in any one of SEQ ID NOS: 17, 18, and 364-395.

In certain embodiments, the extracellular domain of CARs provided in the present disclosure optionally comprises an extracellular, non-signaling spacer or linker domain between the binding domain and the transmembrane domain. Where included, such a spacer or linker domain may position the binding domain away from the host cell surface to further enable proper cell to cell contact, binding, and activation. An extracellular spacer domain is generally located between the extracellular binding domain and the transmembrane domain of the CAR. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG₄ hinge region is described in PCT Publication No. WO 2014/031687, which hinge region is incorporated herein by reference in its entirety. In some embodiments, an extracellular spacer domain comprises a modified IgG4 hinge region having an amino acid sequence of ESKYGPPCPPCP (SEQ ID NO:21).

Other examples of hinge regions that may be used in the CARs described herein include the hinge region from the extracellular regions of type 1 membrane proteins, such as CD8a, CD4, CD28 and CD7, which may be wild-type or variants thereof. An exemplary CD8a hinge region comprises the amino acid sequence set forth in SEQ ID NO:22. An exemplary CD28 hinge region comprises the amino acid sequence set forth in SEQ ID NO:23. In some embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D.

CARs of the present disclosure comprise a transmembrane domain that connects and is positioned between the extracellular domain and the intracellular signaling domain. The transmembrane domain ranges in length from about 15 amino acids to about 30 amino acids. The transmembrane domain is a hydrophobic alpha helix that transverses the host cell membrane and anchors the CAR in the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). The transmembrane domain can be selected from the same molecule as the extracellular domain or the intracellular signaling domain (e.g., a CAR that comprises a CD28 costimulatory signaling domain and a CD28 transmembrane domain). In some embodiments, the transmembrane domain and the extracellular domain are each selected from different molecules. In some embodiments, the transmembrane domain and the intracellular signaling domain are each selected from different molecules. In yet other embodiments, the transmembrane domain, the extracellular domain, and the intracellular signaling domain are each selected from different molecules.

Exemplary transmembrane domains for use in CARs of the present disclosure include a CD28, CD2, CD4, CD8a, CD5, CD38, CD38, CD3ζ, CD9, CD16, CD22, CD25, CD27, CD33, CD37, CD40, CD45, CD64, CD79A, CD79B, CD80, CD86, CD95 (Fas), CD134 (OX40), CD137 (4-1BB), CD150 (SLAMF1), CD152 (CTLA4), CD154 (CD40L), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GAL9, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5, and Zap70 transmembrane domain. An exemplary CD8a transmembrane domain comprises an amino acid sequence of SEQ ID NO:24. An exemplary CD28 transmembrane domain comprises an amino acid sequence of SEQ ID NO:25 or SEQ ID NO:437.

The intracellular signaling domain of a CAR is an intracellular effector domain and is capable of transmitting functional signals to a cell in response to binding of the extracellular domain of the CAR to a target molecule (e.g., CD72) and activates at least one of the normal effector functions or responses of the immune cell, e.g., T cell engineered to express the CAR. In some embodiments, the CAR induces a function of a T cell such as cytolytic activity or T helper activity, such as secretion of cytokines or other factors. The intracellular signaling domain may be any portion of an intracellular signaling molecule that retains sufficient signaling activity. In some embodiments, the intracellular signaling domain is obtained from an antigen receptor component (e.g., TCR) or costimulatory molecule. In some embodiments, a full length intracellular signaling domain of an antigen receptor or costimulatory molecule is used. In some embodiments, a truncated portion of an intracellular signaling domain of an antigen receptor or costimulatory molecule is used, provided that the truncated portion retains sufficient signal transduction activity. In further embodiments, an intracellular signaling domain is a variant of a full length or truncated portion of an intracellular signaling domain of an antigen receptor co stimulatory molecule, provided that the variant retains sufficient signal transduction activity (i.e., is a functional variant).

In certain embodiments, the intracellular signaling domain of a CAR comprises an immunoreceptor tyrosine-based activation motif (ITAM) containing signaling domain. An ITAM containing signaling domain generally contains at least one (one, two, three, four, or more) ITAMs, which refer to a conserved motif of YXXL/I-X₆₋₈-YXXL/I. An ITAM containing signaling domain may initiate T cell activation signaling following antigen binding or ligand engagement. ITAM-signaling domains include, for example, intracellular signaling domains of CD3γ, CD3δ, CD3ε, CD3ζ, CD5, CD22, CD79a, CD278 (ICOS), DAP10, DAP12, FcRγ, and CD66d. Exemplary CD3ζ signaling domains that may be used in CARs of the present disclosure comprise an amino acid sequence of SEQ ID NO:26 or 27.

CAR intracellular signaling domains optionally comprise a costimulatory signaling domain, which, when activated in conjunction with a primary or classic (e.g., ITAM-driven) activation signal, promotes or enhances T cell response, such as T cell activation, cytokine production, proliferation, differentiation, survival, effector function, or combinations thereof. Costimulatory signaling domains for use in CARs include, for example, CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), CD273 (PD-L2), CD274 (PD-L1), CD278 (ICOS), DAP10, LAT, LFA-1, LIGHT, NKG2C, SLP76, TRIM, ZAP70, or any combination thereof. In a particular embodiment, the costimulatory signaling domain comprises a OX40, CD2, CD27, CD28, ICAM-1, LFA-1 (CD11a/CD18), ICOS (CD278), or 4-1BB (CD137) signaling domain. Exemplary CD28 costimulatory signaling domains that may be used in CARs of the present disclosure comprise an amino acid sequence set forth in SEQ ID NO:28 or SEQ ID NO:29 (variant containing L186G, L187G mutations of the native CD28 protein). An exemplary 4-1BB costimulatory signaling domain comprises an amino acid sequence of SEQ ID NO:30. In certain embodiments, a CAR comprises one, two, or more costimulatory signaling domains.

In some embodiments, a CAR of the present disclosure is a first generation CAR, a second generation CAR, or a third generation CAR. A first generation CAR generally has an intracellular signaling domain comprising an intracellular signaling domain of CD3ζ, FcγRI, or other ITAM-containing activating domain to provide a T cell activation signal. Second generation CARs further comprise a costimulatory signaling domain (e.g., a costimulatory signaling domain from an endogenous T cell costimulatory receptor, such as CD28, 4-1BB, or ICOS). Third generation CARs comprise an ITAM-containing activating domain, a first costimulatory signaling domain and a second costimulatory signaling domain.

In some embodiments, one or more of the extracellular domain, the binding domain, the linker, the transmembrane domain, the intracellular signaling domain, or the costimulatory domain comprises junction amino acids. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a fusion protein (e.g., amino acid residues resulting from the use of a restriction enzyme site or self-cleaving peptide sequences during the construction of a polynucleotide encoding a fusion protein). For example, a transmembrane domain of a fusion protein may have one or more junction amino acids at the amino-terminal end, carboxy-terminal end, or both.

CARs of the present disclosure may comprise polynucleotide sequences derived from any mammalian species, including humans, primates, cows, horses, goats, sheep, dogs, cats, mice, rats, rabbits, guinea pigs, pigs, transgenic species thereof, or any combination thereof. In some embodiments, the chimeric antigen receptor is murine, chimeric, human, or humanized.

An exemplary CAR according to the present disclosure comprises: an extracellular domain comprising an scFv comprising the amino acid sequence set forth in SEQ ID NO: 17 and an extracellular spacer domain comprising an IgG4 hinge, a CD28 transmembrane domain, a CD28 costimulatory signaling domain, and a CD3ζ signaling domain. In one embodiment, the CAR comprises the amino acid sequence set forth in SEQ ID NO:31 or SEQ ID NO:31 without amino acids 1-21.

An exemplary CAR according to the present disclosure comprises: an extracellular domain comprising an scFv comprising the amino acid sequence set forth in SEQ ID NO: 18 and an extracellular spacer domain comprising an IgG4 hinge, a CD28 transmembrane domain, a CD28 costimulatory signaling domain, and a CD3ζ signaling domain. In one embodiment, the CAR comprises the amino acid sequence set forth in SEQ ID NO:32 or SEQ ID NO:32 without amino acids 1-21.

An exemplary CAR according to the present disclosure comprises: an extracellular domain comprising an scFv comprising the amino acid sequence set forth in SEQ ID NO:17 and an extracellular spacer domain comprising an IgG4 hinge, a CD28 transmembrane domain, a 4-1BB costimulatory signaling domain, and a CD3ζ signaling domain. In one embodiment, the CAR comprises the amino acid sequence set forth in SEQ ID NO:33 or SEQ ID NO:33 without amino acids 1-21.

An exemplary CAR according to the present disclosure comprises: an extracellular domain comprising an scFv comprising the amino acid sequence set forth in SEQ ID NO:18 and an extracellular spacer domain comprising an IgG4 hinge, a CD28 transmembrane domain, a 4-1BB costimulatory signaling domain, and a CD3ζ signaling domain. In one embodiment, the CAR comprises the amino acid sequence set forth in SEQ ID NO:34 or SEQ ID NO:34 without amino acids 1-21.

An exemplary CAR according to the present disclosure comprises: an extracellular domain comprising an scFv comprising the amino acid sequence set forth in SEQ ID NO: 17 and an extracellular spacer domain comprising a CD28 hinge, a CD28 transmembrane domain, a CD28 costimulatory signaling domain, and a CD3ζ signaling domain. In one embodiment, the CAR comprises the amino acid sequence set forth in SEQ ID NO:35 or SEQ ID NO:35 without amino acids 1-21.

An exemplary CAR according to the present disclosure comprises: an extracellular domain comprising an scFv comprising the amino acid sequence set forth in SEQ ID NO: 18 and an extracellular spacer domain comprising a CD28 hinge, a CD28 transmembrane domain, a CD28 costimulatory signaling domain, and a CD3ζ signaling domain. In one embodiment, the CAR comprises the amino acid sequence set forth in SEQ ID NO:36 or SEQ ID NO:36 without amino acids 1-21.

An exemplary CAR according to the present disclosure comprises: an extracellular domain comprising an scFv comprising the amino acid sequence set forth in SEQ ID NO: 17 and an extracellular spacer domain comprising a CD28 hinge, a CD28 transmembrane domain, a 4-1BB costimulatory signaling domain, and a CD3ζ signaling domain. In one embodiment, the CAR comprises the amino acid sequence set forth in SEQ ID NO:37 or SEQ ID NO:37 without amino acids 1-21.

An exemplary CAR according to the present disclosure comprises: an extracellular domain comprising an scFv comprising the amino acid sequence set forth in SEQ ID NO: 18 and an extracellular spacer domain comprising a CD28 hinge, a CD28 transmembrane domain, a 4-1BB costimulatory signaling domain, and a CD3ζ signaling domain. In one embodiment, the CAR comprises the amino acid sequence set forth in SEQ ID NO:38 or SEQ ID NO:38 without amino acids 1-21.

An exemplary CAR according to the present disclosure comprises: an extracellular domain comprising an scFv comprising the amino acid sequence set forth in SEQ ID NO:17 and an extracellular spacer domain comprising an IgG4 hinge, a CD8a transmembrane domain, a CD28 costimulatory signaling domain, and a CD3ζ signaling domain. In one embodiment, the CAR comprises the amino acid sequence set forth in SEQ ID NO:39 or SEQ ID NO:39 without amino acids 1-21.

An exemplary CAR according to the present disclosure comprises: an extracellular domain comprising an scFv comprising the amino acid sequence set forth in SEQ ID NO: 18 and an extracellular spacer domain comprising an IgG4 hinge, a CD8a transmembrane domain, a CD28 costimulatory signaling domain, and a CD3ζ signaling domain. In one embodiment, the CAR comprises the amino acid sequence set forth in SEQ ID NO:40 or SEQ ID NO:40 without amino acids 1-21.

An exemplary CAR according to the present disclosure comprises: an extracellular domain comprising an scFv comprising the amino acid sequence set forth in SEQ ID NO:17 and an extracellular spacer domain comprising a CD8a hinge, a CD8a transmembrane domain, a CD28 costimulatory signaling domain, and a CD3ζ signaling domain. In one embodiment, the CAR comprises the amino acid sequence set forth in SEQ ID NO:41 or SEQ ID NO:41 without amino acids 1-21.

An exemplary CAR according to the present disclosure comprises: an extracellular domain comprising an scFv comprising the amino acid sequence set forth in SEQ ID NO: 18 and an extracellular spacer domain comprising a CD8a hinge, a CD8a transmembrane domain, a CD28 costimulatory signaling domain, and a CD3ζ signaling domain. In one embodiment, the CAR comprises the amino acid sequence set forth in SEQ ID NO:42 or SEQ ID NO:42 without amino acids 1-21.

An exemplary CAR according to the present disclosure comprises: an extracellular domain comprising an scFv comprising the amino acid sequence set forth in SEQ ID NO:17 and an extracellular spacer domain comprising an IgG4 hinge, a CD8a transmembrane domain, a 4-1BB costimulatory signaling domain, and a CD3ζ signaling domain. In one embodiment, the CAR comprises the amino acid sequence set forth in SEQ ID NO:43 or SEQ ID NO:43 without amino acids 1-21.

An exemplary CAR according to the present disclosure comprises: an extracellular domain comprising an scFv comprising the amino acid sequence set forth in SEQ ID NO:18 and an extracellular spacer domain comprising an IgG4 hinge, a CD8a transmembrane domain, a 4-1BB costimulatory signaling domain, and a CD3ζ signaling domain. In one embodiment, the CAR comprises the amino acid sequence set forth in SEQ ID NO:44 or SEQ ID NO:44 without amino acids 1-21.

An exemplary CAR according to the present disclosure comprises: an extracellular domain comprising an scFv comprising the amino acid sequence set forth in SEQ ID NO: 17 and an extracellular spacer domain comprising a CD8a hinge, a CD8a transmembrane domain, a 4-1BB costimulatory signaling domain, and a CD3ζ signaling domain. In one embodiment, the CAR comprises the amino acid sequence set forth in SEQ ID NO:45 or SEQ ID NO:45 without amino acids 1-21.

An exemplary CAR according to the present disclosure comprises: an extracellular domain comprising an scFv comprising the amino acid sequence set forth in SEQ ID NO:18 and an extracellular spacer domain comprising a CD8a hinge, a CD8a transmembrane domain, a 4-1BB costimulatory signaling domain, and a CD3ζ signaling domain. In one embodiment, the CAR comprises the amino acid sequence set forth in SEQ ID NO:46 or SEQ ID NO:46 without amino acids 1-21.

An exemplary CAR according to the present disclosure comprises: an extracellular domain comprising an scFv comprising the amino acid sequence set forth in SEQ ID NO:18 and an extracellular spacer domain comprising an IgG4 hinge, a CD28 transmembrane domain, a 4-1BB costimulatory signaling domain, and a CD3ζ signaling domain. In one embodiment, the CAR comprises the amino acid sequence set forth in SEQ ID NO:400 or SEQ ID NO:400 without amino acids 1-21.

An exemplary CAR according to the present disclosure comprises: an extracellular domain comprising an scFv comprising the amino acid sequence set forth in SEQ ID NO:18 and an extracellular spacer domain comprising an IgG4 hinge, a CD28 transmembrane domain, a 4-1BB costimulatory signaling domain, and a CD3ζ signaling domain. In one embodiment, the CAR comprises the amino acid sequence set forth in SEQ ID NO:401 or SEQ ID NO:401 without amino acids 1-20.

An exemplary CAR according to the present disclosure comprises: an extracellular domain comprising an scFv comprising the amino acid sequence set forth in SEQ ID NO:18 and an extracellular spacer domain comprising an IgG4 hinge, a CD28 transmembrane domain, a 4-1BB costimulatory signaling domain, and a CD3ζ signaling domain. In one embodiment, the CAR comprises the amino acid sequence set forth in SEQ ID NO:402 or SEQ ID NO:402 without amino acids 1-18.

An exemplary CAR according to the present disclosure comprises: an extracellular domain comprising an scFv comprising the amino acid sequence set forth in SEQ ID NO: 18 and an extracellular spacer domain comprising an IgG4 hinge, a CD28 transmembrane domain, a 4-1BB costimulatory signaling domain, and a CD3ζ signaling domain. In one embodiment, the CAR comprises the amino acid sequence set forth in SEQ ID NO:403 or SEQ ID NO:403 without amino acids 1-20.

In some embodiments, the CAR comprises the amino acid sequence of a CAR set forth in Table 1, e.g., any one of SEQ ID NOS:31-46 and 400-436, or the amino acid sequence set forth in any one of SEQ ID NOS:31-46 and 400-436 absent the signal peptide.

Exemplary sequences for signal peptides, binding domains, extracellular spacers, transmembrane domains, and intracellular signaling domains for use in CARs of the present disclosure and exemplary CAR sequences are set forth in Table 1.

TABLE 1

| Name | Amino Acid Sequence | SEQ ID NO: |
|---|---|---|
| Sc02-004 HCDR1 | GYLMS | 1 |
| Sc02-004 HCDR2 | VISYDGSNKYYADSVKG | 2 |
| Sc02-004 HCDR3 | ARRDTNLFDY | 3 |
| Sc02-004 LCDR1 | RASQSISSYLN | 4 |
| Sc02-004 LCDR2 | AASSLQS | 5 |
| Sc02-004 LCDR3 | QQSYSTPPT | 6 |
| Sc02-025 HCDR1 | SYYMH | 7 |
| Sc02-025 HCDR2 | IINPSGGGTSYAQKFQG | 8 |
| Sc02-025 HCDR3 | DYYVTYDSWFDS | 9 |
| Sc02-025 LCDR1 | QGDSLRSYYAS | 10 |
| Sc02-025 LCDR2 | GKNNRPS | 11 |
| Sc02-025 LCDR3 | NSRDSSGNHVV | 12 |
| Sc02-002 HCDR1 | GFTFSD | 302 |
| Sc02-002 HCDR2 | ISYDGSNK | 303 |
| Sc02-002 HCDR3 | AKDRGSAQGYPLDY | 304 |
| Sc02-002 LCDR1 | QSVSSTY | 305 |
| Sc02-002 LCDR2 | GAS | 306 |
| Sc02-002 LCDR3 | QQGSAFP | 3070 |
| Sc02-003 HCDR1 | GFTFSNYV | 308 |
| Sc02-003 HCDR2 | ISYDGSNK | 309 |
| Sc02-003 HCDR3 | AKSHSNMSFDY | 310 |
| Sc02-003 LCDR1 | FTEHFQ | 311 |
| Sc02-003 LCDR2 | AAS | 312 |
| Sc02-003 LCDR3 | QQSYSTP | 313 |
| Sc02-004 HCDR1 | GFTFSGYL | 314 |
| Sc02-004 HCDR2 | ISYDGSNK | 315 |
| Sc02-004 HCDR3 | ARARRDTNLFDY | 316 |
| Sc02-004 LCDR1 | QSISSY | 317 |
| Sc02-004 LCDR2 | AAS | 318 |
| Sc02-004 LCDR3 | QQSYSTPPT | 319 |
| Sc02-024 HCDR1 | GYTFTSYY | 320 |
| Sc02-024 HCDR2 | INPSGGST | 321 |
| Sc02-024 HCDR3 | ARDYVRTSHSGFD | 322 |
| Sc02-024 LCDR1 | QSISSY | 323 |
| Sc02-024 LCDR2 | AAS | 324 |
| Sc02-024 LCDR3 | QQSYSTPPT | 325 |
| Sc02-025 HCDR1 | GYTFTSYY | 326 |
| Sc02-025 HCDR2 | INPSGGGT | 327 |
| Sc02-025 HCDR3 | ARDYYVTYDSWFDS | 328 |
| Sc02-025 LCDR1 | SLRSYY | 329 |
| Sc02-025 LCDR2 | GKN | 330 |
| Sc02-025 LCDR3 | NSRDSSGNHVV | 331 |
| Sc02-041 HCDR1 | GFTFSNYV | 332 |
| Sc02-041 HCDR2 | ISYDGSNK | 333 |
| Sc02-041 HCDR3 | AKSHSNMAFDY | 334 |
| Sc02-041 LCDR1 | QSISSY | 335 |
| Sc02-041 LCDR2 | AAS | 336 |
| Sc02-041 LCDR3 | QQSYSTPPT | 337 |
| Sc02-057 HCDR1 | GYTFTSYY | 338 |
| Sc02-057 HCDR2 | INPSGGST | 339 |
| Sc02-057 HCDR3 | ARDYQLSMTSGFDY | 340 |
| Sc02-057 LCDR1 | QSISSY | 341 |
| Sc02-057 LCDR2 | AAS | 342 |
| Sc02-057 LCDR3 | QQSYSTPPT | 343 |
| Sc02-132 HCDR1 | GFTFSNYV | 344 |
| Sc02-132 HCDR2 | ISYDGSNK | 345 |
| Sc02-132 HCDR3 | VKSHSNMAFDY | 346 |
| Sc02-132 LCDR1 | QSISSY | 347 |
| Sc02-132 LCDR2 | AAS | 348 |
| Sc02-132 LCDR3 | QQSYSTPPT | 349 |
| Sc02-004 VH | LVESGGGLVQPGGSLRLSCAASGFTFSGYLMSWVRQAP | 13 |
| | GKGLEWVAVISYDGSNKYYADSVKGRFTISRDNSKNTL | |
| | YLQMDSLRAEDTAVYYCARARRDTNLFDYWGQGTLVT | |
| | V | |
| Sc02-004 VL | ELTQSPSSLSASVGDRVTITCRASQSISSYLNWYQQKPGK | 14 |
| | APKLLIYAASSLQSGVPSRFSGSGSGTDFTLTISSLQPEDF | |
| | ATYYCQQSYSTPPTFGQGTKVEI | |
| Sc02-025 VH | LVQSGAEVKKPGASVKVSCKASGYTFTSYYMHWVRQA | 15 |
| | PGQGLEWMGIINPSGGGTSYAQKFQGRVTMTRDTSTSTV | |
| | YMELSSLRSEDTAVYYCARDYYVTYDSWFDSWGQGTL | |
| | VTVS | |
| Sc02-025 VL | VSVALGQTVRITCQGDSLRSYYASWYQQKPGQAPVLVI | 16 |
| | YGKNNRPSGIPDRFSGSSSGNTASLTITGAQAEDEADYYC | |
| | NSRDSSGNHVVFGGGTKLTV | |
| Sc02-002 VH | AEVQLVESGGGVVQPGRSLRLSCAASGFTFSDYTMSWV | 350 |
| | RQAPGKGLEWVAVISYDGSNKYYADSVKGRFTISRDNS | |
| | KNTLYLQMNSLRAEDTAVYYCAKDRGSAQGYPLDYWG | |
| | QGTLVTV | |
| Sc02-002 VL | SELDIQMTQSPPTLSLSPGERATLSCRASQSVSSTYLTWY | 351 |
| | QQRPGQAPRLLIYGASSRATGIPDRFSGSGSGTDFTLTISR | |
| | LEPEDVAVYYCQQGSAFPPTFGQGTKVEIKRAAA | |
| Sc02-003 VH | AEVQLVESGGGLVQPGGSLRLPCAASGFTFSNYVMHWV | 352 |
| | RQAPGKGLEWVAVISYDGSNKYYADSVKGRFTISRDNS | |
| | KNTLYLQMNSLRAEDTAVYYCAKSHSNMSFDYWGQGT | |
| | LVTV | |
| Sc02-003 VL | SELTQSPSSLSASVGDRVTITCRASQSISSYLNWYQQKPG | 353 |
| | KAPKLLIYAASSLQSGVPSRFSGSGSGTDFTLTISSLQPED | |
| | FATYYCQQSYSTPPTFGQGTKVEIKRAAA | |
| Sc02-024 VH | AQVQLVQSGPEVKKPGASVKVSCKASGYTFTSYYMHW | 354 |
| | VRQAPGQGLEWMGIINPSGGSTSYAQKFQGRVTMTRDT | |
| | STSTVYMELSSLRSEDAAVYYCARDYVRTSHSGFDYWG | |
| | QGTLVTV | |
| Sc02-024 VL | SEIELTQSPSSLSASVGDRVTITCRASQSISSYLNWYQQKP | 355 |
| | GKAPKLLIYAASSLQSGVPSRFSGSGSGTDFTLTISSLQPE | |
| | DFATYYCQQSYSTPPTFGQGTKVEIKRAAA | |
| Sc02-025 VH | AQVQLVQSGAEVKKPGASVKVSCKASGYTFTSYYMHW | 356 |
| | VRQAPGQGLEWMGIINPSGGGTSYAQKFQGRVTMTRDT | |
| | STSTVYMELSSLRSEDTAVYYCARDYYVTYDSWFDSWG | |
| | QGTLVTVS | |
| Sc02-025 VL | SELTQDPAVSVALGQTVRITCQGDSLRSYYASWYQQKP | 357 |
| | GQAPVLVIYGKNNRPSGIPDRFSGSSSGNTASLTITGAQA | |
| | EDEADYYCNSRDSSGNHVVFGGGTKLTVLGAAA | |
| Sc02-041 VH | AEVQLVESGGGLVQPGGSLRLPCAASGFTFSNYVMHWV | 358 |
| | RQAPGKGLEWVAVISYDGSNKYYADSVKGRFTISRDNS | |
| | KNTLYLQMNSLRAEDTAVYYCAKSHSNMAFDYWGQGT | |
| | LVTV | |
| Sc02-041 VL | SELTQSPSSLSASVGDRVTITCRASQSISSYLNWYQQKPG | 359 |
| | KAPKLLIYAASSLQSGVPSRFSGSGSGTDFTLTISSLQPED | |
| | FATYYCQQSYSTPPTFGQGTKVEIKRAAA | |
| Sc02-057 VH | AQVQLVQSGAEVKKPGASVKVSCKASGYTFTSYYMHW | 360 |
| | VRQAPGQGLEWMGIINPSGGSTSYAQKFQGRVTMTRDT | |
| | STSTVYMELSSLRSEDTAVYYCARDYQLSMTSGFDYWG | |
| | QGTLVTV | |
| Sc02-057 VL | SEIELTQSPSSLSASVGDRVTITCRASQSISSYLNWYQQKP | 361 |
| | GKAPKLLIYAASSLQSGVPSRFSGSGSGTDFTLTISSLQPE | |
| | DFATYYCQQSYSTPPTFGQGTKVEIKRAAA | |
| Sc02-132 VH | AEVQLVESGGGLVQPGGSLRLPCAASGFTFSNYVMHWV | 362 |
| | RQAPGKGLEWVAVISYDGSNKYYADSVKGRFTISRDNS | |
| | KNTLYLQMNSLRAEDTAVYYCVKSHSNMAFDYWGQGT | |
| | LVTV | |
| Sc02-132 VL | SELTQSPSSLSASVGDRVTITCRASQSISSYLNWYQQKPG | 363 |
| | KAPKLLIYAASSLQSGVPSRFSGSGSGTDFTLTISSLQPED | |
| | FATYYCQQSYSTPPTFGQGTKVEIKRAAA | |
| Sc02-004 scFv | AEVQLVESGGGLVQPGGSLRLSCAASGFTFSGYLMSWV | 17 |
| | RQAPGKGLEWVAVISYDGSNKYYADSVKGRFTISRDNS | |
| | KNTLYLQMDSLRAEDTAVYYCARARRDTNLFDYWGQG | |
| | TLVTVLEGTGGSGGTGSGTGTSELTQSPSSLSASVGDRVT | |
| | ITCRASQSISSYLNWYQQKPGKAPKLLIYAASSLQSGVPS | |
| | RFSGSGSGTDFTLTISSLQPEDFATYYCQQSYSTPPTFGQG | |
| | TKVEIKRAAA | |
| Sc02-025 scFv | AQVQLVQSGAEVKKPGASVKVSCKASGYTFTSYYMHW | 18 |
| | VRQAPGQGLEWMGIINPSGGGTSYAQKFQGRVTMTRDT | |
| | STSTVYMELSSLRSEDTAVYYCARDYYVTYDSWFDSWG | |
| | QGTLVTVSRGGGGSGGGGSGGGGSSELTQDPAVSVALG | |
| | QTVRITCQGDSLRSYYASWYQQKPGQAPVLVIYGKNNR | |
| | PSGIPDRFSGSSSGNTASLTITGAQAEDEADYYCNSRDSS | |
| | GNHVVFGGGTKLTVLGAAA | |
| sc02-002 VL-VH | SELDIQMTQSPPTLSLSPGERATLSCRASQSVSSTYLTWY | 364 |
| W linker scFv | QQRPGQAPRLLIYGASSRATGIPDRESGSGSGTDFTLTISR | |
| | LEPEDVAVYYCQQGSAFPPTFGQGTKVEIKRAAAGSTSG | |
| | SGKPGSGEGSTKGAEVQLVESGGGVVQPGRSLRLSCAAS | |
| | GFTFSDYTMSWVRQAPGKGLEWVAVISYDGSNKYYAD | |
| | SVKGRFTISRDNSKNTLYLQMNSLRAEDTAVYYCAKDR | |
| | GSAQGYPLDYWGQGTLVTV | |
| sc02-002 VH-VL | AEVQLVESGGGVVQPGRSLRLSCAASGFTFSDYTMSWV | 365 |
| W linker scFv | RQAPGKGLEWVAVISYDGSNKYYADSVKGRFTISRDNS | |
| | KNTLYLQMNSLRAEDTAVYYCAKDRGSAQGYPLDYWG | |
| | QGTLVTVGSTSGSGKPGSGEGSTKGSELDIQMTQSPPTLS | |
| | LSPGERATLSCRASQSVSSTYLTWYQQRPGQAPRLLIYG | |
| | ASSRATGIPDRFSGSGSGTDFTLTISRLEPEDVAVYYCQQ | |
| | GSAFPPTFGQGTKVEIKRAAA | |
| sc02-003 VL-VH | SELTQSPSSLSASVGDRVTITCRASQSISSYLNWYQQKPG | 366 |
| W linker scFv | KAPKLLIYAASSLQSGVPSRFSGSGSGTDFTLTISSLQPED | |
| | FATYYCQQSYSTPPTFGQGTKVEIKRAAAGSTSGSGKPG | |
| | SGEGSTKGAEVQLVESGGGLVQPGGSLRLPCAASGFTFS | |
| | NYVMHWVRQAPGKGLEWVAVISYDGSNKYYADSVKG | |
| | RFTISRDNSKNTLYLQMNSLRAEDTAVYYCAKSHSNMSF | |
| | DYWGQGTLVTV | |
| sc02-003 VH-VL | AEVQLVESGGGLVQPGGSLRLPCAASGFTFSNYVMHWV | 367 |
| W linker scFv | RQAPGKGLEWVAVISYDGSNKYYADSVKGRFTISRDNS | |
| | KNTLYLQMNSLRAEDTAVYYCAKSHSNMSFDYWGQGT | |
| | LVTVGSTSGSGKPGSGEGSTKGSELTQSPSSLSASVGDRV | |
| | TITCRASQSISSYLNWYQQKPGKAPKLLIYAASSLQSGVP | |
| | SRFSGSGSGTDFTLTISSLQPEDFATYYCQQSYSTPPTFGQ | |
| | GTKVEIKRAAA | |
| sc02-004 VL-VH | SELTQSPSSLSASVGDRVTITCRASQSISSYLNWYQQKPG | 368 |
| W linker scFv | KAPKLLIYAASSLQSGVPSRFSGSGSGTDFTLTISSLQPED | |
| | FATYYCQQSYSTPPTFGQGTKVEIKRAAAGSTSGSGKPG | |
| | SGEGSTKGAEVQLVESGGGLVQPGGSLRLSCAASGFTFS | |
| | GYLMSWVRQAPGKGLEWVAVISYDGSNKYYADSVKGR | |
| | FTISRDNSKNTLYLQMDSLRAEDTAVYYCARARRDTNLF | |
| | DYWGQGTLVTV | |
| sc02-004 VH-VL | AEVQLVESGGGLVQPGGSLRLSCAASGFTFSGYLMSWV | 369 |
| W linker scFv | RQAPGKGLEWVAVISYDGSNKYYADSVKGRFTISRDNS | |
| | KNTLYLQMDSLRAEDTAVYYCARARRDTNLFDYWGQG | |
| | TLVTVGSTSGSGKPGSGEGSTKGSELTQSPSSLSASVGDR | |
| | VTITCRASQSISSYLNWYQQKPGKAPKLLIYAASSLQSGV | |
| | PSRFSGSGSGTDFTLTISSLQPEDFATYYCQQSYSTPPTFG | |
| | QGTKVEIKRAAA | |
| sc02-024 VL-VH | SEIELTQSPSSLSASVGDRVTITCRASQSISSYLNWYQQKP | 370 |
| W linker scFv | GKAPKLLIYAASSLQSGVPSRFSGSGSGTDFTLTISSLQPE | |
| | DFATYYCQQSYSTPPTFGQGTKVEIKRAAAGSTSGSGKP | |
| | GSGEGSTKGAQVQLVQSGPEVKKPGASVKVSCKASGYT | |
| | FTSYYMHWVRQAPGQGLEWMGIINPSGGSTSYAQKFQG | |
| | RVTMTRDTSTSTVYMELSSLRSEDAAVYYCARDYVRTS | |
| | HSGFDYWGQGTLVTV | |
| sc02-024 VH-VL | AQVQLVQSGPEVKKPGASVKVSCKASGYTFTSYYMHW | 371 |
| W linker scFv | VRQAPGQGLEWMGIINPSGGSTSYAQKFQGRVTMTRDT | |
| | STSTVYMELSSLRSEDAAVYYCARDYVRTSHSGFDYWG | |
| | QGTLVTVGSTSGSGKPGSGEGSTKGSEIELTQSPSSLSAS | |
| | VGDRVTITCRASQSISSYLNWYQQKPGKAPKLLIYAASSL | |
| | QSGVPSRFSGSGSGTDFTLTISSLQPEDFATYYCQQSYSTP | |
| | PTFGQGTKVEIKRAAA | |
| sc02-025 VL-VH | SELTQDPAVSVALGQTVRITCQGDSLRSYYASWYQQKP | 372 |
| W linker scFv | GQAPVLVIYGKNNRPSGIPDRFSGSSSGNTASLTITGAQA | |
| | EDEADYYCNSRDSSGNHVVFGGGTKLTVLGAAAGSTSG | |
| | SGKPGSGEGSTKGAQVQLVQSGAEVKKPGASVKVSCKA | |
| | SGYTFTSYYMHWVRQAPGQGLEWMGIINPSGGGTSYAQ | |
| | KFQGRVTMTRDTSTSTVYMELSSLRSEDTAVYYCARDY | |
| | YVTYDSWFDSWGQGTLVTVS | |
| sc02-025 VH-VL | AQVQLVQSGAEVKKPGASVKVSCKASGYTFTSYYMHW | 373 |
| W linker scFv | VRQAPGQGLEWMGIINPSGGGTSYAQKFQGRVTMTRDT | |
| | STSTVYMELSSLRSEDTAVYYCARDYYVTYDSWFDSWG | |
| | QGTLVTVSGSTSGSGKPGSGEGSTKGSELTQDPAVSVAL | |
| | GQTVRITCQGDSLRSYYASWYQQKPGQAPVLVIYGKNN | |
| | RPSGIPDRFSGSSSGNTASLTITGAQAEDEADYYCNSRDS | |
| | SGNHVVFGGGTKLTVLGAAA | |
| sc02-041 VL-VH | SELTQSPSSLSASVGDRVTITCRASQSISSYLNWYQQKPG | 374 |
| W linker scFv | KAPKLLIYAASSLQSGVPSRFSGSGSGTDFTLTISSLQPED | |
| | FATYYCQQSYSTPPTFGQGTKVEIKRAAAGSTSGSGKPG | |
| | SGEGSTKGAEVQLVESGGGLVQPGGSLRLPCAASGFTFS | |
| | NYVMHWVRQAPGKGLEWVAVISYDGSNKYYADSVKG | |
| | RFTISRDNSKNTLYLQMNSLRAEDTAVYYCAKSHSNMA | |
| | FDYWGQGTLVTV | |
| sc02-041 VH-VL | AEVQLVESGGGLVQPGGSLRLPCAASGFTFSNYVMHWV | 375 |
| W linker scFv | RQAPGKGLEWVAVISYDGSNKYYADSVKGRFTISRDNS | |
| | KNTLYLQMNSLRAEDTAVYYCAKSHSNMAFDYWGQGT | |
| | LVTVGSTSGSGKPGSGEGSTKGSELTQSPSSLSASVGDRV | |
| | TITCRASQSISSYLNWYQQKPGKAPKLLIYAASSLQSGVP | |
| | SRFSGSGSGTDFTLTISSLQPEDFATYYCQQSYSTPPTFGQ | |
| | GTKVEIKRAAA | |
| sc02-057 VL-VH | SEIELTQSPSSLSASVGDRVTITCRASQSISSYLNWYQQKP | 376 |
| W linker scFv | GKAPKLLIYAASSLQSGVPSRFSGSGSGTDFTLTISSLQPE | |
| | DFATYYCQQSYSTPPTFGQGTKVEIKRAAAGSTSGSGKP | |
| | GSGEGSTKGAQVQLVQSGAEVKKPGASVKVSCKASGYT | |
| | FTSYYMHWVRQAPGQGLEWMGIINPSGGSTSYAQKFQG | |
| | RVTMTRDTSTSTVYMELSSLRSEDTAVYYCARDYQLSM | |
| | TSGFDYWGQGTLVTV | |
| sc02-057 VH-VL | AQVQLVQSGAEVKKPGASVKVSCKASGYTFTSYYMHW | 377 |
| W linker scFv | VRQAPGQGLEWMGIINPSGGSTSYAQKFQGRVTMTRDT | |
| | STSTVYMELSSLRSEDTAVYYCARDYQLSMTSGFDYWG | |
| | QGTLVTVGSTSGSGKPGSGEGSTKGSEIELTQSPSSLSAS | |
| | VGDRVTITCRASQSISSYLNWYQQKPGKAPKLLIYAASSL | |
| | QSGVPSRFSGSGSGTDFTLTISSLQPEDFATYYCQQSYSTP | |
| | PTFGQGTKVEIKRAAA | |
| sc02-132 VL-VH | SELTQSPSSLSASVGDRVTITCRASQSISSYLNWYQQKPG | 378 |
| W linker scFv | KAPKLLIYAASSLQSGVPSRFSGSGSGTDFTLTISSLQPED | |
| | FATYYCQQSYSTPPTFGQGTKVEIKRAAAGSTSGSGKPG | |
| | SGEGSTKGAEVQLVESGGGLVQPGGSLRLPCAASGFTFS | |
| | NYVMHWVRQAPGKGLEWVAVISYDGSNKYYADSVKG | |
| | RFTISRDNSKNTLYLQMNSLRAEDTAVYYCVKSHSNMA | |
| | FDYWGQGTLVTV | |
| sc02-132 VH-VL | AEVQLVESGGGLVQPGGSLRLPCAASGFTFSNYVMHWV | 379 |
| W linker scFv | RQAPGKGLEWVAVISYDGSNKYYADSVKGRFTISRDNS | |
| | KNTLYLQMNSLRAEDTAVYYCVKSHSNMAFDYWGQGT | |
| | LVTVGSTSGSGKPGSGEGSTKGSELTQSPSSLSASVGDRV | |
| | TITCRASQSISSYLNWYQQKPGKAPKLLIYAASSLQSGVP | |
| | SRFSGSGSGTDFTLTISSLQPEDFATYYCQQSYSTPPTFGQ | |
| | GTKVEIKRAAA | |
| sc02-002 VL-VH P | SELDIQMTQSPPTLSLSPGERATLSCRASQSVSSTYLTWY | 380 |
| 002 linker scFv | QQRPGQAPRLLIYGASSRATGIPDRESGSGSGTDFTLTISR | |
| | LEPEDVAVYYCQQGSAFPPTFGQGTKVEIKRAAALEGTG | |
| | GSGGTGSGTGT | |
| | AEVQLVESGGGVVQPGRSLRLSCAASGFTFSDYTMSWV | |
| | RQAPGKGLEWVAVISYDGSNKYYADSVKGRFTISRDNS | |
| | KNTLYLQMNSLRAEDTAVYYCAKDRGSAQGYPLDYWG | |
| | QGTLVTV | |
| sc02-002 VH-VL P | AEVQLVESGGGVVQPGRSLRLSCAASGFTFSDYTMSWV | 381 |
| 002 linker scFv | RQAPGKGLEWVAVISYDGSNKYYADSVKGRFTISRDNS | |
| | KNTLYLQMNSLRAEDTAVYYCAKDRGSAQGYPLDYWG | |
| | QGTLVTVLEGTGGSGGTGSGTGTSELDIQMTQSPPTLSLS | |
| | PGERATLSCRASQSVSSTYLTWYQQRPGQAPRLLIYGAS | |
| | SRATGIPDRESGSGSGTDFTLTISRLEPEDVAVYYCQQGS | |
| | AFPPTFGQGTKVEIKRAAA | |
| sc02-003 VL-VH P | SELTQSPSSLSASVGDRVTITCRASQSISSYLNWYQQKPG | 382 |
| 002 linker scFv | KAPKLLIYAASSLQSGVPSRFSGSGSGTDFTLTISSLQPED | |
| | FATYYCQQSYSTPPTFGQGTKVEIKRAAALEGTGGSGGT | |
| | GSGTGTAEVQLVESGGGLVQPGGSLRLPCAASGFTFSNY | |
| | VMHWVRQAPGKGLEWVAVISYDGSNKYYADSVKGRFT | |
| | ISRDNSKNTLYLQMNSLRAEDTAVYYCAKSHSNMSFDY | |
| | WGQGTLVTV | |
| sc02-003 VH-VL P | AEVQLVESGGGLVQPGGSLRLPCAASGFTFSNYVMHWV | 383 |
| 002 linker scFv | RQAPGKGLEWVAVISYDGSNKYYADSVKGRFTISRDNS | |
| | KNTLYLQMNSLRAEDTAVYYCAKSHSNMSFDYWGQGT | |
| | LVTVLEGTGGSGGTGSGTGTSELTQSPSSLSASVGDRVTI | |
| | TCRASQSISSYLNWYQQKPGKAPKLLIYAASSLQSGVPSR | |
| | FSGSGSGTDFTLTISSLQPEDFATYYCQQSYSTPPTFGQGT | |
| | KVEIKRAAA | |
| sc02-004 VL-VH P | SELTQSPSSLSASVGDRVTITCRASQSISSYLNWYQQKPG | 384 |
| 002 linker scFv | KAPKLLIYAASSLQSGVPSRFSGSGSGTDFTLTISSLQPED | |
| | FATYYCQQSYSTPPTFGQGTKVEIKRAAALEGTGGSGGT | |
| | GSGTGTAEVQLVESGGGLVQPGGSLRLSCAASGFTFSGY | |
| | LMSWVRQAPGKGLEWVAVISYDGSNKYYADSVKGRFTI | |
| | SRDNSKNTLYLQMDSLRAEDTAVYYCARARRDTNLFDY | |
| | WGQGTLVTV | |
| sc02-004 VH-VL P | AEVQLVESGGGLVQPGGSLRLSCAASGFTFSGYLMSWV | 385 |
| 002 linker scFv | RQAPGKGLEWVAVISYDGSNKYYADSVKGRFTISRDNS | |
| | KNTLYLQMDSLRAEDTAVYYCARARRDTNLFDYWGQG | |
| | TLVTVLEGTGGSGGTGSGTGTSELTQSPSSLSASVGDRVT | |
| | ITCRASQSISSYLNWYQQKPGKAPKLLIYAASSLQSGVPS | |
| | RFSGSGSGTDFTLTISSLQPEDFATYYCQQSYSTPPTFGQG | |
| | TKVEIKRAAA | |
| sc02-024 VL-VH P | SEIELTQSPSSLSASVGDRVTITCRASQSISSYLNWYQQKP | 386 |
| 024 linker scFv | GKAPKLLIYAASSLQSGVPSRFSGSGSGTDFTLTISSLQPE | |
| | DFATYYCQQSYSTPPTFGQGTKVEIKRAAASSGGGGSGG | |
| | GGSGGGGAQVQLVQSGPEVKKPGASVKVSCKASGYTFT | |
| | SYYMHWVRQAPGQGLEWMGIINPSGGSTSYAQKFQGR | |
| | VTMTRDTSTSTVYMELSSLRSEDAAVYYCARDYVRTSH | |
| | SGFDYWGQGTLVTV | |
| sc02-024 VH-VL P | AQVQLVQSGPEVKKPGASVKVSCKASGYTFTSYYMHW | 387 |
| 024 linker scFv | VRQAPGQGLEWMGIINPSGGSTSYAQKFQGRVTMTRDT | |
| | STSTVYMELSSLRSEDAAVYYCARDYVRTSHSGFDYWG | |
| | QGTLVTVSSGGGGSGGGGSGGGGSEIELTQSPSSLSASVG | |
| | DRVTITCRASQSISSYLNWYQQKPGKAPKLLIYAASSLQS | |
| | GVPSRFSGSGSGTDFTLTISSLQPEDFATYYCQQSYSTPPT | |
| | FGQGTKVEIKRAAA | |
| sc02-025 VL-VH P | SELTQDPAVSVALGQTVRITCQGDSLRSYYASWYQQKP | 388 |
| 025 linker scFv | GQAPVLVIYGKNNRPSGIPDRFSGSSSGNTASLTITGAQA | |
| | EDEADYYCNSRDSSGNHVVFGGGTKLTVLGAAARGGG | |
| | GSGGGGSGGGGS | |
| | AQVQLVQSGAEVKKPGASVKVSCKASGYTFTSYYMHW | |
| | VRQAPGQGLEWMGIINPSGGGTSYAQKFQGRVTMTRDT | |
| | STSTVYMELSSLRSEDTAVYYCARDYYVTYDSWFDSWG | |
| | QGTLVTVS | |
| sc02-025 VH-VL P | AQVQLVQSGAEVKKPGASVKVSCKASGYTFTSYYMHW | 389 |
| 025 linker scFv | VRQAPGQGLEWMGIINPSGGGTSYAQKFQGRVTMTRDT | |
| | STSTVYMELSSLRSEDTAVYYCARDYYVTYDSWFDSWG | |
| | QGTLVTVSRGGGGSGGGGSGGGGSSELTQDPAVSVALG | |
| | QTVRITCQGDSLRSYYASWYQQKPGQAPVLVIYGKNNR | |
| | PSGIPDRFSGSSSGNTASLTITGAQAEDEADYYCNSRDSS | |
| | GNHVVFGGGTKLTVLGAAA | |
| sc02-041 VL-VH P | SELTQSPSSLSASVGDRVTITCRASQSISSYLNWYQQKPG | 390 |
| 002 linker scFv | KAPKLLIYAASSLQSGVPSRFSGSGSGTDFTLTISSLQPED | |
| | FATYYCQQSYSTPPTFGQGTKVEIKRAAALEGTGGSGGT | |
| | GSGTGTAEVQLVESGGGLVQPGGSLRLPCAASGFTFSNY | |
| | VMHWVRQAPGKGLEWVAVISYDGSNKYYADSVKGRFT | |
| | ISRDNSKNTLYLQMNSLRAEDTAVYYCAKSHSNMAFDY | |
| | WGQGTLVTV | |
| sc02-041 VH-VL P | AEVQLVESGGGLVQPGGSLRLPCAASGFTFSNYVMHWV | 391 |
| 002 linker scFv | RQAPGKGLEWVAVISYDGSNKYYADSVKGRFTISRDNS | |
| | KNTLYLQMNSLRAEDTAVYYCAKSHSNMAFDYWGQGT | |
| | LVTVLEGTGGSGGTGSGTGTSELTQSPSSLSASVGDRVTI | |
| | TCRASQSISSYLNWYQQKPGKAPKLLIYAASSLQSGVPSR | |
| | FSGSGSGTDFTLTISSLQPEDFATYYCQQSYSTPPTFGQGT | |
| | KVEIKRAAA | |
| sc02-057 VL-VH P | SEIELTQSPSSLSASVGDRVTITCRASQSISSYLNWYQQKP | 392 |
| 024 linker scFv | GKAPKLLIYAASSLQSGVPSRFSGSGSGTDFTLTISSLQPE | |
| | DFATYYCQQSYSTPPTFGQGTKVEIKRAAASSGGGGSGG | |
| | GGSGGGGAQVQLVQSGAEVKKPGASVKVSCKASGYTFT | |
| | SYYMHWVRQAPGQGLEWMGIINPSGGSTSYAQKFQGR | |
| | VTMTRDTSTSTVYMELSSLRSEDTAVYYCARDYQLSMT | |
| | SGFDYWGQGTLVTV | |
| sc02-057 VH-VL P | AQVQLVQSGAEVKKPGASVKVSCKASGYTFTSYYMHW | 393 |
| 024 linker scFv | VRQAPGQGLEWMGIINPSGGSTSYAQKFQGRVTMTRDT | |
| | STSTVYMELSSLRSEDTAVYYCARDYQLSMTSGFDYWG | |
| | QGTLVTVSSGGGGSGGGGSGGGGSEIELTQSPSSLSASVG | |
| | DRVTITCRASQSISSYLNWYQQKPGKAPKLLIYAASSLQS | |
| | GVPSRFSGSGSGTDFTLTISSLQPEDFATYYCQQSYSTPPT | |
| | FGQGTKVEIKRAAA | |
| sc02-132 VL-VH P | SELTQSPSSLSASVGDRVTITCRASQSISSYLNWYQQKPG | 394 |
| 002 linker scFv | KAPKLLIYAASSLQSGVPSRFSGSGSGTDFTLTISSLQPED | |
| | FATYYCQQSYSTPPTFGQGTKVEIKRAAALEGTGGSGGT | |
| | GSGTGTAEVQLVESGGGLVQPGGSLRLPCAASGFTFSNY | |
| | VMHWVRQAPGKGLEWVAVISYDGSNKYYADSVKGRFT | |
| | ISRDNSKNTLYLQMNSLRAEDTAVYYCVKSHSNMAFDY | |
| | WGQGTLVTV | |
| sc02-132 VH-VL P | AEVQLVESGGGLVQPGGSLRLPCAASGFTFSNYVMHWV | 395 |
| 002 linker scFv | RQAPGKGLEWVAVISYDGSNKYYADSVKGRFTISRDNS | |
| | KNTLYLQMNSLRAEDTAVYYCVKSHSNMAFDYWGQGT | |
| | LVTVLEGTGGSGGTGSGTGTSELTQSPSSLSASVGDRVTI | |
| | TCRASQSISSYLNWYQQKPGKAPKLLIYAASSLQSGVPSR | |
| | FSGSGSGTDFTLTISSLQPEDFATYYCQQSYSTPPTFGQGT | |
| | KVEIKRAAA | |
| Flexible scFv linker | GGGGSGGGGSGGGGS | 19 |
| Flexible scFv linker | GTGGSGGTGSGTGTS | 20 |
| W scFv linker | GSTSGSGKPGSGEGSTKG | 396 |
| P 002 linker | LEGTGGSGGTGSGTGT | 397 |
| P 024 linker | SSGGGGSGGGGSGGGG | 398 |
| P 025 linker | RGGGGSGGGGSGGGGS | 399 |
| IgG4 hinge region | ESKYGPPCPPCP | 21 |
| (short) | | |
| CD8a hinge region | TTTPAPRPPTPAPTIASQPLSLRPEACRPAAGGAVHTRGL | 22 |
| | DFACD | |
| CD28 hinge region | IEVMYPPPYLDNEKSNGTIIHVKGKHLCPSPLFPGPSKP | 23 |
| CD8a | IYIWAPLAGTCGVLLLSLVITLYC | 24 |
| transmembrane | | |
| region | | |
| CD28 | FWVLVVVGGVLACYSLLVTVAFIIFWV | 25 |
| transmembrane | | |
| region | | |
| CD28 | MFWVLVVVGGVLACYSLLVTVAFIIFWV | 437 |
| transmembrane | | |
| domain | | |
| wildtype CD35 | RVKFSRSADAPAYQQGQNQLYNELNLGRREEYDVLDKR | 26 |
| signaling domain | RGRDPEMGGKPQRRKNPQEGLYNELQKDKMAEAYSEIG | |
| | MKGERRRGKGHDGLYQGLSTATKDTYDALHMQALPPR | |
| variant CD35 | RVKFSRSADAPAYQQGQNQLYNELNLGRREEYDVLDKR | 27 |
| signaling domain | RGRDPEMGGKPRRKNPQEGLYNELQKDKMAEAYSEIG | |
| | MKGERRRGKGHDGLYQGLSTATKDTYDALHMQALPPR | |
| wildtype CD28 | RSKRSRLLHSDYMNMTPRRPGPTRKHYQPYAPPRDFAA | 28 |
| costimulatory | YRS | |
| signaling domain | | |
| variant CD28 | RSKRSRGGHSDYMNMTPRRPGPTRKHYQPYAPPRDFAA | 29 |
| costimulatory | YRS | |
| signaling domain | | |
| with L186G/L187G | | |
| substitutions with | | |
| positions in | | |
| reference to full | | |
| length protein | | |
| 4-1BB | KRGRKKLLYIFKQPFMRPVQTTQEEDGCSCRFPEEEEGG | 30 |
| costimulatory | CEL | |
| signaling domain | | |
| pCTX206 CAR: | MLLVTSLLLCELPHPAFLLIPAEVQLVESGGGLVQPGGSL | 31 |
| GMSCFR signal | RLSCAASGFTFSGYLMSWVRQAPGKGLEWVAVISYDGS | |
| peptide (amino | NKYYADSVKGRFTISRDNSKNTLYLQMDSLRAEDTAVY | |
| acids 1-21)-sc02- | YCARARRDTNLFDYWGQGTLVTVLEGTGGSGGTGSGT | |
| 004 scFv-IgG4 | GTSELTQSPSSLSASVGDRVTITCRASQSISSYLNWYQQK | |
| Hinge-CD28 TM- | PGKAPKLLIYAASSLQSGVPSRFSGSGSGTDFTLTISSLQP | |
| CD28 ICD-CD3z | EDFATYYCQQSYSTPPTFGQGTKVEIKRAAAESKYGPPC | |
| | PPCPFWVLVVVGGVLACYSLLVTVAFIIFWVRSKRSRLL | |
| | HSDYMNMTPRRPGPTRKHYQPYAPPRDFAAYRSRVKFS | |
| | RSADAPAYQQGQNQLYNELNLGRREEYDVLDKRRGRD | |
| | PEMGGKPQRRKNPQEGLYNELQKDKMAEAYSEIGMKG | |
| | ERRRGKGHDGLYQGLSTATKDTYDALHMQALPPR | |
| | 31 | |
| pCTX208 CAR: | MLLVTSLLLCELPHPAFLLIPAQVQLVQSGAEVKKPGAS | 32 |
| GMSCFR signal | VKVSCKASGYTFTSYYMHWVRQAPGQGLEWMGIINPSG | |
| peptide (amino | GGTSYAQKFQGRVTMTRDTSTSTVYMELSSLRSEDTAV | |
| acids 1-21)-sc02- | YYCARDYYVTYDSWFDSWGQGTLVTVSRGGGGSGGGG | |
| 025 scFv-IgG4 | SGGGGSSELTQDPAVSVALGQTVRITCQGDSLRSYYASW | |
| Hinge-CD28 TM- | YQQKPGQAPVLVIYGKNNRPSGIPDRFSGSSSGNTASLTI | |
| CD28 ICD-CD3z | TGAQAEDEADYYCNSRDSSGNHVVFGGGTKLTVLGAA | |
| | AESKYGPPCPPCPFWVLVVVGGVLACYSLLVTVAFIIFW | |
| | VRSKRSRLLHSDYMNMTPRRPGPTRKHYQPYAPPRDFA | |
| | AYRSRVKFSRSADAPAYQQGQNQLYNELNLGRREEYDV | |
| | LDKRRGRDPEMGGKPQRRKNPQEGLYNELQKDKMAEA | |
| | YSEIGMKGERRRGKGHDGLYQGLSTATKDTYDALHMQ | |
| | ALPPR | |
| CAR: GMSCFR | MLLVTSLLLCELPHPAFLLIPAEVQLVESGGGLVQPGGSL | 33 |
| signal peptide | RLSCAASGFTFSGYLMSWVRQAPGKGLEWVAVISYDGS | |
| (amino acids 1-21)- | NKYYADSVKGRFTISRDNSKNTLYLQMDSLRAEDTAVY | |
| sc02-004 scFv- | YCARARRDTNLFDYWGQGTLVTVLEGTGGSGGTGSGT | |
| IgG4 Hinge- | GTSELTQSPSSLSASVGDRVTITCRASQSISSYLNWYQQK | |
| CD28 TM-4-1BB | PGKAPKLLIYAASSLQSGVPSRFSGSGSGTDFTLTISSLQP | |
| ICD-CD3z | EDFATYYCQQSYSTPPTFGQGTKVEIKRAAAESKYGPPC | |
| | PPCPFWVLVVVGGVLACYSLLVTVAFIIFWVKRGRKKLL | |
| | YIFKQPFMRPVQTTQEEDGCSCRFPEEEEGGCELRVKFSR | |
| | SADAPAYQQGQNQLYNELNLGRREEYDVLDKRRGRDPE | |
| | MGGKPQRRKNPQEGLYNELQKDKMAEAYSEIGMKGER | |
| | RRGKGHDGLYQGLSTATKDTYDALHMQALPPR | |
| CAR: GMSCFR | MLLVTSLLLCELPHPAFLLIPAQVQLVQSGAEVKKPGAS | 34 |
| signal peptide | VKVSCKASGYTFTSYYMHWVRQAPGQGLEWMGIINPSG | |
| (amino acids 1-21)- | GGTSYAQKFQGRVTMTRDTSTSTVYMELSSLRSEDTAV | |
| sc02-025 scFv- | YYCARDYYVTYDSWFDSWGQGTLVTVSRGGGGSGGGG | |
| IgG4 Hinge- | SGGGGSSELTQDPAVSVALGQTVRITCQGDSLRSYYASW | |
| CD28 TM-4-1BB | YQQKPGQAPVLVIYGKNNRPSGIPDRFSGSSSGNTASLTI | |
| ICD-CD3z | TGAQAEDEADYYCNSRDSSGNHVVFGGGTKLTVLGAA | |
| | AESKYGPPCPPCPFWVLVVVGGVLACYSLLVTVAFIIFW | |
| | VKRGRKKLLYIFKQPFMRPVQTTQEEDGCSCRFPEEEEGG | |
| | CELRVKFSRSADAPAYQQGQNQLYNELNLGRREEYDVL | |
| | DKRRGRDPEMGGKPQRRKNPQEGLYNELQKDKMAEAY | |
| | SEIGMKGERRRGKGHDGLYQGLSTATKDTYDALHMQA | |
| | LPPR | |
| CAR: GMSCFR | MLLVTSLLLCELPHPAFLLIPAEVQLVESGGGLVQPGGSL | 35 |
| signal peptide | RLSCAASGFTFSGYLMSWVRQAPGKGLEWVAVISYDGS | |
| (amino acids 1-21)- | NKYYADSVKGRFTISRDNSKNTLYLQMDSLRAEDTAVY | |
| sc02-004 scFv- | YCARARRDTNLFDYWGQGTLVTVLEGTGGSGGTGSGT | |
| CD28 Hinge- | GTSELTQSPSSLSASVGDRVTITCRASQSISSYLNWYQQK | |
| CD28 TM-CD28 | PGKAPKLLIYAASSLQSGVPSRFSGSGSGTDFTLTISSLQP | |
| ICD-CD3z | EDFATYYCQQSYSTPPTFGQGTKVEIKRAAAIEVMYPPP | |
| | YLDNEKSNGTIIHVKGKHLCPSPLFPGPSKPFWVLVVVG | |
| | GVLACYSLLVTVAFIIFWVRSKRSRLLHSDYMNMTPRRP | |
| | GPTRKHYQPYAPPRDFAAYRSRVKFSRSADAPAYQQGQ | |
| | NQLYNELNLGRREEYDVLDKRRGRDPEMGGKPQRRKN | |
| | PQEGLYNELQKDKMAEAYSEIGMKGERRRGKGHDGLY | |
| | QGLSTATKDTYDALHMQALPPR | |
| CAR: GMSCFR | MLLVTSLLLCELPHPAFLLIPAQVQLVQSGAEVKKPGAS | 36 |
| signal peptide | VKVSCKASGYTFTSYYMHWVRQAPGQGLEWMGIINPSG | |
| (amino acids 1-21)- | GGTSYAQKFQGRVTMTRDTSTSTVYMELSSLRSEDTAV | |
| sc02-025 scFv- | YYCARDYYVTYDSWFDSWGQGTLVTVSRGGGGSGGGG | |
| CD28 Hinge- | SGGGGSSELTQDPAVSVALGQTVRITCQGDSLRSYYASW | |
| CD28 TM-CD28 | YQQKPGQAPVLVIYGKNNRPSGIPDRESGSSSGNTASLTI | |
| ICD-CD3z | TGAQAEDEADYYCNSRDSSGNHVVFGGGTKLTVLGAA | |
| | AIEVMYPPPYLDNEKSNGTIIHVKGKHLCPSPLFPGPSKPF | |
| | WVLVVVGGVLACYSLLVTVAFIIFWVRSKRSRLLHSDY | |
| | MNMTPRRPGPTRKHYQPYAPPRDFAAYRSRVKFSRSAD | |
| | APAYQQGQNQLYNELNLGRREEYDVLDKRRGRDPEMG | |
| | GKPQRRKNPQEGLYNELQKDKMAEAYSEIGMKGERRR | |
| | GKGHDGLYQGLSTATKDTYDALHMQALPPR | |
| CAR: GMSCFR | MLLVTSLLLCELPHPAFLLIPAEVQLVESGGGLVQPGGSL | 37 |
| signal peptide | RLSCAASGFTFSGYLMSWVRQAPGKGLEWVAVISYDGS | |
| (amino acids 1-21)- | NKYYADSVKGRFTISRDNSKNTLYLQMDSLRAEDTAVY | |
| sc02-004 scFv- | YCARARRDTNLFDYWGQGTLVTVLEGTGGSGGTGSGT | |
| CD28 Hinge- | GTSELTQSPSSLSASVGDRVTITCRASQSISSYLNWYQQK | |
| CD28 TM-4-1BB | PGKAPKLLIYAASSLQSGVPSRFSGSGSGTDFTLTISSLQP | |
| ICD-CD3z | EDFATYYCQQSYSTPPTFGQGTKVEIKRAAAIEVMYPPP | |
| | YLDNEKSNGTIIHVKGKHLCPSPLFPGPSKPFWVLVVVG | |
| | GVLACYSLLVTVAFIIFWVKRGRKKLLYIFKQPFMRPVQT | |
| | TQEEDGCSCRFPEEEEGGCELRVKESRSADAPAYQQGQN | |
| | QLYNELNLGRREEYDVLDKRRGRDPEMGGKPQRRKNP | |
| | QEGLYNELQKDKMAEAYSEIGMKGERRRGKGHDGLYQ | |
| | GLSTATKDTYDALHMQALPPR | |
| CAR: GMSCFR | MLLVTSLLLCELPHPAFLLIPAQVQLVQSGAEVKKPGAS | 38 |
| signal peptide- | VKVSCKASGYTFTSYYMHWVRQAPGQGLEWMGIINPSG | |
| (amino acids 1-21)- | GGTSYAQKFQGRVTMTRDTSTSTVYMELSSLRSEDTAV | |
| sc02-025 scFv- | YYCARDYYVTYDSWFDSWGQGTLVTVSRGGGGSGGGG | |
| CD28 Hinge- | SGGGGSSELTQDPAVSVALGQTVRITCQGDSLRSYYASW | |
| CD28 TM-4-1BB | YQQKPGQAPVLVIYGKNNRPSGIPDRFSGSSSGNTASLTI | |
| ICD-CD3z | TGAQAEDEADYYCNSRDSSGNHVVFGGGTKLTVLGAA | |
| | AIEVMYPPPYLDNEKSNGTIIHVKGKHLCPSPLFPGPSKPF | |
| | WVLVVVGGVLACYSLLVTVAFIIFWVKRGRKKLLYIFKQ | |
| | PFMRPVQTTQEEDGCSCRFPEEEEGGCELRVKFSRSADAP | |
| | AYQQGQNQLYNELNLGRREEYDVLDKRRGRDPEMGGK | |
| | PQRRKNPQEGLYNELQKDKMAEAYSEIGMKGERRRGK | |
| | GHDGLYQGLSTATKDTYDALHMQALPPR | |
| CAR: GMSCFR | MLLVTSLLLCELPHPAFLLIPAEVQLVESGGGLVQPGGSL | 39 |
| signal peptide | RLSCAASGFTFSGYLMSWVRQAPGKGLEWVAVISYDGS | |
| (amino acids 1-21)- | NKYYADSVKGRFTISRDNSKNTLYLQMDSLRAEDTAVY | |
| sc02-004 scFv- | YCARARRDTNLFDYWGQGTLVTVLEGTGGSGGTGSGT | |
| IgG4 Hinge- | GTSELTQSPSSLSASVGDRVTITCRASQSISSYLNWYQQK | |
| CD8a TM-CD28 | PGKAPKLLIYAASSLQSGVPSRFSGSGSGTDFTLTISSLQP | |
| ICD-CD3z | EDFATYYCQQSYSTPPTFGQGTKVEIKRAAAESKYGPPC | |
| | PPCPIYIWAPLAGTCGVLLLSLVITLYCRSKRSRLLHSDYMN | |
| | MTPRRPGPTRKHYQPYAPPRDFAAYRSRVKFSRSADAPA | |
| | YQQGQNQLYNELNLGRREEYDVLDKRRGRDPEMGGKP | |
| | QRRKNPQEGLYNELQKDKMAEAYSEIGMKGERRRGKG | |
| | HDGLYQGLSTATKDTYDALHMQALPPR | |
| CAR: GMSCFR | MLLVTSLLLCELPHPAFLLIPAQVQLVQSGAEVKKPGAS | 40 |
| signal peptide | VKVSCKASGYTFTSYYMHWVRQAPGQGLEWMGIINPSG | |
| (amino acids 1-21)- | GGTSYAQKFQGRVTMTRDTSTSTVYMELSSLRSEDTAV | |
| sc02-025 scFv- | YYCARDYYVTYDSWFDSWGQGTLVTVSRGGGGSGGGG | |
| IgG4 Hinge- | SGGGGSSELTQDPAVSVALGQTVRITCQGDSLRSYYASW | |
| CD8a TM-CD28 | YQQKPGQAPVLVIYGKNNRPSGIPDRFSGSSSGNTASLTI | |
| ICD-CD3z | TGAQAEDEADYYCNSRDSSGNHVVFGGGTKLTVLGAA | |
| | AESKYGPPCPPCPIYIWAPLAGTCGVLLLSLVITLYCRSKRSR | |
| | LLHSDYMNMTPRRPGPTRKHYQPYAPPRDFAAYRSRVK | |
| | FSRSADAPAYQQGQNQLYNELNLGRREEYDVLDKRRGR | |
| | DPEMGGKPQRRKNPQEGLYNELQKDKMAEAYSEIGMK | |
| | GERRRGKGHDGLYQGLSTATKDTYDALHMQALPPR | |
| CAR: GMSCFR | MLLVTSLLLCELPHPAFLLIPAEVQLVESGGGLVQPGGSL | 41 |
| signal peptide | RLSCAASGFTFSGYLMSWVRQAPGKGLEWVAVISYDGS | |
| (amino acids 1-21)- | NKYYADSVKGRFTISRDNSKNTLYLQMDSLRAEDTAVY | |
| sc02-004 scFv- | YCARARRDTNLFDYWGQGTLVTVLEGTGGSGGTGSGT | |
| CD8a Hinge- | GTSELTQSPSSLSASVGDRVTITCRASQSISSYLNWYQQK | |
| CD8a TM-CD28 | PGKAPKLLIYAASSLQSGVPSRFSGSGSGTDFTLTISSLQP | |
| ICD-CD3z | EDFATYYCQQSYSTPPTFGQGTKVEIKRAAATTTPAPRPP | |
| | TPAPTIASQPLSLRPEACRPAAGGAVHTRGLDFACDIYIWAP | |
| | LAGTCGVLLLSLVITLYCRSKRSRLLHSDYMNMTPRRPGPT | |
| | RKHYQPYAPPRDFAAYRSRVKFSRSADAPAYQQGQNQL | |
| | YNELNLGRREEYDVLDKRRGRDPEMGGKPQRRKNPQE | |
| | GLYNELQKDKMAEAYSEIGMKGERRRGKGHDGLYQGL | |
| | STATKDTYDALHMQALPPR | |
| CAR: GMSCFR | MLLVTSLLLCELPHPAFLLIPAQVQLVQSGAEVKKPGAS | 42 |
| signal peptide | VKVSCKASGYTFTSYYMHWVRQAPGQGLEWMGIINPSG | |
| (amino acids 1-21)- | GGTSYAQKFQGRVTMTRDTSTSTVYMELSSLRSEDTAV | |
| sc02-025 scFv- | YYCARDYYVTYDSWFDSWGQGTLVTVSRGGGGSGGGG | |
| CD8a Hinge- | SGGGGSSELTQDPAVSVALGQTVRITCQGDSLRSYYASW | |
| CD8a TM-CD28 | YQQKPGQAPVLVIYGKNNRPSGIPDRFSGSSSGNTASLTI | |
| ICD-CD3z | TGAQAEDEADYYCNSRDSSGNHVVFGGGTKLTVLGAA | |
| | ATTTPAPRPPTPAPTIASQPLSLRPEACRPAAGGAVHTRGLD | |
| | FACDIYIWAPLAGTCGVLLLSLVITLYCRSKRSRLLHSDYMN | |
| | MTPRRPGPTRKHYQPYAPPRDFAAYRSRVKFSRSADAPA | |
| | YQQGQNQLYNELNLGRREEYDVLDKRRGRDPEMGGKP | |
| | QRRKNPQEGLYNELQKDKMAEAYSEIGMKGERRRGKG | |
| | HDGLYQGLSTATKDTYDALHMQALPPR | |
| CAR: GMSCFR | MLLVTSLLLCELPHPAFLLIPAEVQLVESGGGLVQPGGSL | 43 |
| signal peptide | RLSCAASGFTFSGYLMSWVRQAPGKGLEWVAVISYDGS | |
| (amino acids 1-21)- | NKYYADSVKGRFTISRDNSKNTLYLQMDSLRAEDTAVY | |
| sc02-004 scFv- | YCARARRDTNLFDYWGQGTLVTVLEGTGGSGGTGSGT | |
| IgG4 Hinge- | GTSELTQSPSSLSASVGDRVTITCRASQSISSYLNWYQQK | |
| CD8a TM-4-1BB | PGKAPKLLIYAASSLQSGVPSRFSGSGSGTDFTLTISSLQP | |
| ICD-CD3z | EDFATYYCQQSYSTPPTFGQGTKVEIKRAAAESKYGPPC | |
| | PPCPIYIWAPLAGTCGVLLLSLVITLYCKRGRKKLLYIFKQPF | |
| | MRPVQTTQEEDGCSCRFPEEEEGGCELRVKFSRSADAPA | |
| | YQQGQNQLYNELNLGRREEYDVLDKRRGRDPEMGGKP | |
| | QRRKNPQEGLYNELQKDKMAEAYSEIGMKGERRRGKG | |
| | HDGLYQGLSTATKDTYDALHMQALPPR | |
| CAR: GMSCFR | MLLVTSLLLCELPHPAFLLIPAQVQLVQSGAEVKKPGAS | 44 |
| signal peptide | VKVSCKASGYTFTSYYMHWVRQAPGQGLEWMGIINPSG | |
| (amino acids 1-21)- | GGTSYAQKFQGRVTMTRDTSTSTVYMELSSLRSEDTAV | |
| sc02-025 scFv- | YYCARDYYVTYDSWFDSWGQGTLVTVSRGGGGSGGGG | |
| IgG4 Hinge- | SGGGGSSELTQDPAVSVALGQTVRITCQGDSLRSYYASW | |
| CD8a TM-4-1BB | YQQKPGQAPVLVIYGKNNRPSGIPDRFSGSSSGNTASLTI | |
| ICD-CD3z | TGAQAEDEADYYCNSRDSSGNHVVFGGGTKLTVLGAA | |
| | AESKYGPPCPPCPIYIWAPLAGTCGVLLLSLVITLYCKRGRK | |
| | KLLYIFKQPFMRPVQTTQEEDGCSCRFPEEEEGGCELRVK | |
| | FSRSADAPAYQQGQNQLYNELNLGRREEYDVLDKRRGR | |
| | DPEMGGKPQRRKNPQEGLYNELQKDKMAEAYSEIGMK | |
| | GERRRGKGHDGLYQGLSTATKDTYDALHMQALPPR | |
| CAR: GMSCFR | MLLVTSLLLCELPHPAFLLIPAEVQLVESGGGLVQPGGSL | 45 |
| signal peptide | RLSCAASGFTFSGYLMSWVRQAPGKGLEWVAVISYDGS | |
| (amino acids 1-21)- | NKYYADSVKGRFTISRDNSKNTLYLQMDSLRAEDTAVY | |
| sc02-004 scFv- | YCARARRDTNLFDYWGQGTLVTVLEGTGGSGGTGSGT | |
| CD8a Hinge- | GTSELTQSPSSLSASVGDRVTITCRASQSISSYLNWYQQK | |
| CD8a TM-4-1BB | PGKAPKLLIYAASSLQSGVPSRFSGSGSGTDFTLTISSLQP | |
| ICD-CD3z | EDFATYYCQQSYSTPPTFGQGTKVEIKRAAATTTPAPRPP | |
| | TPAPTIASQPLSLRPEACRPAAGGAVHTRGLDFACDIYIWAP | |
| | LAGTCGVLLLSLVITLYCKRGRKKLLYIFKQPFMRPVQTTQE | |
| | EDGCSCRFPEEEEGGCELRVKFSRSADAPAYQQGQNQLY | |
| | NELNLGRREEYDVLDKRRGRDPEMGGKPQRRKNPQEGL | |
| | YNELQKDKMAEAYSEIGMKGERRRGKGHDGLYQGLST | |
| | ATKDTYDALHMQALPPR | |
| CAR: GMSCFR | MLLVTSLLLCELPHPAFLLIPAQVQLVQSGAEVKKPGAS | 46 |
| signal peptide | VKVSCKASGYTFTSYYMHWVRQAPGQGLEWMGIINPSG | |
| (amino acids 1-21)- | GGTSYAQKFQGRVTMTRDTSTSTVYMELSSLRSEDTAV | |
| sc02-025 scFv- | YYCARDYYVTYDSWFDSWGQGTLVTVSRGGGGSGGGG | |
| CD8a Hinge- | SGGGGSSELTQDPAVSVALGQTVRITCQGDSLRSYYASW | |
| CD8a TM-4-1BB | YQQKPGQAPVLVIYGKNNRPSGIPDRFSGSSSGNTASLTI | |
| ICD-CD3z | TGAQAEDEADYYCNSRDSSGNHVVFGGGTKLTVLGAA | |
| | ATTTPAPRPPTPAPTIASQPLSLRPEACRPAAGGAVHTRGLD | |
| | FACDIYIWAPLAGTCGVLLLSLVITLYCKRGRKKLLYIFKQPF | |
| | MRPVQTTQEEDGCSCRFPEEEEGGCELRVKFSRSADAPA | |
| | YQQGQNQLYNELNLGRREEYDVLDKRRGRDPEMGGKP | |
| | QRRKNPQEGLYNELQKDKMAEAYSEIGMKGERRRGKG | |
| | HDGLYQGLSTATKDTYDALHMQALPPR | |
| CAR: GMCSFR | MLLLVTSLLLCELPHPAFLLIP | 400 |
| signal peptide | EQKLISEEDLAQVQLVQSGAEVKKPGASVKVSCKASGYTFT | |
| (amino acids 1-21)- | SYYMHWVRQAPGQGLEWMGIINPSGGGTSYAQKFQGRVTM | |
| Myc tag (amino | TRDTSTSTVYMELSSLRSEDTAVYYCARDYYVTYDSWFDSWG | |
| acids 22-32)- | QGTLVTVSRGGGGSGGGGSGGGGSSELTQDPAVSVALGQT | |
| Sc02-025 scFv- | VRITCQGDSLRSYYASWYQQKPGQAPVLVIYGKNNRPSGIPD | |
| IgG4 hinge-CD28 | RFSGSSSGNTASLTITGAQAEDEADYYCNSRDSSGNHVVFGG | |
| TM-41BB-CD3z | GTKLTVLGAAAESKYGPPCPPCPMFWVLVVVGGVLACYS | |
| | LLVTVAFIIFWVKRGRKKLLYIFKQPFMRPVQTTQEEDG | |
| | CSCRFPEEEEGGCELRVKFSRSADAPAYQQGQNQLYNEL | |
| | NLGRREEYDVLDKRRGRDPEMGGKPRRKNPQEGLYNEL | |
| | QKDKMAEAYSEIGMKGERRRGKGHDGLYQGLSTATKD | |
| | TYDALHMQALPPR | |
| CAR: IgK signal | MVLQTQVFISLLLWISGAYG | 401 |
| peptide (amino | EQKLISEEDLAQVQLVQSGAEVKKPGASVKVSCKASGYTFT | |
| acids 1-20)-Myc | SYYMHWVRQAPGQGLEWMGIINPSGGGTSYAQKFQGRVTM | |
| tag (amino acids | TRDTSTSTVYMELSSLRSEDTAVYYCARDYYVTYDSWFDSWG | |
| 21-30)-Sc02-025 | QGTLVTVSRGGGGSGGGGSGGGGSSELTQDPAVSVALGQT | |
| scFv-IgG4 hinge- | VRITCQGDSLRSYYASWYQQKPGQAPVLVIYGKNNRPSGIPD | |
| CD28 TM- | RFSGSSSGNTASLTITGAQAEDEADYYCNSRDSSGNHVVFGG | |
| 41BB-CD3z | GTKLTVLGAAAESKYGPPCPPCPMFWVLVVVGGVLACYS | |
| | LLVTVAFIIFWVKRGRKKLLYIFKQPFMRPVQTTQEEDG | |
| | CSCRFPEEEEGGCELRVKFSRSADAPAYQQGQNQLYNEL | |
| | NLGRREEYDVLDKRRGRDPEMGGKPRRKNPQEGLYNEL | |
| | QKDKMAEAYSEIGMKGERRRGKGHDGLYQGLSTATKD | |
| | TYDALHMQALPPR | |
| CAR: CD8a signal | MALPVTALLLPLALLLHA | 402 |
| peptide (amino | EQKLISEEDLAQVQLVQSGAEVKKPGASVKVSCKASGYTFT | |
| acids 1-18)-Myc | SYYMHWVRQAPGQGLEWMGIINPSGGGTSYAQKFQGRVTM | |
| tag (amino acids | TRDTSTSTVYMELSSLRSEDTAVYYCARDYYVTYDSWFDSWG | |
| 19-28)-Sc02-025 | QGTLVTVSRGGGGSGGGGSGGGGSSELTQDPAVSVALGQT | |
| scFv-IgG4 hinge- | VRITCQGDSLRSYYASWYQQKPGQAPVLVIYGKNNRPSGIPD | |
| CD28 TM- | RFSGSSSGNTASLTITGAQAEDEADYYCNSRDSSGNHVVFGG | |
| 41BB-CD3z | GTKLTVLGAAAESKYGPPCPPCPMFWVLVVVGGVLACYS | |
| | LLVTVAFIIFWVKRGRKKLLYIFKQPFMRPVQTTQEEDG | |
| | CSCRFPEEEEGGCELRVKFSRSADAPAYQQGQNQLYNEL | |
| | NLGRREEYDVLDKRRGRDPEMGGKPRRKNPQEGLYNEL | |
| | QKDKMAEAYSEIGMKGERRRGKGHDGLYQGLSTATKD | |
| | TYDALHMQALPPR | |
| CAR: IL2 signal | MYRMQLLSCIALSLALVTNS | 403 |
| peptide (amino | EQKLISEEDLAQVQLVQSGAEVKKPGASVKVSCKASGYTFT | |
| acids 1-20)-Myc | SYYMHWVRQAPGQGLEWMGIINPSGGGTSYAQKFQGRVTM | |
| tag (amino acids | TRDTSTSTVYMELSSLRSEDTAVYYCARDYYVTYDSWFDSWG | |
| 21-30)-Sc02-025 | QGTLVTVSRGGGGSGGGGSGGGGSSELTQDPAVSVALGQT | |
| scFv-IgG4 hinge- | VRITCQGDSLRSYYASWYQQKPGQAPVLVIYGKNNRPSGIPD | |
| CD28 TM- | RFSGSSSGNTASLTITGAQAEDEADYYCNSRDSSGNHVVFGG | |
| 41BB-CD3z | GTKLTVLGAAAESKYGPPCPPCPMFWVLVVVGGVLACYS | |
| | LLVTVAFIIFWVKRGRKKLLYIFKQPFMRPVQTTQEEDG | |
| | CSCRFPEEEEGGCELRVKFSRSADAPAYQQGQNQLYNEL | |
| | NLGRREEYDVLDKRRGRDPEMGGKPRRKNPQEGLYNEL | |
| | QKDKMAEAYSEIGMKGERRRGKGHDGLYQGLSTATKD | |
| | TYDALHMQALPPR | |
| CAR: GMSCFR | MLLLVTSLLLCELPHPAFLLIPEQKLISEEDLAQVQLVQS | 404 |
| Signal peptide | GAEVKKPGASVKVSCKASGYTFTSYYMHWVRQAPGQG | |
| (amino acids 1-22)- | LEWMGIINPSGGGTSYAQKFQGRVTMTRDTSTSTVYME | |
| Myc tag-Sc025 | LSSLRSEDTAVYYCARDYYVTYDSWFDSWGQGTLVTVS | |
| scFv-IgG4 hinge- | RGGGGSGGGGSGGGGSSELTQDPAVSVALGQTVRITCQ | |
| CD28 TM- | GDSLRSYYASWYQQKPGQAPVLVIYGKNNRPSGIPDRFS | |
| 41BB-CD3z | GSSSGNTASLTITGAQAEDEADYYCNSRDSSGNHVVFGG | |
| | GTKLTVLGAAAESKYGPPCPPCPMFWVLVVVGGVLACY | |
| | SLLVTVAFIIFWVKRGRKKLLYIFKQPFMRPVQTTQEEDG | |
| | CSCRFPEEEEGGCELRVKFSRSADAPAYQQGQNQLYNEL | |
| | NLGRREEYDVLDKRRGRDPEMGGKPRRKNPQEGLYNEL | |
| | QKDKMAEAYSEIGMKGERRRGKGHDGLYQGLSTATKD | |
| | TYDALHMQALPPR | |
| pCTX 831 CAR | sc02-002 VL-VH W linker scFv: | 405 |
| | SELDIQMTQSPPTLSLSPGERATLSCRASQSVSSTYLTWY | |
| | QQRPGQAPRLLIYGASSRATGIPDRFSGSGSGTDFTLTISR | |
| | LEPEDVAVYYCQQGSAFPPTFGQGTKVEIKRAAAGSTSG | |
| | SGKPGSGEGSTKGAEVQLVESGGGVVQPGRSLRLSCAAS | |
| | GFTFSDYTMSWVRQAPGKGLEWVAVISYDGSNKYYAD | |
| | SVKGRFTISRDNSKNTLYLQMNSLRAEDTAVYYCAKDR | |
| | GSAQGYPLDYWGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 832 CAR | sc02-002 VH-VL W linker scFv: | 406 |
| | AEVQLVESGGGVVQPGRSLRLSCAASGFTFSDYTMSWV | |
| | RQAPGKGLEWVAVISYDGSNKYYADSVKGRFTISRDNS | |
| | KNTLYLQMNSLRAEDTAVYYCAKDRGSAQGYPLDYWG | |
| | QGTLVTVGSTSGSGKPGSGEGSTKGSELDIQMTQSPPTLS | |
| | LSPGERATLSCRASQSVSSTYLTWYQQRPGQAPRLLIYG | |
| | ASSRATGIPDRFSGSGSGTDFTLTISRLEPEDVAVYYCQQ | |
| | GSAFPPTFGQGTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 833 CAR | sc02-003 VL-VH W linker scFv: | 407 |
| | SELTQSPSSLSASVGDRVTITCRASQSISSYLNWYQQKPG | |
| | KAPKLLIYAASSLQSGVPSRFSGSGSGTDFTLTISSLQPED | |
| | FATYYCQQSYSTPPTFGQGTKVEIKRAAAGSTSGSGKPG | |
| | SGEGSTKGAEVQLVESGGGLVQPGGSLRLPCAASGFTFS | |
| | NYVMHWVRQAPGKGLEWVAVISYDGSNKYYADSVKG | |
| | RFTISRDNSKNTLYLQMNSLRAEDTAVYYCAKSHSNMSF | |
| | DYWGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 834 CAR | sc02-003 VH-VL W linker scFv: | 408 |
| | AEVQLVESGGGLVQPGGSLRLPCAASGFTFSNYVMHWV | |
| | RQAPGKGLEWVAVISYDGSNKYYADSVKGRFTISRDNS | |
| | KNTLYLQMNSLRAEDTAVYYCAKSHSNMSFDYWGQGT | |
| | LVTVGSTSGSGKPGSGEGSTKGSELTQSPSSLSASVGDRV | |
| | TITCRASQSISSYLNWYQQKPGKAPKLLIYAASSLQSGVP | |
| | SRFSGSGSGTDFTLTISSLQPEDFATYYCQQSYSTPPTFGQ | |
| | GTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 835 CAR | sc02-004 VL-VH W linker scFv: | 409 |
| | SELTQSPSSLSASVGDRVTITCRASQSISSYLNWYQQKPG | |
| | KAPKLLIYAASSLQSGVPSRFSGSGSGTDFTLTISSLQPED | |
| | FATYYCQQSYSTPPTFGQGTKVEIKRAAAGSTSGSGKPG | |
| | SGEGSTKGAEVQLVESGGGLVQPGGSLRLSCAASGFTFS | |
| | GYLMSWVRQAPGKGLEWVAVISYDGSNKYYADSVKGR | |
| | FTISRDNSKNTLYLQMDSLRAEDTAVYYCARARRDTNLF | |
| | DYWGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 836 CAR | sc02-004 VH-VL W linker scFv: | 410 |
| | AEVQLVESGGGLVQPGGSLRLSCAASGFTFSGYLMSWV | |
| | RQAPGKGLEWVAVISYDGSNKYYADSVKGRFTISRDNS | |
| | KNTLYLQMDSLRAEDTAVYYCARARRDTNLFDYWGQG | |
| | TLVTVGSTSGSGKPGSGEGSTKGSELTQSPSSLSASVGDR | |
| | VTITCRASQSISSYLNWYQQKPGKAPKLLIYAASSLQSGV | |
| | PSRFSGSGSGTDFTLTISSLQPEDFATYYCQQSYSTPPTFG | |
| | QGTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 837 CAR | sc02-024 VL-VH W linker scFv: | 411 |
| | SEIELTQSPSSLSASVGDRVTITCRASQSISSYLNWYQQKP | |
| | GKAPKLLIYAASSLQSGVPSRFSGSGSGTDFTLTISSLQPE | |
| | DFATYYCQQSYSTPPTFGQGTKVEIKRAAAGSTSGSGKP | |
| | GSGEGSTKGAQVQLVQSGPEVKKPGASVKVSCKASGYT | |
| | FTSYYMHWVRQAPGQGLEWMGIINPSGGSTSYAQKFQG | |
| | RVTMTRDTSTSTVYMELSSLRSEDAAVYYCARDYVRTS | |
| | HSGFDYWGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 838 CAR | sc02-024 VH-VL W linker scFv: | 412 |
| | AQVQLVQSGPEVKKPGASVKVSCKASGYTFTSYYMHW | |
| | VRQAPGQGLEWMGIINPSGGSTSYAQKFQGRVTMTRDT | |
| | STSTVYMELSSLRSEDAAVYYCARDYVRTSHSGFDYWG | |
| | QGTLVTVGSTSGSGKPGSGEGSTKGSEIELTQSPSSLSAS | |
| | VGDRVTITCRASQSISSYLNWYQQKPGKAPKLLIYAASSL | |
| | QSGVPSRFSGSGSGTDFTLTISSLQPEDFATYYCQQSYSTP | |
| | PTFGQGTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 839 CAR | sc02-025 VL-VH W linker scFv: | 413 |
| | SELTQDPAVSVALGQTVRITCQGDSLRSYYASWYQQKP | |
| | GQAPVLVIYGKNNRPSGIPDRFSGSSSGNTASLTITGAQA | |
| | EDEADYYCNSRDSSGNHVVFGGGTKLTVLGAAAGSTSG | |
| | SGKPGSGEGSTKGAQVQLVQSGAEVKKPGASVKVSCKA | |
| | SGYTFTSYYMHWVRQAPGQGLEWMGIINPSGGGTSYAQ | |
| | KFQGRVTMTRDTSTSTVYMELSSLRSEDTAVYYCARDY | |
| | YVTYDSWFDSWGQGTLVTVS | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 840 CAR | sc02-025 VH-VL W linker scFv: | 414 |
| | AQVQLVQSGAEVKKPGASVKVSCKASGYTFTSYYMHW | |
| | VRQAPGQGLEWMGIINPSGGGTSYAQKFQGRVTMTRDT | |
| | STSTVYMELSSLRSEDTAVYYCARDYYVTYDSWFDSWG | |
| | QGTLVTVSGSTSGSGKPGSGEGSTKGSELTQDPAVSVAL | |
| | GQTVRITCQGDSLRSYYASWYQQKPGQAPVLVIYGKNN | |
| | RPSGIPDRFSGSSSGNTASLTITGAQAEDEADYYCNSRDS | |
| | SGNHVVFGGGTKLTVLGAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 841 CAR | sc02-041 VL-VH W linker scFv: | 415 |
| | SELTQSPSSLSASVGDRVTITCRASQSISSYLNWYQQKPG | |
| | KAPKLLIYAASSLQSGVPSRFSGSGSGTDFTLTISSLQPED | |
| | FATYYCQQSYSTPPTFGQGTKVEIKRAAAGSTSGSGKPG | |
| | SGEGSTKGAEVQLVESGGGLVQPGGSLRLPCAASGFTFS | |
| | NYVMHWVRQAPGKGLEWVAVISYDGSNKYYADSVKG | |
| | RFTISRDNSKNTLYLQMNSLRAEDTAVYYCAKSHSNMA | |
| | FDYWGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 842 CAR | sc02-041 VH-VL W linker scFv: | 416 |
| | AEVQLVESGGGLVQPGGSLRLPCAASGFTFSNYVMHWV | |
| | RQAPGKGLEWVAVISYDGSNKYYADSVKGRFTISRDNS | |
| | KNTLYLQMNSLRAEDTAVYYCAKSHSNMAFDYWGQGT | |
| | LVTVGSTSGSGKPGSGEGSTKGSELTQSPSSLSASVGDRV | |
| | TITCRASQSISSYLNWYQQKPGKAPKLLIYAASSLQSGVP | |
| | SRFSGSGSGTDFTLTISSLQPEDFATYYCQQSYSTPPTFGQ | |
| | GTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 843 CAR | sc02-057 VL-VH W linker scFv: | 417 |
| | SEIELTQSPSSLSASVGDRVTITCRASQSISSYLNWYQQKP | |
| | GKAPKLLIYAASSLQSGVPSRFSGSGSGTDFTLTISSLQPE | |
| | DFATYYCQQSYSTPPTFGQGTKVEIKRAAAGSTSGSGKP | |
| | GSGEGSTKGAQVQLVQSGAEVKKPGASVKVSCKASGYT | |
| | FTSYYMHWVRQAPGQGLEWMGIINPSGGSTSYAQKFQG | |
| | RVTMTRDTSTSTVYMELSSLRSEDTAVYYCARDYQLSM | |
| | TSGFDYWGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 844 CAR | sc02-057 VH-VL W linker scFv: | 418 |
| | AQVQLVQSGAEVKKPGASVKVSCKASGYTFTSYYMHW | |
| | VRQAPGQGLEWMGIINPSGGSTSYAQKFQGRVTMTRDT | |
| | STSTVYMELSSLRSEDTAVYYCARDYQLSMTSGFDYWG | |
| | QGTLVTVGSTSGSGKPGSGEGSTKGSEIELTQSPSSLSAS | |
| | VGDRVTITCRASQSISSYLNWYQQKPGKAPKLLIYAASSL | |
| | QSGVPSRFSGSGSGTDFTLTISSLQPEDFATYYCQQSYSTP | |
| | PTFGQGTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 845 CAR | sc02-132 VL-VH W linker scFv: | 419 |
| | SELTQSPSSLSASVGDRVTITCRASQSISSYLNWYQQKPG | |
| | KAPKLLIYAASSLQSGVPSRFSGSGSGTDFTLTISSLQPED | |
| | FATYYCQQSYSTPPTFGQGTKVEIKRAAAGSTSGSGKPG | |
| | SGEGSTKGAEVQLVESGGGLVQPGGSLRLPCAASGFTFS | |
| | NYVMHWVRQAPGKGLEWVAVISYDGSNKYYADSVKG | |
| | RFTISRDNSKNTLYLQMNSLRAEDTAVYYCVKSHSNMA | |
| | FDYWGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 846 CAR | sc02-132 VH-VL W linker scFv: | 420 |
| | AEVQLVESGGGLVQPGGSLRLPCAASGFTFSNYVMHWV | |
| | RQAPGKGLEWVAVISYDGSNKYYADSVKGRFTISRDNS | |
| | KNTLYLQMNSLRAEDTAVYYCVKSHSNMAFDYWGQGT | |
| | LVTVGSTSGSGKPGSGEGSTKGSELTQSPSSLSASVGDRV | |
| | TITCRASQSISSYLNWYQQKPGKAPKLLIYAASSLQSGVP | |
| | SRFSGSGSGTDFTLTISSLQPEDFATYYCQQSYSTPPTFGQ | |
| | GTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 849 CAR | sc02-002 VL-VH P 002 linker scFv: | 421 |
| | SELDIQMTQSPPTLSLSPGERATLSCRASQSVSSTYLTWY | |
| | QQRPGQAPRLLIYGASSRATGIPDRFSGSGSGTDFTLTISR | |
| | LEPEDVAVYYCQQGSAFPPTFGQGTKVEIKRAAALEGTG | |
| | GSGGTGSGTGT | |
| | AEVQLVESGGGVVQPGRSLRLSCAASGFTFSDYTMSWV | |
| | RQAPGKGLEWVAVISYDGSNKYYADSVKGRFTISRDNS | |
| | KNTLYLQMNSLRAEDTAVYYCAKDRGSAQGYPLDYWG | |
| | QGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 850 CAR | sc02-002 VH-VL P 002 linker scFv: | 422 |
| | AEVQLVESGGGVVQPGRSLRLSCAASGFTFSDYTMSWV | |
| | RQAPGKGLEWVAVISYDGSNKYYADSVKGRFTISRDNS | |
| | KNTLYLQMNSLRAEDTAVYYCAKDRGSAQGYPLDYWG | |
| | QGTLVTVLEGTGGSGGTGSGTGTSELDIQMTQSPPTLSLS | |
| | PGERATLSCRASQSVSSTYLTWYQQRPGQAPRLLIYGAS | |
| | SRATGIPDRFSGSGSGTDFTLTISRLEPEDVAVYYCQQGS | |
| | AFPPTFGQGTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 851 CAR | sc02-003 VL-VH P 002 linker scFv: | 423 |
| | SELTQSPSSLSASVGDRVTITCRASQSISSYLNWYQQKPG | |
| | KAPKLLIYAASSLQSGVPSRFSGSGSGTDFTLTISSLQPED | |
| | FATYYCQQSYSTPPTFGQGTKVEIKRAAALEGTGGSGGT | |
| | GSGTGTAEVQLVESGGGLVQPGGSLRLPCAASGFTFSNY | |
| | VMHWVRQAPGKGLEWVAVISYDGSNKYYADSVKGRFT | |
| | ISRDNSKNTLYLQMNSLRAEDTAVYYCAKSHSNMSFDY | |
| | WGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 852 CAR | sc02-003 VH-VL P 002 linker scFv: | 424 |
| | AEVQLVESGGGLVQPGGSLRLPCAASGFTFSNYVMHWV | |
| | RQAPGKGLEWVAVISYDGSNKYYADSVKGRFTISRDNS | |
| | KNTLYLQMNSLRAEDTAVYYCAKSHSNMSFDYWGQGT | |
| | LVTVLEGTGGSGGTGSGTGTSELTQSPSSLSASVGDRVTI | |
| | TCRASQSISSYLNWYQQKPGKAPKLLIYAASSLQSGVPSR | |
| | FSGSGSGTDFTLTISSLQPEDFATYYCQQSYSTPPTFGQGT | |
| | KVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 853 CAR | sc02-004 VL-VH P 002 linker scFv: | 425 |
| | SELTQSPSSLSASVGDRVTITCRASQSISSYLNWYQQKPG | |
| | KAPKLLIYAASSLQSGVPSRFSGSGSGTDFTLTISSLQPED | |
| | FATYYCQQSYSTPPTFGQGTKVEIKRAAALEGTGGSGGT | |
| | GSGTGTAEVQLVESGGGLVQPGGSLRLSCAASGFTFSGY | |
| | LMSWVRQAPGKGLEWVAVISYDGSNKYYADSVKGRFTI | |
| | SRDNSKNTLYLQMDSLRAEDTAVYYCARARRDTNLFDY | |
| | WGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 854 CAR | sc02-004 VH-VL P 002 linker scFv: | 426 |
| | AEVQLVESGGGLVQPGGSLRLSCAASGFTFSGYLMSWV | |
| | RQAPGKGLEWVAVISYDGSNKYYADSVKGRFTISRDNS | |
| | KNTLYLQMDSLRAEDTAVYYCARARRDTNLFDYWGQG | |
| | TLVTVLEGTGGSGGTGSGTGTSELTQSPSSLSASVGDRVT | |
| | ITCRASQSISSYLNWYQQKPGKAPKLLIYAASSLQSGVPS | |
| | RFSGSGSGTDFTLTISSLQPEDFATYYCQQSYSTPPTFGQG | |
| | TKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 855 CAR | sc02-024 VL-VH P 024 linker scFv: | 427 |
| | SEIELTQSPSSLSASVGDRVTITCRASQSISSYLNWYQQKP | |
| | GKAPKLLIYAASSLQSGVPSRFSGSGSGTDFTLTISSLQPE | |
| | DFATYYCQQSYSTPPTFGQGTKVEIKRAAASSGGGGSGG | |
| | GGSGGGGAQVQLVQSGPEVKKPGASVKVSCKASGYTFT | |
| | SYYMHWVRQAPGQGLEWMGIINPSGGSTSYAQKFQGR | |
| | VTMTRDTSTSTVYMELSSLRSEDAAVYYCARDYVRTSH | |
| | SGFDYWGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 856 CAR | sc02-024 VH-VL P 024 linker scFv: | 428 |
| | AQVQLVQSGPEVKKPGASVKVSCKASGYTFTSYYMHW | |
| | VRQAPGQGLEWMGIINPSGGSTSYAQKFQGRVTMTRDT | |
| | STSTVYMELSSLRSEDAAVYYCARDYVRTSHSGFDYWG | |
| | QGTLVTVSSGGGGSGGGGSGGGGSEIELTQSPSSLSASVG | |
| | DRVTITCRASQSISSYLNWYQQKPGKAPKLLIYAASSLQS | |
| | GVPSRFSGSGSGTDFTLTISSLQPEDFATYYCQQSYSTPPT | |
| | FGQGTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 857 CAR | sc02-025 VL-VH P 025 linker scFv: | 429 |
| | SELTQDPAVSVALGQTVRITCQGDSLRSYYASWYQQKP | |
| | GQAPVLVIYGKNNRPSGIPDRFSGSSSGNTASLTITGAQA | |
| | EDEADYYCNSRDSSGNHVVFGGGTKLTVLGAAARGGG | |
| | GSGGGGSGGGGS | |
| | AQVQLVQSGAEVKKPGASVKVSCKASGYTFTSYYMHW | |
| | VRQAPGQGLEWMGIINPSGGGTSYAQKFQGRVTMTRDT | |
| | STSTVYMELSSLRSEDTAVYYCARDYYVTYDSWFDSWG | |
| | QGTLVTVS | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 858 CAR | sc02-025 VH-VL P 025 linker scFv: | 430 |
| | AQVQLVQSGAEVKKPGASVKVSCKASGYTFTSYYMHW | |
| | VRQAPGQGLEWMGIINPSGGGTSYAQKFQGRVTMTRDT | |
| | STSTVYMELSSLRSEDTAVYYCARDYYVTYDSWFDSWG | |
| | QGTLVTVSRGGGGSGGGGSGGGGSSELTQDPAVSVALG | |
| | QTVRITCQGDSLRSYYASWYQQKPGQAPVLVIYGKNNR | |
| | PSGIPDRFSGSSSGNTASLTITGAQAEDEADYYCNSRDSS | |
| | GNHVVFGGGTKLTVLGAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 859 CAR | sc02-041 VL-VH P 002 linker scFv: | 431 |
| | SELTQSPSSLSASVGDRVTITCRASQSISSYLNWYQQKPG | |
| | KAPKLLIYAASSLQSGVPSRFSGSGSGTDFTLTISSLQPED | |
| | FATYYCQQSYSTPPTFGQGTKVEIKRAAALEGTGGSGGT | |
| | GSGTGTAEVQLVESGGGLVQPGGSLRLPCAASGFTFSNY | |
| | VMHWVRQAPGKGLEWVAVISYDGSNKYYADSVKGRFT | |
| | ISRDNSKNTLYLQMNSLRAEDTAVYYCAKSHSNMAFDY | |
| | WGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 860 CAR | sc02-041 VH-VL P 002 linker scFv: | 432 |
| | AEVQLVESGGGLVQPGGSLRLPCAASGFTFSNYVMHWV | |
| | RQAPGKGLEWVAVISYDGSNKYYADSVKGRFTISRDNS | |
| | KNTLYLQMNSLRAEDTAVYYCAKSHSNMAFDYWGQGT | |
| | LVTVLEGTGGSGGTGSGTGTSELTQSPSSLSASVGDRVTI | |
| | TCRASQSISSYLNWYQQKPGKAPKLLIYAASSLQSGVPSR | |
| | FSGSGSGTDFTLTISSLQPEDFATYYCQQSYSTPPTFGQGT | |
| | KVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 861 CAR | sc02-057 VL-VH P 024 linker scFv: | 433 |
| | SEIELTQSPSSLSASVGDRVTITCRASQSISSYLNWYQQKP | |
| | GKAPKLLIYAASSLQSGVPSRFSGSGSGTDFTLTISSLQPE | |
| | DFATYYCQQSYSTPPTFGQGTKVEIKRAAASSGGGGSGG | |
| | GGSGGGGAQVQLVQSGAEVKKPGASVKVSCKASGYTFT | |
| | SYYMHWVRQAPGQGLEWMGIINPSGGSTSYAQKFQGR | |
| | VTMTRDTSTSTVYMELSSLRSEDTAVYYCARDYQLSMT | |
| | SGFDYWGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 862 CAR | sc02-057 VH-VL P 024 linker scFv: | 434 |
| | AQVQLVQSGAEVKKPGASVKVSCKASGYTFTSYYMHW | |
| | VRQAPGQGLEWMGIINPSGGSTSYAQKFQGRVTMTRDT | |
| | STSTVYMELSSLRSEDTAVYYCARDYQLSMTSGFDYWG | |
| | QGTLVTVSSGGGGSGGGGSGGGGSEIELTQSPSSLSASVG | |
| | DRVTITCRASQSISSYLNWYQQKPGKAPKLLIYAASSLQS | |
| | GVPSRFSGSGSGTDFTLTISSLQPEDFATYYCQQSYSTPPT | |
| | FGQGTKVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 863 CAR | sc02-132 VL-VH P 002 linker scFv: | 435 |
| | SELTQSPSSLSASVGDRVTITCRASQSISSYLNWYQQKPG | |
| | KAPKLLIYAASSLQSGVPSRFSGSGSGTDFTLTISSLQPED | |
| | FATYYCQQSYSTPPTFGQGTKVEIKRAAALEGTGGSGGT | |
| | GSGTGTAEVQLVESGGGLVQPGGSLRLPCAASGFTFSNY | |
| | VMHWVRQAPGKGLEWVAVISYDGSNKYYADSVKGRFT | |
| | ISRDNSKNTLYLQMNSLRAEDTAVYYCVKSHSNMAFDY | |
| | WGQGTLVTV | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| pCTX 864 CAR | sc02-132 VH-VL P 002 linker scFv: | 436 |
| | AEVQLVESGGGLVQPGGSLRLPCAASGFTFSNYVMHWV | |
| | RQAPGKGLEWVAVISYDGSNKYYADSVKGRFTISRDNS | |
| | KNTLYLQMNSLRAEDTAVYYCVKSHSNMAFDYWGQGT | |
| | LVTVLEGTGGSGGTGSGTGTSELTQSPSSLSASVGDRVTI | |
| | TCRASQSISSYLNWYQQKPGKAPKLLIYAASSLQSGVPSR | |
| | FSGSGSGTDFTLTISSLQPEDFATYYCQQSYSTPPTFGQGT | |
| | KVEIKRAAA | |
| | IgG4 short hinge: | |
| | ESKYGPPCPPCP | |
| | CD28 transmembrane: | |
| | MFWVLVVVGGVLACYSLLVTVAFIIFWV | |
| | 4-1BB: | |
| | KRGRKKLLYIFKQPFMRPVQTTQEEDGCSCRFPEEEEGG | |
| | CEL | |
| | CD3z: | |
| | RVKFSRSADAPAYQQGQNQLYNELNLGRREEYDVLDKR | |
| | RGRDPEMGGKPRRKNPQEGLYNELQKDKMAEAYSEIG | |
| | MKGERRRGKGHDGLYQGLSTATKDTYDALHMQALPPR | |
| GMCSFR_Signal | MLLVTSLLLCELPHPAFLLIP | 47 |
| Peptide | | |
| IgK signal peptide | MVLQTQVFISLLLWISGAYG | 438 |
| CD8a signal | MALPVTALLLPLALLLHA | 439 |
| peptide | | |
| IL-2 signal peptide | MYRMQLLSCIALSLALVTNS | 440 |
| Myc tag | EQKLISEEDL | 441 |
| GMSCFR Signl | MLLLVTSLLLCELPHPAFLLIP | 254 |
| peptide | | |

In some embodiments, a CD72 CAR of the present disclosure contains a peptide tag, such as for example a Myc tag (SEQ ID NO:441). In some embodiments, the Myc tag is inserted into the CD72 CAR following the signal peptide but before the first variable region of the scFv binding domain. It is understood that for CAR sequences provided herein that include a Myc tag, the same CAR sequence is contemplated without the Myc tag inserted within the sequence.

### II. Chimeric Engulfment Receptors

In some embodiments, a chimeric engulfment receptor may be administered in combination with a CD72 CAR of the present disclosure. A “chimeric engulfment receptor” (CER) refers to a single chain chimeric protein that is capable of conferring a targeted engulfment phenotype to a host cell that is engineered to express said chimeric engulfment receptor. In some embodiments, expression of a CER as described herein confers an engulfment phenotype to an engineered cell that does not naturally exhibit an engulfment phenotype. In other such embodiments, expression of a CER as described herein by an engineered cell confers an engulfment phenotype specific to a pro-engulfment marker or antigenic marker not naturally targeted by the host cell. In still other such embodiments, expression of a CER as described herein by a host cell confers an engulfment phenotype specific to a pro-engulfment marker or antigenic marker naturally targeted by the host cell and expression of the CER by the host cell enhances engulfment activity by the host cell of cells, microbes, or particles exhibiting the targeted pro-engulfment or antigenic marker.

CERs of the present disclosure comprise an extracellular domain comprising a binding domain that binds to a target antigen, such as a pro-engulfment marker (e.g., phosphatidylserine); an engulfment signaling domain; and a transmembrane domain positioned between and connecting the extracellular domain and the engulfment signaling domain.

A binding domain may be any polypeptide or peptide that specifically binds a target molecule of interest. Sources of binding domains include receptor binding domains, ligand binding domains, and antibodies or antigen binding portions, such as antibody variable regions from various species (which can be in the form of antibodies, sFvs, scFvs, Fabs, scFv-based grababody, or soluble VH domain or domain antibodies), including human, rodent, avian, or ovine. Additional sources of binding domains include variable regions of antibodies from other species, such as camelid (from camels, dromedaries, or llamas; Ghahroudi et al., FEBS Lett. 414:521, 1997; Vincke et al., J. Biol. Chem. 284:3273, 2009; Hamers-Casterman et al., Nature 363:446, 1993 and Nguyen et al., J. Mol. Biol. 275:413, 1998), nurse sharks (Roux et al., Proc. Nat'l. Acad. Sci. (USA) 95:11804, 1998), spotted ratfish (Nguyen et al., Immunogen. 54:39, 2002), or lamprey (Herrin et al., Proc. Nat'l. Acad. Sci. (USA) 105:2040, 2008 and Alder et al. Nat. Immunol. 9:319, 2008). These antibodies can form antigen-binding regions using only a heavy chain variable region, i.e., these functional antibodies are homodimers of heavy chains only (referred to as “heavy chain antibodies”) (Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008).

In some embodiments, the extracellular domain binds to a pro-engulfment marker. As used herein, the term “pro-engulfment marker” refers to a moiety (e.g., protein, lipid, or polysaccharide) that an apoptotic, necrotic, pyroptotic, or infected cell exhibits on its surface that distinguishes it from a non-apoptotic, non-necrotic, non-pyroptotic, oncotic, or uninfected cell, respectively. A pro-engulfment marker can be an intracellular moiety that is surface exposed on an apoptotic or necrotic cell, a moiety that has altered glycosylation or altered surface charge on an apoptotic or necrotic cell, or a serum moiety that is bound to an apoptotic, necrotic, pyroptotic, or oncotic cell. Examples of pro-engulfment markers for apoptotic cells include phosphatidylserine (PtdSer), ICAM-3, oxidized low density lipoprotein, calreticulin, annexin I, complement C1q, and thrombospondin. Necrotic, oncotic, and pyroptotic cells also expose PtdSer pro-engulfment markers on the cell surface. Engulfment receptors can detect (or bind) a pro-engulfment marker on a target cell (e.g., a damaged, infected, apoptotic, necrotic, pyroptotic, or oncotic cell) directly or indirectly using soluble bridging molecules as intermediaries that bind to the pro-engulfment marker. In some embodiments, the pro-engulfment marker targeted by the extracellular domain is phosphatidylserine (PtdSer), ICAM-3, oxidized low density lipoprotein, calreticulin, annexin I, complement C1q, or thrombospondin. In some embodiments, the pro-engulfment marker targeted by a CER of the present disclosure is phosphatidylserine.

In some embodiments, the extracellular domain that binds to a pro-engulfment marker is derived from an endogenous engulfment receptor or a soluble bridging molecule for an engulfment receptor (e.g., GAS6, Protein S, MFG-E8). In some embodiments, the entire extracellular portion (for membrane spanning molecules), the entire bridging molecule, or a truncated portion of an engulfment receptor or bridging molecule is used, provided that the truncated portion retains sufficient binding activity to the pro-engulfment marker (i.e., is a functional variant). In further embodiments, the extracellular portion of an engulfment receptor or bridging molecule used for the extracellular domain is a variant of the entire extracellular portion (for membrane spanning molecules), the entire bridging molecule, or a truncated portion of the engulfment receptor or bridging molecule, provided that the variant retains sufficient binding activity to the pro-engulfment marker (i.e., is a functional variant).

In some embodiments, the extracellular domain includes a T-cell immunoglobulin and mucin domain 1 (Tim1), T-cell immunoglobulin and mucin domain 4 (Tim4), T-cell immunoglobulin and mucin domain 3 (Tim3), stabilin-2, RAGE, or Fc receptor (FcR) extracellular domain. In specific embodiments, an FcR extracellular domain can include a binding domain from FcγR1, FcγR2A, FcγR2B2, FcγR2C, FcγR3A, FcεR1, or FcαR1. In further embodiments, the extracellular domain can include a PtdSer binding domain obtained or derived from Tim1, Tim4, Tim3, stabilin-2, receptor for advanced glycation end products (RAGE), brain-specific angiogenesis inhibitor 1 (BAI1), Milk Fat Globule-EGF Factor 8 Protein (MFG-E8) (e.g., a FA58C2 domain that mediates high affinity binding to PtdSer), Growth Arrest Specific 6 (GAS6), protein S, protein C, Factor II, Factor VII, Factor IX, Factor X, Beta 2-glycoprotein I, α5β3 integrin and other integrins, CR3 complement receptor, CR4 complement receptor, CD14, CD93, annexin V, phosphatidylserine receptor (PSr), prothrombin, or scavenger receptors such as scavenger receptor B (SRB) (e.g., SRB1 (CD36)), scavenger receptor C (SRC) (e.g., LOX-1, SRCL), scavenger receptor D (SRD) (e.g., CD68, macrosialin), and PSOX,

In some embodiments, the extracellular domain comprises or is a sequence that is at least 90%, at least 91%, at least 92%, at least 93%, at least 94%, at least 95%, at least 96%, at least 97%, at least 98%, at least 99%, at least 99.5%, or 100% identical to a FcγRI binding domain comprising an amino acid sequence of SEQ ID NO:62 or amino acids 16-292 of SEQ ID NO:62, TIM1 binding domain comprising an amino acid sequence of SEQ ID NO:57 or amino acids 21-295 of SEQ ID NO:57 or SEQ ID NO:63 or amino acids 21-290 of SEQ ID NO:63, a TIM4 binding domain comprising an amino acid sequence of SEQ ID NO:64 or amino acids 25-314 of SEQ ID NO:64, a TIM3 binding domain comprising an amino acid sequence of SEQ ID NO:65 or amino acids 22-202 of SEQ ID NO:65, a FA58C2 binding domain comprising an amino acid sequence of SEQ ID NO:66, a GAS6 binding domain comprising an amino acid sequence of SEQ ID NO:67 or amino acids 31-94 of SEQ ID NO:67, a BAI1 binding domain comprising an amino acid sequence of SEQ ID NO:69; or a protein S binding domain comprising an amino acid sequence of SEQ ID NO:68 or amino acids 25-87 of SEQ ID NO:68.

In other embodiments, the extracellular domain is derived or obtained from least one of the following: CD14, which binds to ICAM3; a scavenger receptor extracellular domain, which binds to oxidized LDL; a lectin, which binds to altered sugars; CD36, which binds to thrombospondin; or LRP1/CD91 or a lectin moiety, which binds to calreticulin.

A target molecule, which is specifically bound by an extracellular domain of a CER of the present disclosure, may be found on or in association with a cell of interest (“target cell”). Exemplary target cells include a cancer cell, a cell associated with an autoimmune disease or disorder or with an inflammatory disease or disorder, and an infectious microbe (e.g., bacteria, virus, or fungi), or infected cell (e.g., virus-infected cell). A cell of an infectious organism, such as a mammalian parasite, is also contemplated as a target cell.

In some embodiments, the extracellular domain of the CER optionally comprises an extracellular, non-signaling spacer or linker domain. Where included, such a spacer or linker domain may position the binding domain away from the host cell surface to further enable proper cell/cell contact, binding, activation, and expansion. An extracellular spacer domain is generally located between the extracellular binding domain and the transmembrane domain. The length of the extracellular spacer may be varied to optimize target molecule binding based on the selected target molecule, selected binding epitope, binding domain size and affinity (see, e.g., Guest et al., J. Immunother. 28:203-11, 2005; Hudecek et al., Clin. Cancer Res. 19:3153-64, 2013; Hudecek et al., Cancer Immunol. Res. 3:125-35, 2015; PCT Publication No. WO 2014/031687; each of which is incorporated by reference in its entirety). In certain embodiments, an extracellular spacer domain comprises a TLR juxtamembrane domain (e.g., TLR1, TLR2, TLR3, TLR4, TLR5, TLR6, TLR7, TLR8, or TLR9 juxtamembrane domain). In a particular embodiment, an extracellular spacer domain comprises a TLR4 juxtamembrane domain comprising an amino acid sequence of SEQ ID NO:70. In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG₄ hinge region is described in PCT Publication No. WO 2014/031687, which hinge region is incorporated herein by reference in its entirety. In a particular embodiment, an extracellular spacer domain comprises a modified IgG4 hinge region having an amino acid sequence of ESKYGPPCPPCP (SEQ ID NO:21).

Other examples of hinge regions that may be used in the CERs described herein include the hinge region present in the extracellular regions of type 1 membrane proteins, such as CD8a, CD4, CD28 and CD7, which may be wild-type or variants thereof. An exemplary CD8a hinge region comprises the amino acid sequence set forth in SEQ ID NO:22. An exemplary CD28 hinge region comprises the amino acid sequence set forth in SEQ ID NO:23. In some embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In a particular embodiment, the Fc domain is modified to prevent in vivo interactions with cells expressing FcγRs that may result in off-target activation of CER-modified cells. In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D. In yet further embodiments, an extracellular spacer domain may be derived from MERTK.

CERs of the present disclosure comprise a transmembrane domain that connects and is positioned between the extracellular domain and the intracellular signaling domain. In some embodiments, the transmembrane domain ranges in length from about 15 amino acids to about 30 amino acids. The transmembrane domain is a hydrophobic alpha helix that transverses the host cell membrane and anchors the CER in the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). The transmembrane domain can be selected from the same molecule as the extracellular domain or the engulfment signaling domain (e.g., a CER comprises a Tim4 binding domain and a Tim4 transmembrane domain). In some embodiments, the transmembrane domain and the extracellular domain are each selected from different molecules. In some embodiments, the transmembrane domain and the engulfment signaling domain are each selected from different molecules. In yet other embodiments, the transmembrane domain, the extracellular domain, and the engulfment signaling domain are each selected from different molecules.

In some embodiments, the transmembrane domain comprises a TLR transmembrane domain (e.g., TLR1, TLR2, TLR3, TLR4, TLR5, TLR6, TLR7, TLR8, or TLR9 transmembrane domain), a Tim1 transmembrane domain, a Tim4 transmembrane domain, an FcR transmembrane domain (e.g., FcγR1, FcγR2A, FcγR2B2, FcγR2C, FcγR3A, FcεR1, or FcαR 1 transmembrane domain), a CD8a transmembrane domain, a MERTK transmembrane domain, an Ax1 transmembrane domain, a Tyro3 transmembrane domain, a BAI1 transmembrane domain, a CD4 transmembrane domain, a CD28 transmembrane domain, a MRC1 transmembrane domain, or a DAP12 transmembrane domain.

In specific embodiments, the transmembrane domain comprises or is a sequence that is at least 90%, at least 91%, at least 92%, at least 93%, at least 94%, at least 95%, at least 96%, at least 97%, at least 98%, at least 99%, at least 99.5%, or 100% identical to a TLR1 transmembrane domain comprising an amino acid sequence of SEQ ID NO:71, a TLR2 transmembrane domain comprising an amino acid sequence of SEQ ID NO:72, a TLR3 transmembrane domain comprising an amino acid sequence of SEQ ID NO:73, a TLR4 transmembrane domain comprising an amino acid sequence of SEQ ID NO:74, a TLR5 transmembrane domain comprising an amino acid sequence of SEQ ID NO:75, a TLR6 transmembrane domain comprising an amino acid sequence of SEQ ID NO:76, a TLR7 transmembrane domain comprising an amino acid sequence of SEQ ID NO:77, a TLR8 transmembrane domain comprising an amino acid sequence of SEQ ID NO:78, a TLR9 transmembrane domain comprising an amino acid sequence of SEQ ID NO:79, a Tim1 transmembrane domain comprising an amino acid sequence of SEQ ID NO:80, a Tim4 transmembrane domain comprising an amino acid sequence of SEQ ID NO:81, an FcγRI transmembrane domain comprising an amino acid sequence of SEQ ID NO:82, a FcεRIγ transmembrane domain comprising an amino acid sequence of SEQ ID NO:83, a CD8a transmembrane domain comprising an amino acid sequence of SEQ ID NO:84, a MERTK transmembrane domain comprising an amino acid sequence of SEQ ID NO:85, an Ax1 transmembrane domain comprising an amino acid sequence of SEQ ID NO:86, a Tyro3 transmembrane domain comprising an amino acid sequence of SEQ ID NO:87, a BAI1 transmembrane domain comprising an amino acid sequence of SEQ ID NO:91, a CD28 transmembrane domain as set forth in an amino acid sequence of SEQ ID NO:88, a CD4 transmembrane domain comprising an amino acid sequence of SEQ ID NO: 89, a MRC1 transmembrane domain comprising an amino acid sequence of SEQ ID NO:92, or a DAP12 transmembrane domain comprising an amino acid sequence of SEQ ID NO:90.

The engulfment signaling domain of a CER is an intracellular effector domain and is capable of transmitting functional signals to a cell in response to binding of the extracellular domain of the CER to a target molecule. CERs of the present disclosure may include one or more engulfment signaling domains as described herein. An “engulfment signaling domain” refers to an intracellular effector domain, which upon binding of the target molecule (e.g., pro-engulfment marker or antigenic marker) targeted by the extracellular domain of a CER expressed by a host cell, activates one or more signaling pathways in the host cell resulting in engulfment, including, in specific embodiments, cytoskeletal rearrangement of the host cell and internalization of the target cell, microbe, or particle associated with the marker or antigen. In certain embodiments, an engulfment signaling domain activates one or more signaling pathways resulting in phagocytosis of the target cell, microbe, or particle.

In some embodiments, the engulfment signaling domain comprises an FcR signaling domain (including an FcγRI signaling domain, an FcγR2A signaling domain, an FcγR2C signaling domain, FcγR2B2 signaling domain, an FcγR3A signaling domain, FcγR2C signaling domain, FcγR3A signaling domain, FcεRIγ signaling domain, and FcαRI signaling domain), a MyD88 signaling domain, a Zap70 signaling domain, a Syk signaling domain, a PI3K signaling domain, a B-cell activating factor receptor (BAFF-R) signaling domain, a DAP12 (also referred to as TYRO Protein Tyrosine Kinase Binding Protein (TYROBP)) signaling domain, an NFAT Activating Protein With ITAM Motif 1 (NFAM1) signaling domain, a MERTK signaling domain, a TLR1 signaling domain, a TLR2 signaling domain, a TLR3 signaling domain, a TLR4 signaling domain, a TLR5 signaling domain, a TLR6 signaling domain, a TLR7 signaling domain, a TLR8 signaling domain, a TLR9 signaling domain, a Traf6 signaling domain, a Traf2 signaling domain, a Traf3 signaling domain, a CD79b signaling domain, a MRC1 signaling domain, an ItgB4 signaling domain, a Tyro3 signaling domain, an Ax1 signaling domain, a BAI1 signaling domain, or an ELMO signaling domain.

In some embodiments, the engulfment signaling domain comprises or is a sequence that is at least 90%, at least 91%, at least 92%, at least 93%, at least 94%, at least 95%, at least 96%, at least 97%, at least 98%, at least 99%, at least 99.5%, or 100% identical to a FcεRIγ signaling domain comprising an amino acid sequence of SEQ ID NO:93, an FcγRI signaling domain comprising an amino acid sequence of SEQ ID NO:94, an FcγR2A signaling domain comprising an amino acid sequence of SEQ ID NO:95, an FcγR2C signaling domain comprising an amino acid sequence of SEQ ID NO:96, an FcγR3A signaling domain comprising an amino acid sequence of SEQ ID NO:97, a MyD88 signaling domain comprising an amino acid sequence of SEQ ID NO:98 or 99, a Zap70 signaling domain comprising an amino acid sequence of SEQ ID NO:101, a Syk signaling domain comprising an amino acid sequence of SEQ ID NO: 100, a BAFF-R signaling domain comprising an amino acid sequence of SEQ ID NO: 102, a DAP12 signaling domain comprising an amino acid sequence of SEQ ID NO: 103, a NFAM1 signaling domain comprising an amino acid sequence of SEQ ID NO: 104, a truncated NFAM1 signaling domain comprising an amino acid sequence of SEQ ID NO: 105, a CD79b signaling domain comprising an amino acid sequence of SEQ ID NO:109, a truncated CD79b signaling domain comprising an amino acid sequence of SEQ ID NO:106, a MERTK signaling domain comprising an amino acid sequence of SEQ ID NO: 111, a TLR1 signaling domain comprising an amino acid sequence of SEQ ID NO: 112, a TLR2 signaling domain comprising an amino acid sequence of SEQ ID NO: 113, a TLR3 signaling domain comprising an amino acid sequence of SEQ ID NO: 114, a TLR4 signaling domain comprising an amino acid sequence of SEQ ID NO:115, a TLR5 signaling domain comprising an amino acid sequence of SEQ ID NO: 116, a TLR6 signaling domain comprising an amino acid sequence of SEQ ID NO:117, a TLR7 signaling domain comprising an amino acid sequence of SEQ ID NO: 118, a TLR8 signaling domain comprising an amino acid sequence of SEQ ID NO: 119, a TLR9 signaling domain comprising an amino acid sequence of SEQ ID NO: 120, a Traf6 signaling domain comprising an amino acid sequence of SEQ ID NO: 121, a truncated Traf6 signaling domain comprising an amino acid sequence of SEQ ID NO: 122, a Traf2 signaling domain comprising an amino acid sequence of SEQ ID NO:123, a Traf3 signaling domain comprising an amino acid sequence of SEQ ID NO: 124, a MRC1 signaling domain comprising an amino acid sequence of SEQ ID NO:125, an ItgB4 signaling domain comprising an amino acid sequence of SEQ ID NO:126, a Tyro3 signaling domain comprising an amino acid sequence of SEQ ID NO: 127, an Ax1 signaling domain comprising an amino acid sequence of SEQ ID NO: 128, a BAI1 signaling domain comprising an amino acid sequence of SEQ ID NO: 129, or an ELMO signaling domain comprising an amino acid sequence of SEQ ID NO: 130.

The engulfment signaling domain may be any portion of an engulfment signaling molecule that retains sufficient signaling activity. In some embodiments, the engulfment signaling domain is obtained from an engulfment receptor component or associated signaling molecule. In some embodiments, a full length intracellular portion of an engulfment signaling domain of an engulfment receptor or associated signaling molecule is used. In some embodiments, a truncated portion of an intracellular engulfment signaling domain of an engulfment receptor or associated signaling molecule is used, provided that the truncated portion retains sufficient signal transduction activity. In further embodiments, an engulfment signaling domain is a variant of full length or truncated portion of an engulfment signaling domain of an engulfment receptor or associated signaling molecule, provided that the variant retains sufficient signal transduction activity (i.e., is a functional variant).

In some embodiments, the engulfment signaling domain comprises a primary engulfment signaling domain and a secondary engulfment signaling domain. In some embodiments, the primary engulfment signaling domain comprise an FcR signaling domain (including an FcγRI signaling domain, an FcγR2A signaling domain, an FcγR2C signaling domain, FcγR2B2 signaling domain, an FcγR3A signaling domain, FcγR2C signaling domain, FcγR3A signaling domain, FcεRIγ signaling domain, and FcαR1 signaling domain), a MyD88 signaling domain, a Zap70 signaling domain, a Syk signaling domain, a PI3K signaling domain, a B-cell activating factor receptor (BAFF-R) signaling domain, a DAP12 (also referred to as TYRO Protein Tyrosine Kinase Binding Protein (TYROBP)) signaling domain, an NFAT Activating Protein With ITAM Motif 1 (NFAM1) signaling domain, a MERTK signaling domain, a TLR1 signaling domain, a TLR2 signaling domain, a TLR3 signaling domain, a TLR4 signaling domain, a TLR5 signaling domain, a TLR6 signaling domain, a TLR7 signaling domain, a TLR8 signaling domain, a TLR9 signaling domain, a Traf6 signaling domain, a Traf2 signaling domain, a Traf3 signaling domain, a CD79b signaling domain, a MRC1 signaling domain, an ItgB4 signaling domain, a Tyro3 signaling domain, an Ax1 signaling domain, a BAI1 signaling domain, or an ELMO signaling domain.

In some embodiments, the primary engulfment signaling domain comprises or is a sequence that is at least 90%, at least 91%, at least 92%, at least 93%, at least 94%, at least 95%, at least 96%, at least 97%, at least 98%, at least 99%, at least 99.5%, or 100% identical to a FcαRIy signaling domain comprising an amino acid sequence of SEQ ID NO:93, an FcγR1 signaling domain comprising an amino acid sequence of SEQ ID NO:94, an FcγR2A signaling domain comprising an amino acid sequence of SEQ ID NO:95, an FcγR2C signaling domain comprising an amino acid sequence of SEQ ID NO:96, an FcγR3A signaling domain comprising an amino acid sequence of SEQ ID NO:97, a MyD88 signaling domain comprising an amino acid sequence of SEQ ID NO:98 or 99, a Zap70 signaling domain comprising an amino acid sequence of SEQ ID NO: 101, a Syk signaling domain comprising an amino acid sequence of SEQ ID NO: 100, a BAFF-R signaling domain comprising an amino acid sequence of SEQ ID NO: 102, a DAP12 signaling domain comprising an amino acid sequence of SEQ ID NO: 103, a NFAM1 signaling domain comprising an amino acid sequence of SEQ ID NO:104, a truncated NFAM1 signaling domain comprising an amino acid sequence of SEQ ID NO: 105, a CD79b signaling domain comprising an amino acid sequence of SEQ ID NO: 109, a truncated CD79b signaling domain comprising an amino acid sequence of SEQ ID NO: 106, a MERTK signaling domain comprising an amino acid sequence of SEQ ID NO:111: a TLR1 signaling domain comprising an amino acid sequence of SEQ ID NO: 112, a TLR2 signaling domain comprising an amino acid sequence of SEQ ID NO: 113, a TLR3 signaling domain comprising an amino acid sequence of SEQ ID NO: 114, a TLR4 signaling domain comprising an amino acid sequence of SEQ ID NO: 115, a TLR5 signaling domain comprising an amino acid sequence of SEQ ID NO:116, a TLR6 signaling domain comprising an amino acid sequence of SEQ ID NO: 117, a TLR7 signaling domain comprising an amino acid sequence of SEQ ID NO:118, a TLR8 signaling domain comprising an amino acid sequence of SEQ ID NO: 119, a TLR9 signaling domain comprising an amino acid sequence of SEQ ID NO: 120, a Traf6 signaling domain comprising an amino acid sequence of SEQ ID NO: 121, a truncated Traf6 signaling domain comprising an amino acid sequence of SEQ ID NO: 122, a Traf2 signaling domain comprising an amino acid sequence of SEQ ID NO:107, a Traf3 signaling domain comprising an amino acid sequence of SEQ ID NO: 108, a MRC1 signaling domain comprising an amino acid sequence of SEQ ID NO:125, an ItgB4 signaling domain comprising an amino acid sequence of SEQ ID NO:126, a Tyro3 signaling domain comprising an amino acid sequence of SEQ ID NO: 127, an Ax1 signaling domain comprising an amino acid sequence of SEQ ID NO: 128, a BAI1 signaling domain comprising an amino acid sequence of SEQ ID NO: 129, or an ELMO signaling domain comprising an amino acid sequence of SEQ ID NO: 130.

In some embodiments, the secondary engulfment signaling domain comprise an FcR signaling domain (including an FcγRI signaling domain, an FcγR2A signaling domain, an FcγR2C signaling domain, FcγR2B2 signaling domain, an FcγR3A signaling domain, FcγR2C signaling domain, FcγR3A signaling domain, FcεRIγ signaling domain, and FcαRI signaling domain), a MyD88 signaling domain, a Zap70 signaling domain, a Syk signaling domain, a PI3K signaling domain, a B-cell activating factor receptor (BAFF-R) signaling domain, a DAP12 (also referred to as TYRO Protein Tyrosine Kinase Binding Protein (TYROBP)) signaling domain, an NFAT Activating Protein With ITAM Motif 1 (NFAM1) signaling domain, a MERTK signaling domain, a TLR1 signaling domain, a TLR2 signaling domain, a TLR3 signaling domain, a TLR4 signaling domain, a TLR5 signaling domain, a TLR6 signaling domain, a TLR7 signaling domain, a TLR8 signaling domain, a TLR9 signaling domain, a Traf6 signaling domain, a Traf2 signaling domain, a Traf3 signaling domain, a CD79b signaling domain, a MRC1 signaling domain, an ItgB4 signaling domain, a Tyro3 signaling domain, an Ax1 signaling domain a BAI1 signaling domain, or an ELMO signaling domain.

In some embodiments, the secondary engulfment signaling domain comprises or is a sequence that is at least 90%, at least 91%, at least 92%, at least 93%, at least 94%, at least 95%, at least 96%, at least 97%, at least 98%, at least 99%, at least 99.5%, or 100% identical to a FcεRIγ signaling domain comprising an amino acid sequence of SEQ ID NO:93, an FcγRI signaling domain comprising an amino acid sequence of SEQ ID NO:94, an FcγR2A signaling domain comprising an amino acid sequence of SEQ ID NO:95, an FcγR2C signaling domain comprising an amino acid sequence of SEQ ID NO:96, an FcγR3A signaling domain comprising an amino acid sequence of SEQ ID NO:97, a MyD88 signaling domain comprising an amino acid sequence of SEQ ID NO:98 or 99, a Zap70 signaling domain comprising an amino acid sequence of SEQ ID NO: 101, a Syk signaling domain comprising an amino acid sequence of SEQ ID NO: 100, a BAFF-R signaling domain comprising an amino acid sequence of SEQ ID NO:102, a DAP12 signaling domain comprising an amino acid sequence of SEQ ID NO: 103, a NFAM1 signaling domain comprising an amino acid sequence of SEQ ID NO: 104, a truncated NFAM1 signaling domain comprising an amino acid sequence of SEQ ID NO:105, a CD79b signaling domain comprising an amino acid sequence of SEQ ID NO: 109, a truncated CD79b signaling domain comprising an amino acid sequence of SEQ ID NO: 106, a MERTK signaling domain comprising an amino acid sequence of SEQ ID NO:111: a TLR1 signaling domain comprising an amino acid sequence of SEQ ID NO:112, a TLR2 signaling domain comprising an amino acid sequence of SEQ ID NO:113, a TLR3 signaling domain comprising an amino acid sequence of SEQ ID NO:114, a TLR4 signaling domain comprising an amino acid sequence of SEQ ID NO: 115, a TLR5 signaling domain comprising an amino acid sequence of SEQ ID NO:116, a TLR6 signaling domain comprising an amino acid sequence of SEQ ID NO: 117, a TLR7 signaling domain comprising an amino acid sequence of SEQ ID NO:118, a TLR8 signaling domain comprising an amino acid sequence of SEQ ID NO:119, a TLR9 signaling domain comprising an amino acid sequence of SEQ ID NO: 120, a Traf6 signaling domain comprising an amino acid sequence of SEQ ID NO: 121, a truncated Traf6 signaling domain comprising an amino acid sequence of SEQ ID NO: 122, a Traf2 signaling domain comprising an amino acid sequence of SEQ ID NO:107, a Traf3 signaling domain comprising an amino acid sequence of SEQ ID NO:108, a MRC1 signaling domain comprising an amino acid sequence of SEQ ID NO:125, an ItgB4 signaling domain comprising an amino acid sequence of SEQ ID NO:126, a Tyro3 signaling domain comprising an amino acid sequence of SEQ ID NO:127, an Ax1 signaling domain comprising an amino acid sequence of SEQ ID NO: 128, a BAI1 signaling domain comprising an amino acid sequence of SEQ ID NO: 129, or an ELMO signaling domain comprising an amino acid sequence of SEQ ID NO:130.

In some embodiments, the primary engulfment signaling domain is the same as the secondary engulfment signaling domain. In some embodiments, the primary engulfment signaling domain is different than the secondary engulfment signaling domain.

It is understood that in embodiments where a CER comprises a primary engulfment signaling domain and a secondary engulfment signaling domain, different orientations of the primary and secondary engulfment signaling domains are contemplated unless stated otherwise. For example, in some embodiments, the primary engulfment signaling domain is N-terminal to the secondary engulfment signaling domain. In some embodiments, the secondary engulfment signaling domain is N-terminal to the primary engulfment signaling domain.

It is understood that direct fusion of one domain to another domain of a CER described herein does not preclude the presence of intervening junction amino acids. Junction amino acids may be natural or non-natural (e.g., resulting from the construct design of a chimeric protein).

Exemplary CER configurations, components, constructs, and sequences thereof are set forth in Table 2.

TABLE 2
Exemplary Chimeric Engulfment Receptors

| | | | Primary | Secondary | |
|---|---|---|---|---|---|
| | Binding Domain/ | | Engulfment | Engulfment | |
| | Extracellular | Transmembrane | Signaling | Signaling | Exemplary Amino |
| CER Name | Spacer Domain | Domain | Domain | Domain | Acid Sequences |
| CER1 | Tim4 | Tim4 | MERTK | | SEQ ID NO: 131 |
| CER5 | Tim4 | Tim4 | TLR4 | | SEQ ID NO: 132 |
| CER6 | Tim4 | TLR4 | TLR4 | | SEQ ID NO: 133 |
| CER7 | Tim4 + TLR | TLR4 | TLR4 | | SEQ ID NO: 134 |
| | juxtamembrane | | | | |
| | domain | | | | |
| CER8 | Tim4 | Tim4 | Tyro3 | | SEQ ID NO: 135 |
| CER9 | Tim4 | Tim4 | DAP12 | | SEQ ID NO: 136 |
| CER10 | Tim4 | DAP12 | DAP12 | | SEQ ID NO: 137 |
| CER11 | Tim4 | Tim4 | Axl | | SEQ ID NO: 138 |
| CER12 | Tim4 | Tim4 | FcRg | | SEQ ID NO: 139 |
| CER13 | Tim4 | FcεR1 | FcRg | | SEQ ID NO: 140 |
| CER15 | Tim4 | Tim4 | MyD88 | | SEQ ID NO: 141 |
| CER16 | Tim4 | Tim4 | MyD88_TIR | | SEQ ID NO: 142 |
| CER17 | Tim4 | Tim4 | TLR_3 | | SEQ ID NO: 143 |
| CER18 | Tim4 | TLR_3 | TLR_3 | | SEQ ID NO: 144 |
| CER19 | Tim4 | Tim4 | TLR_5 | | SEQ ID NO: 145 |
| CER20 | Tim4 | TLR_5 | TLR_5 | | SEQ ID NO: 146 |
| CER21 | Tim4 | Tim4 | TLR_8 | | SEQ ID NO: 147 |
| CER22 | Tim4 | TLR_8 | TLR_8 | | SEQ ID NO: 148 |
| CER23 | Tim4 | Tim4 | TLR_9 | | SEQ ID NO: 149 |
| CER24 | Tim4 | TLR_9 | TLR_9 | | SEQ ID NO: 150 |
| CER25 | Tim4 | Tim4 | NFAM | | SEQ ID NO: 151 |
| CER26 | Tim4 | Tim4 | TLR_1 | | SEQ ID NO: 152 |
| CER27 | Tim4 | Tim4 | TLR_2 | | SEQ ID NO: 153 |
| CER28 | Tim4 | Tim4 | TLR_7 | | SEQ ID NO: 154 |
| CER29 | Tim4 | Tim4 | TRAF6 | | SEQ ID NO: 155 |
| CER30 | Tim4 | Tim4 | TRAF2 | | SEQ ID NO: 156 |
| CER31 | Tim4 | Tim4 | TRAF3 | | SEQ ID NO: 157 |
| CER85 | Tim4 | Tim4 | MyD88 | Baff-R | SEQ ID NO: 158 |
| CER86 | Tim4 | Tim4 | MyD88 | DAP12 | SEQ ID NO: 159 |
| CER87 | Tim4 | Tim4 | Baff-R | MyD88 | SEQ ID NO: 160 |
| CER88 | Tim4 | Tim4 | DAP12 | MyD88 | SEQ ID NO: 161 |
| CER89 | Tim4 | Tim4 | MyD88 | CD79b (185-229) | SEQ ID NO: 162 |
| CER90 | Tim4 | Tim4 | MyD88 | NFAM1 | SEQ ID NO: 163 |
| CER91 | Tim4 | Tim4 | MyD88 | P2A-Rab | SEQ ID NO: 164 |
| CER92 | Tim4 | Tim4 | MERTK | MyD88 | SEQ ID NO: 165 |
| CER93 | Tim4 | Tim4 | MERTK | Baff-R | SEQ ID NO: 166 |
| CER94 | Tim4 | Tim4 | MERTK | DAP12 | SEQ ID NO: 167 |
| CER95 | Tim4 | Tim4 | MERTK | CD79b (185-229) | SEQ ID NO: 168 |
| CER96 | Tim4 | Tim4 | MERTK | NFAM1 | SEQ ID NO: 169 |
| CER97 | Tim4 | Tim4 | AXL | Dap12 | SEQ ID NO: 170 |
| CER98 | Tim4 | Tim4 | AXL | CD79b | SEQ ID NO: 171 |
| CER99 | Tim4 | Tim4 | AXL | NFAM1 | SEQ ID NO: 172 |
| CER102 | Tim4 | Tim4 | TLR8 | NFAM1 | SEQ ID NO: 173 |
| CER103A | Tim4 | Tim4 | TLR8 | CD79b (185-229) | SEQ ID NO: 174 |
| CER103B | Tim4 | Tim4 | TLR8 | CD79b (185-213) | SEQ ID NO: 175 |
| CER104 | Tim4 | Tim4 | TLR8 | DAP12 | SEQ ID NO: 176 |
| CER105 | Tim4 | Tim4 | TLR8 | Baff-R | SEQ ID NO: 177 |
| CER106 | Tim4 | Tim4 | NFAM1 | TLR8 | SEQ ID NO: 178 |
| CER107 | Tim4 | Tim4 | CD79b (185-213) | TLR8 | SEQ ID NO: 179 |
| CER108 | Tim4 | Tim4 | DAP12 | TLR8 | SEQ ID NO: 180 |
| CER109 | Tim4 | Tim4 | Baff-R | TLR8 | SEQ ID NO: 181 |
| CER110 | Tim4 | Tim4 | TRAF6 | DAP12 | SEQ ID NO: 182 |
| CER111A | Tim4 | Tim4 | TRAF6 | CD79b (185-229) | SEQ ID NO: 183 |
| CER111B | Tim4 | Tim4 | TRAF6 | CD79b (185-213) | SEQ ID NO: 184 |
| CER112 | Tim4 | Tim4 | TRAF6 | NFAM1 | SEQ ID NO: 185 |
| CER113 | Tim4 | Tim4 | TRAF6 | Baff-R | SEQ ID NO: 186 |
| CER114 | Tim4 | Tim4 | TRAF6 | MERTK | SEQ ID NO: 187 |
| CER115 | Tim4 | Tim4 | MERTK | TRAF6 | SEQ ID NO: 188 |
| CER116 | Tim4 | Tim4 | TRAF6 | TLR8 | SEQ ID NO: 189 |
| CER117 | Tim4 | Tim4 | TLR8 | TRAF6 | SEQ ID NO: 190 |
| CER118 | Tim4 | Tim4 | TLR1 | NFAM1 | SEQ ID NO: 191 |
| CER119A | Tim4 | Tim4 | TLR1 | CD79b (185-229) | SEQ ID NO: 192 |
| CER119B | Tim4 | Tim4 | TLR1 | CD79b (185-213) | SEQ ID NO: 193 |
| CER120 | Tim4 | Tim4 | TLR1 | DAP12 | SEQ ID NO: 194 |
| CER121 | Tim4 | Tim4 | TLR1 | TRAF6 | SEQ ID NO: 195 |
| CER122 | Tim4 | Tim4 | TLR2 | DAP12 | SEQ ID NO: 196 |
| CER123 | Tim4 | Tim4 | TLR2 | TRAF6 | SEQ ID NO: 197 |
| CER124 | Tim4 | Tim4 | TLR2 | NFAM1 | SEQ ID NO: 198 |
| CER125A | Tim4 | Tim4 | TLR2 | CD79b (185-229) | SEQ ID NO: 199 |
| CER125B | Tim4 | Tim4 | TLR2 | CD79b (185-213_{—} | SEQ ID NO: 200 |
| CER126 | Tim4 | Tim4 | TLR2 | TRAF2 | SEQ ID NO: 201 |
| CER127 | Tim4 | Tim4 | TRAF2 | TLR2 | SEQ ID NO: 202 |
| CER128 | Tim4 | Tim4 | TRAF2 | TLR8 | SEQ ID NO: 203 |
| CER129 | Tim4 | Tim4 | TLR8 | TRAF2 | SEQ ID NO: 204 |
| hCER21 | Tim4 | Tim4 | TLR8 | | SEQ ID NO: 205 |
| hCER29 | Tim4 | Tim4 | TRAF6 | | SEQ ID NO: 206 |
| hCER104 | Tim4 | Tim4 | TLR8 | Dap12 | SEQ ID NO: 207 |
| hCER116 | Tim4 | Tim4 | TRAF6 | TLR8 | SEQ ID NO: 208 |
| hCER117 | Tim4 | Tim4 | TLR8 | TRAF6 | SEQ ID NO: 209 |
| hCER122 | Tim4 | Tim4 | TLR2 | Dap12 | SEQ ID NO: 210 |
| hCER123 | Tim4 | Tim4 | TLR2 | TRAF6 | SEQ ID NO: 211 |
| hCER126 | Tim4 | Tim4 | TLR2 | TRAF2 | SEQ ID NO: 212 |

CERs of the present disclosure may comprise polynucleotide sequences derived from any mammalian species, including humans, primates, cows, horses, goats, sheep, dogs, cats, mice, rats, rabbits, guinea pigs, pigs, transgenic species thereof, or any combination thereof. In some embodiments, the CER is murine, chimeric, human, or humanized.

Exemplary CER components, CER constructs, methods of making, and methods of using are described in International Application Publication Nos. WO2018/064076; WO2019/067328; WO2019/191339; and WO2019/191340; each of which is herein incorporated by reference in its entirety.

### III. Chimeric Tim Receptors

In some embodiments, a chimeric T-cell membrane protein (Tim) receptor may be administered in combination with an anti-CD72 CAR of the present disclosure. Chimeric Tim receptors of the present disclosure confer engulfment and/or cytotoxic activity to chimeric Tim receptor-modified host cells, with the cytolytic activity being induced upon binding of the chimeric Tim receptor to its target antigen, phosphatidylserine.

### Chimeric Tim4 Receptors

In certain embodiments, a chimeric Tim receptor is a chimeric Tim4 receptor comprising a single chain chimeric protein, the single chain chimeric protein comprising: an extracellular domain comprising a Tim4 binding domain; an intracellular signaling domain comprising a first costimulatory signaling domain; and a transmembrane domain positioned between and connecting the extracellular domain and intracellular signaling domain. In certain embodiments, the extracellular domain of the chimeric Tim4 receptors described herein optionally includes an extracellular spacer domain positioned between and connecting the binding domain and transmembrane domain. When expressed in a host cell, chimeric Tim4 receptors of the present disclosure can confer a phosphatidylserine-specific, cytotoxic phenotype to the modified host cell (e.g., the host cell becomes cytotoxic to a stressed, damaged, injured, apoptotic, or necrotic cell expressing phosphatidylserine on its surface). In certain embodiments, the chimeric Tim4 receptors induce apoptosis in targeted cells via release of granzymes, perforin, granulysin, or any combination thereof. In further embodiments, cells expressing a chimeric Tim4 receptor according to the present description exhibit an engulfment phenotype specific to phosphatidylserine presenting cells.

The intracellular signaling domain can include one or more effector (also referred to as “costimulatory signaling”) domains that costimulate the modified host cell. Signaling by the costimulatory signaling domain(s) is triggered by binding of the extracellular domain to phosphatidylserine. In certain embodiments, the intracellular signaling domain comprises a first costimulatory signaling domain. In further embodiments, the intracellular signaling domain comprises a first costimulatory signaling domain and a second costimulatory signaling domain. Chimeric Tim4 receptors according to the present disclosure can be used in a variety of therapeutic methods where clearance of apoptotic, necrotic, damaged, or stressed cells is beneficial, while providing costimulation that enhances cellular immune response, reduces immune cell exhaustion, or both.

A Tim4 binding domain suitable for use in a chimeric Tim4 receptor of the present disclosure may be any polypeptide or peptide derived from a Tim4 molecule that specifically binds phosphatidylserine. In certain embodiments, the Tim4 binding domain is derived from human Tim4. An exemplary human Tim4 molecule is provided in Uniprot. Ref. Q96H15 (SEQ ID NO:49). An exemplary human Tim4 binding domain comprises or consists of an amino acid sequence of SEQ ID NO:50 or amino acids 25-314 of SEQ ID NO:50. An exemplary mouse Tim4 binding domain comprises or consists of an amino acid sequence of SEQ ID NO:51 or amino acids 23-279 of SEQ ID NO:51. In certain embodiments, the Tim4 binding domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:50 or amino acids 25-314 of SEQ ID NO:50, or SEQ ID NO:51 or amino acids 23-279 of SEQ ID NO:51. In certain embodiments, the Tim4 binding domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:50 or amino acids 25-314 of SEQ ID NO:50, or SEQ ID NO:51 or amino acids 23-279 of SEQ ID NO:51.

In certain embodiments, the extracellular domain optionally comprises an extracellular, non-signaling spacer or linker domain. Where included, such a spacer or linker domain may position the binding domain away from the host cell surface to further enable proper cell/cell contact, binding, and activation. When included in a chimeric receptor as described herein, an extracellular spacer domain is generally located between the extracellular binding domain and the transmembrane domain of the chimeric Tim4 receptor. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG4 hinge region is described in PCT Publication No. WO 2014/031687, which hinge region is incorporated herein by reference in its entirety. In some embodiments, an extracellular spacer domain comprises a modified IgG4 hinge region having an amino acid sequence of ESKYGPPCPPCP (SEQ ID NO:21). In some embodiments, a CD28 hinge region comprises the amino acid sequence of SEQ ID NO:23. Other examples of hinge regions that may be used in the chimeric Tim4 receptors described herein include the hinge region from the extracellular regions of in type 1 membrane proteins, such as CD8a, CD4, CD28 and CD7, which may be wild-type or variants thereof. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D.

In certain embodiments, an extracellular domain is encoded by polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, an extracellular domain is murine, human, or chimeric.

The intracellular signaling domain of a chimeric Tim4 receptor as described herein is an intracellular effector domain and is capable of transmitting functional signals to a cell in response to binding of the extracellular domain of the chimeric Tim4 receptor and phosphatidylserine. The signals transduced by the intracellular signaling domain promote effector function of the chimeric Tim4 receptor containing cell. Examples of effector function include cytotoxic activity, secretion of cytokines, proliferation, anti-apoptotic signaling, persistence, expansion, engulfment of a target cell or particle expressing phosphatidylserine on its surface, or any combination thereof.

In certain embodiments, an intracellular signaling domain comprises a costimulatory signaling domain. The costimulatory signaling domain may be any portion of a costimulatory signaling molecule that retains sufficient signaling activity. In some embodiments, a full length or full length intracellular component of a costimulatory signaling molecule is used. In some embodiments, a truncated portion of a costimulatory signaling molecule or intracellular component of a costimulatory signaling molecule is used, provided that the truncated portion retains sufficient signal transduction activity. In further embodiments, a costimulatory signaling domain is a variant of a whole or truncated portion of a costimulatory signaling molecule, provided that the variant retains sufficient signal transduction activity (i.e., is a functional variant).

In certain embodiments, the costimulatory signaling domain comprises a CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), PD-1, CD273 (PD-L2), CD274 (PD-L1), B7-H3 (CD276), ICOS (CD278), DAP10, LAT, LFA-1 (CD11a/CD18), LIGHT, NKG2C, SLP76, TRIM, or ZAP70 signaling domain. In particular embodiments, the costimulatory signaling domain comprises an OX40, CD2, CD27, CD28, ICAM-1, LFA-1 (CD11a/CD18), ICOS (CD278), or 4-1BB (CD137) signaling domain. An exemplary CD28 costimulatory signaling domain comprises or consists of an amino acid sequence of SEQ ID NO:28 or 29. An exemplary OX40 costimulatory signaling domain comprises or consists of an amino acid sequence of SEQ ID NO:213. An exemplary CD2 costimulatory signaling domain comprises or consists of an amino acid sequence of SEQ ID NO:214. An exemplary 4-1BB costimulatory signaling domain comprises or consists of an amino acid sequence of SEQ ID NO:215. An exemplary CD27 costimulatory signaling domain comprises or consists of an amino acid sequence of SEQ ID NO:216. An exemplary ICAM-1 costimulatory signaling domain comprises or consists of an amino acid sequence of SEQ ID NO:217. An exemplary LFA-1 costimulatory signaling domain comprises or consists of an amino acid sequence of SEQ ID NO:218. An exemplary ICOS costimulatory signaling domain comprises or consists of an amino acid sequence of SEQ ID NO:219. An exemplary CD30 costimulatory signaling domain comprises or consists of an amino acid sequence of SEQ ID NO:220. An exemplary CD40 costimulatory signaling domain comprises or consists of an amino acid sequence of SEQ ID NO:221. An exemplary PD-1 costimulatory signaling domain comprises or consists of an amino acid sequence of SEQ ID NO:222. An exemplary CD7 costimulatory signaling domain comprises or consists of an amino acid sequence of SEQ ID NO:223. An exemplary LIGHT costimulatory signaling domain comprises or consists of an amino acid sequence of SEQ ID NO:224. An exemplary NKG2C costimulatory signaling domain comprises or consists of an amino acid sequence of SEQ ID NO:225. An exemplary B7-H3 costimulatory signaling domain comprises or consists of an amino acid sequence of SEQ ID NO:226. In certain embodiments, the costimulatory signaling domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:213-226. In certain embodiments, the costimulatory signaling domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of any one of SEQ ID NOS:213-226. In certain embodiments, the intracellular signaling comprises a second costimulatory signaling domain. In preferred embodiments, the first costimulatory signaling domain and second costimulatory signaling domain are the same or different

In certain embodiments, the intracellular signaling domain further comprises an ITAM-containing activating domain. The ITAM-containing activating domain may recapitulate TCR signaling independently of endogenous TCR complexes. In certain embodiments, signaling via the ITAM-containing activating domain leads to mediation of a T cell response, including, but not limited to, proliferation, activation, differentiation, and the like. The ITAM-containing activating domain may be any portion of an ITAM-containing activating domain molecule that retains sufficient signaling activity. In some embodiments, a full length or full length intracellular component of an ITAM-containing activating domain molecule is used. In some embodiments, a truncated portion of an ITAM-containing activating domain molecule or intracellular component of an ITAM-containing activating domain molecule is used, provided that the truncated portion retains sufficient signal transduction activity. In further embodiments, an ITAM-containing activating domain is a variant of a whole or truncated portion of an ITAM-containing activating domain molecule, provided that the variant retains sufficient signal transduction activity (i.e., is a functional variant).

Examples of ITAM-containing activating domains that may be used in the chimeric Tim4 receptors of the present disclosure include those derived from CD3ζ, CD3γ, CD3δ, CD3ε, CD5, CD22, CD79a, CD278 (ICOS), DAP10, and CD66d. In specific embodiments, the ITAM-containing activating domain is a CD3ζ signaling domain. An exemplary CD3ζ signaling domain comprises or consists of an amino acid sequence of SEQ ID NO:26 or 27. An exemplary CD3γ signaling domain comprises or consists of an amino acid sequence of SEQ ID NO:227. An exemplary CD38 signaling domain comprises or consists of an amino acid sequence of SEQ ID NO:228. An exemplary CD38 signaling domain comprises or consists of an amino acid sequence of SEQ ID NO:229. An exemplary CD5 signaling domain comprises or consists of an amino acid sequence of SEQ ID NO:230. An exemplary CD22 signaling domain comprises or consists of an amino acid sequence of SEQ ID NO:231. An exemplary CD79a signaling domain comprises or consists of an amino acid sequence of SEQ ID NO:232. An exemplary DAP10 signaling domain comprises or consists of an amino acid sequence of SEQ ID NO:233. An exemplary CD66d signaling domain comprises or consists of an amino acid sequence of SEQ ID NO:234. In certain embodiments, the ITAM-containing activating domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:26, 27, and 227-234. In certain embodiments, the CD33 signaling domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) of an amino acid sequence to any one of SEQ ID NOS:26, 27, and 227-234. In another embodiment, an intracellular signaling domain comprises a CD28 costimulatory signaling domain and a CD3ζ signaling domain. In another embodiment, an intracellular signaling domain comprises a 4-1BB costimulatory signaling domain and a CD3ζ signaling domain. In yet another embodiment, an intracellular signaling domain comprises a CD27 costimulatory signaling domain and a CD3ζ signaling domain. In another embodiment, an intracellular signaling domain comprises a ICOS costimulatory signaling domain and a CD3ζ signaling domain. In another embodiment, an intracellular signaling domain comprises a LFA-1 costimulatory signaling domain and a CD3ζ signaling domain. In another embodiment, an intracellular signaling domain comprises an OX40 costimulatory signaling domain and a CD3ζ signaling domain. In yet another embodiment, an intracellular signaling domain comprises a CD2 costimulatory signaling domain and a CD3ζ signaling domain. In still another embodiment, an intracellular signaling domain comprises an ICAM-1 costimulatory signaling domain and a CD3ζ signaling domain.

Intracellular signaling domains may be derived from a mammalian species, including humans, primates, cows, horses, goats, sheep, dogs, cats, mice, rats, rabbits, guinea pigs, pigs, and transgenic species thereof.

The transmembrane domain of a chimeric Tim4 receptor connects and is positioned between the extracellular domain and the intracellular signaling domain. The transmembrane domain is a hydrophobic alpha helix that transverses the host cell membrane. The transmembrane domain may be directly fused to the binding domain or to the extracellular spacer domain if present. In certain embodiments, the transmembrane domain is derived from an integral membrane protein (e.g., receptor, cluster of differentiation (CD) molecule, enzyme, transporter, cell adhesion molecule, or the like). In one embodiment, the transmembrane domain is selected from the same molecule as the molecule from which the extracellular domain is derived. In another embodiment, the transmembrane domain is selected from the same molecule as the molecule from which the intracellular signaling domain is derived. For example, a chimeric Tim4 receptor may comprise a Tim4 binding domain and a Tim4 transmembrane domain. In another example, a chimeric Tim4 receptor may comprise a CD28 transmembrane domain and a CD28 costimulatory signaling domain. In certain embodiments, the transmembrane domain and the extracellular domain are derived from different molecules; the transmembrane domain and the intracellular signaling domain are derived from different molecules; or the transmembrane domain, extracellular domain, and intracellular signaling domain are all derived from different molecules.

Examples of transmembrane domains that may be used in chimeric Tim4 receptors of the present disclosure include transmembrane domains from Tim1, Tim4, CD3ζ, CD3γ, CD3δ, CD3ε, CD28, CD45, CD4, CD5, CD8, CD9, CD16, CD22, CD33, CD37, CD64, CD80, CD86, CD134, CD137, CD154, CD27, CD28, 4-1BB, OX40, CD30, CD40, PD-1, ICOS, LFA-1, CD2, CD7, LIGHT, NKG2C, and B7-H3. An exemplary Tim4 transmembrane domain comprises or consists of an amino acid sequence of SEQ ID NO:81 or 255. An exemplary Tim transmembrane domain comprises or consists of an amino acid sequence of SEQ ID NO:80. An exemplary CD28 transmembrane domain comprises or consists of an amino acid sequence of SEQ ID NO:25. An exemplary 4-1BB transmembrane domain comprises or consists of an amino acid sequence of SEQ ID NO:237. An exemplary OX40 transmembrane domain comprises or consists of an amino acid sequence of SEQ ID NO:238. An exemplary CD27 transmembrane domain comprises or consists of an amino acid sequence of SEQ ID NO:239. An exemplary ICOS transmembrane domain comprises or consists of an amino acid sequence of SEQ ID NO:240. An exemplary CD2 transmembrane domain comprises or consists of an amino acid sequence of SEQ ID NO:241. An exemplary LFA-1 transmembrane domain comprises or consists of an amino acid sequence of SEQ ID NO:242. An exemplary CD30 transmembrane domain comprises or consists of an amino acid sequence of SEQ ID NO:243. An exemplary CD40 transmembrane domain comprises or consists of an amino acid sequence of SEQ ID NO:244. An exemplary PD-1 transmembrane domain comprises or consists of an amino acid sequence of SEQ ID NO:245. An exemplary CD7 transmembrane domain comprises or consists of an amino acid sequence of SEQ ID NO:246. An exemplary LIGHT transmembrane domain comprises or consists of an amino acid sequence of SEQ ID NO:247. An exemplary NKG2C transmembrane domain comprises or consists of an amino acid sequence of SEQ ID NO:248. An exemplary B7-H3 transmembrane domain comprises or consists of an amino acid sequence of SEQ ID NO:249. In certain embodiments, the transmembrane domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:25, 80, 81, 237-249, and 255. In certain embodiments, the transmembrane domain comprises an amino acid sequence having at least about 1, 2, 3, 4, 5, 6, 7, 8, 9, or 10 amino acid modifications (e.g., deletion, additions, substitutions) to an amino acid sequence of any one of SEQ ID NOS:25, 80, 81, 237-249, and 255.

Transmembrane domains may derived from any mammalian species, including humans, primates, cows, horses, goats, sheep, dogs, cats, mice, rats, rabbits, guinea pigs, pigs, and transgenic species thereof.

Exemplary components, configurations, and chimeric Tim4 receptor sequences of the present disclosure are provided in Table 3.

TABLE 3

| Name | Amino Acid Sequence | SEQ ID NO: |
|---|---|---|
| Tim4 binding | MSKEPLILWLMIEFWWLYLTPVTSETVVTEVLGHRVTLPCLYSSW | 50 |
| domain (amino | SHNSNSMCWGKDQCPYSGCKEALIRTDGMRVTSRKSAKYRLQGT | |
| acids 1-24 signal | IPRGDVSLTILNPSESDSGVYCCRIEVPGWFNDVKINVRLNLQRAST | |
| peptide) | TTHRTATTTTRRTTTTSPTTTRQMTTTPAALPTTVVTTPDLTTGTPL | |
| | QMTTIAVFTTANTCLSLTPSTLPEEATGLLTPEPSKEGPILTAESETV | |
| | LPSDSWSSVESTSADTVLLTSKESKVWDLPSTSHVSMWKTSDSVS | |
| | SPQPGASDTAVPEQNKTTKTGQMDGIPMSMKNEMPISQ | |
| Modified IgG4 | ESKYGPPCPPCP | 21 |
| hinge | | |
| Wildtype CD28 | RSKRSRLLHSDYMNMTPRRPGPTRKHYQPYAPPRDFAAYRS | 28 |
| costimulatory | | |
| signaling domain | | |
| CD28 | RSKRSRGGHSDYMNMTPRRPGPTRKHYQPYAPPRDFAAYRS | 29 |
| costimulatory | | |
| signaling domain | | |
| with | | |
| L186G/L187G | | |
| substitutions | | |
| (positions | | |
| reference to full | | |
| length protein) | | |
| OX40 | ALYLLRRDQRLPPDAHKPPGGGSFRTPIQEEQADAHSTLAKI | 213 |
| costimulatory | | |
| signaling domain | | |
| CD2 | KRKKQRSRRNDEELETRAHRVATEERGRKPHQIPASTPQNPATSQ | 214 |
| costimulatory | HPPPPPGHRSQAPSHRPPPPGHRVQHQPQKRPPAPSGTQVHQQKGP | |
| signaling domain | PLPRPRVQPKPPHGAAENSLSPSSN | |
| 4-1BB | KRGRKKLLYIFKQPFMRPVQTTQEEDGCSCRFPEEEEGGCEL | 215 |
| costimulatory | | |
| signaling domain | | |
| CD27 | QRRKYRSNKGESPVEPAEPCHYSCPREEEGSTIPIQEDYRKPEPACS | 216 |
| costimulatory | P | |
| signaling domain | | |
| ICAM-1 | NRQRKIKKYRLQQAQKGTPMKPNTQATPP | 217 |
| costimulatory | | |
| signaling domain | | |
| LFA-1 | KALIHLSDLREYRRFEKEKLKSQWNNDNPLFKSATTTVMNPKFAE | 218 |
| costimulatory | S | |
| signaling domain | | |
| ICOS | CWLTKKKYSSSVHDPNGEYMFMRAVNTAKKSRLTDVTL | 219 |
| costimulatory | | |
| signaling domain | | |
| CD30 | HRRACRKRIRQKLHLCYPVQTSQPKLELVDSRPRRSSTQLRSGASV | 220 |
| costimulatory | TEPVAEERGLMSQPLMETCHSVGAAYLESLPLQDASPAGGPSSPR | |
| signaling domain | DLPEPRVSTEHTNNKIEKIYIMKADTVIVGTVKAELPEGRGLAGPA | |
| | EPELEEELEADHTPHYPEQETEPPLGSCSDVMLSVEEEGKEDPLPT | |
| | AASGK | |
| CD40 | KKVAKKPTNKAPHPKQEPQEINFPDDLPGSNTAAPVQETLHGCQP | 221 |
| costimulatory | VTQEDGKESRISVQERQ | |
| signaling domain | | |
| PD-1 | CSRAARGTIGARRTGQPLKEDPSAVPVFSVDYGELDFQWREKTPE | 222 |
| costimulatory | PPVPCVPEQTEYATIVFPSGMGTSSPARRGSADGPRSAQPLRPEDG | |
| signaling domain | HCSWPL | |
| CD7 | RTQIKKLCSWRDKNSAACVVYEDMSHSRCNTLSSPNQYQ | 223 |
| costimulatory | | |
| signaling domain | | |
| LIGHT | MEESVVRPSVFVVDGQTDIPFTRLGRSHRRQSCSVAR | 224 |
| costimulatory | | |
| signaling domain | | |
| NKG2C | MSKQRGTFSEVSLAQDPKRQQRKPKGNKSSISGTEQEIFQVELNLQ | 225 |
| costimulatory | NPSLNHQGIDKIYDCQGLLPPPEK | |
| signaling domain | | |
| B7-H3 | CWRKIKQSCEEENAGAEDQDGEGEGSKTALQPLKHSDSKEDDGQ | 226 |
| costimulatory | EIA | |
| signaling domain | | |
| wildtype CD3ζ | RVKFSRSADAPAYQQGQNQLYNELNLGRREEYDVLDKRRGRDPE | 26 |
| activating domain | MGGKPQRRKNPQEGLYNELQKDKMAEAYSEIGMKGERRRGKGH | |
| | DGLYQGLSTATKDTYDALHMQALPPR | |
| mutated human | RVKFSRSADAPAYQQGQNQLYNELNLGRREEYDVLDKRRGRDPE | 27 |
| CD3ζ ITAM- | MGGKPRRKNPQEGLYNELQKDKMAEAYSEIGMKGERRRGKGHD | |
| containing | GLYQGLSTATKDTYDALHMQALPPR | |
| activating domain | | |
| CD3γ ITAM- | GQDGVRQSRASDKQTLLPNDQLYQPLKDREDDQYSHLQGNQLRR | 227 |
| containing | N | |
| activating domain | | |
| CD3δ ITAM- | GHETGRLSGAADTQALLRNDQVYQPLRDRDDAQYSHLGGNWAR | 228 |
| containing | NK | |
| activating domain | | |
| CD3ϵ ITAM- | KNRKAKAKPVTRGAGAGGRQRGQNKERPPPVPNPDYEPIRKGQR | 229 |
| containing | DLYSGLNQRRI | |
| activating domain | | |
| CD5 ITAM- | KKLVKKFRQKKQRQWIGPTGMNQNMSFHRNHTATVRSHAENPT | 230 |
| containing | ASHVDNEYSQPPRNSHLSAYPALEGALHRSSMQPDNSSDSDYDLH | |
| activating domain | GAQRL | |
| CD22 ITAM- | KLQRRWKRTQSQQGLQENSSGQSFFVRNKKVRRAPLSEGPHSLGC | 231 |
| containing | YNPMMEDGISYTTLRFPEMNIPRTGDAESSEMQRPPPDCDDTVTYS | |
| activating domain | ALHKRQVGDYENVIPDFPEDEGIHYSELIQFGVGERPQAQENVDY | |
| | VILKH | |
| CD79a ITAM- | RKRWQNEKLGLDAGDEYEDENLYEGLNLDDCSMYEDISRGLQGT | 232 |
| containing | YQDVGSLNIGDVQLEKP | |
| activating domain | | |
| DAP10 ITAM- | LCARPRRSPAQEDGKVYINMPGRG | 233 |
| containing | | |
| activating domain | | |
| CD66d ITAM- | AKTGRTSIQRDLKEQQPQALAPGRGPSHSSAFSMSPLSTAQAPLPN | 234 |
| containing | PRTAASIYEELLKHDTNIYCRMDHKAEVAS | |
| activating domain | | |
| Tim4 | LLMIIAPSLGFVLFALFVAFL | 235 |
| transmembrane | | |
| domain | | |
| CD28 | FWVLVVVGGVLACYSLLVTVAFIIFWV | 236 |
| transmembrane | | |
| domain | | |
| CD28 | MFWVLVVVGGVLACYSLLVTVAFIIFWV | |
| transmembrane | | |
| domain | | |
| 4-1BB | IISFFLALTSTALLFLLFFLTLRFSVV | 237 |
| transmembrane | | |
| domain | | |
| OX40 | VAAILGLGLVLGLLGPLAILL | 238 |
| transmembrane | | |
| domain | | |
| CD27 | ILVIFSGMFLVFTLAGALFLH | 239 |
| transmembrane | | |
| domain | | |
| ICOS | FWLPIGCAAFVVVCILGCILI | 240 |
| transmembrane | | |
| domain | | |
| CD2 | IYLIIGICGGGSLLMVFVALLVFYIT | 241 |
| transmembrane | | |
| domain | | |
| LFA-1 | IAAIVGGTVAGIVLIGILLLVIW | 242 |
| transmembrane | | |
| domain | | |
| CD30 | PVLDAGPVLFWVILVLVVVVGSSAFLLC | 243 |
| transmembrane | | |
| domain | | |
| CD40 | ALVVIPIIFGILFAILLVLVFI | 244 |
| transmembrane | | |
| domain | | |
| PD-1 | VGVVGGLLGSLVLLVWVLAVI | 245 |
| transmembrane | | |
| domain | | |
| CD7 | AALAVISFLLGLGLGVACVLA | 246 |
| transmembrane | | |
| domain | | |
| LIGHT | VGLGLLLLLMGAGLAVQGWFL | 247 |
| transmembrane | | |
| domain | | |
| NKG2C | LTAEVLGIICIVLMATVLKTIVL | 248 |
| transmembrane | | |
| domain | | |
| B7-H3 | LWVTVGLSVCLIALLVALAFV | 249 |
| transmembrane | | |
| domain | | |
| Tim4 binding | MSKGLLLLWLVTELWWLYLTPAASEDTIIGFLGQPVTLPCHYLSW | 250 |
| domain-CD28 | SQSRNSMCWGKGSCPNSKCNAELLRTDGTRIISRKSTKYTLLGKV | |
| transmembrane- | QFGEVSLTISNTNRGDSGVYCCRIEVPGWFNDVKKNVRLELRRAT | |
| CD28 | TTKKPTTTTRPTTTPYVTTTTPELLPTTVMTTSVLPTTTPPQTLATT | |
| costimulatory | AFSTAVTTCPSTTPGSFSQETTKGSAFTTESETLPASNHSQRSMMTI | |
| signaling domain, | STDIAVLRPTGSNPGILPSTSQLTTQKTTLTTSESLQKTTKSHQINSR | |
| amino acids 1-22 | QTFWVLVVVGGVLACYSLLVTVAFIIFWVRSKRSRGGHSDYMNM | |
| are signal peptide | TPRRPGPTRKHYQPYAPPRDFAAYRS | |
| Tim4 binding | MSKGLLLLWLVTELWWLYLTPAASEDTIIGFLGQPVTLPCHYLSW | 251 |
| domain-Tim4 | SQSRNSMCWGKGSCPNSKCNAELLRTDGTRIISRKSTKYTLLGKV | |
| transmembrane- | QFGEVSLTISNTNRGDSGVYCCRIEVPGWFNDVKKNVRLELRRAT | |
| CD28 | TTKKPTTTTRPTTTPYVTTTTPELLPTTVMTTSVLPTTTPPQTLATT | |
| costimulatory | AFSTAVTTCPSTTPGSFSQETTKGSAFTTESETLPASNHSQRSMMTI | |
| signaling domain, | STDIAVLRPTGSNPGILPSTSQLTTQKTTLTTSESLQKTTKSHQINSR | |
| amino acids 1-22 | QTILIIACCVGFVLMVLLFLAFLRSKRSRGGHSDYMNMTPRRPGPT | |
| are signal peptide | RKHYQPYAPPRDFAAYRS | |
| Tim4 binding | MSKGLLLLWLVTELWWLYLTPAASEDTIIGFLGQPVTLPCHYLSW | 252 |
| domain-Tim4 | SQSRNSMCWGKGSCPNSKCNAELLRTDGTRIISRKSTKYTLLGKV | |
| transmembrane-4- | QFGEVSLTISNTNRGDSGVYCCRIEVPGWFNDVKKNVRLELRRAT | |
| 1BB | TTKKPTTTTRPTTTPYVTTTTPELLPTTVMTTSVLPTTTPPQTLATT | |
| costimulatory | AFSTAVTTCPSTTPGSFSQETTKGSAFTTESETLPASNHSQRSMMTI | |
| signaling domain- | STDIAVLRPTGSNPGILPSTSQLTTQKTTLTTSESLQKTTKSHQINSR | |
| CD3ζ ITAM | QTILIIACCVGFVLMVLLFLAFLKRGRKKLLYIFKQPFMRPVQTTQE | |
| containing | EDGCSCRFPEEEEGGCELRVKFSRSADAPAYQQGQNQLYNELNLG | |
| activating domain, | RREEYDVLDKRRGRDPEMGGKPRRKNPQEGLYNELQKDKMAEA | |
| amino acids 1-22 | YSEIGMKGERRRGKGHDGLYQGLSTATKDTYDALHMQALPPR | |
| are signal peptide | | |
| “CER136” | MSKEPLILWLMIEFWWLYLTPVTS | 253 |
| TIM4 binding | ETVVTEVLGHRVTLPCLYSSWSHNSNSMCWGKDQCPYSGCKEAL | |
| domain-CD28 TM- | IRTDGMRVTSRKSAKYRLQGTIPRGDVSLTILNPSESDSGVYCCRIE | |
| CD28 costim- | VPGWFNDVKINVRLNLQRASTTTHRTATTTTRRTTTTSPTTTRQM | |
| CD3ζ (amino | TTTPAALPTTVVTTPDLTTGTPLQMTTIAVFTTANTCLSLTPSTLPE | |
| acids 1-24 signal | EATGLLTPEPSKEGPILTAESETVLPSDSWSSVESTSADTVLLTSKES | |
| peptide) | KVWDLPSTSHVSMWKTSDSVSSPQPGASDTAVPEQNKTTKTGQM | |
| | DGIPMSMKNEMPISQ | |
| | FWVLVVVGGVLACYSLLVTVAFIIFWV | |
| | RSKRSRLLHSDYMNMTPRRPGPTRKHYQPYAPPRDFAAYRSSRVKFSR | |
| | SADAPAYQQGQNQLYNELNLGRREEYDVLDKRRGRDPEMGGKP | |
| | QRRKNPQEGLYNELQKDKMAEAYSEIGMKGERRRGKGHDGLYQ | |
| | GLSTATKDTYDALHMQALPPR | |
| TIM4-CD28 CER: | MSKEPLILWLMIEFWWLYLTPVTSETVVTEVLGHRVTLPCLYSSW | 557 |
| Tim4 signal | SHNSNSMCWGKDQCPYSGCKEALIRTDGMRVTSRKSAKYRLQG | |
| peptide, TIM4 | TIPRGDVSLTILNPSESDSGVYCCRIEVPGWFNDVKINVRLNLQRA | |
| binding domain, | STTTHRTATTTTRRTTTTSPTTTRQMTTTPAALPTTVVTTPDLTTG | |
| CD28 TM, CD28 | TPLQMTTIAVFTTANTCLSLTPSTLPEEATGLLTPEPSKEGPILTAE | |
| signaling | SETVLPSDSWSSVESTSADTVLLTSKESKVWDLPSTSHVSMWKT | |
| | SDSVSSPQPGASDTAVPEQNKTTKTGQMDGIPMSMKNEMPISQ | |
| | MFWVLVVVGGVLACYSLLVTVAFIIFWVRSKRSRLLHSDYMNMTP | |
| | RRPGPTRKHYQPYAPPRDFAAYRS | |
| GM-CSF signal | MLLLVTSLLLCELPHPAFLLIP | 254 |
| peptide | | |
| Tim4 signal | MSKEPLILWLMIEFWWLYLTPVTS | 54 |
| peptide | | |
| Mouse Tim4 | ILIIACCVGFVLMVLLFLAFL | 255 |
| transmembrane | | |
| domain | | |
| Mouse Tim4 | MSKGLLLLWLVTELWWLYLTPAASEDTIIGFLGQPVTLPCHYLSW | 256 |
| binding domain, | SQSRNSMCWGKGSCPNSKCNAELLRTDGTRIISRKSTKYTLLGKV | |
| amino acids 1-22 | QFGEVSLTISNTNRGDSGVYCCRIEVPGWFNDVKKNVRLELRRAT | |
| are signal peptide | TTKKPTTTTRPTTTPYVTTTTPELLPTTVMTTSVLPTTTPPQTLATT | |
| | AFSTAVTTCPSTTPGSFSQETTKGSAFTTESETLPASNHSQRSMMTI | |
| | STDIAVLRPTGSNPGILPSTSQLTTQKTTLTTSESLQKTTKSHQINSR | |
| | QT | |
| Mouse Tim4 | MSKGLLLLWLVTELWWLYLTPA | 55 |
| signal peptide | | |

Further embodiments of the chimeric Tim4 receptors described herein comprise a single chain chimeric protein, the single chain chimeric protein comprising: an extracellular domain comprising a Tim4 binding domain; an intracellular signaling domain comprising a first costimulatory signaling domain and an ITAM-containing activating domain, wherein the ITAM-containing activating domain comprises a DAP12 signaling domain; and a transmembrane domain positioned between and connecting the extracellular domain and the intracellular signaling domain.

A Tim4 binding domain suitable for use in a chimeric Tim4 receptor of the present disclosure may be any polypeptide or peptide derived from a Tim4 molecule that specifically binds phosphatidylserine. In certain embodiments, the Tim4 binding domain is derived from human Tim4. An exemplary human Tim4 molecule is provided in Uniprot. Ref. Q96H15 (SEQ ID NO:49). An exemplary human Tim4 binding domain comprises or consists of an amino acid sequence of SEQ ID NO:50 or amino acids 25-314 of SEQ ID NO:50. An exemplary mouse Tim4 binding domain comprises or consists of an amino acid sequence of SEQ ID NO:51 or amino acids 23-279 of SEQ ID NO:51. In certain embodiments, the Tim4 binding domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:50 or amino acids 25-314 of SEQ ID NO:50, or SEQ ID NO:51 or amino acids 23-279 of SEQ ID NO:51. In certain embodiments, the Tim4 binding domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:50 or amino acids 25-314 of SEQ ID NO:50, or SEQ ID NO:51 or amino acids 23-279 of SEQ ID NO:51.

In certain embodiments, the extracellular domain optionally comprises an extracellular, non-signaling spacer or linker domain. Where included, such a spacer or linker domain may position the binding domain away from the host cell surface to further enable proper cell/cell contact, binding, and activation. When included in a chimeric receptor as described herein, an extracellular spacer domain is generally located between the extracellular binding domain and the transmembrane domain of the chimeric Tim4 receptor. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG₄ hinge region is described in PCT Publication No. WO 2014/031687, which hinge region is incorporated herein by reference in its entirety. In a particular embodiment, an extracellular spacer domain comprises a modified IgG4 hinge region having the amino acid sequence of ESKYGPPCPPCP (SEQ ID NO:21). Other examples of hinge regions that may be used in the chimeric Tim4 receptors described herein include the hinge region from the extracellular regions of type 1 membrane proteins, such as CD8a, CD4, CD28 and CD7, which may be wild-type or variants thereof. In some embodiments a CD8a hinge region comprises the amino acid sequence of SEQ ID NO:22. In some embodiments, a CD28 hinge region comprises the amino acid sequence of SEQ ID NO:23. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D.

In certain embodiments, an extracellular domain is encoded by polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, an extracellular domain is marine, human, or chimeric.

In certain embodiments, an intracellular signaling domain comprises a costimulatory signaling domain and an ITAM-containing activating domain, wherein the ITAM-containing activating domain comprises a DAP12 signaling domain. The costimulatory signaling domain may be any portion of a costimulatory signaling molecule that retains sufficient signaling activity. In some embodiments, a full length or full length intracellular component of a costimulatory signaling molecule is used. In some embodiments, a truncated portion of a costimulatory signaling molecule or intracellular component of a costimulatory signaling molecule is used, provided that the truncated portion retains sufficient signal transduction activity. In further embodiments, a costimulatory signaling domain is a variant of a whole or truncated portion of a costimulatory signaling molecule, provided that the variant retains sufficient signal transduction activity (i.e., is a functional variant).

In certain embodiments, the costimulatory signaling domain comprises a CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), PD-1, CD273 (PD-L2), CD274 (PD-L1), B7-H3 (CD276), ICOS (CD278), DAP10, LAT, LFA-1 (CD11a/CD18), LIGHT, NKG2C, SLP76, or TRIM signaling domain. In particular embodiments, the costimulatory signaling domain comprises an OX40, CD2, CD27, CD28, ICAM-1, LFA-1 (CD11a/CD18), ICOS (CD278), or 4-1BB (CD137) signaling domain. An exemplary CD28 costimulatory signaling domain comprises or consists of an amino acid sequence of SEQ ID NO:28 or 29. An exemplary OX40 costimulatory signaling domain comprises or consists of an amino acid sequence of SEQ ID NO:213. An exemplary CD2 costimulatory signaling domain comprises or consists of an amino acid sequence of SEQ ID NO:214. An exemplary 4-1BB costimulatory signaling domain comprises or consists of an amino acid sequence of SEQ ID NO:215. An exemplary CD27 costimulatory signaling domain comprises or consists of an amino acid sequence of SEQ ID NO:216. An exemplary ICAM-1 costimulatory signaling domain comprises or consists of an amino acid sequence of SEQ ID NO:217. An exemplary LFA-1 costimulatory signaling domain comprises or consists of an amino acid sequence of SEQ ID NO:218. An exemplary ICOS costimulatory signaling domain comprises or consists of an amino acid sequence of SEQ ID NO:219. An exemplary CD30 costimulatory signaling domain comprises or consists of an amino acid sequence of SEQ ID NO:220. An exemplary CD40 costimulatory signaling domain comprises or consists of an amino acid sequence of SEQ ID NO:221. An exemplary PD-1 costimulatory signaling domain comprises or consists of an amino acid sequence of SEQ ID NO:222. An exemplary CD7 costimulatory signaling domain comprises or consists of an amino acid sequence of SEQ ID NO:223. An exemplary LIGHT costimulatory signaling domain comprises or consists of an amino acid sequence of SEQ ID NO:224. An exemplary NKG2C costimulatory signaling domain comprises or consists of an amino acid sequence of SEQ ID NO:225. An exemplary B7-H3 costimulatory signaling domain comprises or consists of an amino acid sequence of SEQ ID NO:226. In certain embodiments, the costimulatory signaling domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:28, 29, and 213-226. In certain embodiments, the costimulatory signaling domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of any one of SEQ ID NOS:28, 29, and 213-226. In certain embodiments, the intracellular signaling comprises a second costimulatory signaling domain. In preferred embodiments, the first costimulatory signaling domain and second costimulatory signaling domain are different.

The DAP12 signaling domain may recapitulate TCR signaling independently of endogenous TCR complexes. In certain embodiments, signaling via the DAP12 signaling domain leads to mediation of a T cell response, including, but not limited to, proliferation, activation, differentiation, and the like. The DAP12 signaling domain may be any portion of the DAP12 molecule that retains sufficient signaling activity. In some embodiments, a full length or full length intracellular component of the DAP12 molecule is used. In some embodiments, a truncated portion of DAP12 or intracellular component of a DAP12 is used, provided that the truncated portion retains sufficient signal transduction activity. In further embodiments, the DAP12 signaling domain is a variant of a whole or truncated portion of DAP12, provided that the variant retains sufficient signal transduction activity (i.e., is a functional variant).

An exemplary human DAP12 molecule is provided in Uniprot. Ref. 043914. An exemplary DAP12 signaling domain comprises or consists of an amino acid sequence of SEQ ID NO:103. In certain embodiments, the DAP12 signaling domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO: 103. In certain embodiments, the DAP12 signaling domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence to SEQ ID NO: 103.

In another embodiment, an intracellular signaling domain comprises a CD28 costimulatory signaling domain and a DAP12 signaling domain. In another embodiment, an intracellular signaling domain comprises a 4-1BB costimulatory signaling domain and a DAP12 signaling domain. In yet another embodiment, an intracellular signaling domain comprises a CD27 costimulatory signaling domain and a DAP12 signaling domain. In another embodiment, an intracellular signaling domain comprises a ICOS costimulatory signaling domain and a DAP12 signaling domain. In another embodiment, an intracellular signaling domain comprises a LFA-1 costimulatory signaling domain and a DAP12 signaling domain. In another embodiment, an intracellular signaling domain comprises an OX40 costimulatory signaling domain and a DAP12 signaling domain. In yet another embodiment, an intracellular signaling domain comprises a CD2 costimulatory signaling domain and a DAP12 signaling domain. In still another embodiment, an intracellular signaling domain comprises an ICAM-1 costimulatory signaling domain and a DAP12 signaling domain.

Intracellular signaling domains may be derived from a mammalian species, including humans, primates, cows, horses, goats, sheep, dogs, cats, mice, rats, rabbits, guinea pigs, pigs, and transgenic species thereof.

In certain embodiments, the transmembrane domain is derived from an integral membrane protein (e.g., receptor, cluster of differentiation (CD) molecule, enzyme, transporter, cell adhesion molecule, or the like). In one embodiment, the transmembrane domain is selected from the same molecule as the molecule from which the extracellular domain is derived. In another embodiment, the transmembrane domain is selected from the same molecule as the molecule from which the intracellular signaling domain is derived. For example, a chimeric Tim4 receptor may comprise a Tim4 binding domain and a Tim4 transmembrane domain. In another example, a chimeric Tim4 receptor may comprise a CD28 transmembrane domain and a CD28 costimulatory signaling domain. In certain embodiments, the transmembrane domain and the extracellular domain are derived from different molecules; the transmembrane domain and the intracellular signaling domain are derived from different molecules; or the transmembrane domain, extracellular domain, and intracellular signaling domain are all derived from different molecules. Examples of transmembrane domains that may be used in chimeric Tim4 receptors of the present disclosure include transmembrane domains from Tim4, Tim1, CD3ζ, CD3γ, CD3δ, CD3ε, CD28, CD45, CD4, CD5, CD8, CD9, CD16, CD22, CD33, CD37, CD64, CD80, CD86, CD134, CD137, CD154, CD27, CD28, 4-1BB, OX40, CD30, CD40, PD-1, ICOS, LFA-1, CD2, CD7, LIGHT, NKG2C, and B7-H3. An exemplary Tim4 transmembrane domain comprises or consists of an amino acid sequence of SEQ ID NO:235 or 255. An exemplary Tim1 transmembrane domain comprises or consists of SEQ ID NO:80. An exemplary CD28 transmembrane domain comprises or consists of an amino acid sequence of SEQ ID NO:236. An exemplary 4-1BB transmembrane domain comprises or consists of an amino acid sequence of SEQ ID NO:237. An exemplary OX40 transmembrane domain comprises or consists of an amino acid sequence of SEQ ID NO:238. An exemplary CD27 transmembrane domain comprises or consists of an amino acid sequence of SEQ ID NO:239. An exemplary ICOS transmembrane domain comprises or consists of an amino acid sequence of SEQ ID NO:240. An exemplary CD2 transmembrane domain comprises or consists of an amino acid sequence of SEQ ID NO:241. An exemplary LFA-1 transmembrane domain comprises or consists of an amino acid sequence of SEQ ID NO:242. An exemplary CD30 transmembrane domain comprises or consists of an amino acid sequence of SEQ ID NO:243. An exemplary CD40 transmembrane domain comprises or consists of an amino acid sequence of SEQ ID NO:244. An exemplary PD-1 transmembrane domain comprises or consists of an amino acid sequence of SEQ ID NO:245. An exemplary CD7 transmembrane domain comprises or consists of an amino acid sequence of SEQ ID NO:246. An exemplary LIGHT transmembrane domain comprises or consists of an amino acid sequence of SEQ ID NO:247. An exemplary NKG2C transmembrane domain comprises or consists of an amino acid sequence of SEQ ID NO:248. An exemplary B7-H3 transmembrane domain comprises or consists of an amino acid sequence of SEQ ID NO:249. In certain embodiments, the transmembrane domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:235-249 and 255. In certain embodiments, the transmembrane domain comprises an amino acid sequence having at least about 1, 2, 3, 4, 5, 6, 7, 8, 9, or 10 amino acid modifications (e.g., deletion, additions, substitutions) to an amino acid sequence of any one of SEQ ID NOS:235-249 and 255.

Transmembrane domains may derived from any mammalian species, including humans, primates, cows, horses, goats, sheep, dogs, cats, mice, rats, rabbits, guinea pigs, pigs, and transgenic species thereof.

In certain embodiments, a chimeric Tim4 receptor is encoded by polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, a chimeric Tim4 receptor is murine, chimeric, human, or humanized.

It is understood that direct fusion of one domain to another domain of a chimeric Tim4 receptor described herein does not preclude the presence of intervening junction amino acids. Junction amino acids may be natural or non-natural (e.g., resulting from the construct design of a chimeric protein). For example, junction amino acids may result from restriction enzyme sites used for joining one domain to another domain or cloning polynucleotides encoding chimeric Tim4 receptors into vectors.

Exemplary configurations, components, chimeric Tim4 receptors, and sequences thereof of the present disclosure are provided in Table 4.

TABLE 4

| Name | Amino Acid Sequence | SEQ ID NO: |
|---|---|---|
| human Tim4 | MSKEPLILWLMIEFWWLYLTPVTSETVVTEVLGHRVTLPCLYSS | 50 |
| binding domain | WSHNSNSMCWGKDQCPYSGCKEALIRTDGMRVTSRKSAKYRLQ | |
| (amino acids 1-24 | GTIPRGDVSLTILNPSESDSGVYCCRIEVPGWFNDVKINVRLNLQR | |
| signal peptide) | ASTTTHRTATTTTRRTTTTSPTTTRQMTTTPAALPTTVVTTPDLTT | |
| | GTPLQMTTIAVFTTANTCLSLTPSTLPEEATGLLTPEPSKEGPILTA | |
| | ESETVLPSDSWSSVESTSADTVLLTSKESKVWDLPSTSHVSMWK | |
| | TSDSVSSPQPGASDTAVPEQNKTTKTGQMDGIPMSMKNEMPISQ | |
| mouse Tim4 | MSKGLLLLWLVTELWWLYLTPAASEDTIIGFLGQPVTLPCHYLS | 51 |
| binding domain | WSQSRNSMCWGKGSCPNSKCNAELLRTDGTRIISRKSTKYTLLG | |
| (amino acids 1-22 | KVQFGEVSLTISNTNRGDSGVYCCRIEVPGWFNDVKKNVRLELR | |
| signal peptide) | RATTTKKPTTTTRPTTTPYVTTTTPELLPTTVMTTSVLPTTTPPQT | |
| | LATTAFSTAVTTCPSTTPGSFSQETTKGSAFTTESETLPASNHSQR | |
| | SMMTISTDIAVLRPTGSNPGILPSTSQLTTQKTTLTTSESLQKTTKS | |
| | HQINSRQT | |
| Modified IgG4 | ESKYGPPCPPCP | 21 |
| hinge | | |
| CD8a hinge | TTTPAPRPPTPAPTIASQPLSLRPEACRPAAGGAVHTRGLDFACD | 22 |
| region | | |
| CD28 hinge | IEVMYPPPYLDNEKSNGTIIHVKGKHLCPSPLFPGPSKP | 23 |
| region | | |
| Wildtype CD28 | RSKRSRLLHSDYMNMTPRRPGPTRKHYQPYAPPRDFAAYRS | 28 |
| costimulatory | | |
| signaling domain | | |
| CD28 | RSKRSRGGHSDYMNMTPRRPGPTRKHYQPYAPPRDFAAYRS | 29 |
| costimulatory | | |
| signaling domain | | |
| with | | |
| L186G/L187G | | |
| substitutions | | |
| (positions | | |
| reference to full | | |
| length protein) | | |
| OX40 | ALYLLRRDQRLPPDAHKPPGGGSFRTPIQEEQADAHSTLAKI | 213 |
| costimulatory | | |
| signaling domain | | |
| CD2 | KRKKQRSRRNDEELETRAHRVATEERGRKPHQIPASTPQNPATSQ | 214 |
| costimulatory | HPPPPPGHRSQAPSHRPPPPGHRVQHQPQKRPPAPSGTQVHQQKG | |
| signaling domain | PPLPRPRVQPKPPHGAAENSLSPSSN | |
| 4-1BB | KRGRKKLLYIFKQPFMRPVQTTQEEDGCSCRFPEEEEGGCEL | 215 |
| costimulatory | | |
| signaling domain | | |
| CD27 | QRRKYRSNKGESPVEPAEPCHYSCPREEEGSTIPIQEDYRKPEPAC | 216 |
| costimulatory | SP | |
| signaling domain | | |
| ICAM-1 | NRQRKIKKYRLQQAQKGTPMKPNTQATPP | 217 |
| costimulatory | | |
| signaling domain | | |
| LFA-1 | KALIHLSDLREYRRFEKEKLKSQWNNDNPLFKSATTTVMNPKFA | 218 |
| costimulatory | ES | |
| signaling domain | | |
| ICOS | CWLTKKKYSSSVHDPNGEYMFMRAVNTAKKSRLTDVTL | 219 |
| costimulatory | | |
| signaling domain | | |
| CD30 | HRRACRKRIRQKLHLCYPVQTSQPKLELVDSRPRRSSTQLRSGAS | 220 |
| costimulatory | VTEPVAEERGLMSQPLMETCHSVGAAYLESLPLQDASPAGGPSSP | |
| signaling domain | RDLPEPRVSTEHTNNKIEKIYIMKADTVIVGTVKAELPEGRGLAG | |
| | PAEPELEEELEADHTPHYPEQETEPPLGSCSDVMLSVEEEGKEDP | |
| | LPTAASGK | |
| CD40 | KKVAKKPTNKAPHPKQEPQEINFPDDLPGSNTAAPVQETLHGCQ | 221 |
| costimulatory | PVTQEDGKESRISVQERQ | |
| signaling domain | | |
| PD-1 | CSRAARGTIGARRTGQPLKEDPSAVPVFSVDYGELDFQWREKTP | 222 |
| costimulatory | EPPVPCVPEQTEYATIVFPSGMGTSSPARRGSADGPRSAQPLRPED | |
| signaling domain | GHCSWPL | |
| CD7 | RTQIKKLCSWRDKNSAACVVYEDMSHSRCNTLSSPNQYQ | 223 |
| costimulatory | | |
| signaling domain | | |
| LIGHT | MEESVVRPSVFVVDGQTDIPFTRLGRSHRRQSCSVAR | 224 |
| costimulatory | | |
| signaling domain | | |
| NKG2C | MSKQRGTFSEVSLAQDPKRQQRKPKGNKSSISGTEQEIFQVELNL | 225 |
| costimulatory | QNPSLNHQGIDKIYDCQGLLPPPEK | |
| signaling domain | | |
| B7-H3 | CWRKIKQSCEEENAGAEDQDGEGEGSKTALQPLKHSDSKEDDG | 226 |
| costimulatory | QEIA | |
| signaling domain | | |
| DAP12 | YFLGRLVPRGRGAAEAATRKQRITETESPYQELQGQRSDVYSDL | 103 |
| | NTQRPYYK | |
| Tim4 | LLMIIAPSLGFVLFALFVAFL | 235 |
| transmembrane | | |
| domain | | |
| CD28 | FWVLVVVGGVLACYSLLVTVAFIIFWV | 236 |
| transmembrane | | |
| domain | | |
| 4-1BB | IISFFLALTSTALLFLLFFLTLRFSVV | 237 |
| transmembrane | | |
| domain | | |
| OX40 | VAAILGLGLVLGLLGPLAILL | 238 |
| transmembrane | | |
| domain | | |
| CD27 | ILVIFSGMFLVFTLAGALFLH | 239 |
| transmembrane | | |
| domain | | |
| ICOS | FWLPIGCAAFVVVCILGCILI | 240 |
| transmembrane | | |
| domain | | |
| CD2 | IYLIIGICGGGSLLMVFVALLVFYIT | 241 |
| transmembrane | | |
| domain | | |
| LFA-1 | IAAIVGGTVAGIVLIGILLLVIW | 242 |
| transmembrane | | |
| domain | | |
| CD30 | PVLDAGPVLFWVILVLVVVVGSSAFLLC | 243 |
| transmembrane | | |
| domain | | |
| CD40 | ALVVIPIIFGILFAILLVLVFI | 244 |
| transmembrane | | |
| domain | | |
| PD-1 | VGVVGGLLGSLVLLVWVLAVI | 245 |
| transmembrane | | |
| domain | | |
| CD7 | AALAVISFLLGLGLGVACVLA | 246 |
| transmembrane | | |
| domain | | |
| LIGHT | VGLGLLLLLMGAGLAVQGWFL | 247 |
| transmembrane | | |
| domain | | |
| NKG2C | LTAEVLGIICIVLMATVLKTIVL | 248 |
| transmembrane | | |
| domain | | |
| B7-H3 | LWVTVGLSVCLIALLVALAFV | 249 |
| transmembrane | | |
| domain | | |
| Tim4 binding | MSKEPLILWLMIEFWWLYLTPVTSETVVTEVLGHRVTLPCLYSS | 257 |
| domain-CD28 | WSHNSNSMCWGKDQCPYSGCKEALIRTDGMRVTSRKSAKYRLQ | |
| hinge-CD28 | GTIPRGDVSLTILNPSESDSGVYCCRIEVPGWFNDVKINVRLNLQR | |
| transmembrane- | ASTTTHRTATTTTRRTTTTSPTTTRQMTTTPAALPTTVVTTPDLTT | |
| CD28 | GTPLQMTTIAVFTTANTCLSLTPSTLPEEATGLLTPEPSKEGPILTA | |
| costimulatory | ESETVLPSDSWSSVESTSADTVLLTSKESKVWDLPSTSHVSMWK | |
| signaling domain- | TSDSVSSPQPGASDTAVPEQNKTTKTGQMDGIPMSMKNEMPISQI | |
| DAP12 signaling | EVMYPPPYLDNEKSNGTIIHVKGKHLCPSPLFPGPSKPFWVLVVVG | |
| domain, amino | GVLACYSLLVTVAFIIFWVRSKRSRLLHSDYMNMTPRRPGPTRKHY | |
| acids 1-24 are | QPYAPPRDFAAYRSYFLGRLVPRGRGAAEAATRKQRITETESPYQE | |
| signal peptide | LQGQRSDVYSDLNTQRPYYK | |
| (CER140) | | |
| Tim4 binding | MSKEPLILWLMIEFWWLYLTPVTSETVVTEVLGHRVTLPCLYSS | 258 |
| domain-modified | WSHNSNSMCWGKDQCPYSGCKEALIRTDGMRVTSRKSAKYRLQ | |
| IgG4 hinge-CD28 | GTIPRGDVSLTILNPSESDSGVYCCRIEVPGWFNDVKINVRLNLQR | |
| transmembrane- | ASTTTHRTATTTTRRTTTTSPTTTRQMTTTPAALPTTVVTTPDLTT | |
| CD28 | GTPLQMTTIAVFTTANTCLSLTPSTLPEEATGLLTPEPSKEGPILTA | |
| costimulatory | ESETVLPSDSWSSVESTSADTVLLTSKESKVWDLPSTSHVSMWK | |
| signaling domain- | TSDSVSSPQPGASDTAVPEQNKTTKTGQMDGIPMSMKNEMPISQ | |
| DAP12 signaling | ESKYGPPCPPCPFWVLVVVGGVLACYSLLVTVAFIIFWVRSKRSRL | |
| domain, amino | LHSDYMNMTPRRPGPTRKHYQPYAPPRDFAAYRSYFLGRLVPRGRG | |
| acids 1-24 are | AAEAATRKQRITETESPYQELQGQRSDVYSDLNTQRPYYK | |
| signal peptide | | |
| (CER143) | | |
| Tim4 binding | MSKEPLILWLMIEFWWLYLTPVTSETVVTEVLGHRVTLPCLYSS | 259 |
| domain-CD28 | WSHNSNSMCWGKDQCPYSGCKEALIRTDGMRVTSRKSAKYRLQ | |
| transmembrane- | GTIPRGDVSLTILNPSESDSGVYCCRIEVPGWFNDVKINVRLNLQR | |
| CD28 | ASTTTHRTATTTTRRTTTTSPTTTRQMTTTPAALPTTVVTTPDLTT | |
| costimulatory | GTPLQMTTIAVFTTANTCLSLTPSTLPEEATGLLTPEPSKEGPILTA | |
| signaling domain- | ESETVLPSDSWSSVESTSADTVLLTSKESKVWDLPSTSHVSMWK | |
| DAP12 signaling | TSDSVSSPQPGASDTAVPEQNKTTKTGQMDGIPMSMKNEMPISQ | |
| domain, amino | FWVLVVVGGVLACYSLLVTVAFIIFWVRSKRSRLLHSDYMNMTPR | |
| acids 1-24 are | RPGPTRKHYQPYAPPRDFAAYRSYFLGRLVPRGRGAAEAATRKQRI | |
| signal peptide | TETESPYQELQGQRSDVYSDLNTQRPYYK | |
| (CER137) | | |
| Tim4 binding | MSKEPLILWLMIEFWWLYLTPVTSETVVTEVLGHRVTLPCLYSS | 449 |
| domain-Tim4 | WSHNSNSMCWGKDQCPYSGCKEALIRTDGMRVTSRKSAKYRLQ | |
| transmembrane | GTIPRGDVSLTILNPSESDSGVYCCRIEVPGWFNDVKINVRLNLQR | |
| domain-CD28 | ASTTTHRTATTTTRRTTTTSPTTTRQMTTTPAALPTTVVTTPDLTT | |
| costimulatory | GTPLQMTTIAVFTTANTCLSLTPSTLPEEATGLLTPEPSKEGPILTA | |
| signaling domain- | ESETVLPSDSWSSVESTSADTVLLTSKESKVWDLPSTSHVSMWK | |
| DAP12 signaling | TSDSVSSPQPGASDTAVPEQNKTTKTGQMDGIPMSMKNEMPISQ | |
| domain, amino | LLMIIAPSLGFVLFALFVAFLRSKRSRLLHSDYMNMTPRRPGPTR | |
| acids 1-24 are | KHYQPYAPPRDFAAYRSYFLGRLVPRGRGAAEAATRKQRITETE | |
| signal peptide | SPYQELQGQRSDVYSDLNTQRPYYK | |
| (CER256) | | |

### Chimeric Tim Receptors

In some embodiments, a chimeric Tim receptor comprises a single chain chimeric protein, the single chain chimeric protein comprising: (a) an extracellular domain comprising a binding domain comprising: (i) a Tim4 IgV domain and a Tim1 mucin domain; or (ii) a Tim1 IgV domain and a Tim4 mucin domain; (b) an intracellular signaling domain, wherein the intracellular signaling domain comprises a primary intracellular signaling domain and optionally a secondary intracellular signaling domain; and (c) a transmembrane domain positioned between and connecting the extracellular domain and the intracellular signaling domain.

In some embodiments, a chimeric Tim receptor comprises a single chain chimeric protein, the single chain chimeric protein comprising: (a) an extracellular domain comprising a binding domain comprising a Tim1 IgV domain and a Tim1 mucin domain; (b) an intracellular signaling domain, wherein the intracellular signaling domain comprises a primary intracellular signaling domain and optionally a secondary intracellular signaling domain; and (c) a transmembrane domain positioned between and connecting the extracellular domain and the intracellular signaling domain.

In some embodiments, a chimeric Tim receptor comprises a single chain chimeric protein, the single chain chimeric protein comprising: (a) an extracellular domain comprising a binding domain comprising: (i) a Tim1 IgV domain and a Tim1 mucin domain; (ii) a Tim4 IgV domain and a Tim4 mucin domain; (iii) a Tim1 IgV domain and a Tim4 mucin domain; or (iv) a Tim4 IgV domain and a Tim1 mucin domain; (b) an intracellular signaling domain, wherein the intracellular signaling domain comprises a primary intracellular signaling domain selected from a Tim1 signaling domain or a Tim4 signaling domain, and optionally a secondary intracellular signaling domain; and (c) a transmembrane domain positioned between and connecting the extracellular domain and the intracellular signaling domain.

In some embodiments, the present disclosure provides chimeric Tim receptors comprising a single chain chimeric protein, the single chain chimeric protein comprising: (a) an extracellular domain comprising a binding domain comprising: (i) a Tim4 IgV domain and a Tim4 mucin domain; (b) an intracellular signaling domain, wherein the intracellular signaling domain comprises a primary intracellular signaling domain selected from a CD28 signaling domain, a CD3ζ signaling domain, and a 4-1BB signaling domain, and a secondary intracellular signaling domain selected from a TLR signaling domain; and (c) a transmembrane domain positioned between and connecting the extracellular domain and the intracellular signaling domain.

In some embodiments, the present disclosure provides chimeric Tim receptors comprising a single chain chimeric protein, the single chain chimeric protein comprising: (a) an extracellular domain comprising a binding domain comprising: (i) a Tim4 IgV domain and a Tim4 mucin domain; (b) an intracellular signaling domain, wherein the intracellular signaling domain comprises a primary intracellular signaling domain comprises an immunoreceptor tyrosine-based activation motif (ITAM) containing signaling domain; the secondary intracellular signaling domain comprises a costimulatory signaling domain, Tim1 signaling domain, or Tim4 signaling domain; and the tertiary intracellular signaling domain comprises a TLR signaling domain (e.g., a TLR2 or TLR8 signaling domain).

A Tim binding domain suitable for use in a chimeric Tim receptor of the present disclosure may be any polypeptide or peptide derived from a Tim1 and/or Tim4 molecule that specifically binds phosphatidylserine. In embodiments, a Tim binding domain comprises an IgV domain from Tim1 or Tim4, and a mucin domain from Tim1 or Tim4. For example, a Tim binding domain may comprise a Tim1 IgV domain and a Tim1 mucin domain. In another example, a Tim binding domain may comprise a Tim1 IgV domain and a Tim4 mucin domain. In another example, a Tim binding domain may comprise a Tim4 IgV domain and a Tim 1 mucin domain. In another example, a Tim binding domain may comprise a Tim4 IgV domain and a Tim4 mucin domain.

Phosphatidylserine binding is generally regulated by the IgV domain. The core phosphatidylserine binding domain is a four amino acid sequence in the IgV domain (e.g., amino acids 95-98 of SEQ ID NO:52 or amino acids 92-95 of SEQ ID NO:58). A Tim4 binding domain binds minimally to cells with low phosphatidylserine density. A Tim1 binding domain binds more strongly to a lower phosphatidylserine density, resulting in a lower threshold for response. A summary of Tim1 and Tim4 binding to phosphatidylserine is provided in Table 5. By combining Tim1 IgV domain and Tim4 mucin domain, or Tim4 IgV domain and Tim1 mucin domain, the binding affinity of the binding domain to phosphatidylserine can be modulated. Further, such a combination in the Tim binding domain also provides a combination of the sensitivity to phosphatidylserine of Tim4 and the stability in protein expression of Tim1.

TABLE 5

| | Low PtdSer | Moderate PtdSer | High PtdSer | Cooperativity |
|---|---|---|---|---|
| Tim-1 | Moderate binding | Strong binding | Strong binding | |
| Tim-4 | Minimal binding | Moderate binding | Strong binding | Yes |

Additionally, an RGD domain (e.g., amino acids 68-70 of SEQ ID NO:52) in an IgV domain may regulate integrin binding as a co-receptor for engulfment.

In certain embodiments, the Tim binding domain is derived from human Tim1 and/or Tim4. An exemplary human Tim1 molecule is provided in Uniprot. Ref. Q96D42 (SEQ ID NO:56). An exemplary human Tim1 binding domain comprises or consists of an amino acid sequence of SEQ ID NO:57 or amino acids 21-295 of SEQ ID NO:57. In certain embodiments, the Tim1 binding domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:57 or amino acids 21-295 of SEQ ID NO:57. In certain embodiments, the Tim1 binding domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:57 or amino acids 21-295 of SEQ ID NO:57.

An exemplary human Tim4 molecule is provided in Uniprot. Ref. Q96H15 (SEQ ID NO:49). An exemplary human Tim4 binding domain comprises or consists of an amino acid sequence of SEQ ID NO:50 or amino acids 25-314 of SEQ ID NO:50. An exemplary mouse Tim4 binding domain comprises or consists of an amino acid sequence of SEQ ID NO:51 or amino acids 23-279 of SEQ ID NO:51. In certain embodiments, the Tim4 binding domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:50 or amino acids 25-314 of SEQ ID NO:50, or SEQ ID NO:51 or amino acids 23-279 of SEQ ID NO:51. In certain embodiments, the Tim4 binding domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:50 or amino acids 25-314 of SEQ ID NO:50, or SEQ ID NO:51 or amino acids 23-279 of SEQ ID NO:51.

In embodiments, the Tim binding domain comprises an IgV domain from Tim1. An exemplary human Tim1 IgV domain is provided in SEQ ID NO:58. In some embodiments, the Tim1 IgV domain is a modified Tim1 IgV domain comprising a R66G substitution in SEQ ID NO:58. The R66G substitution (e.g., amino acids 68-70 of SEQ ID NO:52 (Tim4 IgV)) confers a RGD domain in Tim1 IgV domain, which may regulate integrin binding as a co-receptor for engulfment. In particular embodiments, the modified Tim1 IgV domain comprises the amino acid sequence of SEQ ID NO:260. In some embodiments, this modified Tim1 domain may increase phagocytic activity while preserving Tim1 sensitivity. In certain embodiments, the Tim1 IgV domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:58, SEQ ID NO:38 with a R66G substitution, or SEQ ID NO:260. In certain embodiments, the Tim1 IgV comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:58, SEQ ID NO:58 with a R66G substitution, or SEQ ID NO:260.

In other embodiments, the Tim binding domain comprises an IgV domain from Tim4. An exemplary human Tim4 IgV domain is provided in SEQ ID NO:52. In certain embodiments, the Tim4 IgV domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:52. In certain embodiments, the Tim4 IgV domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:52.

In embodiments, the Tim binding domain comprises a mucin domain from Tim1. An exemplary human Tim1 mucin domain is provided in SEQ ID NO:59. In certain embodiments, the Tim1 mucin domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:59. In certain embodiments, the Tim1 mucin domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:59.

In other embodiments, the Tim binding domain comprises a mucin domain from Tim4. An exemplary human Tim4 mucin domain is provided in SEQ ID NO:53. In certain embodiments, the Tim4 mucin domain comprises or consists of an amino acid sequence having at least about 75%, 80%, 85%, 90%, 91%, 92%, 93%, 94%, 95%, 96%, 97%, 98%, 99%, 99.5%, or 100% identity to SEQ ID NO:53. In certain embodiments, the Tim4 mucin domain comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of SEQ ID NO:53.

In embodiments, the Tim binding domain comprises a Tim1 IgV domain and a Tim1 mucin domain. In a particular embodiment, the Tim1 IgV domain comprises the amino acid sequence set forth in SEQ ID NO:58 and the Tim1 mucin domain comprises the amino acid sequence set forth in SEQ ID NO:59. In another particular embodiment, the Tim1 IgV domain comprises the amino acid sequence set forth in SEQ ID NO:58 with a R66G substitution and the Tim1 mucin domain comprises the amino acid sequence set forth in SEQ ID NO:59. In a further particular embodiment, the Tim1 IgV domain comprises the amino acid sequence set forth in SEQ ID NO:260 and the Tim1 mucin domain comprises the amino acid sequence set forth in SEQ ID NO:59. In some embodiments, the Tim1 IgV domain and Tim1 mucin domain together comprise or consist of the amino acid sequence set forth in SEQ ID NO:57 or SEQ ID NO:57 absent amino acids 1-20.

In other embodiments, the Tim binding domain comprises a Tim4 IgV domain and a Tim4 mucin domain. In a particular embodiment, the Tim4 IgV domain comprises the amino acid sequence set forth in SEQ ID NO:52 and the Tim4 mucin domain comprises the amino acid sequence set forth in SEQ ID NO:53. In some embodiments, the Tim4 IgV domain and Tim4 mucin domain together comprise or consist of the amino acid sequence set forth in SEQ ID NO:50 or SEQ ID NO:50 absent amino acids 1-24.

In further embodiments, the Tim binding domain comprises a Tim1 IgV domain and a Tim4 mucin domain. In a particular embodiment, the Tim1 IgV domain comprises the amino acid sequence set forth in SEQ ID NO:58 and the Tim4 mucin domain comprises the amino acid sequence set forth in SEQ ID NO:53. In another particular embodiment, the Tim1 IgV domain comprises the amino acid sequence set forth in SEQ ID NO:58 with a R66G substitution and the Tim4 mucin domain comprises the amino acid sequence set forth in SEQ ID NO:53. In a further particular embodiment, the Tim1 IgV domain comprises the amino acid sequence set forth in SEQ ID NO:260 and the Tim4 mucin domain comprises the amino acid sequence set forth in SEQ ID NO:53. In some embodiments, the Tim1 IgV domain further comprises the Tim1 signal sequence of SEQ ID NO:60.

In still further embodiments, the Tim binding domain comprises a Tim4 IgV domain and a Tim1 mucin domain. In a particular embodiment, the Tim4 IgV domain comprises the amino acid sequence set forth in SEQ ID NO:52 and the Tim1 mucin domain comprises the amino acid sequence set forth in SEQ ID NO:59. In some embodiments, the Tim4 IgV domain further comprises the Tim4 signal sequence of SEQ ID NO:54.

In certain embodiments, the extracellular domain optionally comprises an extracellular, non-signaling spacer or linker domain. Where included, such a spacer or linker domain may position the binding domain away from the host cell surface to further enable proper cell/cell contact, binding, and activation. When included in a chimeric receptor as described herein, an extracellular spacer domain is generally located between the extracellular binding domain and the transmembrane domain of the chimeric Tim receptor. The length of the extracellular spacer may be varied to optimize target molecule binding based on the selected target molecule, selected binding epitope, binding domain size and affinity (see, e.g., Guest et al., J. Immunother. 28:203-11, 2005; PCT Publication No. WO 2014/031687). In certain embodiments, an extracellular spacer domain is an immunoglobulin hinge region (e.g., IgG1, IgG2, IgG3, IgG4, IgA, IgD). An immunoglobulin hinge region may be a wild type immunoglobulin hinge region or an altered wild type immunoglobulin hinge region. An altered IgG₄ hinge region is described in PCT Publication No. WO 2014/031687, which hinge region is incorporated herein by reference in its entirety. In a particular embodiment, an extracellular spacer domain comprises a modified IgG4 hinge region having an amino acid sequence of ESKYGPPCPPCP (SEQ ID NO:21). Other examples of hinge regions that may be used in the chimeric Tim receptors described herein include the hinge region from the extracellular regions of type 1 membrane proteins, such as CD8a, CD4, CD28 and CD7, which may be wild-type or variants thereof. In a particular embodiment, an extracellular spacer domain comprises a CD8a hinge region having an amino acid sequence of SEQ ID NO:22 or a CD28 hinge region having an amino acid sequence of SEQ ID NO:23. In further embodiments, an extracellular spacer domain comprises all or a portion of an immunoglobulin Fc domain selected from: a CH1 domain, a CH2 domain, a CH3 domain, or combinations thereof (see, e.g., PCT Publication WO2014/031687, which spacers are incorporated herein by reference in their entirety). In yet further embodiments, an extracellular spacer domain may comprise a stalk region of a type II C-lectin (the extracellular domain located between the C-type lectin domain and the transmembrane domain). Type II C-lectins include CD23, CD69, CD72, CD94, NKG2A, and NKG2D.

In certain embodiments, an extracellular domain comprises an amino acid sequences derived from any mammalian species, including humans, primates, cows, horses, goats, sheep, dogs, cats, mice, rats, rabbits, guinea pigs, pigs, transgenic species thereof, or any combination thereof. In certain embodiments, an extracellular domain is murine, human, or chimeric.

An intracellular signaling domain comprises a primary intracellular signaling domain. In some embodiments, an intracellular signaling domain comprises a primary intracellular signaling domain, a secondary intracellular signaling domain. In some embodiments, an intracellular signaling domain comprises a primary intracellular signaling domain, a secondary intracellular signaling domain, and a tertiary intracellular signaling domain. The primary, secondary, and/or tertiary intracellular signaling domains may independently be any portion of a signaling molecule that retains sufficient signaling activity. In some embodiments, a full length signaling molecule or full length intracellular component of a signaling molecule is used. In some embodiments, a truncated portion of a signaling molecule or intracellular component of a signaling molecule is used, provided that the truncated portion retains sufficient signal transduction activity. In some embodiments, a signaling domain is a variant of a whole or truncated portion of a signaling molecule, provided that the variant retains sufficient signal transduction activity (i.e., is a functional variant).

In some some embodiments, the primary intracellular signaling domain comprises a Tim1 signaling domain, a Tim4 signaling domain, a TRAF2 signaling domain, a TRAF6 signaling domain, a CD28 signaling domain, a DAP12 signaling domain, a CD3ζ signaling domain, 4-1BB signaling domain, TLR2 signaling domain, or a TLR8 signaling domain.

In some embodiments, the secondary intracellular signaling domain comprises a Tim1 signaling domain, a Tim4 signaling domain, a TRAF2 signaling domain, a TRAF6 signaling domain, a CD28 signaling domain, a DAP12 signaling domain, a CD3ζ signaling domain, 4-1BB signaling domain, TLR2 signaling domain or a TLR8 signaling domain.

In some embodiments, the tertiary intracellular signaling domain comprises a Tim1 signaling domain, a Tim4 signaling domain, a TRAF2 signaling domain, a TRAF6 signaling domain, a CD28 signaling domain, a DAP12 signaling domain, a CD33 signaling domain, 4-1BB signaling domain, TLR2 signaling domain or a TLR8 signaling domain.

In some embodiments, the primary intracellular signaling domain comprises an immunoreceptor tyrosine-based activation motif (ITAM) containing signaling domain; the secondary intracellular signaling domain comprises a costimulatory signaling domain, Tim1 signaling domain or Tim4 signaling domain; and the tertiary intracellular signaling domain comprises a TLR (e.g., TLR2 or TLR8) signaling domain. An ITAM containing signaling domain generally contains at least one (one, two, three, four, or more) ITAMs, which refer to a conserved motif of YXXL/I-X₆₋₈-YXXL/I. An ITAM containing signaling domain may initiate T cell activation signaling following antigen binding or ligand engagement. ITAM-signaling domains include, for example, intracellular signaling domains of CD3γ, CD3δ, CD3ε, CD3ζ, CD5, CD22, CD79a, CD278 (ICOS), DAP12, FcRγ, and CD66d. A costimulatory signaling domain, which, when activated in conjunction with a primary or classic (e.g., ITAM-driven) activation signal, promotes or enhances T cell response, such as T cell activation, cytokine production, proliferation, differentiation, survival, effector function, or combinations thereof. Costimulatory signaling domains for use in chimeric Tim receptors include, for example, CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), CD273 (PD-L2), CD274 (PD-L1), CD278 (ICOS), DAP10, LAT, LFA-1, LIGHT, NKG2C, SLP76, TRIM, ZAP70, or any combination thereof. In some embodiments, the costimulatory signaling domain comprises a OX40, CD2, CD27, CD28, ICAM-1, LFA-1 (CD11a/CD18), ICOS (CD278), or 4-1BB (CD137) signaling domain. A TLR signaling domain may be a TLR1, TLR2, TLR3, TLR4, TLR5, TLR6, TLR7, TLR8, or TLR9 signaling domain. In some embodiments, the TLR signaling domain is a TLR2 signaling domain or TLR8 signaling domain.

As used herein, the designation of primary, secondary, and tertiary intracellular signaling domains includes but is not limited to arrangements of the primary intracellular signaling domain at the N-terminus, secondary intracellular signaling domain in the middle, and tertiary intracellular signaling domain at the C-terminus of the intracellular portion of the chimeric Tim receptor. Thus, designation of the primary intracellular signaling domain does not limit the use of the selected intracellular signaling domain at the N-terminus of the intracellular portion of the chimeric Tim receptor. Designation of the secondary intracellular signaling domain does not limit the use of the selected intracellular signaling domain in the middle (or at the C-terminus for those chimeric Tim receptors only having two intracellular signaling domains) of the intracellular portion of the chimeric Tim receptor. Designation of the tertiary intracellular signaling domain does not limit the use of the selected intracellular signaling domain at the C-terminus of the intracellular portion of the chimeric Tim receptor. Thus, different arrangements of the primary, secondary, and/or tertiary intracellular signaling domains within the intracellular portion of the chimeric Tim receptor are contemplated.

An exemplary Tim1 signaling domain comprises or consists of an amino acid sequence of SEQ ID NO:261. An exemplary Tim4 signaling domain comprises or consists of an amino acid sequence of SEQ ID NO:262. An exemplary TRAF2 signaling domain comprises or consists of an amino acid sequence of SEQ ID NO:263. An exemplary TRAF6 signaling domain comprises or consists of an amino acid sequence of SEQ ID NO:264. An exemplary CD28 signaling domain comprises or consists of an amino acid sequence of SEQ ID NO:28 or 29. An exemplary DAP12 signaling domain comprises or consists of an amino acid sequence of SEQ ID NO: 103. An exemplary CD3 signaling domain comprises or consists of an amino acid sequence of SEQ ID NO:26 or 27. An exemplary 4-1BB signaling domain comprises or consists of the amino acid sequence of SEQ ID NO:215. An exemplary TLR2 signaling domain comprises or consists of the amino acid sequence of SEQ ID NO:522. An exemplary TLR8 signaling domain comprises or consists of an amino acid sequence of SEQ ID NO:265.

In a particular embodiment, the Tim1 signaling domain comprises the amino acid sequence set forth in SEQ ID NO:261. In another particular embodiment, the Tim4 signaling domain comprises the amino acid sequence set forth in SEQ ID NO:262. In another particular embodiment, the TRAF2 signaling domain comprises the amino acid sequence set forth in SEQ ID NO:263. In another particular embodiment, the TRAF6 signaling domain comprises the amino acid sequence set forth in SEQ ID NO:264. In another particular embodiment, the CD28 signaling domain comprises the amino acid sequence set forth in SEQ ID NO:28. In another particular embodiment, the CD28 signaling domain comprises the amino acid sequence set forth in SEQ ID NO:29. In another particular embodiment, the DAP12 signaling domain comprises the amino acid sequence set forth in SEQ ID NO:103. In another particular embodiment, the CD3ζ signaling domain comprises the amino acid sequence set forth in SEQ ID NO:27. In another particular embodiment, the CD3ζ signaling domain comprises the amino acid sequence set forth in SEQ ID NO:26. In another particular embodiment, the 4-1BB signaling domain comprises the amino acid sequence of SEQ ID NO:215. In another particular embodiment, the TLR2 signaling domain comprises the amino acid sequence of SEQ ID NO:522. In another particular embodiment, or the TLR8 signaling domain comprises the amino acid sequence set forth in SEQ ID NO:265.

In embodiments, the primary and/or secondary signaling domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS: 26-29, 103, 261-265, 215, and 522. In certain embodiments, the primary and/or secondary signaling domains comprises an amino acid sequence having at least about 1, 2, 3, 4, 5, 6, 7, 8, 9, 10, 11, 12, 13, 14, 15, 16, 17, 18, 19, or 20 amino acid modifications (e.g., deletions, additions, substitutions) to an amino acid sequence of any one of SEQ ID NOS:26-29, 103, 261-265, 215, and 522. In embodiments, the primary signaling domain and secondary signaling domain are the same or different.

In certain embodiments, an intracellular signaling domain comprises a Tim1 intracellular signaling domain. In some embodiments, an intracellular signaling domain comprises a Tim4 intracellular signaling domain. In some embodiments, an intracellular signaling domain comprises a CD3ζ intracellular signaling domain. In some embodiments, an intracellular signaling domain comprises a CD28 intracellular signaling domain. In some embodiments, an intracellular signaling domain comprises a TRAF6 intracellular signaling domain. In some embodiments, an intracellular signaling domain comprises a TRAF2 intracellular signaling domain. In some embodiments, an intracellular signaling domain comprises a 4-1BB intracellular signaling domain. In some embodiments, an intracellular signaling domain comprises a TLR2 intracellular signaling domain. In some embodiments, an intracellular signaling domain comprises a TLR8 intracellular signaling domain.

In certain embodiments, an intracellular signaling domain comprises a Tim1 primary intracellular signaling domain and a CD3ζ secondary intracellular signaling domain. In other embodiments, an intracellular signaling domain comprises a Tim4 primary intracellular signaling domain and a CD33 secondary intracellular signaling domain. In other embodiments, an intracellular signaling domain comprises a TLR8 primary intracellular signaling domain and a CD3ζ secondary intracellular signaling domain. In other embodiments, an intracellular signaling domain comprises a CD28 primary intracellular signaling domain and a DAP12 secondary intracellular signaling domain. In some embodiments, an intracellular signaling domain comprises a CD28 primary intracellular signaling domain and a CD3ζ secondary intracellular signaling domain. In some embodiments, an intracellular signaling domain comprises a CD28 primary intracellular signaling domain, a TLR2 secondary intracellular signaling domain, and a CD3ζ tertiary intracellular signaling domain. In some embodiments, an intracellular signaling domain comprises a CD28 primary intracellular signaling domain, a CD3 secondary intracellular signaling domain, and a TLR2 tertiary intracellular signaling domain. In some embodiments, an intracellular signaling domain comprises a CD28 primary intracellular signaling domain, a TLR8 secondary intracellular signaling domain, and a CD3ζ tertiary intracellular signaling domain. In some embodiments, an intracellular signaling domain comprises a CD28 primary intracellular signaling domain, a CD3ζ secondary intracellular signaling domain, and a TLR8 tertiary intracellular signaling domain. In some embodiments, an intracellular signaling domain comprises a TLR2 primary intracellular signaling domain and a CD3ζ secondary intracellular signaling domain. In some embodiments, an intracellular signaling domain comprises a CD3ζ primary intracellular signaling domain and a TLR2 secondary intracellular signaling domain. In some embodiments, an intracellular signaling domain comprises a TLR8 primary intracellular signaling domain and a CD3ζ secondary intracellular signaling domain. In some embodiments, an intracellular signaling domain comprises a CD3ζ primary intracellular signaling domain and a TLR8 secondary intracellular signaling domain. In some embodiments, an intracellular signaling domain comprises a TRAF6 primary intracellular signaling domain and a CD3ζ secondary intracellular signaling domain. In some embodiments, an intracellular signaling domain comprises a CD3ζ primary intracellular signaling domain and a TRAF6 secondary intracellular signaling domain. In some embodiments, an intracellular signaling domain comprises a CD28 primary intracellular signaling domain and a CD3ζ secondary intracellular signaling domain. In some embodiments, an intracellular signaling domain comprises a CD28 primary intracellular signaling domain, a TLR2 secondary intracellular signaling domain, and a CD3ζ tertiary intracellular signaling domain. In some embodiments, an intracellular signaling domain comprises a CD28 primary intracellular signaling domain, a CD3ζ secondary intracellular signaling domain, and a TLR2 tertiary intracellular signaling domain. In some embodiments, an intracellular signaling domain comprises a CD28 primary intracellular signaling domain, a TLR8 secondary intracellular signaling domain, and a CD3ζ tertiary intracellular signaling domain. In some embodiments, an intracellular signaling domain comprises a CD28 primary intracellular signaling domain, a CD3ζ secondary intracellular signaling domain, and a TLR8 tertiary intracellular signaling domain. In some embodiments, an intercellular signaling domain comprises a CD3ζ primary intracellular signaling domain and a TLR2 secondary intracellular signaling domain. In some embodiments, an intercellular signaling domain comprises a TLR2 primary intracellular signaling domain and a CD3ζ secondary intracellular signaling domain. In some embodiments, an intercellular signaling domain comprises a CD3ζ primary intracellular signaling domain and a TLR8 secondary intracellular signaling domain. In some embodiments, an intercellular signaling domain comprises a TLR8 primary intracellular signaling domain and a CD3ζ secondary intracellular signaling domain. In some embodiments, an intercellular signaling domain comprises a CD3ζ primary intracellular signaling domain and a TRAF6 secondary intracellular signaling domain. In some embodiments, an intercellular signaling domain comprises a TRAF6 primary intracellular signaling domain and a CD3ζ secondary intracellular signaling domain. In some embodiments, an intracellular signaling domain comprises a Tim4 primary intracellular signaling domain, a TLR2 secondary intracellular signaling domain, and a CD3ζ tertiary intracellular signaling domain. In some embodiments, an intracellular signaling domain comprises a Tim4 primary intracellular signaling domain, a CD3ζ secondary intracellular signaling domain, and a TLR2 tertiary intracellular signaling domain. In some embodiments, an intracellular signaling domain comprises a Tim4 primary intracellular signaling domain, a TLR8 secondary intracellular signaling domain, and a CD3ζ tertiary intracellular signaling domain. In some embodiments, an intracellular signaling domain comprises a Tim4 primary intracellular signaling domain, a CD3ζ secondary intracellular signaling domain, and a TLR8 tertiary intracellular signaling domain.

In a particular embodiment, an intracellular signaling domain comprises a Tim1 primary intracellular signaling domain comprising an amino acid sequence of SEQ ID NO:261 and a CD3ζ secondary intracellular signaling domain comprising an amino acid sequence of SEQ ID NO:26 or 27. In another particular embodiment, an intracellular signaling domain comprises a Tim4 primary intracellular signaling domain comprising an amino acid sequence of SEQ ID NO:262 and a CD3ζ secondary intracellular signaling domain comprising an amino acid sequence of SEQ ID NO:26 or 27. In a further particular embodiment, an intracellular signaling domain comprises a TLR8 primary intracellular signaling domain comprising an amino acid sequence of SEQ ID NO:265 and a CD3ζ secondary intracellular signaling domain comprising an amino acid sequence of SEQ ID NO:26 or 27. In another particular embodiment, an intracellular signaling domain comprises a CD28 primary intracellular signaling domain comprising an amino acid sequence of SEQ ID NO:28 or 29 and a DAP12 secondary intracellular signaling domain comprising an amino acid sequence of SEQ ID NO:103.

Intracellular signaling domains may be derived from a mammalian species, including humans, primates, cows, horses, goats, sheep, dogs, cats, mice, rats, rabbits, guinea pigs, pigs, and transgenic species thereof. In some embodiments, an intracellular signaling domain comprises a combination of primary, secondary, and optionally tertiary intracellular signaling domain as shown in Table 7.

In certain embodiments, the transmembrane domain is derived from an integral membrane protein (e.g., receptor, cluster of differentiation (CD) molecule, enzyme, transporter, cell adhesion molecule, or the like). In one embodiment, the transmembrane domain is selected from the same molecule as the molecule from which the extracellular domain is derived. In another embodiment, the transmembrane domain is selected from the same molecule as the molecule from which the intracellular signaling domain is derived. For example, a chimeric Tim receptor may comprise a Tim4 binding domain and a Tim4 transmembrane domain. In another example, a chimeric Tim receptor may comprise a CD28 transmembrane domain and a CD28 costimulatory signaling domain. In certain embodiments, the transmembrane domain and the extracellular domain are derived from different molecules; the transmembrane domain and the intracellular signaling domain are derived from different molecules; or the transmembrane domain, extracellular domain, and intracellular signaling domain are all derived from different molecules. Examples of transmembrane domains that may be used in chimeric Tim receptors of the present disclosure include transmembrane domains from Tim1, Tim4, and CD28. An exemplary Tim1 transmembrane domain comprises or consists of an amino acid sequence of SEQ ID NO:80. An exemplary Tim4 transmembrane domain comprises or consists of an amino acid sequence of SEQ ID NO:81 or 255. An exemplary CD28 transmembrane domain comprises or consists of an amino acid sequence of SEQ ID NO:25. In certain embodiments, the transmembrane domain comprises or consists of an amino acid sequence having at least about 75%, 80%, 85%, 90%, 91%, 92%, 93%, 94%, 95%, 96%, 97%, 98%, 99%, 99.5%, or 100% identity to any one of SEQ ID NOS:80, 81, 255, or 25. In certain embodiments, the transmembrane domain comprises an amino acid sequence having at least about 1, 2, 3, 4, 5, 6, 7, 8, 9, or 10 amino acid modifications (e.g., deletion, additions, substitutions) to an amino acid sequence of any one of SEQ ID NOS:80, 81, 255, or 25.

Transmembrane domains may derived from any mammalian species, including humans, primates, cows, horses, goats, sheep, dogs, cats, mice, rats, rabbits, guinea pigs, pigs, and transgenic species thereof.

In certain embodiments, a chimeric Tim receptor is encoded by polynucleotide sequences derived from any mammalian species, including humans, primates, cows, horses, goats, sheep, dogs, cats, mice, rats, rabbits, guinea pigs, pigs, transgenic species thereof, or any combination thereof. In certain embodiments, a chimeric Tim receptor is murine, chimeric, human, or humanized.

It is understood that direct fusion of one domain to another domain of a chimeric Tim receptor described herein does not preclude the presence of intervening junction amino acids. Junction amino acids may be natural or non-natural (e.g., resulting from the construct design of a chimeric protein). For example, junction amino acids may result from restriction enzyme sites used for joining one domain to another domain or cloning polynucleotides encoding chimeric Tim receptors into vectors.

Exemplary chimeric Tim receptors and components thereof of the present disclosure are described in Tables 6A-6F.

TABLE 6A

| | | | | Primary | Secondary |
|---|---|---|---|---|---|
| Construct | IgV | Mucin | Transmembrane | Signaling | Signaling |
| No. | Domain | Domain | Domain | Domain | Domain |
| 1 | Tim1 | Tim1 | Tim1 | Tim1 | CD3ζ |
| 2 | Tim1 | Tim1 | Tim1 | Tim4 | CD3ζ |
| 3 | Tim1 | Tim1 | CD28 | CD28 | |
| 4 | Tim1 | Tim1 | Tim1 | TRAF6 | |
| 5 | Tim1 | Tim1 | CD28 | TRAF6 | |
| 6 | Tim1 | Tim1 | Tim1 | TRAF2 | |
| 7 | Tim1 | Tim1 | CD28 | TRAF2 | |
| 8 | Tim1 | Tim1 | Tim1 | TLR8 | CD3ζ |
| 9 | Tim1 | Tim1 | CD28 | CD28 | DAP12 |
| 10 | Tim1 | Tim1 | Tim1 | CD28 | DAP12 |

TABLE 6B

| | | | | Primary | Secondary |
|---|---|---|---|---|---|
| Construct | IgV | Mucin | Transmembrane | Signaling | Signaling |
| No. | Domain | Domain | Domain | Domain | Domain |
| 1′ | SEQ ID | SEQ ID | SEQ ID | SEQ ID | SEQ ID |
| | NO: 58 | NO: 59 | NO: 80 | NO: 261 | NO: 27 |
| 2′ | SEQ ID | SEQ ID | SEQ ID | SEQ ID | SEQ ID |
| | NO: 58 | NO: 59 | NO: 80 | NO: 262 | NO: 27 |
| 3′ | SEQ ID | SEQ ID | SEQ ID | SEQ ID | |
| | NO: 58 | NO: 59 | NO: 25 | NO: 28 | |
| 4′ | SEQ ID | SEQ ID | SEQ ID | SEQ ID | |
| | NO: 58 | NO: 59 | NO: 80 | NO: 264 | |
| 5′ | SEQ ID | SEQ ID | SEQ ID | SEQ ID | |
| | NO: 58 | NO: 59 | NO: 25 | NO: 264 | |
| 6′ | SEQ ID | SEQ ID | SEQ ID | SEQ ID | |
| | NO: 58 | NO: 59 | NO: 80 | NO: 263 | |
| 7′ | SEQ ID | SEQ ID | SEQ ID | SEQ ID | |
| | NO: 58 | NO: 59 | NO: 25 | NO: 263 | |
| 8′ | SEQ ID | SEQ ID | SEQ ID | SEQ ID | SEQ ID |
| | NO: 58 | NO: 59 | NO: 80 | NO: 265 | NO: 27 |
| 9′ | SEQ ID | SEQ ID | SEQ ID | SEQ ID | SEQ ID |
| | NO: 58 | NO: 59 | NO: 25 | NO: 28 | NO: 103 |
| 10′ | SEQ ID | SEQ ID | SEQ ID | SEQ ID | SEQ ID |
| | NO: 58 | NO: 59 | NO: 80 | NO: 28 | NO: 103 |

In embodiments, a chimeric Tim receptor of the present disclosure comprises a construct of Table 6A. In some embodiments, a chimeric Tim receptor of the present disclosure comprises a construct of Table 6B.

In a particular embodiment, a chimeric Tim receptor of Construct 1 or Construct 1′ comprises amino acids 21-456 of SEQ ID NO:266. In a specific embodiment, a chimeric Tim receptor of Construct 1 or Construct 1′ comprises an amino acid sequence of SEQ ID NO:266.

In a particular embodiment, a chimeric Tim receptor of Construct 2 or Construct 2′ comprises amino acids 21-471 of SEQ ID NO:267. In a specific embodiment, a chimeric Tim receptor of Construct 2 or Construct 2′ comprises an amino acid sequence of SEQ ID NO:267.

In a particular embodiment, a chimeric Tim receptor of Construct 3 or Construct 3′ comprises amino acids 21-363 of SEQ ID NO:268. In a specific embodiment, a chimeric Tim receptor of Construct 3 or Construct 3′ comprises an amino acid sequence of SEQ ID NO:268.

In a particular embodiment, a chimeric Tim receptor of Construct 4 or Construct 4′ comprises amino acids 21-590 of SEQ ID NO:269. In a specific embodiment, a chimeric Tim receptor of Construct 4 or Construct 4′ comprises an amino acid sequence of SEQ ID NO:269.

In a particular embodiment, a chimeric Tim receptor of Construct 5 or Construct 5′ comprises amino acids 21-596 of SEQ ID NO:270. In a specific embodiment, a chimeric Tim receptor of Construct 5 or Construct 5′ comprises an amino acid sequence of SEQ ID NO:270.

In a particular embodiment, a chimeric Tim receptor of Construct 6 or Construct 6′ comprises amino acids 21-619 of SEQ ID NO:271. In a specific embodiment, a chimeric Tim receptor of Construct 6 or Construct 6′ comprises an amino acid sequence of SEQ ID NO:271.

In a particular embodiment, a chimeric Tim receptor of Construct 7 or Construct 7′ comprises amino acids 21-625 of SEQ ID NO:272. In a specific embodiment, a chimeric Tim receptor of Construct 7 or Construct 7′ comprises an amino acid sequence of SEQ ID NO:272.

In a particular embodiment, a chimeric Tim receptor of Construct 8 or Construct 8′ comprises amino acids 21-621 of SEQ ID NO:273. In a specific embodiment, a chimeric Tim receptor of Construct 8 or Construct 8′ comprises an amino acid sequence of SEQ ID NO:273.

In a particular embodiment, a chimeric Tim receptor of Construct 9 or Construct 9′ comprises amino acids 21-415 of SEQ ID NO:274. In a specific embodiment, a chimeric Tim receptor of Construct 9 or Construct 9′ comprises an amino acid sequence of SEQ ID NO:274.

In a particular embodiment, a chimeric Tim receptor of Construct 10 or Construct 10′ comprises amino acids 21-409 of SEQ ID NO:275. In a specific embodiment, a chimeric Tim receptor of Construct 10 or Construct 10′ comprises an amino acid sequence of SEQ ID NO:275.

TABLE 6C

| | | | | Primary | Secondary |
|---|---|---|---|---|---|
| Construct | IgV | Mucin | Transmembrane | Signaling | Signaling |
| No. | Domain | Domain | Domain | Domain | Domain |
| 11 | Tim4 | Tim4 | Tim4 | Tim4 | CD3ζ |
| 12 | Tim4 | Tim4 | Tim4 | Tim1 | CD3ζ |
| 13 | Tim4 | Tim4 | CD28 | CD28 | |
| 14 | Tim4 | Tim4 | Tim4 | TRAF6 | |
| 15 | Tim4 | Tim4 | CD28 | TRAF6 | |
| 16 | Tim4 | Tim4 | Tim4 | TRAF2 | |
| 17 | Tim4 | Tim4 | CD28 | TRAF2 | |
| 18 | Tim4 | Tim4 | Tim1 | TLR8 | CD3ζ |

TABLE 6D

| | | | | Primary | Secondary |
|---|---|---|---|---|---|
| Construct | IgV | Mucin | Transmembrane | Signaling | Signaling |
| No. | Domain | Domain | Domain | Domain | Domain |
| 11′ | SEQ ID | SEQ ID | SEQ ID | SEQ ID | SEQ ID |
| | NO: 52 | NO: 53 | NO: 81 | NO: 262 | NO: 27 |
| 12′ | SEQ ID | SEQ ID | SEQ ID | SEQ ID | SEQ ID |
| | NO: 52 | NO: 53 | NO: 81 | NO: 261 | NO: 27 |
| 13′ | SEQ ID | SEQ ID | SEQ ID | SEQ ID | |
| | NO: 52 | NO: 53 | NO: 25 | NO: 28 | |
| 14′ | SEQ ID | SEQ ID | SEQ ID | SEQ ID | |
| | NO: 52 | NO: 53 | NO: 81 | NO: 264 | |
| 15′ | SEQ ID | SEQ ID | SEQ ID | SEQ ID | |
| | NO: 52 | NO: 53 | NO: 25 | NO: 264 | |
| 16′ | SEQ ID | SEQ ID | SEQ ID | SEQ ID | |
| | NO: 52 | NO: 53 | NO: 81 | NO: 263 | |
| 17′ | SEQ ID | SEQ ID | SEQ ID | SEQ ID | |
| | NO: 52 | NO: 53 | NO: 25 | NO: 263 | |
| 18′ | SEQ ID | SEQ ID | SEQ ID | SEQ ID | SEQ ID |
| | NO: 52 | NO: 53 | NO: 80 | NO: 265 | NO: 27 |

In embodiments, a chimeric Tim receptor of the present disclosure comprises a construct of Table 6C. In some embodiments, a chimeric Tim receptor of the present disclosure comprises a construct of Table 6D.

In a particular embodiment, a chimeric Tim receptor of Construct 11 or Construct 11′ comprises amino acids 25-490 of SEQ ID NO:276. In a specific embodiment, a chimeric Tim receptor of Construct 11 or Construct 11′ comprises an amino acid sequence of SEQ ID NO:276.

In a particular embodiment, a chimeric Tim receptor of Construct 12 or Construct 12′ comprises amino acids 25-495 of SEQ ID NO:277. In a specific embodiment, a chimeric Tim receptor of Construct 12 or Construct 12′ comprises an amino acid sequence of SEQ ID NO:277.

In a particular embodiment, a chimeric Tim receptor of Construct 13 or Construct 13′ comprises amino acids 25-382 of SEQ ID NO:278. In a specific embodiment, a chimeric Tim receptor of Construct 13 or Construct 13′ comprises an amino acid sequence of SEQ ID NO:278.

In a particular embodiment, a chimeric Tim receptor of Construct 14 or Construct 14′ comprises amino acids 25-609 of SEQ ID NO:279. In a specific embodiment, a chimeric Tim receptor of Construct 14 or Construct 14′ comprises an amino acid sequence of SEQ ID NO:279.

In a particular embodiment, a chimeric Tim receptor of Construct 15 or Construct 15′ comprises amino acids 25-615 of SEQ ID NO:280. In a specific embodiment, a chimeric Tim receptor of Construct 15 or Construct 15′ comprises an amino acid sequence of SEQ ID NO:280.

In a particular embodiment, a chimeric Tim receptor of Construct 16 or Construct 16′ comprises amino acids 25-638 of SEQ ID NO:281. In a specific embodiment, a chimeric Tim receptor of Construct 16 or Construct 16′ comprises an amino acid sequence of SEQ ID NO:281.

In a particular embodiment, a chimeric Tim receptor of Construct 17 or Construct 17′ comprises amino acids 25-644 of SEQ ID NO:282. In a specific embodiment, a chimeric Tim receptor of Construct 17 or Construct 17′ comprises an amino acid sequence of SEQ ID NO:282.

In a particular embodiment, a chimeric Tim receptor of Construct 18 or Construct 18′ comprises amino acids 25-640 of SEQ ID NO:283. In a specific embodiment, a chimeric Tim receptor of Construct 18 or Construct 18′ comprises an amino acid sequence of SEQ ID NO:283.

TABLE 6E

| | | | | Primary | Secondary |
|---|---|---|---|---|---|
| Construct | IgV | Mucin | Transmembrane | Signaling | Signaling |
| No. | Domain | Domain | Domain | Domain | Domain |
| 19 | Tim4 | Tim1 | Tim1 | TLR8 | CD3ζ |
| 20 | Tim4 | Tim1 | Tim1 | CD28 | DAP12 |
| 21 | Tim4 | Tim1 | CD28 | CD28 | DAP12 |

TABLE 6F

| | | | | Primary | Secondary |
|---|---|---|---|---|---|
| Construct | IgV | Mucin | Transmembrane | Signaling | Signaling |
| No. | Domain | Domain | Domain | Domain | Domain |
| 19 | SEQ ID | SEQ ID | SEQ ID | SEQ ID | SEQ ID |
| | NO: 52 | NO: 59 | NO: 80 | NO: 265 | NO: 27 |
| 20 | SEQ ID | SEQ ID | SEQ ID | SEQ ID | SEQ ID |
| | NO: 52 | NO: 59 | NO: 80 | NO: 28 | NO: 103 |
| 21 | SEQ ID | SEQ ID | SEQ ID | SEQ ID | SEQ ID |
| | NO: 52 | NO: 59 | NO: 25 | NO: 28 | NO: 103 |

In embodiments, a chimeric Tim receptor of the present disclosure comprises a construct of Table 6E. In some embodiments, a chimeric Tim receptor of the present disclosure comprises a construct of Table 6F.

In a particular embodiment, a chimeric Tim receptor of Construct 19 or Construct 19′ comprises amino acids 25-628 of SEQ ID NO:284. In a specific embodiment, a chimeric Tim receptor of Construct 19 or Construct 19′ comprises an amino acid sequence of SEQ ID NO:284.

In a particular embodiment, a chimeric Tim receptor of Construct 20 or Construct 20′ comprises amino acids 25-416 of SEQ ID NO:285. In a specific embodiment, a chimeric Tim receptor of Construct 20 or Construct 20′ comprises an amino acid sequence of SEQ ID NO:285.

In a particular embodiment, a chimeric Tim receptor of Construct 21 or Construct 21′ comprises amino acids 25-422 of SEQ ID NO:286. In a specific embodiment, a chimeric Tim receptor of Construct 21 or Construct 21′ comprises an amino acid sequence of SEQ ID NO:286.

Further exemplary chimeric Tim receptors are described in Table 7. In some embodiments, a chimeric Tim receptor of the present disclosure comprises a construct of Table 7.

In some embodiments, a chimeric Tim 4 receptor comprises the amino acid sequence of SEQ ID NO:527 or the amino acid sequence of SEQ ID NO:527 absent the signal sequence (amino acids 1-24). In some embodiments, a chimeric Tim 4 receptor comprises the amino acid sequence of SEQ ID NO:528 or the amino acid sequence of SEQ ID NO:528 absent the signal sequence (amino acids 1-24). In some embodiments, a chimeric Tim 4 receptor comprises the amino acid sequence of SEQ ID NO:529 or the amino acid sequence of SEQ ID NO:529 absent the signal sequence (amino acids 1-24). In some embodiments, a chimeric Tim 4 receptor comprises the amino acid sequence of SEQ ID NO:530 or the amino acid sequence of SEQ ID NO:530 absent the signal sequence (amino acids 1-24). In some embodiments, a chimeric Tim 4 receptor comprises the amino acid sequence of SEQ ID NO:531 or the amino acid sequence of SEQ ID NO:531 absent the signal sequence (amino acids 1-24). In some embodiments, a chimeric Tim 4 receptor comprises the amino acid sequence of SEQ ID NO:532 or the amino acid sequence of SEQ ID NO:532 absent the signal sequence (amino acids 1-24). In some embodiments, a chimeric Tim 4 receptor comprises the amino acid sequence of SEQ ID NO:533 or the amino acid sequence of SEQ ID NO:533 absent the signal sequence (amino acids 1-24). In some embodiments, a chimeric Tim 4 receptor comprises the amino acid sequence of SEQ ID NO:534 or the amino acid sequence of SEQ ID NO:534 absent the signal sequence (amino acids 1-24). In some embodiments, a chimeric Tim 4 receptor comprises the amino acid sequence of SEQ ID NO:535 or the amino acid sequence of SEQ ID NO:535 absent the signal sequence (amino acids 1-24). In some embodiments, a chimeric Tim 4 receptor comprises the amino acid sequence of SEQ ID NO:536 or the amino acid sequence of SEQ ID NO:536 absent the signal sequence (amino acids 1-24). In some embodiments, a chimeric Tim 4 receptor comprises the amino acid sequence of SEQ ID NO:537 or the amino acid sequence of SEQ ID NO:537 absent the signal sequence (amino acids 1-24). In some embodiments, a chimeric Tim 4 receptor comprises the amino acid sequence of SEQ ID NO:538 or the amino acid sequence of SEQ ID NO:538 absent the signal sequence (amino acids 1-24). In some embodiments, a chimeric Tim 4 receptor comprises the amino acid sequence of SEQ ID NO:539 or the amino acid sequence of SEQ ID NO:539 absent the signal sequence (amino acids 1-24). In some embodiments, a chimeric Tim 4 receptor comprises the amino acid sequence of SEQ ID NO:540 or the amino acid sequence of SEQ ID NO:540 absent the signal sequence (amino acids 1-24). In some embodiments, a chimeric Tim 4 receptor comprises the amino acid sequence of SEQ ID NO:541 or the amino acid sequence of SEQ ID NO:541 absent the signal sequence (amino acids 1-24). In some embodiments, a chimeric Tim 4 receptor comprises the amino acid sequence of SEQ ID NO:542 or the amino acid sequence of SEQ ID NO:542 absent the signal sequence (amino acids 1-24). In some embodiments, a chimeric Tim 4 receptor comprises the amino acid sequence of SEQ ID NO:543 or the amino acid sequence of SEQ ID NO:543 absent the signal sequence (amino acids 1-24). In some embodiments, a chimeric Tim 4 receptor comprises the amino acid sequence of SEQ ID NO:544 or the amino acid sequence of SEQ ID NO:544 absent the signal sequence (amino acids 1-24). In some embodiments, a chimeric Tim 4 receptor comprises the amino acid sequence of SEQ ID NO:545 or the amino acid sequence of SEQ ID NO:545 absent the signal sequence (amino acids 1-24). In some embodiments, a chimeric Tim 4 receptor comprises the amino acid sequence of SEQ ID NO:546 or the amino acid sequence of SEQ ID NO:546 absent the signal sequence (amino acids 1-24). In some embodiments, a chimeric Tim 4 receptor comprises the amino acid sequence of SEQ ID NO:547 or the amino acid sequence of SEQ ID NO:547 absent the signal sequence (amino acids 1-24). In some embodiments, a chimeric Tim 4 receptor comprises the amino acid sequence of SEQ ID NO:548 or the amino acid sequence of SEQ ID NO:548 absent the signal sequence (amino acids 1-24). In some embodiments, a chimeric Tim 4 receptor comprises the amino acid sequence of SEQ ID NO:149 or the amino acid sequence of SEQ ID NO:549 absent the signal sequence (amino acids 1-24). In some embodiments, a chimeric Tim 4 receptor comprises the amino acid sequence of SEQ ID NO:550 or the amino acid sequence of SEQ ID NO:550 absent the signal sequence (amino acids 1-24). In some embodiments, a chimeric Tim 4 receptor comprises the amino acid sequence of SEQ ID NO:551 or the amino acid sequence of SEQ ID NO:551 absent the signal sequence (amino acids 1-24). In some embodiments, a chimeric Tim 4 receptor comprises the amino acid sequence of SEQ ID NO:552 or the amino acid sequence of SEQ ID NO:552 absent the signal sequence (amino acids 1-24). In some embodiments, a chimeric Tim 4 receptor comprises the amino acid sequence of SEQ ID NO:553 or the amino acid sequence of SEQ ID NO:553 absent the signal sequence (amino acids 1-24). In some embodiments, a chimeric Tim 4 receptor comprises the amino acid sequence of SEQ ID NO:450 or the amino acid sequence of SEQ ID NO:450 absent the signal sequence (amino acids 1-24). In some embodiments, a chimeric Tim 4 receptor comprises the amino acid sequence of SEQ ID NO:451 or the amino acid sequence of SEQ ID NO:451 absent the signal sequence (amino acids 1-24)

TABLE 7

| Construct | Construct | Signal | Extracellular | Transmembrane | Intracellular | Intracellular | Intracellular |
|---|---|---|---|---|---|---|---|
| Number | Description | Peptide | Domain | Domain | Domain #1 | Domain #2 | Domain #3 |
| pCTX1161 | hTIM4-TIM4- | hTIM4 | TIM4 | CD28 | CD28 | CD3z | |
| [SEQ ID | CD28tm-CD28icd- | [SEQ ID | [SEQ ID | [SEQ ID | [SEQ ID | [SEQ ID | |
| NO: 527] | CD3zICD | NO: 518] | NO: 519] | NO: 520] | NO: 28] | NO: 26] | |
| pCTX1162 | hTIM4-TIM4- | hTIM4 | TIM4 | CD28 | CD28 | TLR2 | CD3z |
| [SEQ ID | CD28tm-CD28icd- | [SEQ ID | [SEQ ID | [SEQ ID | [SEQ ID | [SEQ ID | [SEQ ID |
| NO: 528] | TLR2ICD-CD3zICD | NO: 518] | NO: 519] | NO: 520] | NO: 28] | NO: 522] | NO: 26] |
| pCTX1163 | hTIM4-TIM4- | hTIM4 | TIM4 | CD28 | CD28 | CD3z | TLR2 |
| [SEQ ID | CD28tm-CD28icd- | [SEQ ID | [SEQ ID | [SEQ ID | [SEQ ID | [SEQ ID | [SEQ ID |
| NO: 529] | CD3zICD-TLR2ICD | NO: 518] | NO: 519] | NO: 520] | NO: 28] | NO: 26] | NO: 522] |
| pCTX1164 | hTIM4-TIM4- | hTIM4 | TIM4 | CD28 | CD28 | TLR8 | CD3z |
| [SEQ ID | CD28tm-CD28icd- | [SEQ ID | [SEQ ID | [SEQ ID | [SEQ ID | [SEQ ID | [SEQ ID |
| NO: 530] | TLR8ICD-CD3zICD | NO: 518] | NO: 519] | NO: 520] | NO: 28] | NO: 119] | NO: 26] |
| pCTX1165 | hTIM4-TIM4- | hTIM4 | TIM4 | CD28 | CD28 | CD3z | TLR8 |
| [SEQ ID | CD28tm-CD28icd- | [SEQ ID | [SEQ ID | [SEQ ID | [SEQ ID | [SEQ ID | [SEQ ID |
| NO: 531] | CD3zICD-TLR8ICD | NO: 518] | NO: 519] | NO: 520] | NO: 4] | NO: 26] | NO: 119] |
| pCTX1166 | hTIM4-TIM4- | hTIM4 | TIM4 | CD28 | TLR2 | CD3z | |
| [SEQ ID | CD28tm-TLR2ICD- | [SEQ ID | [SEQ ID | [SEQ ID | [SEQ ID | [SEQ ID | |
| NO: 532] | CD3zICD | NO: 518] | NO: 519] | NO: 520] | NO: 522] | NO: 26] | |
| pCTX1167 | hTIM4-TIM4- | hTIM4 | TIM4 | CD28 | CD3z | TLR2 | |
| [SEQ ID | CD28tm-CD3zICD- | [SEQ ID | [SEQ ID | [SEQ ID | [SEQ ID | [SEQ ID | |
| NO: 533] | TLR2ICD | NO: 518] | NO: 519] | NO: 520] | NO: 26] | NO: 522] | |
| pCTX1168 | hTIM4-TIM4- | hTIM4 | TIM4 | CD28 | TLR8 | CD3z | |
| [SEQ ID | CD28tm-TLR8ICD- | [SEQ ID | [SEQ ID | [SEQ ID | [SEQ ID | [SEQ ID | |
| NO: 534] | CD3zICD | NO: 518] | NO: 519] | NO: 520] | NO: 119] | NO: 26] | |
| pCTX1169 | hTIM4-TIM4- | hTIM4 | TIM4 | CD28 | CD3z | TLR8 | |
| [SEQ ID | CD28tm-CD3zICD- | [SEQ ID | [SEQ ID | [SEQ ID | [SEQ ID | [SEQ ID | |
| NO: 535] | TLR8ICD | NO: 518] | NO: 519] | NO: 520] | NO: 26] | NO: 119] | |
| pCTX1170 | hTIM4-TIM4- | hTIM4 | TIM4 | CD28 | TRAF6 | CD3z | |
| [SEQ ID | CD28tm-TRAF6ICD- | [SEQ ID | [SEQ ID | [SEQ ID | [SEQ ID | [SEQ ID | |
| NO: 536] | CD3zICD | NO: 518] | NO: 519] | NO: 520] | NO: 122] | NO: 26] | |
| pCTX1171 | hTIM4-TIM4- | hTIM4 | TIM4 | CD28 | CD3z | TRAF6 | |
| [SEQ ID | CD28tm-CD3zICD- | [SEQ ID | [SEQ ID | [SEQ ID | [SEQ ID | [SEQ ID | |
| NO: 537] | TRAF6ICD | NO: 518] | NO: 519] | NO: 520] | NO: 26] | NO: 122] | |
| pCTX1172 | hTIM4-TIM4- | hTIM4 | TIM4 | TIM4 | CD28 | CD3z | |
| [SEQ ID | TIM4tm-CD28icd- | [SEQ ID | [SEQ ID | [SEQ ID | [SEQ ID | [SEQ ID | |
| NO: 538] | CD3zICD | NO: 518] | NO: 519] | NO: 521] | NO: 28] | NO: 26] | |
| pCTX1173 | hTIM4-TIM4- | hTIM4 | TIM4 | TIM4 | CD28 | TLR2 | CD3z |
| [SEQ ID | TIM4tm-CD28icd- | [SEQ ID | [SEQ ID | [SEQ ID | [SEQ ID | [SEQ ID | [SEQ ID |
| NO: 539] | TLR2ICD-CD3zICD | NO: 518] | NO: 519] | NO: 521] | NO: 28] | NO: 522] | NO: 26] |
| pCTX1174 | hTIM4-TIM4- | hTIM4 | TIM4 | TIM4 | CD28 | CD3z | TLR2 |
| [SEQ ID | TIM4tm-CD28icd- | [SEQ ID | [SEQ ID | [SEQ ID | [SEQ ID | [SEQ ID | [SEQ ID |
| NO: 540] | CD3zICD-TLR2ICD | NO: 518] | NO: 519] | NO: 521] | NO: 28] | NO: 26] | NO: 522] |
| pCTX1175 | hTIM4-TIM4- | hTIM4 | TIM4 | TIM4 | CD28 | TLR8 | CD3z |
| [SEQ ID | TIM4tm-CD28icd- | [SEQ ID | [SEQ ID | [SEQ ID | [SEQ ID | [SEQ ID | [SEQ ID |
| NO: 541] | TLR8ICD-CD3zICD | NO: 518] | NO: 519] | NO: 521] | NO: 28] | NO: 119] | NO: 26] |
| pCTX1176 | hTIM4-TIM4- | hTIM4 | TIM4 | TIM4 | CD28 | CD3z | TLR8 |
| [SEQ ID | TIM4tm-CD28icd- | [SEQ ID | [SEQ ID | [SEQ ID | [SEQ ID | [SEQ ID | [SEQ ID |
| NO: 542] | CD3zICD-TLR8ICD | NO: 518] | NO: 519] | NO: 521] | NO: 28] | NO: 26] | NO: 119] |
| pCTX1177 | hTIM4-TIM4- | hTIM4 | TIM4 | TIM4 | TLR2 | CD3z | |
| [SEQ ID | TIM4tm-TLR2ICD- | [SEQ ID | [SEQ ID | [SEQ ID | [SEQ ID | [SEQ ID | |
| NO: 543] | CD3zICD | NO: 518] | NO: 519] | NO: 521] | NO: 522] | NO: 26] | |
| pCTX1178 | hTIM4-TIM4- | hTIM4 | TIM4 | TIM4 | CD3z | TLR2 | |
| [SEQ ID | TIM4tm-CD3zICD- | [SEQ ID | [SEQ ID | [SEQ ID | [SEQ ID | [SEQ ID | |
| NO: 544] | TLR2ICD | NO: 518] | NO: 519] | NO: 521] | NO: 26] | NO: 522] | |
| pCTX1179 | hTIM4-TIM4- | hTIM4 | TIM4 | TIM4 | TLR8 | CD3z | |
| [SEQ ID | TIM4tm-TLR8ICD- | [SEQ ID | [SEQ ID | [SEQ ID | [SEQ ID | [SEQ ID | |
| NO: 545] | CD3zICD | NO: 518] | NO: 519] | NO: 521] | NO: 119] | NO: 26] | |
| pCTX1180 | hTIM4-TIM4- | hTIM4 | TIM4 | TIM4 | CD3z | TLR8 | |
| [SEQ ID | TIM4tm-CD3zICD- | [SEQ ID | [SEQ ID | [SEQ ID | [SEQ ID | [SEQ ID | |
| NO: 546] | TLR8ICD | NO: 518] | NO: 519] | NO: 521] | NO: 26] | NO: 119] | |
| pCTX1181 | hTIM4-TIM4- | hTIM4 | TIM4 | TIM4 | TRAF6 | CD3z | |
| [SEQ ID | TIM4tm-TRAF6ICD- | [SEQ ID | [SEQ ID | [SEQ ID | [SEQ ID | [SEQ ID | |
| NO: 547] | CD3zICD | NO: 518] | NO: 519] | NO: 521] | NO: 122] | NO: 26] | |
| pCTX1182 | hTIM4-TIM4- | hTIM4 | TIM4 | TIM4 | CD3z | TRAF6 | |
| [SEQ ID | TIM4tm-CD3zICD- | [SEQ ID | [SEQ ID | [SEQ ID | [SEQ ID | [SEQ ID | |
| NO: 548] | TRAF6ICD | NO: 518] | NO: 519] | NO: 521] | NO: 26] | NO: 122] | |
| pCTX1183 | hTIM4-TIM4- | hTIM4 | TIM4 | TIM4 | TIM4 | | |
| [SEQ ID | TIM4tm- | [SEQ ID | [SEQ ID | [SEQ ID | [SEQ ID | | |
| NO: 549] | TIM4ICD | NO: 518] | NO: 519] | NO: 521] | NO: 525] | | |
| pCTX1184 | hTIM4-TIM4- | hTIM4 | TIM4 | TIM4 | TIM4 | TLR2 | CD3z |
| [SEQ ID | TIM4tm-TIM4ICD- | [SEQ ID | [SEQ ID | [SEQ ID | [SEQ ID | [SEQ ID | [SEQ ID |
| NO: 550] | TLR2-CD3z | NO: 518] | NO: 519] | NO: 521] | NO: 524] | NO: 522] | NO: 26] |
| pCTX1185 | hTIM4-TIM4- | hTIM4 | TIM4 | TIM4 | TIM4 | CD3z | TLR2 |
| [SEQ ID | TIM4tm-TIM4ICD- | [SEQ ID | [SEQ ID | [SEQ ID | [SEQ ID | [SEQ ID | [SEQ ID |
| NO: 551] | CD3z-TLR2 | NO: 518] | NO: 519] | NO: 521] | NO: 524] | NO: 26] | NO: 522] |
| pCTX1186 | hTIM4-TIM4- | hTIM4 | TIM4 | TIM4 | TIM4 | TLR8 | CD3z |
| [SEQ ID | TIM4tm-TIM4ICD- | [SEQ ID | [SEQ ID | [SEQ ID | [SEQ ID | [SEQ ID | [SEQ ID |
| NO: 552] | TLR8-CD3z | NO: 518] | NO: 519] | NO: 521] | NO: 524] | NO: 119] | NO: 26] |
| pCTX1187 | hTIM4-TIM4- | hTIM4 | TIM4 | TIM4 | TIM4 | CD3z | TLR8 |
| [SEQ ID | TIM4tm-TIM4ICD- | [SEQ ID | [SEQ ID | [SEQ ID | [SEQ ID | [SEQ ID | [SEQ ID |
| NO: 553] | CD3z-TLR8 | NO: 518] | NO: 519] | NO: 521] | NO: 524] | NO: 26] | NO: 119] |
| pCTX1189 | hTIM4-TIM4- | hTIM4 | TIM4 | TIM4 | TLR2 | | |
| [SEQ ID | TIM4tm- | [SEQ ID | [SEQ ID | [SEQ ID | [SEQ ID | | |
| NO: 450] | TLR2ICD | NO: 518] | NO: 519] | NO: 521] | NO: 522] | | |
| pCTX1190 | hTIM4-TIM4- | hTIM4 | TIM4 | TIM4 | TLR8 | | |
| [SEQ ID | TIM4tm- | [SEQ ID | [SEQ ID | [SEQ ID | [SEQ ID | | |
| NO: 451] | TLR8ICD | NO: 518] | NO: 519] | NO: 521] | NO: 119] | | |

In some embodiments, an anti-CD72 CAR combination therapy composition and/or method comprises a chimeric engulfment receptor selected from Table 2, and/or a chimeric Tim receptor selected from any one of Tables 3, 4, 6A-6F, and 7.

In some embodiments, an anti-CD72 CAR combination therapy composition and/or method comprises a chimeric engulfment receptor comprising the amino acid sequence set forth in any one of SEQ ID NOS: 131-212 or a chimeric Tim receptor comprising the amino acid sequence set forth in any one of SEQ ID NOS:250-253, 257-259, and 266-286.

Exemplary chimeric Tim receptor components, constructs, vectors, engineered host cells, methods of making, and methods of using are described in International Application Publication No. WO2019/191334; U.S. Provisional Application No. 62/910,133 filed on Oct. 3, 2019; and U.S. Provisional Application filed on Aug. 14, 2020 titled “Chimeric Tim Receptors and Uses Thereof,” each of which is herein incorporated by reference in its entirety.

Without wishing to be bound by theory. CARs and CERs of the present disclosure may act together to cooperatively clear target tumor cells. The CD72 specific CAR engages high density target antigen on target cells (e.g., lymphoma cells), resulting in T cell activation, indication of cytotoxicity, secretion of pro-inflammatory cytokines, and phosphatidylserine induction on target cells. The CER engages exposed phosphatidyl serine on target cells (e.g., lymphoma), resulting in maintenance of T cell activation and cytotoxicity and uptake of target cell antigens. CER modified cells present target cell antigens and enhance clearance and recruit additional immune response to additional target cell antigens.

### IV. Polynucleotides, Vectors, and Engineered Host Cells

In certain aspects, the present disclosure provides nucleic acid molecules that encode any one or more of the anti-CD72 CARs described herein, and optionally a CER or chimeric Tim receptor described herein. A nucleic acid may refer to a single- or double-stranded DNA, cDNA, or RNA, and may include a positive and a negative strand of the nucleic acid which complement one another, including antisense DNA, cDNA, and RNA. A nucleic acid may be naturally occurring or synthetic forms of DNA or RNA. The nucleic acid sequences encoding an anti-CD72 CAR can be obtained or produced using recombinant methods known in the art using standard techniques, such as by screening libraries from cells expressing the desired sequence or a portion thereof, by deriving the sequence from a vector known to include the same, or by isolating the sequence or a portion thereof directly from cells or tissues containing the same as described in, for example, Sambrook et al. (1989 and 2001 editions; Molecular Cloning: A Laboratory Manual, Cold Spring Harbor Laboratory Press, NY) and Ausubel et al. (Current Protocols in Molecular Biology, 2003). Alternatively, the sequence of interest can be produced synthetically, rather than being cloned.

Polynucleotides encoding the anti-CD72 CAR compositions provided herein may be derived from any animal, such as humans, primates, cows, horses, sheep, dogs, cats, mice, rats, rabbits, guinea pigs, pigs, or a combination thereof. In certain embodiments, a polynucleotide encoding the chimeric Tim receptor is from the same animal species as the host cell into which the polynucleotide is inserted.

The polynucleotides encoding anti-CD72 CAR of the present disclosure may be operatively linked to expression control sequences. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion.

In certain embodiments, a polynucleotide encoding an anti-CD72 CAR comprises a sequence encoding a signal peptide (also referred to as leader peptide or signal sequence) at the 5′-end for targeting of the precursor protein to the secretory pathway. The signal peptide is optionally cleaved from the N-terminus of the extracellular domain during cellular processing and localization of the anti-CD72 CAR to the host cell membrane. A polypeptide from which a signal peptide sequence has been cleaved or removed may also be called a mature polypeptide. Examples of signal peptides that may be used in the anti-CD72 CARs of the present disclosure include signal peptides derived from endogenous secreted proteins, including, e.g., GM-CSF (amino acid sequence of SEQ ID NO:254), GMCSFR (SEQ ID NO:47), IgK (SEQ ID NO:438), CD8a (SEQ ID NO:439), and IL-2 (SEQ ID NO:440). In certain embodiments, a polynucleotide sequence encodes a mature chimeric Tim receptor polypeptide, or a polypeptide sequence comprises a mature chimeric Tim receptor polypeptide. It is understood by persons of skill in the art that for sequences disclosed herein that include a signal peptide sequence, the signal peptide sequence may be replaced with another signal peptide that is capable of trafficking the encoded protein to the extracellular membrane.

In certain embodiments, an anti-CD72 CAR encoding polynucleotide of the present disclosure is codon optimized for efficient expression in a target host cell comprising the polynucleotide (see, e.g, Scholten et al., Clin. Immunol. 119: 135-145 (2006)). As used herein, a “codon optimized” polynucleotide comprises a heterologous polynucleotide having codons modified with silent mutations corresponding to the abundances of tRNA in a host cell of interest.

A single polynucleotide molecule may encode one, two, or more anti-CD72 CARs according to any of the embodiments disclosed herein. A polynucleotide encoding more than one transgene may comprise a sequence (e.g., IRES, viral 2A peptide) disposed between each gene for multicistronic expression.

Polynucleotides encoding at least two transgenes (e.g., anti-CD72 CAR and CER or chimeric Tim receptor) provided in the present disclosure may be used to compose tandem expression cassettes. A tandem expression cassette refers to a component of a vector nucleic acid comprising at least two transgenes under the control of, or operatively linked to, the same set of regulatory sequences for tandem or co-expression of the at least two transgenes. Regulatory sequences that may be used in tandem expression cassettes of the present disclosure include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; sequences that enhance protein secretion, or any combination thereof.

In one aspect, the present disclosure provides a tandem expression cassette comprising a polynucleotide encoding an anti-CD72 CAR of the present disclosure and a polynucleotide encoding a CER or chimeric Tim receptor.

In certain embodiments, a tandem expression cassette can be constructed to optimize spatial and temporal control. For example, a tandem expression cassette can include promoter elements to optimize spatial and temporal control. In some embodiments, a tandem expression cassette includes tissue specific promoters or enhancers that enable specific induction of a tandem expression cassette to an organ, a cell type (e.g., immune cell), or a pathologic microenvironment, such as a tumor or infected tissue. An “enhancer” is an additional promoter element that can function either cooperatively or independently to activate transcription. In certain embodiments, a tandem expression cassette includes a constitutive promoter. An exemplary constitutive promoter for use in tandem expression cassettes of the present disclosure is an EF-1α promoter. In certain embodiments, a tandem expression cassette includes an inducible promoter. In certain embodiments, a tandem expression cassette includes a tissue specific promoter.

The at least two transgenes contained within the tandem expression cassettes may be in any order. For example, a tandem expression cassette comprising a polynucleotide encoding a chimeric Tim receptor and a polynucleotide encoding an anti-CD72 CAR may be arranged from 5′ to 3′: chimeric Tim receptor-CAR, or CAR-chimeric Tim receptor. In another example, a tandem expression cassette comprising a polynucleotide encoding a CER and a polynucleotide encoding an anti-CD72 CAR may be arranged from 5′ to 3′: chimeric CER-CAR, or CAR-CER.

In certain embodiments, receptors that comprise two or more polypeptide chains that associate to form a multimer or complex may be encoded by two or more polynucleotide molecules within a tandem expression construct. Exemplary multimeric receptors contemplated for expression in tandem expression constructs of the present disclosure include multichain CARs, TCRs, TCR-CARs, and TRUC™ constructs. Accordingly, exemplary tandem expression cassette embodiments encoding an anti-CD72 CAR and a TCR may comprise a polynucleotide encoding the anti-CD72 CAR, a polynucleotide encoding a TCRα chain polypeptide, and a polynucleotide encoding a TCRβ chain polypeptide.

In certain embodiments, tandem expression cassettes of the present disclosure may comprise an internal ribosome entry site (IRES) or peptide cleavage site such as a furin cleavage site or viral 2A peptide, disposed between each polynucleotide contained within the tandem expression cassette to allow for co-expression of multiple proteins from a single mRNA. For example, an IRES, furin cleavage site, or viral 2A peptide may be disposed between a polynucleotide encoding an anti-CD72 CAR and a polynucleotide encoding a CER or chimeric Tim receptor within a tandem expression cassette. In certain embodiments, a viral 2A peptide is a porcine teschovirus-1 (P2A), Thosea asigna virus (T2A), equine rhinitis A virus (E2A), foot-and-mouth disease virus (F2A), or variant thereof. An exemplary T2A peptide comprises an amino acid sequence of any one of SEQ ID NOs:287-290. An exemplary P2A peptide comprises an amino acid sequence of SEQ ID NO:291, 292, or 442. An exemplary E2A peptide sequence comprises an amino acid sequence of SEQ ID NO:293. An exemplary F2A peptide sequence comprises an amino acid sequence of SEQ ID NO:294.

Certain embodiments of tandem expression cassettes of the present disclosure comprise a polynucleotide encoding an anti-CD72 CAR and a polynucleotide encoding a CER or chimeric Tim receptor of the present disclosure. Upon binding a target cell expressing CD72 by the anti-CD72 CAR, a cell modified to express such a tandem expression cassette induces apoptosis of the target cell. Apoptosis induces exposure of pro-engulfment markers on the target cell, such as phosphatidylserine, which may then target the damaged or apoptotic cells for engulfment by the CER or chimeric Tim receptor.

A polynucleotide encoding a desired anti-CD72 CAR can be inserted into an appropriate vector, e.g., a viral vector, non-viral plasmid vector, and non-viral vectors, such as lipid-based DNA vectors, modified mRNA (modRNA), self-amplifying mRNA, CELiD, and transposon-mediated gene transfer (PiggyBac, Sleeping Beauty), for introduction into a host cell of interest (e.g., an immune cell). Polynucleotides encoding an anti-CD72 CAR of the present disclosure can be cloned into any suitable vector, such as an expression vector, a replication vector, a probe generation vector, or a sequencing vector. In certain embodiments, a polynucleotide encoding the extracellular domain, a polynucleotide encoding the transmembrane domain, and a polynucleotide encoding the intracellular signaling domain are joined together into a single polynucleotide and then inserted into a vector. In other embodiments, a polynucleotide encoding the extracellular domain, a polynucleotide encoding the transmembrane domain, and a polynucleotide encoding the intracellular signaling domain may be inserted separately into a vector such that the expressed amino acid sequence produces a functional anti-CD72 CAR. A vector that encodes an anti-CD72 CAR is referred to herein as a “anti-CD72 CAR vector.”

In certain embodiments, a vector comprises a polynucleotide encoding one anti-CD72 CAR. In certain embodiments, a vector comprises one polynucleotide encoding two or more anti-CD72 CARs. In certain embodiments, a single polynucleotide encoding two or more anti-CD72 CARs is cloned into a cloning site and expressed from a single promoter, with each anti-CD72 CAR sequence separated from each other by an internal ribosomal entry site (IRES), furin cleavage site, or viral 2A peptide to allow for co-expression of multiple genes from a single open reading frame (e.g., a multicistronic vector). In certain embodiments, a viral 2A peptide is a porcine teschovirus-1 (P2A), Thosea asigna virus (T2A), equine rhinitis A virus (E2A), foot-and-mouth disease virus (F2A), or variant thereof. An exemplary T2A peptide comprises an amino acid sequence of SEQ ID NO:287-290. An exemplary P2A peptide comprises an amino acid sequence of SEQ ID NO:291, 292, or 442. An exemplary E2A peptide sequence comprises an amino acid sequence of SEQ ID NO:293. An exemplary F2A peptide sequence comprises an amino acid sequence of SEQ ID NO:294.

In certain embodiments, a vector comprises two or more polynucleotides, each polynucleotide encoding an anti-CD72 CAR. The two or more polynucleotides encoding anti-CD72 CARs may be cloned sequentially into a vector at different cloning sites, with each anti-CD72 CAR expressed under the regulation of different promoters. In certain embodiments, vectors that allow long-term integration of a transgene and propagation to daughter cells are utilized. Examples include viral vectors such as, adenovirus, adeno-associated virus, vaccinia virus, herpes viruses, cytomegalovirus, pox virus, or retroviral vectors, such as lentiviral vectors. Vectors derived from lentivirus can be used to achieve long-term gene transfer and have added advantages over vectors including the ability to transduce non-proliferating cells, such as hepatocytes, and low immunogenicity.

In certain embodiments, a vector comprises a polynucleotide encoding a anti-CD72 CAR and a polynucleotide encoding a CER or chimeric Tim receptor. In certain embodiments, a single polynucleotide encoding the anti-CD72 CAR and a CER or chimeric Tim receptor is cloned into a cloning site and expressed from a single promoter, with the anti-CD72 CAR sequence and CER or chimeric Tim receptor sequence separated from each other by an internal ribosomal entry site (IRES), furin cleavage site, or viral 2A peptide to allow for co-expression of multiple genes from a single open reading frame (e.g., a multicistronic vector). In certain embodiments, a viral 2A peptide is a porcine teschovirus-1 (P2A), Thosea asigna virus (T2A), equine rhinitis A virus (E2A), foot-and-mouth disease virus (F2A), or variant thereof. An exemplary T2A peptide comprises an amino acid sequence of SEQ ID NO:287-290. An exemplary P2A peptide comprises an amino acid sequence of SEQ ID NO:291, 292, or 442. An exemplary E2A peptide sequence comprises an amino acid sequence of SEQ ID NO:293. An exemplary F2A peptide sequence comprises an amino acid sequence of SEQ ID NO:294.

In certain embodiments, a polynucleotide encoding the anti-CD72 CAR and a polynucleotide encoding the CER or chimeric Tim receptor are joined together into a single polynucleotide and then inserted into a vector. In other embodiments, a polynucleotide encoding the anti-CD72 CAR, and a polynucleotide encoding the CER or chimeric Tim receptor may be inserted separately into a vector in the same or different cloning sites, such that the expressed amino acid sequence produces a functional anti-CD72 CAR and functional CER or chimeric Tim receptor. In some embodiments, the anti-CD72 CAR and CER or chimeric Tim receptor are expressed from the same promoter in a tandem expression cassette. A vector that encodes a tandem expression cassette is referred to herein as a “tandem expression vector.”

In certain embodiments, a vector comprises a polynucleotide encoding an anti-CD72 CAR and a polynucleotide encoding a CER or chimeric Tim receptor. The polynucleotides encoding the anti-CD72 CAR and CER or chimeric Tim receptor may be cloned sequentially into a vector at different cloning sites, with the anti-CD72 CAR and CER or chimeric Tim receptor expressed under the regulation of different promoters. A vector that encodes a core virus is referred to herein as a “viral vector.” There are a large number of available viral vectors suitable for use with the compositions of the instant disclosure, including those identified for human gene therapy applications (see Pfeifer and Verme, Ann. Rev. Genomics Hum. Genet. 2:177, 2001). Suitable viral vectors include vectors based on RNA viruses, such as retrovirus-derived vectors, e.g., Maloney murine leukemia virus (MLV)-derived vectors, and include more complex retrovirus-derived vectors, e.g., lentivirus-derived vectors. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing chimeric receptor transgenes are known in the art and have been previous described, for example, in U.S. Pat. No. 8,119,772; Walchli et al., PLOS One 6:327930, 2011; Zhao et al., J. Immunol. 174:4415, 2005; Engels et al., Hum. Gene Ther. 14:1155, 2003; Frecha et al., Mol. Ther. 18:1748, 2010; Verhocyen et al., Methods Mol. Biol. 506:97, 2009. Retroviral and lentiviral vector constructs and expression systems are also commercially available.

In certain embodiments, a viral vector is used to introduce a non-endogenous polynucleotide encoding an anti-CD72 CAR to a host cell. A viral vector may be a retroviral vector or a lentiviral vector. A viral vector may also include a nucleic acid sequence encoding a marker for transduction. Transduction markers for viral vectors are known in the art and include selection markers, which may confer drug resistance, or detectable markers, such as fluorescent markers or cell surface proteins that can be detected by methods such as flow cytometry. In particular embodiments, a viral vector further comprises a gene marker for transduction comprising a fluorescent protein (e.g., green, yellow), an extracellular domain of human CD2, RQR8 tag, or a truncated human EGFR (EGFRt or tEGFR; see Wang et al., Blood 118:1255, 2011). An exemplary tEGFR comprises an amino acid sequence of SEQ ID NO:295 or 443. When a viral vector genome comprises a plurality of genes to be expressed in a host cell as separate proteins from a single transcript, the viral vector may also comprise additional sequences between the two (or more) genes allowing for multicistronic expression. Examples of such sequences used in viral vectors include internal ribosome entry sites (IRES), furin cleavage sites, viral 2A peptides (e.g., T2A, P2A, E2A, F2A), or any combination thereof.

Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5: 1517, 1998).

Other viral vectors recently developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors). In certain embodiments, an anti-CD72 CAR vector can be constructed to optimize spatial and temporal control. For example, an anti-CD72 CAR vector can include promoter elements to optimize spatial and temporal control. In some embodiments, an anti-CD72 CAR vector includes tissue specific promoters or enhancers that enable specific induction of an anti-CD72 CAR to an organ, a cell type (e.g., immune cell), or a pathologic microenvironment, such as a tumor or infected tissue. An “enhancer” is an additional promoter element that can function either cooperatively or independently to activate transcription. In certain embodiments, an anti-CD72 CAR vector includes a constitutive promoter. In certain embodiments, an anti-CD72 CAR vector includes an inducible promoter. In certain embodiments, an anti-CD72 CAR vector includes a tissue specific promoter.

In certain embodiments, an anti-CD72 CAR vector can include a gene encoding a homing receptor, such as CCR4 or CXCR4, to improve homing and antitumor activity in vivo.

Where temporal control is desired, an anti-CD72 CAR vector may include an element that allows for inducible depletion of transduced cells. For example, such a vector may include an inducible suicide gene. A suicide gene may be an apoptotic gene or a gene that confers sensitivity to an agent (e.g., a drug). Exemplary suicide genes include chemically inducible caspase 9 (iCASP9) (U.S. Patent Publication No. 2013/0071414), chemically inducible Fas, or Herpes simplex virus thymidine kinase (HSV-TK), which confers sensitivity to ganciclovir. In further embodiments, an anti-CD72 CAR vector can be designed to express a known cell surface antigen that, upon infusion of an associated antibody, enables depletion of transduced cells. Examples of cell surface antigens and their associated antibodies that may be used for depletion of transduced cells include CD20 and Rituximab, RQR8 (combined CD34 and CD20 epitopes, allowing CD34 selection and anti-CD20 deletion, SEQ ID NO:444) and Rituximab, and EGFR and Cetuximab. Inducible vector systems, such as the tetracycline (Tet)-On vector system which activates transgene expression with doxycycline (Heinz et al., Hum. Gene Ther. 2011, 22:166-76) may also be used for inducible anti-CD72 CAR expression. Inducible anti-CD72 CAR expression may be also accomplished via retention using a selective hook (RUSH) system based on streptavidin anchored to the membrane of the endoplasmic reticulum through a hook and a streptavidin binding protein introduced into the anti-CD72 CAR structure, where addition of biotin to the system leads to the release of the chimeric Tim receptor from the endoplasmic reticulum (Agaugue et al., 2015, Mol. Ther. 23(Suppl. 1):S88).

In certain embodiments, a host cell engineered to express an anti-CD72 CAR and a CER or chimeric Tim receptor may also be modified to co-express one or more small GTPases. Rho GTPases, a family of small (˜21 k Da) signaling G proteins and also a subfamily of the Ras superfamily, regulate actin cytoskeleton organization in various cell types and promote pseudopod extension and phagosome closure during phagocytosis (see, e.g., Castellano et al., 2000, J. Cell Sci. 113:2955-2961). Engulfment requires F-actin recruitment beneath tethered cells or particles, and F-actin rearrangement to allow membrane extension resulting in cell or particle internalization. RhoGTPases include RhoA, Rac1, Rac2, RhoG, and CDC42. Other small GTPases, such as Rap1, is involved in regulation of complement mediated phagocytosis. Co-expression of a small GTPase with the CER or chimeric Tim receptor may promote target cell or particle internalization and/or phagosome formation by the host cell. In some embodiments, a recombinant nucleic acid molecule encoding a GTPase is encoded on a separate vector than the CER or chimeric Tim receptor-containing vector. In other embodiments, a recombinant nucleic acid molecule encoding a GTPasc is encoded on the same vector as the CER or chimeric Tim receptor. The GTPase and CER or chimeric Tim receptor may be expressed under the regulation of different promoters on the same vector (e.g., at different multiple cloning sites). Alternatively, the chimeric Tim receptor and GTPase may be expressed under the regulation of one promoter in a multicistronic vector. The polynucleotide sequence encoding the CER or chimeric Tim receptor and the polynucleotide sequence encoding the small GTPasc(s) may be separated from each other by an IRES or viral 2A peptide in a multicistronic vector. Exemplary 2A peptides include T2A (SEQ ID NOS:287-290), P2A (SEQ ID NOS:291, 292, or 442), E2A (SEQ ID NO:293), F2A (SEQ ID NO:294). Examples of GTPases that may be co-expressed with a CER or chimeric Tim receptor include Rac1, Rac2, Rab5 (also referred to as Rab5a), Rab7, Rap1, RhoA, RhoG, CDC42, or any combination thereof. In specific embodiments, the GTPase comprises or is a sequence that is at least 90%, at least 91%, at least 92%, at least 93%, at least 94%, at least 95%, at least 96%, at least 97%, at least 98%, at least 99%, at least 99.5%, or 100% identical to a Rac1 amino acid sequence of SEQ ID NO:296, a Rab5 amino acid sequence of SEQ ID NO:297, a Rab7 amino acid sequence of SEQ ID NO:298, a Rap1 amino acid sequence of SEQ ID NO:299, a RhoA amino acid sequence of SEQ ID NO:300, a CDC42 amino acid sequence of SEQ ID NO:301, or any combination thereof.

In certain embodiments, a cell, such as an immune cell, obtained from a subject may be engineered into a non-natural or recombinant cell (e.g., a non-natural or recombinant immune cell) by introducing a polynucleotide that encodes an anti-CD72 CAR as described herein, whereby the cell expresses a cell surface localized anti-CD72 CAR. In certain embodiments, a host cell is an immune cell, such as a myeloid progenitor cell or a lymphoid progenitor cell. Exemplary immune cells that may be modified to comprise a polynucleotide encoding an anti-CD72 CAR or a vector comprising a polynucleotide encoding an anti-CD72 CAR include a T cell, a natural killer cell, a B cell, a lymphoid precursor cell, an antigen presenting cell, a dendritic cell, a Langerhans cell, a myeloid precursor cell, a mature myeloid cell, a monocyte, or a macrophage.

In certain embodiments, a B cell is genetically modified to express one or more anti-CD72 CARs. B cells include progenitor or precursor cells committed to the B cell lineage (e.g., pre-pro-B cells, pro-B cells, and pre-B cells); immature and inactivated B cells; or mature and functional or activated B cells. In certain embodiments, B cells may be naïve B cells, plasma cells, regulatory B cells, marginal zone B cells, follicular B cells, lymphoplasmacytoid cell, plasmablast cell, memory B cells, or any combination thereof. Memory B cells may be distinguished from naïve B cells by expression of CD27, which is absent on naïve B cells. In certain embodiments, the B cells can be primary cells or cell lines derived from human, mouse, rat, or other mammals. B cell lines are well known in the art. If obtained from a mammal, a B cell can be obtained from numerous sources, including blood, bone marrow, spleen, lymph node, or other tissues or fluids. A B cell composition may be enriched or purified.

In certain embodiments, a T cell is genetically modified to express one or more anti-CD72 CARs. Exemplary T cells include CD4⁺ helper, CD8⁺ effector (cytotoxic), naïve (CD45 RA+, CCR7+, CD62L+, CD27+, CD45RO−), central memory (CD45RO⁺, CD62L⁺, CD8⁺), effector memory (CD45RA+, CD45RO−, CCR7−, CD62L−, CD27−), T memory stem, regulatory, mucosal-associated invariant (MAIT), γδ (gd), tissue resident T cells, natural killer T cells, or any combination thereof. In certain embodiments, the T cells can be primary cells or cell lines derived from human, mouse, rat, or other mammals. If obtained from a mammal, a T cell can be obtained from numerous sources, including blood, bone marrow, lymph node, thymus, or other tissues or fluids. A T cell composition may be enriched or purified. T cell lines are well known in the art, some of which are described in Sandberg et al., Leukemia 21:230, 2000. In certain embodiments, the T cells lack endogenous expression of a TCRα gene, TCRβ gene, or both. Such T cells may naturally lack endogenous expression of TCRα and β chains, or may have been modified to block expression (e.g., T cells from a transgenic mouse that does not express TCR α and β chains or cells that have been manipulated to inhibit expression of TCR α and β chains) or to knockout a TCRα chain, a TCRβ chain, or both genes.

In certain embodiments, host cells expressing an anti-CD72 CAR of this disclosure on the cell surface are not T cells or cells of a T cell lineage, but cells that are progenitor cells, stem cells or cells that have been modified to express cell surface anti-CD3.

In certain embodiments, an anti-CD72 CAR modified host cell may also be modified to co-express a CER or chimeric Tim receptor.

In certain embodiments, an engineered host cell that co-expresses an anti-CD72 CAR and a CER or chimeric Tim receptor comprises a recombinant nucleic acid encoding the anti-CD72 CAR and a recombinant nucleic acid molecule encoding the a CER or chimeric Tim receptor on separate vectors within the engineered host cell.

In some embodiments, an engineered host cell that co-expresses an anti-CD72 CAR and a CER or chimeric Tim receptor comprises a recombinant nucleic acid encoding the anti-CD72 CAR and a recombinant nucleic acid molecule encoding the CER or chimeric Tim receptor on the same vector within an engineered host cell. The chimeric Tim receptor and cellular immunotherapy agent may be expressed under the regulation of different promoters on the same vector (e.g., at different multiple cloning sites). Alternatively, the chimeric Tim receptor and cellular immunotherapy agent may be expressed under the regulation of one promoter in a multicistronic vector (e.g., tandem expression vector). The polynucleotide sequence encoding the anti-CD72 CAR and the polynucleotide sequence encoding the CER or chimeric Tim receptor may be separated by an IRES or viral 2A peptide in a multicistronic vector.

Tandem expression cassettes, tandem expression vectors, and engineered host cells comprising the same are described in International Application Publication No. WO2019/191339, which is incorporated herein by reference in its entirety.

In certain embodiments, gene editing methods are used to modify the host cell genome to comprise a polynucleotide encoding an anti-CD72 CAR of the present disclosure. Gene editing, or genome editing, is a method of genetic engineering wherein DNA is inserted, replaced, or removed from a host cell's genome using genetically engineered endonucleases. The nucleases create specific double-stranded breaks at targeted loci in the genome. The host cell's endogenous DNA repair pathways then repair the induced break(s), e.g., by non-homologous ending joining (NHEJ) and homologous recombination. Exemplary endonucleases useful for gene editing include a zinc finger nuclease (ZFN), a transcription activator-like effector (TALE) nuclease, a clustered regularly interspaced short palindromic repeats (CRISPR)/Cas nuclease system (e.g., CRISPR-Cas9), a meganuclease, or combinations thereof. Methods of disrupting or knocking out genes or gene expression in immune cells including B cells and T cells, using gene editing endonucleases are known in the art and described, for example, in PCT Publication Nos. WO 2015/066262; WO 2013/074916; WO 2014/059173; Cheong et al., Nat. Comm. 2016 7:10934; Chu et al., Proc. Natl. Acad. Sci. USA 2016 113:12514-12519; methods from each of which are incorporated herein by reference in their entirety.

In certain embodiments, expression of an endogenous gene of the host cell is inhibited, knocked down, or knocked out. Examples of endogenous genes that may be inhibited, knocked down, or knocked out in a B cell include IGH, IGκ, IGλ, or any combination thereof. Examples of endogenous genes that may be inhibited, knocked down, or knocked out in a T cell include a TCR gene (TRA or TRB), an HLA gene (HLA class I gene or HLA class II gene), an immune checkpoint molecule (PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, or PVRIG/CD112R), or any combination thereof. Expression of an endogenous gene may be inhibited, knocked down, or knocked out at the gene level, transcriptional level, translational level, or a combination thereof. Methods of inhibiting, knocking down, or knocking out an endogenous gene may be accomplished, for example, by an RNA interference agent (e.g., siRNA, shRNA, miRNA, etc.) or an engineered endonuclease (e.g., CRISPR/Cas nuclease system, a zinc finger nuclease (ZFN), a Transcription Activator Like Effector nuclease (TALEN), a meganuclease), or any combination thereof. In certain embodiments, an endogenous B cell gene (e.g., IGH, IGκ, or IGλ) is knocked out by insertion of a polynucleotide encoding a chimeric Tim receptor of the present disclosure into the locus of the endogenous B cell gene, such as via an engineered endonuclease. In certain embodiments, an endogenous T cell gene (e.g., a TCR gene, an HLA gene, or an immune checkpoint molecule gene) is knocked out by insertion of a polynucleotide encoding a chimeric Tim receptor of the present disclosure into the locus of the endogenous T cell gene, such as via an engineered endonuclease. In certain embodiments, a host cell may be genetically modified to express one type of anti-CD72 CAR. In other embodiments, a host cell may express at least two or more different anti-CD72 CARs.

The present disclosure also provides a composition comprising a population of anti-CD72 CAR modified host cells. In certain embodiments, the population of anti-CD72 CAR modified host cells may be a population of B cells, a population of T cells, a population of natural killer cells, a population of lymphoid precursor cells, a population of antigen presenting cells, a population of dendritic cells, a population of Langerhans cells, a population of myeloid precursor cells, a population of mature myeloid cells, or any combination thereof. Furthermore, a population of anti-CD72 CAR modified host cells of a particular cell type may be composed of one or more subtypes. For example, a population of B cells may be composed of anti-CD72 CAR modified naïve B cells, plasma cells, regulatory B cells, marginal zone B cells, follicular B cells, lymphoplasmacytoid cells, plasmablast cells, memory B cells, or any combination thereof. In another example, a population of T cells may be composed of anti-CD72 CAR modified CD4⁺ helper T cells, CD8⁺ effector (cytotoxic) T cells, naïve (CD45 RA+, CCR7+, CD62L+, CD27+, CD45RO−) T cells, central memory (CD45RO⁺, CD62L⁺, CD8⁺) T cells, effector memory (CD45RA+, CD45RO−, CCR7−, CD62L−, CD27−) T cells, T memory stem cells, regulatory T cells, mucosal-associated invariant T cells (MAIT), γδ (gd) cells, tissue resident T cells, natural killer T cells, or any combination thereof.

In certain embodiments, a population of host cells is composed of cells that each expresses the same anti-CD72 CAR(s). In other embodiments, a population of host cells is composed of a mixture of two or more subpopulation of host cells, wherein each subpopulation expresses a different anti-CD72 CAR or a set of anti-CD72 CAR.

In certain embodiments, when preparing anti-CD72 CAR modified host cells, e.g., B cells or T cells, one or more growth factor cytokines that promotes proliferation of the host cells, e.g., B cells or T cells, may be added to the cell culture. The cytokines may be human or non-human. Exemplary growth factor cytokines that may be used to promote T cell proliferation include IL-2, IL-15, or the like. Exemplary growth factor cytokines that may be used to promote B cell proliferation include CD40L, IL-2, IL-4, IL-15, IL-21, BAFF, or the like.

Prior to genetic modification of the host cells with a anti-CD72 CAR vector, a source of host cells (e.g., T cells, B cells, natural killer cells, etc.) is obtained from a subject (e.g., whole blood, peripheral blood mononuclear cells, bone marrow, lymph node tissue, cord blood, thymus tissue, tissue from a site of infection, ascites, pleural effusion, spleen tissue), from which host cells are isolated using methods known in the art. Specific host cell subsets can be collected in accordance with known techniques and enriched or depleted by known techniques, such as affinity binding to antibodies, flow cytometry and/or immunomagnetic selection. After enrichment and/or depletion steps and introduction of a anti-CD72 CAR, in vitro expansion of the desired modified host cells can be carried out in accordance with known techniques, or variations thereof that will be apparent those skilled in the art.

Engineered host cells co-expressing an anti-CD72 CAR and CER or chimeric Tim receptor of the present disclosure possess cytotoxic activity that is specific for phosphatidylserine expressing target cells. Thus, upon binding phosphatidylserine exposed on the surface of a target cell, a host cell expressing a chimeric Tim receptor is capable of inducing apoptosis of the target cell. In certain embodiments, the host cell co-expressing an anti-CD72 CAR and CER or chimeric Tim receptor of the present disclosure induces apoptosis of the target cell via: release of granzymes, perforins, granulysin, or any combination thereof; Fas ligand-Fas interaction; or both. In further embodiments, the chimeric Tim receptor further confers phosphatidylserine specific engulfment activity to host cells co-expressing an anti-CD72 CAR and CER or chimeric Tim receptor of the present disclosure. In yet further embodiments, the host cell does not naturally exhibit an engulfment phenotype prior to modification with the anti-CD72 CAR and CER or chimeric Tim receptor.

Engineered host cells co-expressing an anti-CD72 CAR and CER or chimeric Tim receptor of the present disclosure may also be capable of costimulating T cells via at least one signaling pathway. In certain embodiments, chimeric Tim receptors provide costimulatory signals to T cells via at least two distinct signaling pathways (e.g., via the selected costimulatory signaling domain(s) in the chimeric Tim receptor). For example, a chimeric Tim receptor comprising a CD28 costimulatory signaling domain may be capable of providing a costimulatory signal via CD28 and Tim1. In certain embodiments, host immune cells expressing the chimeric Tim receptors exhibit reduction or inhibition of immune cell exhaustion. In certain embodiments, the host immune cell is a T cell or NK cells. In certain embodiments, exhausted T cells exhibit; (a) increased expression of PD-1, TIGIT, LAG3, TIM3, or any combination thereof; (b) decreased production of IFN-γ, IL-2, TNF-α, or any combination thereof; or both (a) and (b). In certain embodiments, exhausted NK cells exhibit; (a) increased expression of PD-1, NKG2A, TIM3, or any combination thereof; (b) decreased production of IFN-γ, TNF-α, or both; or both (a) and (b).

In certain embodiments, anti-CD72 CAR-host cells co-expressing the CER or chimeric Tim receptor of the present disclosure exhibit an enhanced effector response (e.g., tumor specific). In certain embodiments, the effector response is enhanced T cell proliferation, cytokine production (e.g., IFN-γ, IL-2, TNF-α), cytotoxic activity, persistence, or any combination thereof. Host cells expressing anti-CD72 CARs may be administered to a subject alone, or in combination with other therapeutic agents, including for example CAR-T cells, TCRs, antibodies, radiation therapy, chemotherapies, small molecules, oncolytic viruses, electropulse therapy, etc.

In certain embodiments host cells anti-CD72 CAR-host cells co-expressing the CER or chimeric Tim receptor of the present disclosure exhibit a reduced immunosuppressive response to phosphatidylserine. Phosphatidylserine is one of the primary apoptotic cell ligands that signal “eat me” to phagocytes. The removal of apoptotic cells by phagocytes generally reduces or prevents an inflammatory response via secretion of anti-inflammatory cytokines IL-10 and TGF-β and the decrease of secretion of inflammatory cytokines TNF-α, IL-1β, and IL-12. Thus, phosphatidylserine may act as an immunosuppressive signal during the clearance of apoptotic cells. In certain embodiments, upon binding phosphatidylserine, anti-CD72 CAR-host cells co-expressing the CER or chimeric Tim receptor of the present disclosure exhibite increased antigen-specific cytokine production (e.g., IFN-γ, IL-2, TNF-α), thereby reducing the immunosuppressive response to phosphatidylserine.

The expression of CARs, CERs, or chimeric Tim receptors on host cells may be functionally characterized according to any of a large number of art-accepted methodologies for assaying host cell (e.g., T cell) activity, including determination of T cell binding, activation or induction and also including determination of T cell responses that are antigen-specific. Examples include determination of T cell proliferation, T cell cytokine release, antigen-specific T cell stimulation, CTL activity (e.g., by detecting 51Cr or Europium release from pre-loaded target cells), changes in T cell phenotypic marker expression, and other measures of T cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, M A (1986); Mishell and Shigii (eds.) Selected Methods in Cellular Immunology, Freeman Publishing, San Francisco, C A (1979); Green and Reed, Science 281:1309 (1998) and references cited therein. Cytokine levels may be determined according to methods known in the art, including for example, ELISA, ELISPOT, intracellular cytokine staining, flow cytometry, and any combination thereof (e.g., intracellular cytokine staining and flow cytometry). Immune cell proliferation and clonal expansion resulting from an antigen-specific clicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like.

In certain embodiments, a CER or chimeric Tim receptor modified host cell has a phagocytic index of about 20 to about 1,500 for a target cell. A “phagocytic index” is a measure of phagocytic activity of the transduced host cell as determined by counting the number of target cells or particles ingested per chimeric Tim receptor modified host cell during a set period of incubation of a suspension of target cells or particles and chimeric Tim receptor modified host cells in media. Phagocytic index may be calculated by multiplying [total number of engulfed target cells/total number of counted chimeric Tim receptor modified cells (e.g., phagocytic frequency)]×[average area of target cell or particle staining per chimeric Tim receptor⁺ host cell×100 (e.g., hybrid capture)] or [total number of engulfed particles/total number of counted chimeric Tim receptor modified host cells]×[number of chimeric Tim receptor modified host cells containing engulfed particles/total number of counted chimeric Tim receptor cells]×100. In certain embodiments, a chimeric Tim receptor modified cell has a phagocytic index of about 30 to about 1,500; about 40 to about 1,500; about 50 to about 1,500; about 75 to about 1,500; about 100 to about 1,500; about 200 to about 1,500; about 300 to about 1,500; about 400 to about 1,500; about 500 to about 1,500; about 20 to about 1,400; about 30 to about 1,400; about 40 to about 1,400; about 50 to about 1,400; about 100 to about 1,400; about 200 to about 1,400; about 300 to about 1,400; about 400 to about 1,400; about 500 to about 1,400; about 20 to about 1,300; about 30 to about 1,300; about 40 to about 1,300; about 50 to about 1,300; about 100 to about 1,300; about 200 to about 1,300; about 300 to about 1,300; about 400 to about 1,300; about 500 to about 1,300; about 20 to about 1,200; about 30 to about 1,200; about 40 to about 1,200; about 50 to about 1,200; about 100 to about 1,200; about 200 to about 1,200; about 300 to about 1,200; about 400 to about 1,200; about 500 to about 1,200; about 20 to about 1,100; about 30 to about 1,100; about 40 to about 1,100; about 50 to about 1,100; about 100 to about 1,100; about 200 to about 1,100; about 300 to about 1,100; about 400 to about 1,100; or about 500 to about 1,100; about 20 to about 1,000; about 30 to about 1,000; about 40 to about 1,000; about 50 to about 1,000; about 100 to about 1,000; about 200 to about 1,000; about 300 to about 1,000; about 400 to about 1,000; or about 500 to about 1,000; about 20 to about 750; about 30 to about 750; about 40 to about 750; about 50 to about 750; about 100 to about 750; about 200 to about 750; about 300 to about 750; about 400 to about 750; or about 500 to about 750; about 20 to about 500; about 30 to about 500; about 40 to about 500; about 50 to about 500; about 100 to about 500; about 200 to about 500; or about 300 to about 500. In further embodiments, the incubation time is from about 2 hours to about 4 hours, about 2 hours, about 3 hours, or about 4 hours. In yet further embodiments, a chimeric Tim receptor modified cell exhibits phagocytic index that is statistically significantly higher than a cell transduced with truncated EGFR control. Phagocytic index may be calculated using methods known in the art and as further described in the Examples and PCT Application No. PCT/US2017/053553 (incorporated herein by reference in its entirety), including quantification by flow cytometry or fluorescence microscopy.

Host cells may be from an animal, such as a human, primate, cow, horse, sheep, dog, cat, mouse, rat, rabbit, guinea pig, pig, or a combination thereof. In a preferred embodiment, the animal is a human. Host cells may be obtained from a healthy subject or a subject having a disease associated with expression or overexpression of an antigen.

Table 8 presents examples of CD72 CAR/Tim4 CER dual expression cassettes according to the present disclosure.

TABLE 8
CAR/CER Dual Expression Cassettes

| Name | Components | SEQ ID NO: # |
|---|---|---|
| pCTX766: Sc025- | Sc025-BBz (CAR): | 445 |
| BBz-p2A-TIM4- | MLLLVTSLLLCELPHPAFLLIPEQKLISEEDLAQVQLVQSGAEVK | |
| CD28-p2A-EGFRt | KPGASVKVSCKASGYTFTSYYMHWVRQAPGQGLEWMGIINPSG | |
| | GGTSYAQKFQGRVTMTRDTSTSTVYMELSSLRSEDTAVYYCAR | |
| | DYYVTYDSWFDSWGQGTLVTVSRGGGGSGGGGSGGGGSSELT | |
| | QDPAVSVALGQTVRITCQGDSLRSYYASWYQQKPGQAPVLVIY | |
| | GKNNRPSGIPDRFSGSSSGNTASLTITGAQAEDEADYYCNSRDSS | |
| | GNHVVFGGGTKLTVLGAAAESKYGPPCPPCPMFWVLVVVGGV | |
| | LACYSLLVTVAFIIFWVKRGRKKLLYIFKQPFMRPVQTTQEEDG | |
| | CSCRFPEEEEGGCELRVKFSRSADAPAYQQGQNQLYNELNLGRR | |
| | EEYDVLDKRRGRDPEMGGKPRRKNPQEGLYNELQKDKMAEAY | |
| | SEIGMKGERRRGKGHDGLYQGLSTATKDTYDALHMQALPPR | |
| | GSG-p2A “Skip” Sequence: | |
| | GSGATNFSLLKQAGDVEENPGP | |
| | TIM4-CD28 (CER): | |
| | MSKEPLILWLMIEFWWLYLTPVTSETVVTEVLGHRVTLPCLYSS | |
| | WSHNSNSMCWGKDQCPYSGCKEALIRTDGMRVTSRKSAKYRL | |
| | QGTIPRGDVSLTILNPSESDSGVYCCRIEVPGWFNDVKINVRLNL | |
| | QRASTTTHRTATTTTRRTTTTSPTTTRQMTTTPAALPTTVVTTPD | |
| | LTTGTPLQMTTIAVFTTANTCLSLTPSTLPEEATGLLTPEPSKEGPI | |
| | LTAESETVLPSDSWSSVESTSADTVLLTSKESKVWDLPSTSHVS | |
| | MWKTSDSVSSPQPGASDTAVPEQNKTTKTGQMDGIPMSMKNE | |
| | MPISQMFWVLVVVGGVLACYSLLVTVAFIIFWVRSKRSRLLHSD | |
| | YMNMTPRRPGPTRKHYQPYAPPRDFAAYRS | |
| | GSG-p2A ″Skip″ Sequence: | |
| | GSGATNFSLLKQAGDVEENPGP | |
| | EGFRt (Tag) | |
| | MLLLVTSLLLCELPHPAFLLIPRKVCNGIGIGEFKDSLSINATNIK | |
| | HFKNCTSISGDLHILPVAFRGDSFTHTPPLDPQELDILKTVKEITG | |
| | FLLIQAWPENRTDLHAFENLEIIRGRTKQHGQFSLAVVSLNITSL | |
| | GLRSLKEISDGDVIISGNKNLCYANTINWKKLFGTSGQKTKIISNR | |
| | GENSCKATGQVCHALCSPEGCWGPEPRDCVSCRNVSRGRECVD | |
| | KCNLLEGEPREFVENSECIQCHPECLPQAMNITCTGRGPDNCIQC | |
| | AHYIDGPHCVKTCPAGVMGENNTLVWKYADAGHVCHLCHPNC | |
| | TYGCTGPGLEGCPTNGPKIPSIATGMVGALLLLLVVALGIGLFM | |
| pCTX768: TIM4- | TIM4-CD28 (CER): | 446 |
| CD28-p2A-Sc025- | MSKEPLILWLMIEFWWLYLTPVTSETVVTEVLGHRVTLPCLYSS | |
| BBz-p2A-EGFRt | WSHNSNSMCWGKDQCPYSGCKEALIRTDGMRVTSRKSAKYRL | |
| | QGTIPRGDVSLTILNPSESDSGVYCCRIEVPGWFNDVKINVRLNL | |
| | QRASTTTHRTATTTTRRTTTTSPTTTRQMTTTPAALPTTVVTTPD | |
| | LTTGTPLQMTTIAVFTTANTCLSLTPSTLPEEATGLLTPEPSKEGPI | |
| | LTAESETVLPSDSWSSVESTSADTVLLTSKESKVWDLPSTSHVS | |
| | MWKTSDSVSSPQPGASDTAVPEQNKTTKTGQMDGIPMSMKNE | |
| | MPISQMFWVLVVVGGVLACYSLLVTVAFIIFWVRSKRSRLLHSD | |
| | YMNMTPRRPGPTRKHYQPYAPPRDFAAYRS | |
| | GSG-p2A “Skip” Sequence: | |
| | GSGATNFSLLKQAGDVEENPGP | |
| | Sc025-BBz (CAR): | |
| | MLLLVTSLLLCELPHPAFLLIPEQKLISEEDLAQVQLVQSGAEVK | |
| | KPGASVKVSCKASGYTFTSYYMHWVRQAPGQGLEWMGIINPSG | |
| | GGTSYAQKFQGRVTMTRDTSTSTVYMELSSLRSEDTAVYYCAR | |
| | DYYVTYDSWFDSWGQGTLVTVSRGGGGSGGGGSGGGGSSELT | |
| | QDPAVSVALGQTVRITCQGDSLRSYYASWYQQKPGQAPVLVIY | |
| | GKNNRPSGIPDRFSGSSSGNTASLTITGAQAEDEADYYCNSRDSS | |
| | GNHVVFGGGTKLTVLGAAAESKYGPPCPPCPMFWVLVVVGGV | |
| | LACYSLLVTVAFIIFWVKRGRKKLLYIFKQPFMRPVQTTQEEDG | |
| | CSCRFPEEEEGGCELRVKFSRSADAPAYQQGQNQLYNELNLGRR | |
| | EEYDVLDKRRGRDPEMGGKPRRKNPQEGLYNELQKDKMAEAY | |
| | SEIGMKGERRRGKGHDGLYQGLSTATKDTYDALHMQALPPR | |
| | GSG-p2A “Skip” Sequence: | |
| | GSGATNFSLLKQAGDVEENPGP | |
| | EGFRt (Tag): | |
| | MLLLVTSLLLCELPHPAFLLIPRKVCNGIGIGEFKDSLSINATNIK | |
| | HFKNCTSISGDLHILPVAFRGDSFTHTPPLDPQELDILKTVKEITG | |
| | FLLIQAWPENRTDLHAFENLEIIRGRTKQHGQFSLAVVSLNITSL | |
| | GLRSLKEISDGDVIISGNKNLCYANTINWKKLFGTSGQKTKIISNR | |
| | GENSCKATGQVCHALCSPEGCWGPEPRDCVSCRNVSRGRECVD | |
| | KCNLLEGEPREFVENSECIQCHPECLPQAMNITCTGRGPDNCIQC | |
| | AHYIDGPHCVKTCPAGVMGENNTLVWKYADAGHVCHLCHPNC | |
| | TYGCTGPGLEGCPTNGPKIPSIATGMVGALLLLLVVALGIGLFM | |
| pCTX770: Sc025- | Sc025-BBz (CAR): | 447 |
| BBz-p2A-RQR8- | MLLLVTSLLLCELPHPAFLLIPEQKLISEEDLAQVQLVQSGAEVK | |
| p2A-EGFRt | KPGASVKVSCKASGYTFTSYYMHWVRQAPGQGLEWMGIINPSG | |
| | GGTSYAQKFQGRVTMTRDTSTSTVYMELSSLRSEDTAVYYCAR | |
| | DYYVTYDSWFDSWGQGTLVTVSRGGGGSGGGGSGGGGSSELT | |
| | QDPAVSVALGQTVRITCQGDSLRSYYASWYQQKPGQAPVLVIY | |
| | GKNNRPSGIPDRFSGSSSGNTASLTITGAQAEDEADYYCNSRDSS | |
| | GNHVVFGGGTKLTVLGAAAESKYGPPCPPCPMFWVLVVVGGV | |
| | LACYSLLVTVAFIIFWVKRGRKKLLYIFKQPFMRPVQTTQEEDG | |
| | CSCRFPEEEEGGCELRVKFSRSADAPAYQQGQNQLYNELNLGRR | |
| | EEYDVLDKRRGRDPEMGGKPRRKNPQEGLYNELQKDKMAEAY | |
| | SEIGMKGERRRGKGHDGLYQGLSTATKDTYDALHMQALPPR | |
| | GSG-p2A “Skip” Sequence | |
| | GSGATNFSLLKQAGDVEENPGP | |
| | RQR8 Tag | |
| | MGTSLLCWMALCLLGADHADACPYSNPSLCSGGGGSELPTQGT | |
| | FSNVSTNVSPAKPTTTACPYSNPSLCSGGGGSPAPRPPTPAPTIAS | |
| | QPLSLRPEACRPAAGGAVHTRGLDFACDIYIWAPLAGTCGVLLL | |
| | SLVITLYCNHRNRRRVCKCPRPW | |
| | GSG-p2A “Skip” Sequence | |
| | GSGATNFSLLKQAGDVEENPGP | |
| | EGFRt (Tag) | |
| | MLLLVTSLLLCELPHPAFLLIPRKVCNGIGIGEFKDSLSINATNIK | |
| | HFKNCTSISGDLHILPVAFRGDSFTHTPPLDPQELDILKTVKEITG | |
| | FLLIQAWPENRTDLHAFENLEIIRGRTKQHGQFSLAVVSLNITSL | |
| | GLRSLKEISDGDVIISGNKNLCYANTINWKKLFGTSGQKTKIISNR | |
| | GENSCKATGQVCHALCSPEGCWGPEPRDCVSCRNVSRGRECVD | |
| | KCNLLEGEPREFVENSECIQCHPECLPQAMNITCTGRGPDNCIQC | |
| | AHYIDGPHCVKTCPAGVMGENNTLVWKYADAGHVCHLCHPNC | |
| | TYGCTGPGLEGCPTNGPKIPSIATGMVGALLLLLVVALGIGLFM | |
| pCTX771: RQR8- | RQR8 Tag: | 448 |
| p2A-Sc025-BBz- | MGTSLLCWMALCLLGADHADACPYSNPSLCSGGGGSELPTQGT | |
| p2A-EGFRt | FSNVSTNVSPAKPTTTACPYSNPSLCSGGGGSPAPRPPTPAPTIAS | |
| | QPLSLRPEACRPAAGGAVHTRGLDFACDIYIWAPLAGTCGVLLL | |
| | SLVITLYCNHRNRRRVCKCPRPW | |
| | GSG-p2A “Skip” Sequence: | |
| | GSGATNFSLLKQAGDVEENPGP | |
| | Sc025-BBz (CAR): | |
| | MLLLVTSLLLCELPHPAFLLIPEQKLISEEDLAQVQLVQSGAEVK | |
| | KPGASVKVSCKASGYTFTSYYMHWVRQAPGQGLEWMGIINPSG | |
| | GGTSYAQKFQGRVTMTRDTSTSTVYMELSSLRSEDTAVYYCAR | |
| | DYYVTYDSWFDSWGQGTLVTVSRGGGGSGGGGSGGGGSSELT | |
| | QDPAVSVALGQTVRITCQGDSLRSYYASWYQQKPGQAPVLVIY | |
| | GKNNRPSGIPDRFSGSSSGNTASLTITGAQAEDEADYYCNSRDSS | |
| | GNHVVFGGGTKLTVLGAAAESKYGPPCPPCPMFWVLVVVGGV | |
| | LACYSLLVTVAFIIFWVKRGRKKLLYIFKQPFMRPVQTTQEEDG | |
| | CSCRFPEEEEGGCELRVKFSRSADAPAYQQGQNQLYNELNLGRR | |
| | EEYDVLDKRRGRDPEMGGKPRRKNPQEGLYNELQKDKMAEAY | |
| | SEIGMKGERRRGKGHDGLYQGLSTATKDTYDALHMQALPPR | |
| | GSG-p2A “Skip” Sequence: | |
| | GSGATNFSLLKQAGDVEENPGP | |
| | EGFR Tag: | |
| | MLLLVTSLLLCELPHPAFLLIPRKVCNGIGIGEFKDSLSINATNIK | |
| | HFKNCTSISGDLHILPVAFRGDSFTHTPPLDPQELDILKTVKEITG | |
| | FLLIQAWPENRTDLHAFENLEIIRGRTKQHGQFSLAVVSLNITSL | |
| | GLRSLKEISDGDVIISGNKNLCYANTINWKKLFGTSGQKTKIISNR | |
| | GENSCKATGQVCHALCSPEGCWGPEPRDCVSCRNVSRGRECVD | |
| | KCNLLEGEPREFVENSECIQCHPECLPQAMNITCTGRGPDNCIQC | |
| | AHYIDGPHCVKTCPAGVMGENNTLVWKYADAGHVCHLCHPNC | |
| | TYGCTGPGLEGCPTNGPKIPSIATGMVGALLLLLVVALGIGLFM | |

### V. Methods of Use

In one aspect, the present disclosure provides methods of treating a disease associated with CD72 in a subject comprising administering an effective amount of the anti-CD72 chimeric antigen receptor, the polynucleotide encoding the anti-CD72 CAR, the engineered cell comprising the anti-CD72 CAR, or pharmaceutical compositions thereof, optionally, in combination with a CER or chimeric Tim receptor of the present disclosure. In some embodiments, the host cell is an immune cell. In some embodiments, the host cell is a T cell or an NK cell.

In another aspect, an anti-CD72 chimeric antigen receptor, the polynucleotide encoding the anti-CD72 CAR, the engineered cell comprising the anti-CD72 CAR, or pharmaceutical compositions thereof, optionally, in combination with a CER or chimeric Tim receptor of the present disclosure may be used in a method of enhancing effector function of the host cell. In certain embodiments, enhanced effector function comprises increased T cell activation, cytotoxic activity, increased antigen specific cytokine production (e.g., IFN-γ, IL-2, TNF-α, or any combination thereof), increased anti-apoptotic signaling, increased persistence, increased expansion, increased proliferation, or any combination thereof. In certain embodiments, the effector function of the host cell is enhanced at least about 10%, 15%, 20%, 25%, 30%, 35%, 40%, 50%, 55%, 60%, 65%, 70%, 75%, 80%, 85%, 90%, 95%, 100%, 110%, 120%, 130%, 140%, 150%, 160%, 170%, 180%, 190%, 200% or more as compared to a host cell that is not modified with a nucleic acid molecule encoding a chimeric Tim receptor or a chimeric Tim receptor vector. In some embodiments, the host cell is an immune cell. In certain embodiments, the host cell is a T cell or an NK cell.

In another aspect, an anti-CD72 chimeric antigen receptor, the polynucleotide encoding the anti-CD72 CAR, the engineered cell comprising the anti-CD72 CAR, or pharmaceutical compositions thereof, optionally, in combination with a CER or chimeric Tim receptor of the present disclosure can be used in methods enhancing antigen presentation or reducing or inhibiting immune escape by tumor cells. Non-targeted tumor antigens may be endocytosed via CER or chimeric Tim receptor engagement, processed, and presented to clonal T cells to elicit secondary anti-tumor responses. In some embodiments, the host cell is an immune cell. In some embodiments, the immune cell is a T cell or NK cell.

In another aspect, an anti-CD72 chimeric antigen receptor, the polynucleotide encoding the anti-CD72 CAR, the engineered cell comprising the anti-CD72 CAR, or pharmaceutical compositions thereof, optionally, in combination with a CER or chimeric Tim receptor of the present disclosure can be used in methods to enhance the effect of a therapeutic agent that induces cellular stress, damage, necrosis, or apoptosis. Certain therapies, such as chemotherapy, radiation therapy, UV light therapy, electropulse therapy, adoptive cellular immunotherapy (e.g., CAR-T cells, TCRs) and oncolytic viral therapy, can induce cell damage or death to tumor cells, diseased cells, and cells in their surrounding environment. Cells expressing CERs or chimeric Tim receptors can be administered in combination with the cell damaging/cytotoxic therapy to bind to the phosphatidylserine moieties exposed on the outer leaflet of targeted cells and clear stressed, damaged, diseased, apoptotic, necrotic cells.

Diseases that may be treated with cells expressing a chimeric Tim receptor as described in the present disclosure include cancer and autoimmune diseases. Adoptive immune and gene therapies are promising treatments for various types of cancer (Morgan et al., Science 314:126, 2006; Schmitt et al., Hum. Gene Ther. 20:1240, 2009; June, J. Clin. Invest. 117:1466, 2007) and infectious disease (Kitchen et al., PLOS One 4:38208, 2009; Rossi et al., Nat. Biotechnol. 25:1444, 2007; Zhang et al., PLOS Pathog. 6:e1001018, 2010; Luo et al., J. Mol. Med. 89:903, 2011).

A wide variety of cancers, including solid tumors and leukemias are amenable to the compositions and methods disclosed herein. Exemplary cancers that may be treated using the receptors, modified host cells, and composition described herein include adenocarcinoma of the breast, prostate, and colon; all forms of bronchogenic carcinoma of the lung; myeloid leukemia; melanoma; hepatoma; neuroblastoma; papilloma; apudoma; choristoma; branchioma; malignant carcinoid syndrome; carcinoid heart disease; and carcinoma (e.g., Walker, basal cell, basosquamous, Brown-Pearce, ductal, Ehrlich tumor, Krebs 2, Merkel cell, mucinous, non-small cell lung, oat cell, papillary, scirrhous, bronchiolar, bronchogenic, squamous cell, and transitional cell). Additional types of cancers that may be treated using the receptors, modified host cells, and composition described herein include histiocytic disorders; malignant histiocytosis; leukemia; Hodgkin's disease; immunoproliferative small; non-Hodgkin's lymphoma; plasmacytoma; multiple myeloma; chronic myeloid leukemia (CML); acute myeloid leukemia (AML); plasmacytoma; reticuloendotheliosis; melanoma; chondroblastoma; chondroma; chondrosarcoma; fibroma; fibrosarcoma; giant cell tumors; histiocytoma; lipoma; liposarcoma; mesothelioma; myxoma; myxosarcoma; ostcoma; osteosarcoma; chordoma; craniopharyngioma; dysgerminoma; hamartoma; mesenchymoma; mesonephroma; myosarcoma; ameloblastoma; cementoma; odontoma; teratoma; thymoma; trophoblastic tumor. Further, the following types of cancers are also contemplated as amenable to treatment using the receptors, modified host cells, and composition described herein: adenoma; cholangioma; cholesteatoma; cyclindroma; cystadenocarcinoma; cystadenoma; granulosa cell tumor; gynandroblastoma; hepatoma; hidradenoma; islet cell tumor; Leydig cell tumor; papilloma; sertoli cell tumor; theca cell tumor; leimyoma; leiomyosarcoma; myoblastoma; myomma; myosarcoma; rhabdomyoma; rhabdomyosarcoma; ependymoma; ganglioncuroma; glioma; medulloblastoma; meningioma; neurilemmoma; neuroblastoma; neurocpithelioma; neurofibroma; neuroma; paraganglioma; paraganglioma nonchromaffin. The types of cancers that may be treated also include angiokeratoma; angiolymphoid hyperplasia with cosinophilia; angioma sclerosing; angiomatosis; glomangioma; hemangioendothelioma; hemangioma; hemangiopericytoma; hemangiosarcoma; lymphangioma; lymphangiomyoma; lymphangiosarcoma; pincaloma; carcinosarcoma; chondrosarcoma; cystosarcoma phyllodes; fibrosarcoma; hemangiosarcoma; leiomyosarcoma; leukosarcoma; liposarcoma; lymphangiosarcoma; myosarcoma; myxosarcoma; ovarian carcinoma; rhabdomyosarcoma; sarcoma; neoplasms; nerofibromatosis; cervical dysplasia, and peritoneal cancer.

Examples of hyperproliferative disorders amenable to therapy using the receptors, modified host cells, and composition described herein include B-cell cancers (B-cell malignancies), including B-cell lymphomas (such as various forms of Hodgkin's disease, non-Hodgkin's lymphoma (NHL) or central nervous system lymphomas), leukemias (such as acute lymphoblastic leukemia (ALL), chronic lymphocytic leukemia (CLL), Hairy cell leukemia, B cell blast transformation of chronic myeloid leukemia, acute myeloid leukemia (AML), chronic myeloid leukemia, and myelomas (such as multiple myeloma). Additional B cell cancers that may be treated using the receptors, modified host cells, and composition described herein include small lymphocytic lymphoma, B-cell prolymphocytic leukemia, lymphoplasmacytic lymphoma, splenic marginal zone lymphoma, plasma cell myeloma, solitary plasmacytoma of bone, extraosscous plasmacytoma, extra-nodal marginal zone B-cell lymphoma of mucosa-associated (MALT) lymphoid tissue, nodal marginal zone B-cell lymphoma, follicular lymphoma, mantle cell lymphoma, diffuse large B-cell lymphoma, mediastinal (thymic) large B-cell lymphoma, intravascular large B-cell lymphoma, primary effusion lymphoma, Burkitt's lymphoma/leukemia, B-cell proliferations of uncertain malignant potential, lymphomatoid granulomatosis, and post-transplant lymphoproliferative disorder.

In some embodiments, the B-cell malignancy is a refractory B-cell malignancy. In some embodiments, an anti-CD72 chimeric antigen receptor, the polynucleotide encoding the anti-CD72 CAR, the engineered cell comprising the anti-CD72 CAR, or pharmaceutical compositions thereof, optionally, in combination with a CER or chimeric Tim receptor of the present disclosure are administered to the subject following relapse of cellular immunotherapy (e.g., CAR or TCR). In some embodiments, the relapse of cellular immunotherapy comprises CD19 CAR therapy or CD22 CAR therapy.

In some embodiments, an anti-CD72 chimeric antigen receptor, the polynucleotide encoding the anti-CD72 CAR, the engineered cell comprising the anti-CD72 CAR, or pharmaceutical compositions thereof, optionally, in combination with a CER or chimeric Tim receptor of the present disclosure are administered to a subject having acute myeloid leukemia with a c-kit mutation.

In some embodiments, an anti-CD72 chimeric antigen receptor, the polynucleotide encoding the anti-CD72 CAR, the engineered cell comprising the anti-CD72 CAR, or pharmaceutical compositions thereof, optionally, in combination with a CER or chimeric Tim receptor of the present disclosure are administered to a subject having acute myeloid leukemia with a t(8;21) translocation.

An anti-CD72 chimeric antigen receptor of the present disclosure may be administered to a subject in cell-bound form (e.g., gene therapy of target cell population). Thus, for example, an anti-CD72 CAR of the present disclosure may be administered to a subject expressed on the surface of immune cells, e.g., T cells, Natural Killer Cells, Natural Killer T cells, B cells, lymphoid precursor cells, antigen presenting cells, dendritic cells, Langerhans cells, myeloid precursor cells, mature myeloid cells, including subsets thereof, or any combination thereof. In certain embodiments, methods of treating a subject comprise administering an effective amount of anti-CD72 CAR modified cells (i.e., recombinant cells that express one or more anti-CD72 CARs). The anti-CD72 CAR modified cells may be xenogeneic, syngeneic, allogeneic, or autologous to the subject.

Pharmaceutical compositions including anti-CD72 CAR engineered cells may be administered in a manner appropriate to the disease or condition to be treated (or prevented) as determined by persons skilled in the medical art. An appropriate dose, suitable duration, and frequency of administration of the compositions will be determined by such factors as the condition of the patient, size, weight, body surface area, age, sex, type and severity of the disease, particular therapy to be administered, particular form of the active ingredient, time and the method of administration, and other drugs being administered concurrently. The present disclosure provides pharmaceutical compositions comprising anti-CD72 CAR engineered cells and a pharmaceutically acceptable carrier, diluent, or excipient. Suitable excipients include water, saline, dextrose, glycerol, or the like and combinations thereof. Other suitable infusion medium can be any isotonic medium formulation, including saline, Normosol R (Abbott), Plasma-Lyte A (Baxter), 5% dextrose in water, or Ringer's lactate.

A treatment effective amount of cells in a pharmaceutical composition is at least one cell (for example, one anti-CD72 CAR modified T cell) or is more typically greater than 10² cells, for example, up to 10⁶, up to 10⁷, up to 10⁸ cells, up to 10⁹ cells, up to 10¹⁰ cells, or up to 10¹¹ cells or more. In certain embodiments, the cells are administered in a range from about 10⁶ to about 10¹⁰ cells/m², preferably in a range of about 10⁷ to about 10⁹ cells/m². The number of cells will depend upon the ultimate use for which the composition is intended as well the type of cells included therein. For example, a composition comprising cells modified to contain an anti-CD72 CAR will comprise a cell population containing from about 5% to about 95% or more of such cells. In certain embodiments, a composition comprising anti-CD72 CAR modified cells comprises a cell population comprising at least 5%, 10%, 15%, 20%, 25%, 30%, 35%, 40%, 45%, 50%, 55%, 60%, 65%, 70%, 75%, 80%, 85%, 90%, 95% or more of such cells. For uses provided herein, the cells are generally in a volume of a liter or less, 500 mls or less, 250 mls or less, or 100 mls or less. Hence the density of the desired cells is typically greater than 10⁺ cells/ml and generally is greater than 10⁷ cells/ml, generally 10⁸ cells/ml or greater. The cells may be administered as a single infusion or in multiple infusions over a range of time. Repeated infusions of anti-CD72 CAR modified cells may be separated by days, weeks, months, or even years if relapses of disease or disease activity are present. A clinically relevant number of immune cells can be apportioned into multiple infusions that cumulatively equal or exceed 10⁶, 10⁷, 10⁸, 10⁹, 10¹⁰, or 10¹¹ cells. A preferred dose for administration of a host cell comprising a recombinant expression vector as described herein is about 10⁷ cells/m², about 5×10⁷ cells/m², about 10⁸ cells/m², about 5×10⁸ cells/m², about 10⁹ cells/m², about 5×10⁹ cells/m², about 10¹⁰ cells/m², about 5×10¹⁰ cells/m², or about 10¹¹ cells/m².

Anti-CD72 CAR compositions as described herein may be administered intravenously, intraperitoneally, intranasally, intratumorly, into the bone marrow, into the lymph node, and/or into cerebrospinal fluid.

Anti-CD72 CAR compositions may be administered to a subject in combination with one or more additional therapeutic agents. Examples of therapeutic agents that may be administered in combination with a chimeric Tim compositions according to the present description include radiation therapy, adoptive cellular immunotherapy agent (e.g., CER, chimeric Tim receptor, recombinant TCR, enhanced affinity TCR, CAR, TCR-CAR, scTCR fusion protein, dendritic cell vaccine), antibody therapy, immune checkpoint molecule inhibitor therapy, UV light therapy, electric pulse therapy, high intensity focused ultrasound therapy, oncolytic virus therapy, or a pharmaceutical therapy, such as a chemotherapeutic agent, a therapeutic peptide, a hormone, an aptamer, antibiotic, anti-viral agent, anti-fungal agent, anti-inflammatory agent, a small molecule therapy, or any combination thereof. In certain embodiments, the anti-CD72 CAR modified host cells stresses, damages, or kill cells and induces display of surface phosphatidylserine, and a CER or chimeric Tim receptor clears stressed, damaged, apoptotic, necrotic, infected, dead cells displaying surface phosphatidylscrinc.

In certain embodiments, the anti-CD72 CAR and adoptive cellular immunotherapy agent (e.g., a CER, chimeric Tim receptor, CAR, TCR-CAR, TCR, etc. described above) are administered to the subject expressed in the same host cell or different host cells. In certain embodiments, the anti-CD72 CAR and adoptive cellular immunotherapy agent are expressed in the same host cell from the same vector or from separate vectors. In certain embodiments, the anti-CD72 CAR and adoptive cellular immunotherapy agent are expressed in the same host cell from a multicistronic vector (e.g., tandem expression cassette). In certain embodiments, the anti-CD72 CAR is expressed in the same host cell type as the adoptive cellular immunotherapy agent (e.g., the anti-CD72 CAR is expressed CD4 T cells and the CER or chimeric Tim receptor is expressed in CD4 T cells, or the anti-CD72 CAR is expressed CD8 T cells and the CER or chimeric Tim receptor is expressed in CD8 T cells). In other embodiments, the anti-CD72 CAR is expressed in a different host cell type as the adoptive immunotherapy agent (e.g., the anti-CD72 CAR is expressed CD4 T cells and the CER or chimeric Tim receptor is expressed in CD8 T cells, or the anti-CD72 CAR is expressed CD8 T cells and the CER or chimeric Tim receptor is expressed in CD4 T cells). Cellular immunotherapy compositions comprising a combination of immune cells or cellular subsets engineered with CER or chimeric Tim receptors and a cellular immunotherapy agent (e.g., CAR, TCR, etc.), methods of making, and methods of use are described in PCT International Publication No. WO2019/191340, which is incorporated herein by reference in its entirety.

Exemplary antigens that a recombinant TCR, enhanced affinity TCR, CAR, TCR-CAR, or scTCR fusion protein may target include WT-1, mesothelin, MART-1, NY-ESO-1, MAGE-A3, HPV E7, survivin, a Fetoprotein, and a tumor-specific neoantigen.

CARs of the present disclosure may target a variety of antigens, including a viral antigen, bacterial antigen, fungal antigen, parasitic antigen, tumor antigen, autoimmune disease antigen. Exemplary antigens that a CAR may target include CD138, CD38, CD33, CD123, CD72, CD79a, CD79b, mesothelin, PSMA, BCMA, ROR1, MUC-16, LICAM, CD22, CD19, CD20, CD23, CD24, CD37, CD30, CA125, CD56, c-Met, EGFR, GD-3, HPV E6, HPV E7, MUC-1, HER2, folate receptor α, CD97, CD171, CD179a, CD44v6, WTI, VEGF-α, VEGFR1, IL-13Rα1, IL-13Rα2, IL-11Rα, PSA, FcRH5, NKG2D ligand, NY-ESO-1, TAG-72, CEA, ephrin A2, ephrin B2, Lewis A antigen, Lewis Y antigen, MAGE, MAGE-A1, RAGE-1, folate receptor β, EGFRviii, VEGFR-2, LGR5, SSX2, AKAP-4, FLT3, fucosyl GM1, GM3, o-acetyl-GD2, and GD2.

Radiation therapy includes external beam radiation therapy (e.g., conventional external beam radiation therapy, stereotactic radiation, 3-dimensional conformal radiation therapy, intensity-modulated radiation therapy, volumetric modulated arc therapy, particle therapy, proton therapy, and auger therapy), brachytherapy, systemic radioisotope therapy, intraoperative radiotherapy, or any combination thereof.

Exemplary antibodies for use in conjunction with the anti-CD72 CAR compositions described herein include rituxmab, pertuzumab, trastuzumab, alemtuzumab, Ibritumomab tiuxetan, Brentuximab vedotin, cetuximab, bevacizumab, abciximab, adalimumab, alefacept, basilizimab, belimumab, bezlotoxumab, canakinumab, certolizumab pegol, daclizumab, denosumab, cfalizumab, golimumab, olaratumab, palivizumab, panitumumab, and tocilizumab.

Exemplary inhibitors of immune checkpoint molecules that may be for use in conjunction with the anti-CD72 CAR compositions described herein include checkpoint inhibitors targeting PD-L1, PD-L2, CD80, CD86, B7-H3, B7-H4, HVEM, adenosine, GAL9, VISTA, CEACAM-1, CEACAM-3, CEACAM-5, PVRL2, PD-1, CTLA-4, BTLA, KIR, LAG3, TIM3, A2aR, CD244/2B4, CD160, TIGIT, LAIR-1, PVRIG/CD112R, or any combination thereof. In certain embodiments, an immune checkpoint inhibitor may be an antibody, a peptide, an RNAi agent, or a small molecule. An antibody specific for CTLA-4 may be ipilimumab or tremelimumab. An antibody specific for PD-1 may be pidilizumab, nivolumab, or pembrolizumab. An antibody specific for PD-L1 may be durvalumab, atczolizumab, or avelumab.

Exemplary chemotherapeutics for use in conjunction with the anti-CD72 CAR compositions compositions described herein may include an alkylating agent, a platinum based agent, a cytotoxic agent, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor.

A chemotherapeutic includes non-specific cytotoxic agents that inhibit mitosis or cell division, as well as molecularly targeted therapy that blocks the growth and spread of cancer cells by targeting specific molecules that are involved in tumor growth, progression, and metastasis (e.g., oncogenes). Exemplary non-specific chemotherapeutics for use in conjunction with the expression cassette compositions described herein may include an alkylating agent, a platinum based agent, a cytotoxic agent, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), hypomethylating agent, and a DNA repair inhibitor.

Examples of chemotherapeutic agents considered for use in combination therapies contemplated herein include vemurafenib, dabrafenib, tramctinib, cobimetinib, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BICNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), fdabra tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), ibrutinib, venetoclax, crizotinib, alectinib, brigatinib, ceritinib, and vinorelbine (Navelbine®).

Exemplary alkylating agents for use in combination therapies contemplated herein include nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Hacmanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BICNUR), lomustine (CecNU®), streptozocin (Zanosar®), and Dacarbazine (DTIC-Dome®). Additional exemplary alkylating agents for use in combination therapies contemplated herein include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BICNUR); Bendamustine (Treanda®); Busulfan (Busulfex® and Myleran®); Carboplatin (Paraplatin®); Lomustine (also known as CCNU, CecNUR); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifcx®); Prednumustine; Procarbazine (Matulane®); Mechlorethamine (also known as nitrogen mustard, mustine and mechlorocthamine hydrochloride, Mustargen®); Streptozocin (Zanosar®); Thiotcpa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Exemplary platinum based agents for use in combination therapies contemplated herein include carboplatin, cisplatin, oxaliplatin, nedaplatin, picoplatin, satraplatin, phenanthriplatin, and triplatin tetranitrate.

Exemplary hypomethylating agents for use in combination therapies include azacitidine and decitabinc.

Exemplary molecularly targeted inhibitors for use in combination therapies contemplated herein include small molecules that target molecules involved in cancer cell growth and survival, including for example, receptor tyrosine kinase inhibitors, RAF inhibitors, BCL-2 inhibitors, ABL inhibitors, TRK inhibitors, c-KIT inhibitors, c-MET inhibitors, CDK4/6 inhibitors, FAK inhibitors, FGFR inhibitors, FLT3 inhibitors, IDH1 inhibitors, IDH2 inhibitors, PDGFRA inhibitors, and RET inhibitors.

Exemplary molecularly targeted therapy includes hormone antagonists, signal transduction inhibitors, gene expression inhibitors (e.g., translation inhibitors), apoptosis inducers, angiogenesis inhibitors (e.g., a VEGF pathway inhibitor), tyrosine kinase inhibitors (e.g., an EGF/EGFR pathway inhibitor), growth factor inhibitors, GTPase inhibitors, serine/threonine kinase inhibitors, transcription factor inhibitors, inhibitors of driver mutations associated with cancer, B-Raf inhibitors, RAF inhibitors, a MEK inhibitors, mTOR inhibitors, adenosine pathway inhibitors, EGFR inhibitors, PI3K inhibitors, BCL2 inhibitors, VEGFR inhibitors, MET inhibitors, MYC inhibitors, BCR-ABL inhibitors, ABL inhibitors, HER2 inhibitors, H-RAS inhibitors, K-RAS inhibitors, PDGFR inhibitors, ALK inhibitors, ROSI inhibitors, BTK inhibitors, TRK inhibitors, c-KIT inhibitors, c-MET inhibitors, CDK4/6 inhibitors, FAK inhibitors, FGFR inhibitors, FLT3 inhibitors, IDH1 inhibitors, IDH2 inhibitors, PARP inhibitors, PARP inhibitors, PDGFRA inhibitors, and RET inhibitors. In certain embodiments, use of molecularly targeted therapy comprises administering a molecularly targeted therapy specific for the molecular target to a subject identified as having a tumor that possesses the molecular target (e.g., driver oncogene). In certain embodiments, the molecular target has an activating mutation. In certain embodiments, use of anti-CD72 CAR modified cells in combination with a molecularly targeted inhibitor increases the magnitude of anti-tumor response, the durability of anti-tumor response, or both. In certain embodiments, a lower than typical dose of molecularly targeted therapy is used in combination with anti-CD72 CAR modified cells.

Exemplary angiogenesis inhibitors include, without limitation A6 (Angstrom Pharmaceuticals), ABT-510 (Abbott Laboratories), ABT-627 (Atrasentan) (Abbott Laboratories/Xinlay), ABT-869 (Abbott Laboratories), Actimid (CC4047, Pomalidomide) (Celgene Corporation), AdGVPEDF.11D (GenVec), ADH-1 (Exherin) (Adherex Technologies), AEE788 (Novartis), AG-013736 (Axitinib) (Pfizer), AG3340 (Prinomastat) (Agouron Pharmaceuticals), AGX1053 (AngioGenex), AGX51 (AngioGenex), ALN-VSP (ALN-VSP 02) (Alnylam Pharmaceuticals), AMG 386 (Amgen), AMG706 (Amgen), Apatinib (YN968D1) (Jiangsu Hengrui Medicine), AP23573 (Ridaforolimus/MK8669) (Ariad Pharmaceuticals), AQ4N (Novavea), ARQ 197 (ArQulc), ASA404 (Novartis/Antisoma), Atiprimod (Callisto Pharmaceuticals), ATN-161 (Attenuon), AV-412 (Avco Pharmaceuticals), AV-951 (Aveo Pharmaceuticals), Avastin (Bevacizumab) (Genentech), AZD2171 (Cediranib/Recentin) (AstraZeneca), BAY 57-9352 (Telatinib) (Bayer), BEZ235 (Novartis), BIBF1120 (Bochringer Ingelheim Pharmaceuticals), BIBW 2992 (Bochringer Ingelheim Pharmaceuticals), BMS-275291 (Bristol-Myers Squibb), BMS-582664 (Brivanib) (Bristol-Myers Squibb), BMS-690514 (Bristol-Myers Squibb), Calcitriol, CCI-779 (Torisel) (Wyeth), CDP-791 (ImClone Systems), Ceflatonin (Homoharringtonine/HHT) (ChemGenex Therapeutics), Celebrex (Celecoxib) (Pfizer), CEP-7055 (Cephalon/Sanofi), CHIR-265 (Chiron Corporation), NGR-TNF, COL-3 (Metastat) (Collagenex Pharmaceuticals), Combretastatin (Oxigenc), CP-751,871(Figitumumab) (Pfizer), CP-547,632 (Pfizer), CS-7017 (Daiichi Sankyo Pharma), CT-322 (Angiocept) (Adnexus), Curcumin, Dalteparin (Fragmin) (Pfizer), Disulfiram (Antabusc), E7820 (Eisai Limited), E7080 (Eisai Limited), EMD 121974 (Cilengitide) (EMD Pharmaceuticals), ENMD-1198 (EntreMed), ENMD-2076 (EntreMed), Endostar (Simcerc), Erbitux (ImClonc/Bristol-Myers Squibb), EZN-2208 (Enzon Pharmaceuticals), EZN-2968 (Enzon Pharmaceuticals), GC1008 (Genzyme), Genistein, GSK1363089 (Forctinib) (GlaxoSmithKline), GW786034 (Pazopanib) (GlaxoSmithKline), GT-111 (Vascular Biogenics Ltd.), IMC-1121B (Ramucirumab) (ImClone Systems), IMC-18F1 (ImClone Systems), IMC-3G3 (ImClone LLC), INCB007839 (Incyte Corporation), INGN 241 (Introgen Therapeutics), Iressa (ZD1839/Gefitinib), LBH589 (Faridak/Panobinostst) (Novartis), Lucentis (Ranibizumab) (Genentech/Novartis), LY317615 (Enzastaurin) (Eli Lilly and Company), Macugen (Pegaptanib) (Pfizer), MEDI522 (Abegrin) (MedImmune), MLN518 (Tandutinib) (Millennium), Neovastat (AE941/Benefin) (Acterna Zentaris), Nexavar (Bayer/Onyx), NM-3 (Genzyme Corporation), Noscapine (Cougar Biotechnology), NPI-2358 (Nereus Pharmaceuticals), OSI-930 (OSI), Palomid 529 (Paloma Pharmaceuticals, Inc.), Panzem Capsules (2ME2) (EntreMed), Panzem NCD (2ME2) (EntreMed), PF-02341066 (Pfizer), PF-04554878 (Pfizer), PI-88 (Progen Industries/Medigen Biotechnology), PKC412 (Novartis), Polyphenon E (Green Tea Extract) (Polypheno E International, Inc.), PPI-2458 (Praecis Pharmaceuticals), PTC299 (PTC Therapeutics), PTK787 (Vatalanib) (Novartis), PXD101 (Belinostat) (CuraGen Corporation), RAD001 (Everolimus) (Novartis), RAF265 (Novartis), Regorafenib (BAY73-4506) (Bayer), Revlimid (Celgene), Retaane (Alcon Research), SN38 (Liposomal) (Neopharm), SNS-032 (BMS-387032) (Sunesis), SOM230 (Pasircotide) (Novartis), Squalamine (Genacra), Suramin, Sutent (Pfizer), Tarceva (Genentech), TB-403 (Thrombogenics), Tempostatin (Collard Biopharmaceuticals), Tetrathiomolybdate (Sigma-Aldrich), TG100801 (TargeGen), Thalidomide (Celgene Corporation), Tinzaparin Sodium, TKI258 (Novartis), TRC093 (Tracon Pharmaceuticals Inc.), VEGF Trap (Aflibercept) (Regeneron Pharmaceuticals), VEGF Trap-Eye (Regeneron Pharmaceuticals), Veglin (VasGene Therapeutics), Bortezomib (Millennium), XL184 (Exclixis), XL647 (Exclixis), XL784 (Exclixis), XL820 (Exelixis), XL999 (Exelixis), ZD6474 (AstraZeneca), Vorinostat (Merck), and ZSTK474.

Exemplary B-Raf inhibitors include vemurafenib, dabrafenib, and encorafenib.

Exemplary MEK inhibitors include binimetinib, cobimetinib, refametinib, selumetinib, and trametinib.

Exemplary BTK inhibitors include ibrutinib, Loxo-305, tirabrutinib, GDC-0853, acalabrutinib, ONO-4059, spebrutinib, BGB-3111, HM71224, and M7583.

Exemplary TRK inhibitors include entrectinib, larotrectinib, CH7057288, ONO-7579, LOXO-101, lestaurtinib, and LOXO-195.

Exemplary c-KIT inhibitors include imatinb, sunitinb, and ponatinib.

Exemplary c-MET inhibitors include capmatinib, crizotinib, tivantinib, onartuzumab, INCB28060, AMG-458, savolitinib, and tepotinib.

Exemplary CDK4/6 inhibitors include palbociclib, ribociclib, abemaciclib, and trilaciclib.

Exemplary FAK inhibitors include defactinib, GSK2256098, BI853520, and PF-00562271.

Exemplary FGFR inhibitors include erdafitinib, pemigatinib, infigratinib, rogaratinib, AZD4547, BGJ398, FP-1039, and ARQ 087.

Exemplary FLT-3 inhibitors include quizartinib, crenolanib, gilteritinib, midostaurin, and lestaurtinib.

Exemplary IDH1 inhibitors include ivosidenib, BAY-1436032, and AGI-5198.

An exemplary IDH2 inhibitor includes enasidenib.

Exemplary PARP inhibitors include talazoparib, niraparib, rucaparib, olaparib, veliparib, CEP 9722, E7016, AG014699, MK4827, BMN-673, and Pamiparib (BGB-290).

Exemplary PDGFRA inhibitors include imatinib, regorafenib, crenolanib, and olaratumab.

Exemplary pan-RAF inhibitors include belvarafenib, LXH254, LY3009120, INU-152, and HM95573.

Exemplary RET inhibitors include lenvatinib, alectinib, vandetanib, cabozantinib, BLU-667, and LOXO-292.

Exemplary ROSI inhibitors include ceritinib, lorlatinib, entrectinib, crizotinib, TPX-0005, and DS-6051b.

Exemplary Vascular Endothelial Growth Factor (VEGF) receptor inhibitors include, but are not limited to, Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Forctinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfanib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1-((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinaminc (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylca®).

Exemplary EGF pathway inhibitors include, without limitation tyrphostin 46, EKB-569, erlotinib (Tarceva®), gefitinib (Iressa®), erbitux, nimotuzumab, lapatinib (Tykerb®), cetuximab (anti-EGFR mAb), ¹⁸⁸Re-labeled nimotuzumab (anti-EGFR mAb), and those compounds that are generically and specifically disclosed in WO 97/02266, EP 0 564 409, WO 99/03854, EP 0 520 722, EP 0 566 226, EP 0 787 722, EP 0 837 063, U.S. Pat. No. 5,747,498, WO 98/10767, WO 97/30034, WO 97/49688, WO 97/38983 and WO 96/33980. Exemplary EGFR antibodies include, but are not limited to, Cetuximab (Erbitux®); Panitumumab (Vectibix®); Matuzumab (EMD-72000); Trastuzumab (Herceptin®); Nimotuzumab (hR3); Zalutumumab; TheraCIM h-R3; MDX0447 (CAS 339151-96-1); and ch806 (mAb-806, CAS 946414-09-1). Exemplary Epidermal growth factor receptor (EGFR) inhibitors include, but not limited to, Erlotinib hydrochloride (Tarceva®); ceritinib; brigatinib; osimcritinib; icotinib; Gefitnib (Iressa®); N-[4-[(3-Chloro-4-fluorophenyl)amino]-7-[[(3″S″)-tetrahydro-3-furanyl]oxy]-6-quinazolinyl]-4(dimethylamino)-2-butenamide, Tovok®); Vandetanib (Caprelsa®); Lapatinib (Tykcrb®); (3R,4R)-4-Amino-1-((4-((3-methoxyphenyl)amino)pyrrolo[2,1-f][1,2,4]triazin-5-yl)methyl)piperidin-3-ol (BMS690514); Canertinib dihydrochloride (CI-1033); 6-[4-[(4-Ethyl-1-piperazinyl)methyl]phenyl]-N-[(IR)-1-phenylethyl]-7H-Pyrrolo[2,3-d]pyrimidin-4-aminc (AEE788, CAS 497839-62-0); Mubritinib (TAK165); Pelitinib (EKB569); Afatinib (BIBW2992); Neratinib (HKI-272); N-[4-[[1-[(3-Fluorophenyl)methyl]-1H-indazol-5-yl]amino]-5-methylpyrrolo[2,1-f][1,2,4]triazin-6-yl]-carbamic acid, (3S)-3-morpholinylmethyl ester (BMS599626); N-(3,4-Dichloro-2-fluorophenyl)-6-methoxy-7-[[(3aα, 5β, 6aα)-octahydro-2-methylcyclopenta[c]pyrrol-5-yl]methoxy]-4-quinazolinaminc (XL647, CAS 781613-23-8); 4-[4-[[(1R)-1-Phenylethyl]amino]-7H-pyrrolo[2,3-d]pyrimidin-6-yl]-phenol (PKI166, CAS 187724-61-4); rocelitinib.

Exemplary mTOR inhibitors include, without limitation, rapamycin (Rapamune®), and analogs and derivatives thereof; SDZ-RAD; Temsirolimus (Torisel®; also known as CCI-779); Ridaforolimus (formally known as deferolimus, (1R,2R,4S)-4-[(2R)-2[(1R,9S,12S,15R,16E,18R,19R,21R,23S,24E,26E,28Z,30S,32S,35R)-1,18-dihydroxy-19,30-dimethoxy-15,17,21,23,29,35-hexamethyl-2,3,10,14,20-pentaoxo-11,36-dioxa-4-azatricyclo[30.3.1.0^{4.9}]hexatriaconta-16,24,26,28-tetraen-12-yl]propyl]-2-methoxycyclohexyl dimethylphosphinate, also known as AP23573 and MK8669, and described in PCT Publication No. WO 03/064383); Everolimus (Afinitor® or RAD001); Rapamycin (AY22989, Sirolimus®); Simapimod (CAS 164301-51-3); (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-onc (PF04691502, CAS 1013101-36-4); and N2-[1,4-dioxo-[[4-(4-oxo-8-phenyl-4H-1-benzopyran-2-yl)morpholinium-4-yl]methoxy]butyl]-L-arginylglycyl-L-α-aspartyIL-serine-, inner salt (SF1126, CAS 936487-67-1).

Exemplary Phosphoinositide 3-kinase (PI3K) inhibitors include, but are not limited to, duvelisib, idelalisib, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730); 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806); 4-(trifluoromethyl)-5-(2,6-dimorpholinopyrimidin-4-yl)pyridin-2-amine (also known as BKM120 or NVP-BKM120, and described in PCT Publication No. WO2007/084786); Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acctyloxy)-1-[(di-2-propenylamino)methylene]-4,4a,5,6,6a,8,9,9a-octahydro-11-hydroxy-4-(methoxymethyl)-4a,6α-dimethyl-cyclopenta[5,6]naphtho[1,2-c]pyran-2,7,10(1H)-trione (PX866, CAS 502632-66-8); and 8-Phenyl-2-(morpholin-4-yl)-chromen-4-one (LY294002, CAS 154447-36-6).

Exemplary Protein Kinase B (PKB) or AKT inhibitors include, but are not limited to. 8-[4-(1-Aminocyclobutyl)phenyl]-9-phenyl-1,2,4-triazolo[3,4-f][1,6]naphthyridin-3(2H)-one (MK-2206, CAS 1032349-93-1); Perifosine (KRX0401); 4-Dodecyl-N-1,3,4-thiadiazol-2-yl-benzenesulfonamide (PHT-427, CAS 1191951-57-1); 4-[2-(4-Amino-1,2,5-oxadiazol-3-yl)-1-ethyl-7-[(3S)-3-piperidinylmethoxy]-1H-imidazo[4,5-c]pyridin-4-yl]-2-methyl-3-butyn-2-ol (GSK690693, CAS 937174-76-0); 8-(1-Hydroxyethyl)-2-methoxy-3-[(4-methoxyphenyl)methoxy]-6H-dibenzo[b,d]pyran-6-one (palomid 529, P529, or SG-00529); Tricirbine (6-Amino-4-methyl-8-(β-D-ribofuranosyl)-4H,8H-pyrrolo[4,3,2-de]pyrimido[4,5-c]pyridazine); (αS)-α-[[[5-(3-Methyl-1H-indazol-5-yl)-3-pyridinyl]oxy]methyl]-benzencethanamine (A674563, CAS 552325-73-2); 4-[(4-Chlorophenyl)methyl]-1-(7H-pyrrolo[2,3-d]pyrimidin-4-yl)-4-piperidinamine (CCT128930, CAS 885499-61-6); 4-(4-Chlorophenyl)-4-[4-(1H pyrazol-4-yl)phenyl]-piperidine (AT7867, CAS 857531-00-1); and Archexin (RX-0201, CAS 663232-27-7).

In certain embodiments, a tyrosine kinase inhibitor used in combination with anti-CD72 CAR modified cells is an anaplastic lymphoma kinase (ALK) inhibitor. Exemplary ALK inhibitors include crizotinib, ceritinib, alectinib, brigatinib, dalantercept, entrectinib, and lorlatinib.

In certain embodiments where anti-CD72 CAR modified cells are administered in combination with one or more additional therapies, the anti-CD72 CAR or one or more additional therapies may be administered at a dose that might otherwise be considered subtherapeutic if administered as a monotherapy. In some embodiments, the one or more additional therapies comprises a CER or a chimeric Tim receptor. In such embodiments, the anti-CD72 CAR combined with CER or a chimeric Tim receptor may provide an additive or synergistic effect such that the one or both therapies can be administered at a lower dose. Combination therapy includes administration of anti-CD72 CAR compositions as described herein before an additional therapy (e.g., 1 day to 30 days or more before the additional therapy), concurrently with an additional therapy (on the same day), or after an additional therapy (e.g., 1 day-30 days or more after the additional therapy). In certain embodiments, the anti-CD72 CAR modified cells are administered concurrently with the one or more additional therapies. In further embodiments, the anti-CD72 CAR modified cells are administered 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days before or after administration of the one or more additional therapies. In still further embodiments, the anti-CD72 CAR modified cells are administered within 4 weeks, within 3 weeks, within 2 weeks, or within 1 week before or after administration of the one or more additional therapies. Where the one or more additional therapies involves multiple doses, the anti-CD72 CAR modified cells may be administered before or after the initial dose of the one or more additional therapies, after the final dose of the one or more additional therapies, or in between multiple doses of the one or more additional therapies.

In certain embodiments, methods of the present disclosure include a depletion step. A depletion step to remove anti-CD72 CAR from the subject may occur after a sufficient amount of time for therapeutic benefit in order to mitigate toxicity to a subject. In such embodiments, the anti-CD72 CAR vector may include an inducible suicide gene, such as iCASP9, inducible Fas, or HSV-TK. Similarly, a chimeric Tim receptor vector may be designed for expression of a known cell surface antigen such as CD20 or truncated EGFR (SEQ ID NO:295 or 443) that facilitates depletion of transduced cells through infusion of an associated monoclonal antibody (mAb), for example, Rituximab for CD20 or Cetuximab for EGFR. Alemtuzumab, which targets CD52 present on the surface of mature lymphocytes, may also be used to deplete transduced B cells, T cells, or natural killer cells.

Subjects that can be treated by the compositions and methods of the present disclosure include animals, such as humans, primates, cows, horses, sheep, dogs, cats, mice, rats, rabbits, guinea pigs, or pigs. The subject may be male or female, and can be any suitable age, including infant, juvenile, adolescent, adult, and geriatric subjects.

### EXAMPLES

### Example 1

### Design and Characterization of Anti-Cd72 Chimeric Antigen Receptor (Car) T Cells

Despite significant progress, high-risk, advanced CLL patients are a distinct B cell malignancy subset with low-moderate CD19 CAR responses and overall poor clinical outcomes. The cell surface protein, CD72, is expressed on normal and neoplastic B cells (Garand R, Robet al. Leuk Res. 1994; 18(8):651-2.) and is further predicted to have symmetric gene expression with CD19 (Sahoo D. Front Physiol. 2012; 3:276). The high B cell restriction of CD72, its broad expression on malignant lymphoma and leukemia B cells and Acute Myelogenous Leukemia (AML) subsets, and its retained expression on relapsed CD19- and CD22-directed therapies, renders CD72 a suitable target for B cell lymphomas and leukemias and AML tumors.

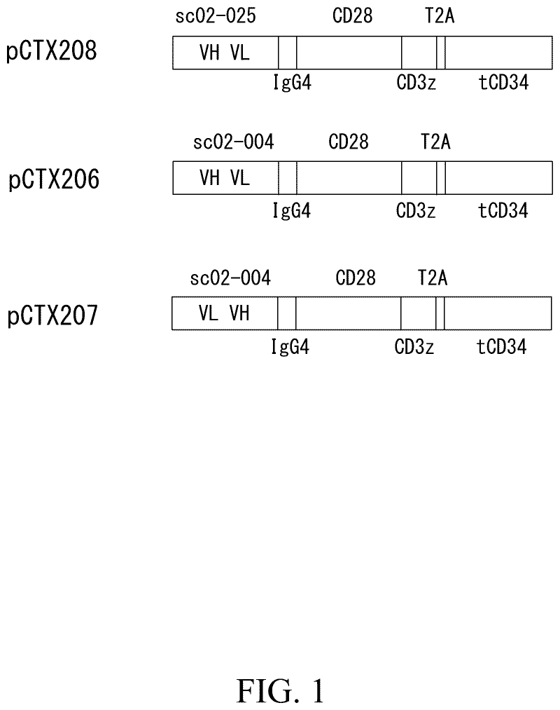

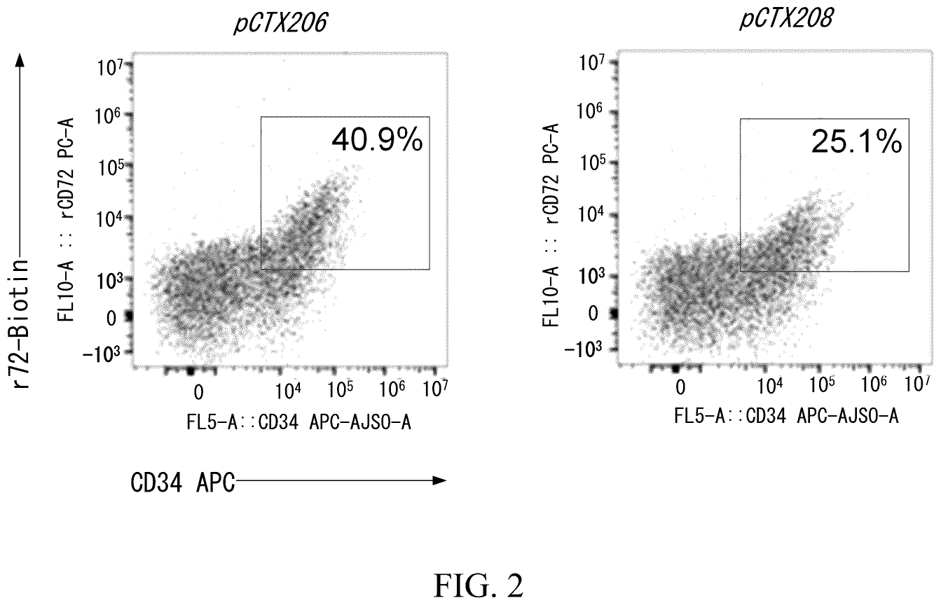

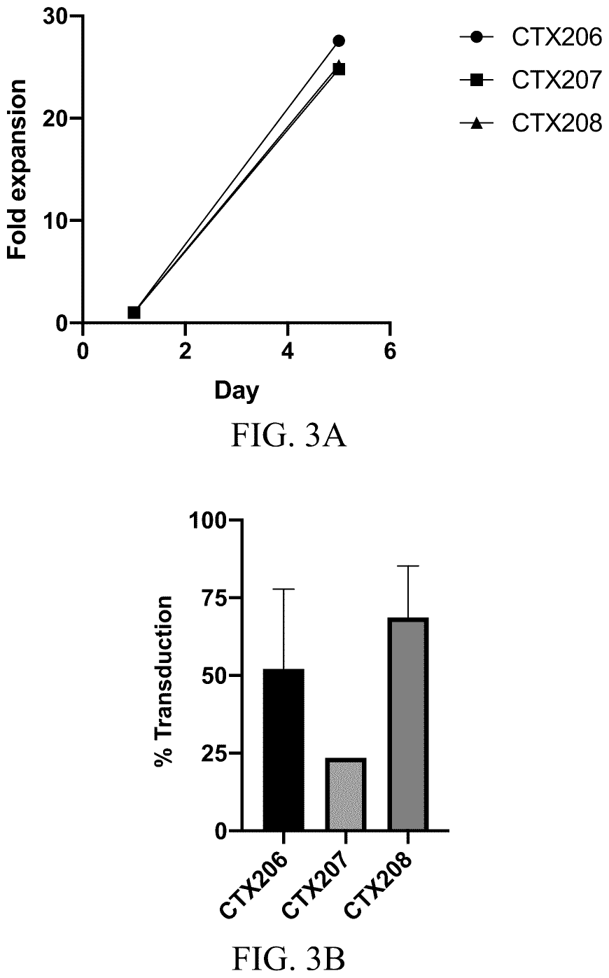

CD72-directed CAR constructs pCTX206 (SEQ ID NO:31), pCTX207 (CTX207 CAR possesses the same components as CTX206, except the sc02-004 scFv is in the VL-VH orientation rather than VH-VL), and pCTX208 (SEQ ID NO:32) were designed, as shown in FIG. 1. Each construct includes either a sc02-004 (SEQ ID NO:17) or sc02-025 anti-CD72 scFv (SEQ ID NO:18), IgG4 hinge region (SEQ ID NO:21), CD28-derived transmembrane (SEQ ID NO:25) and signaling domains (SEQ ID NO:28), CD35-derived signaling domain (SEQ ID NO:26), and T2A ribosome skip element. Truncated CD34 was included in pCTX206 or pCTX208 vectors to serve as a selection marker for successful transduction. Following transduction, co-expression of the CD72 CAR and tCD34 were evaluated. T cells were transduced with pCTX206 or pCTX208 and stained for cell surface expression of the CD72 CAR and tCD34. Coexpression of the CD72 CAR and tCD34 are shown in FIG. 2. Transduction efficiency and fold expansion were evaluated in T cells transduced with pCTX206, pCTX207, or pCTX208 vectors. T cells were stimulated with TransACT in a 24 well gRex plate for 24 hours, after which, cells were counted and transduced with 5 MOI of lentivirus encoding pCTX206, pCTX207, or pCTX208. After 6 d in culture, cells were resuspended, counted, and fold expansion was calculated, as seen in FIG. 3A. At 6 d post transduction, transduced T cells were resuspended and stained for the tCD34 marker gene encoded on pCTX206, pCTX207, and pCTX208. Cell staining was analyzed by flow cytometry and transduction efficiency data is shown in FIG. 3B.

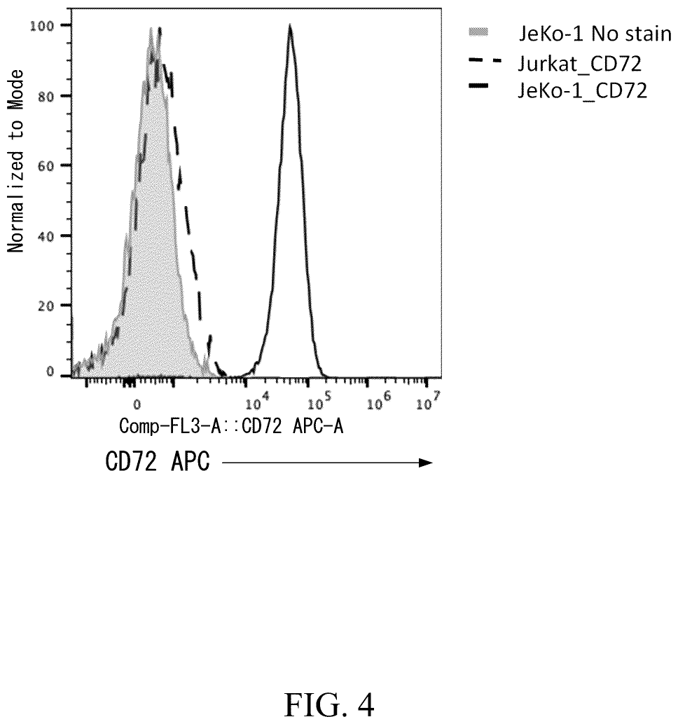

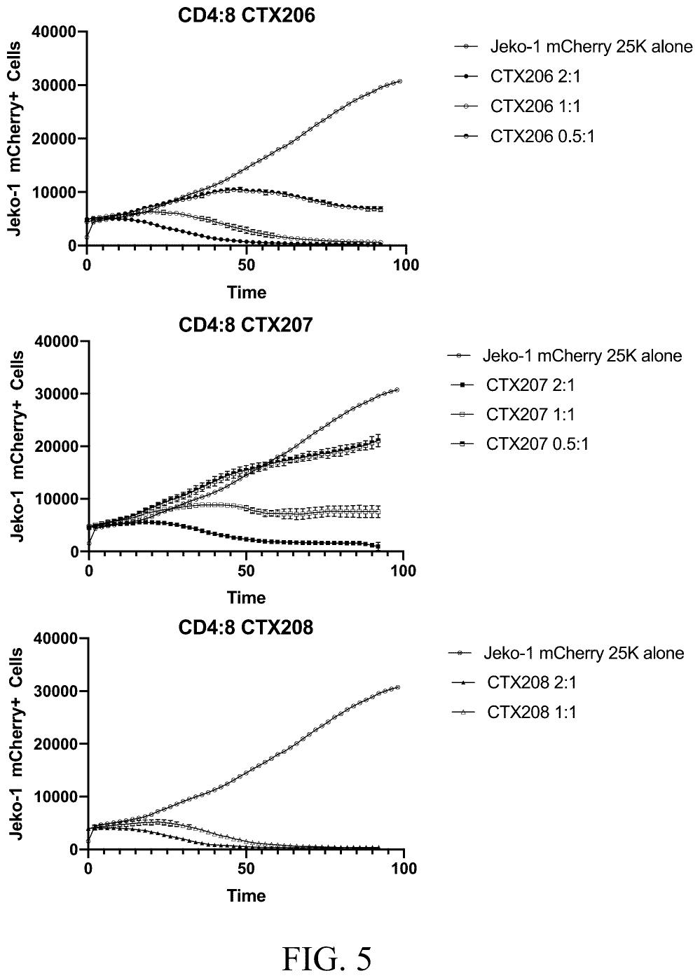

Cytotoxicity of T cells transduced with CD72-directed CAR vectors were evaluated using Jeko-1 mantle cell lymphoma cells. Surface expression of CD72 in Jeko-1 cells was first confirmed, as seen in FIG. 4. Jurkat lymphoma cells, which have a T cell lineage, and unstained cells were used as negative controls. Cells were stained with anti-CD72 antibodies and characterized by FACS. To measure cytotoxicity, Jeko-1 cells were engineered to express mCherry; 25,000 mCherry+JeKo-1 cells were cultured at a 2:1, 1:1, or 0.5:1 effector cell:target cell ratio (E:T ratio) with CTX206, CTX207, or CTX208 CAR-T cells. Cultures were incubated at 37° C. for 4 d on a 96-well fibronectin-coated plate in an Incucyte cell culture and imaging device. The number of mCherry+JeKo-1 cells remaining in culture over time is shown in FIG. 5.

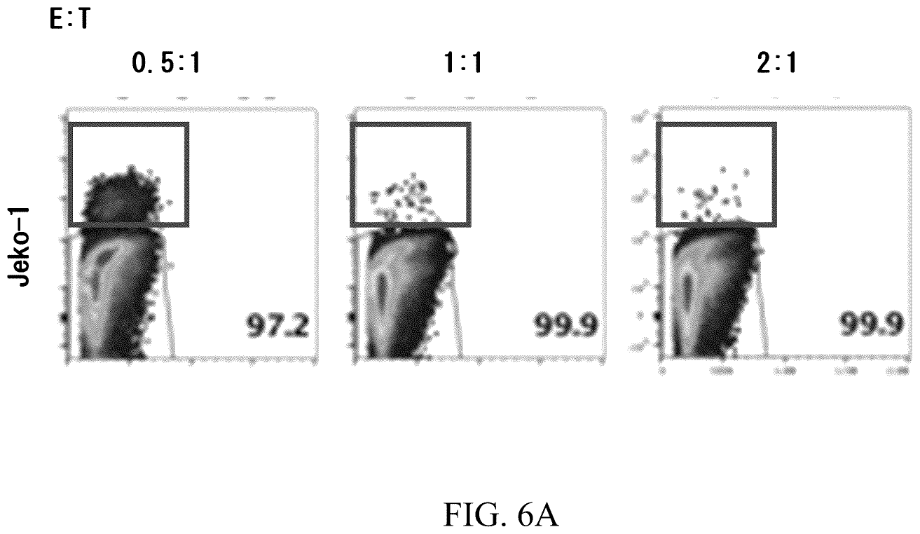

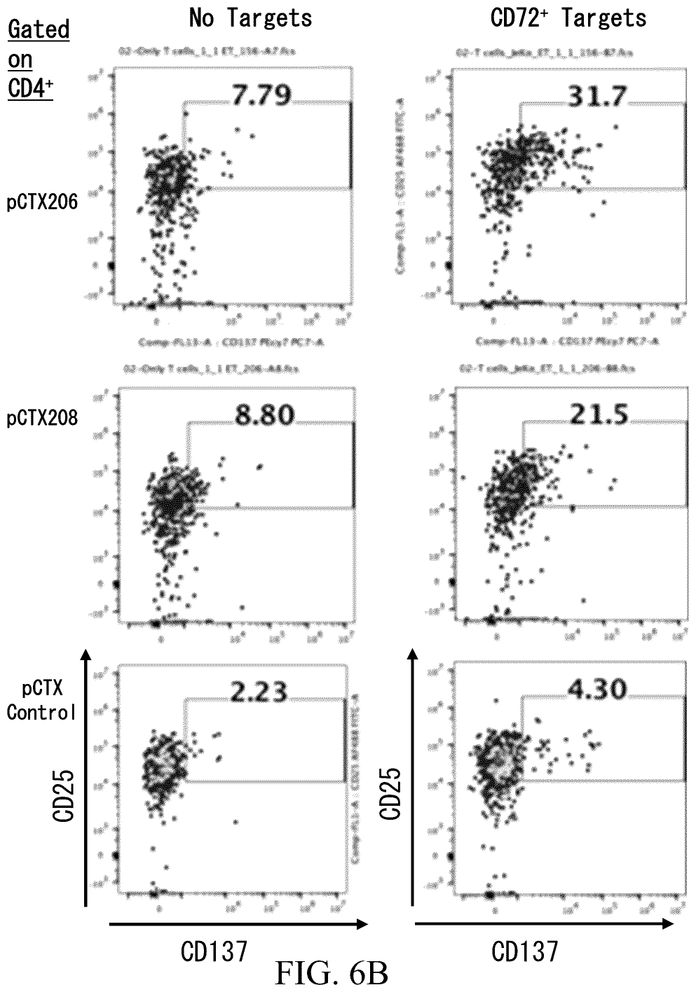

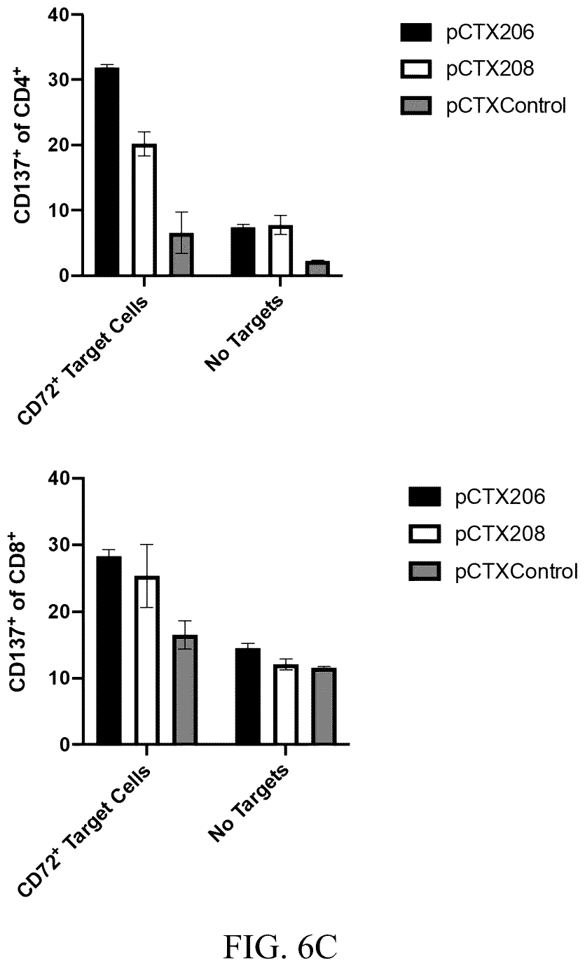

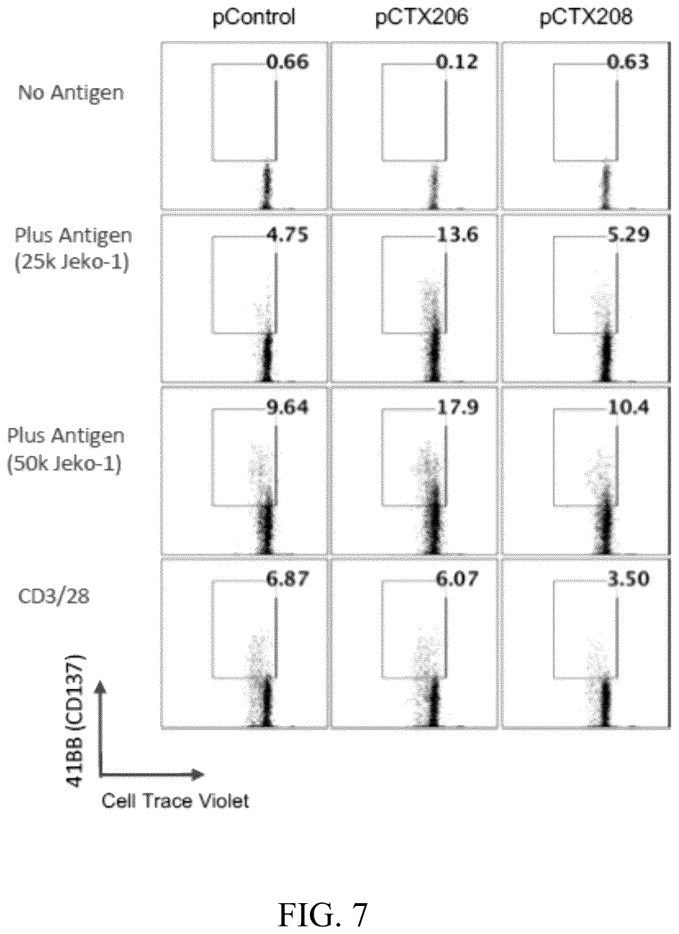

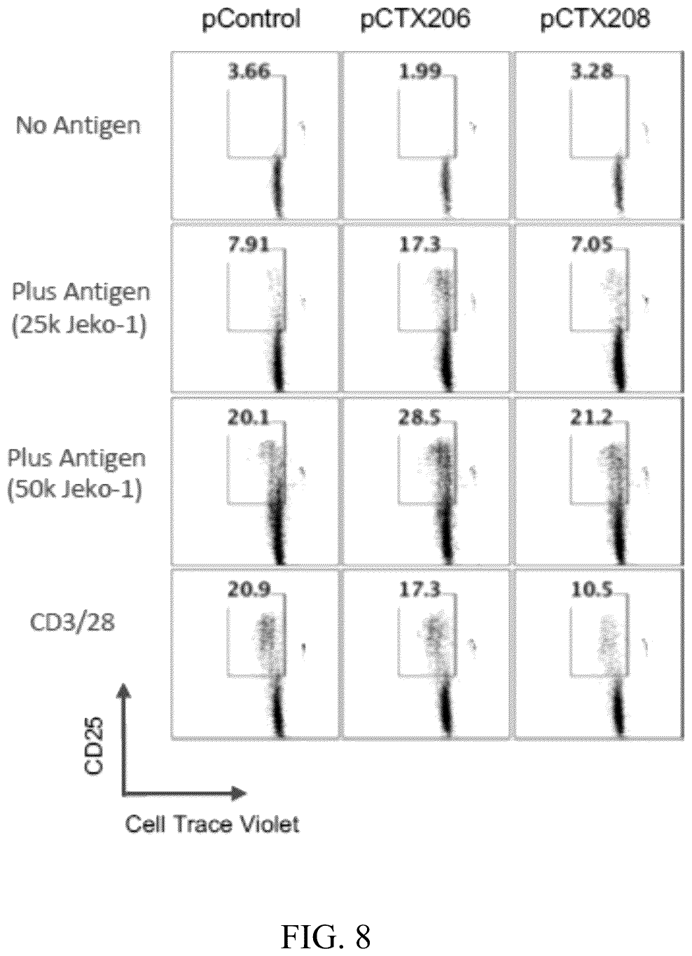

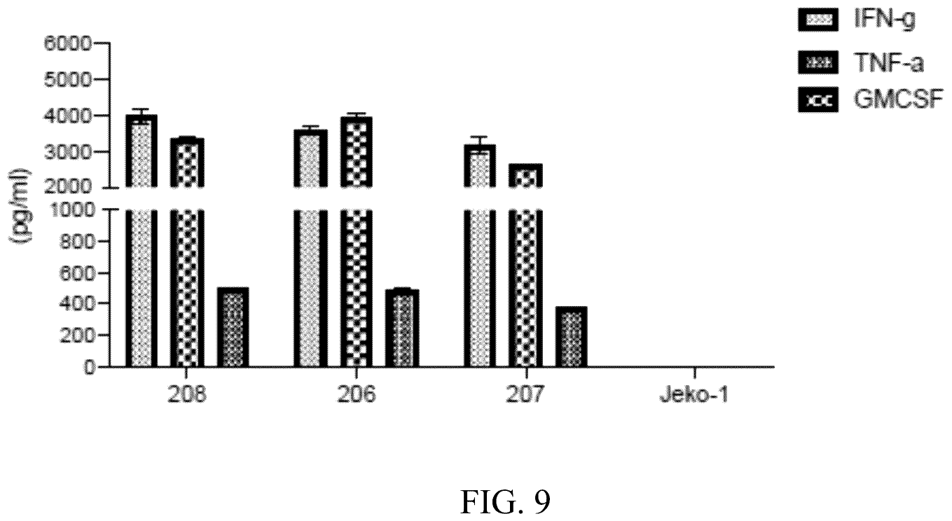

CD72 CAR-T cells were further characterized phenotypically and functionally in the context of CD72-target cell-specific activation. CTX206 CAR-T cells were cultured with Jeko-1 cells for 96 hrs followed by staining with anti-CD19 antibodies to detect remaining Jeko-1 cells and evaluate cytotoxicity, as shown in FIG. 6A. Dose dependent decreases in target cells were observed upon culture with pCTX206, concordant with incucyte imaging data. CTX206 and CTX208 CAR-T cells were assessed for expression and specific upregulation of CD25 and CD137 in response to Jeko-1 (CD72⁺) cells, as shown in FIG. 6B and FIG. 6C. Unstimulated CTX206 or CTX208 CAR-T cells were used as negative controls to assess the presence of constitutive or tonic activation signals in the absence of targets. Proliferation as associated with CD137 expression (FIG. 7) and CD25 expression (FIG. 8) in CTX206 or CTX208 CAR-T cells was evaluated 72 hours-post stimulation with Jeko-1 cells in the absence of cytokines. Cells used in the assay were first stimulated with TransAct (CD3/28) for 21 days before input into the assay. Cell Trace Violet dye dilution and anti-CD137 antibody staining were assessed by FACS. Bulk cytokine secretion from CTX206, CTX207, and CTX208 CAR-T cells is shown in FIG. 9. Multiplex cytokine analysis from bulk supernatants was obtained 48 hrs after co-culture with Jeko-1 targets.

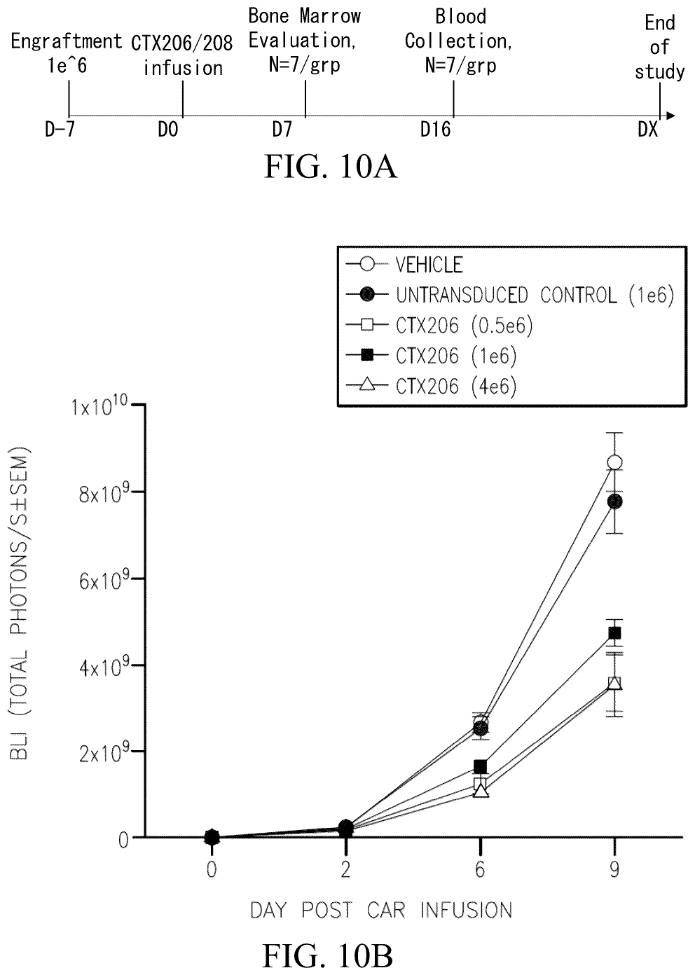

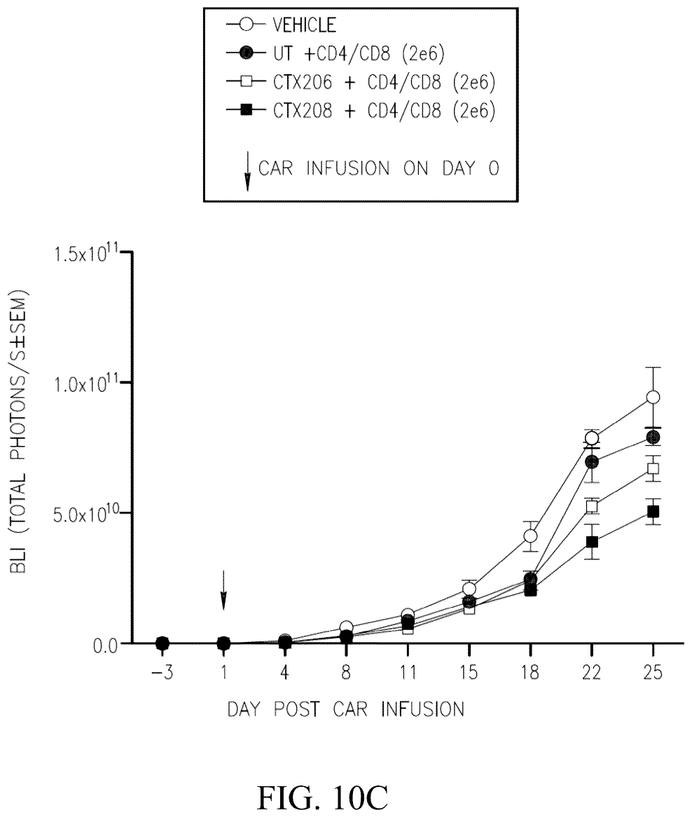

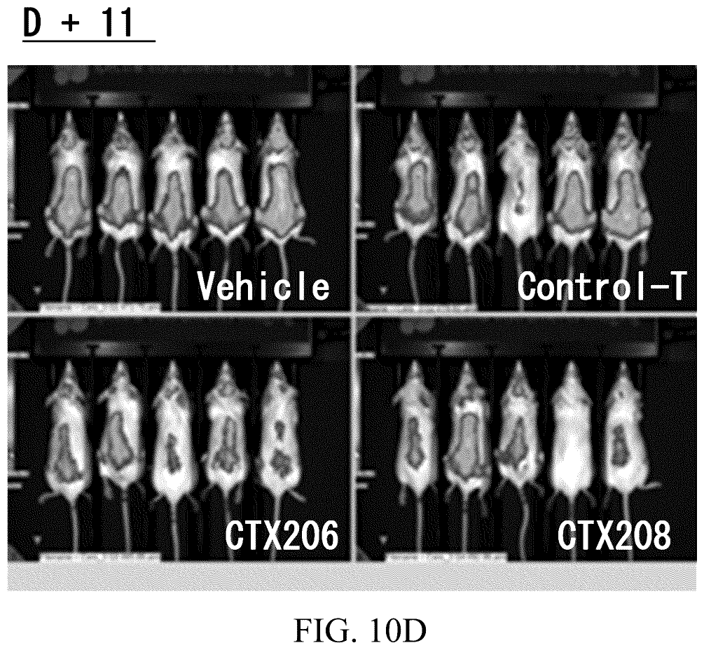

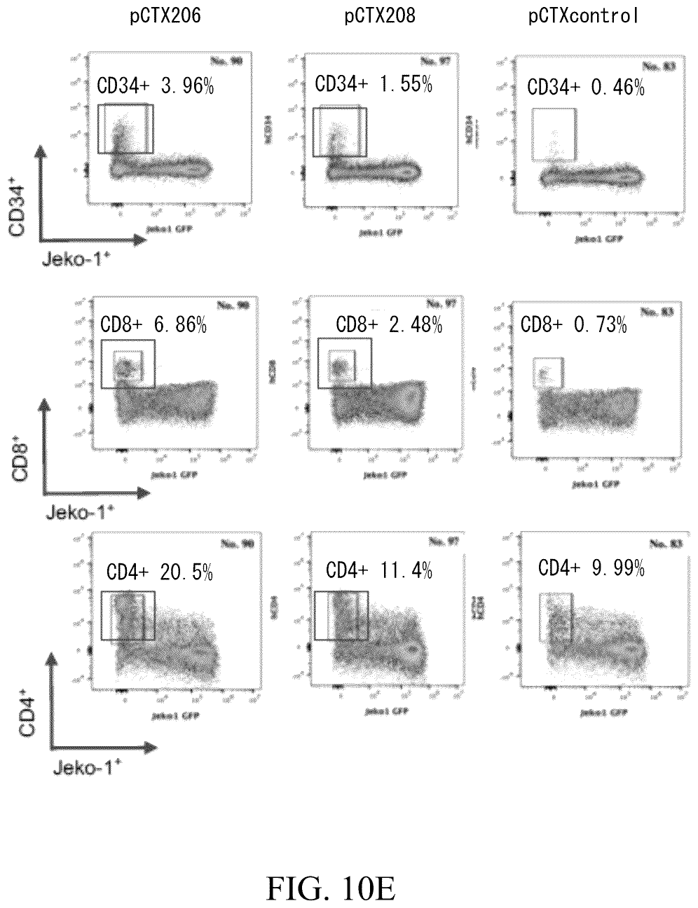

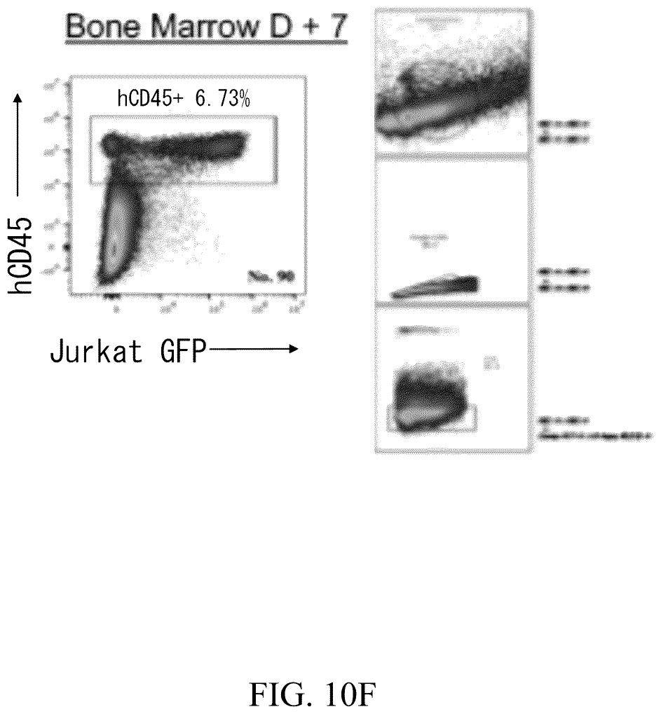

Murine models were used to assess the capacity of CD72 CAR-T cells to reduce tumor burden in vivo. As shown in FIG. 10A, cohorts were inoculated with 1e6 JeKo-1 ffluc cells via tail injection 7 days prior to infusion with CTX206 CAR-T, CTX208 CAR-T, or control untransduced T cells. Serial bioluminescence imaging was performed to quantify tumor burden in mice following CAR-T infusion with 0.5e6, 1e6, or 4e6 CTX206 CAR-T cells (FIG. 10B) and peripheral blood was collected sixteens days post-CAR T cell infusion. In another experiment, the same model was used to deliver, 2e6 CTX206 or 2e6 CTX208 CAR-T cells, in which serial bioluminescence imaging was also used to measure tumor burden (FIG. 10C). Tumor-associated bioluminescence derived from JeKo-1 ffluc cells is shown in FIG. 10D, in which mice were imaged at 11 days post-infusion with 2e6 CTX206 or 2e6 CTX208 CAR-T cells. Bone marrow was collected 7 days following infusion of 2e6 cells and the frequency of CD45+CD8+CD4+CD34+-T cells were quantified, as shown in FIGS. 10E and 10F.

### Example 2

### Combination Use of Anti-Cd72 Car-T Cells and Chimeric Engulfment Receptor (Cer)-Modified T Cells

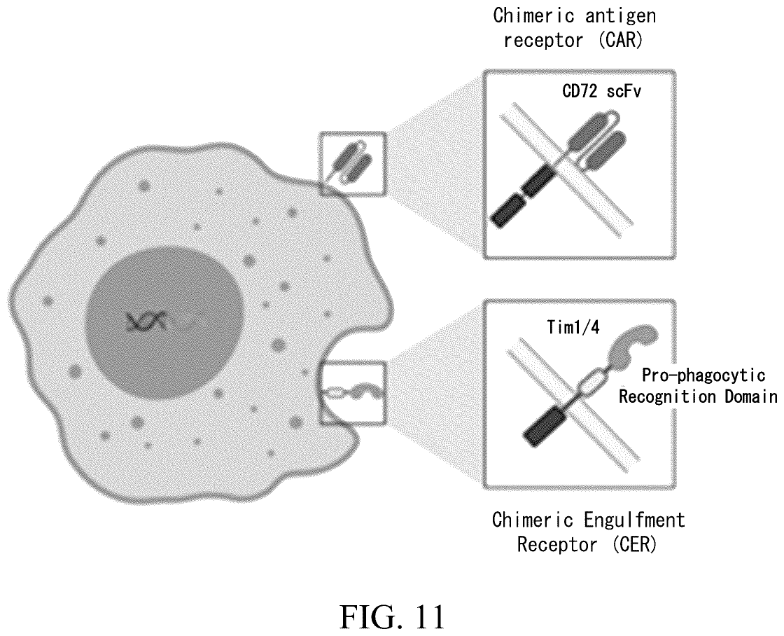

Antigen escape or downregulation is one mechanism for relapse from CAR T-cell therapy. An approach to overcome this is to simultaneously target more than one antigen on cancer cells. Chimeric Engulfment Receptors (CER) are engineered to target phosphatidylserine and direct macrophages to engulf specific targets on cancer cells. CERs may synergize with CD72 CARs cells to enhance overall anti-tumor function when expressed on a single cell, as shown in FIG. 11, or co-infused as CER-modified T cells and CAR-T cells. In this study, CERs and CD72 CAR constructs were co-transduced or co-cultured in vitro to evaluate cytotoxic capacity and co-infused to evaluate anti-tumor efficacy in vivo.

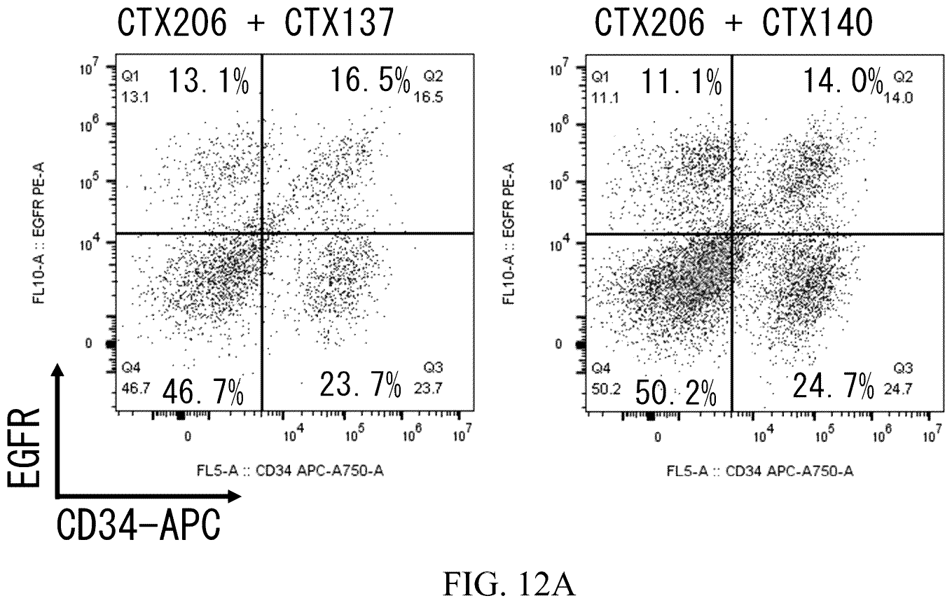

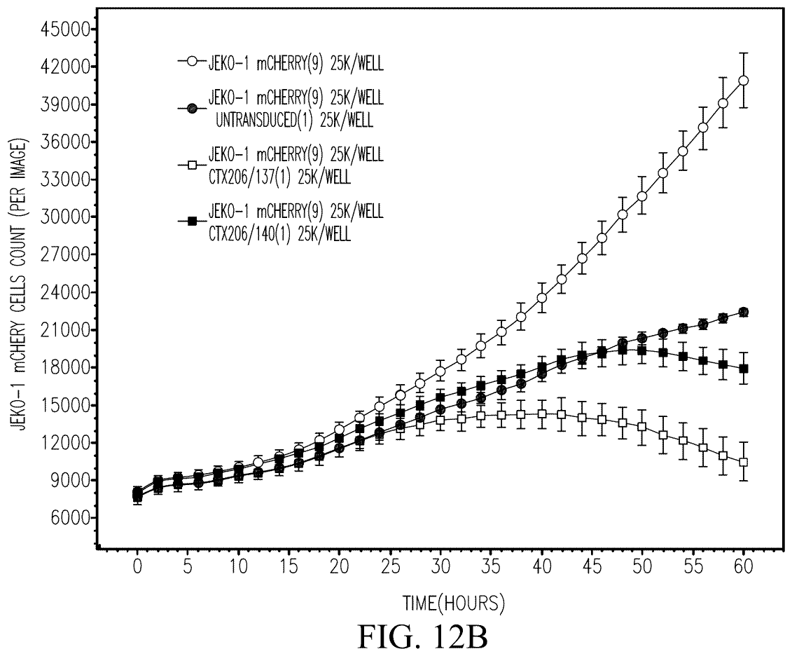

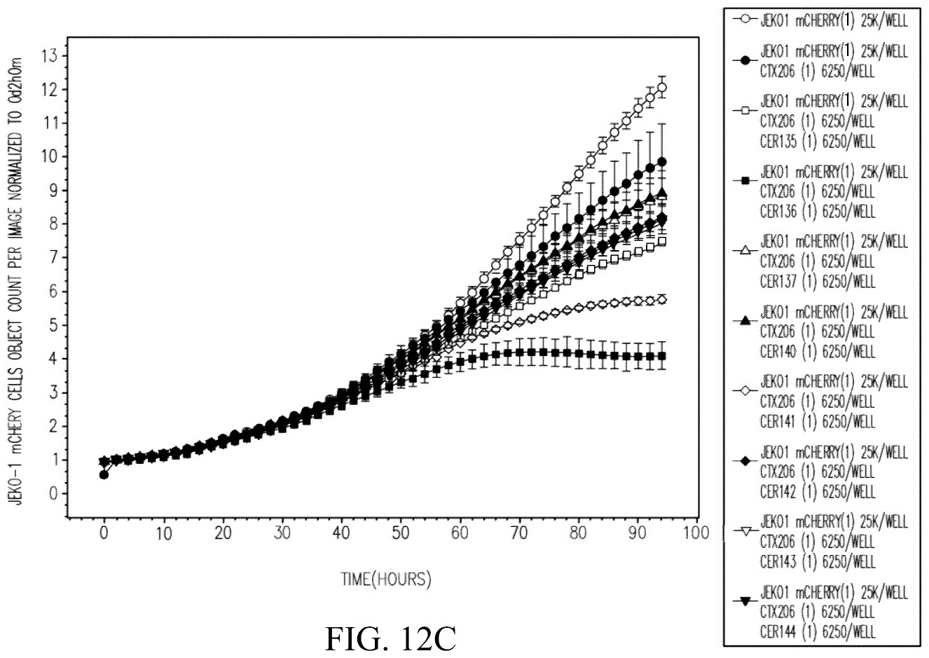

CTX206 and a CER, CTX137 or CTX140 were co-transduced in T cells and transduction efficacy was evaluated. Transduction with CTX137 or CTX140 is indicated by surface expression of EGFR and transduction with CTX206 is indicated by surface expression of tCD34, as shown in FIG. 12A. To evaluate cytotoxicity mediated by these co-expressing T cells, cells transduced with both CTX206+CTX137 or CTX206+CTX140 were incubated at a 1:1 ratio with mCherry+JeKo-1 target cells for 2.5 d in an Incucyte live-cell imaging system. The number of mCherry+JeKo-1 cells is shown at each time point in FIG. 12B. Additive toxicity of mixed CAR-T and CER populations in co-culture was next evaluated in vitro. CTX206 CAR-T cells were mixed with CER-transduced T cells (CER135, CER136, CER/CTX137, CER/CTX140, CER141, CER142, CER143, CER144) in a 1:1 ratio and cultured with target Jekol mCherry T cells at a 0.25:1 effector:target ratio. FIG. 12C shows the number of mCherry+JeKo-1 cells at each time point.

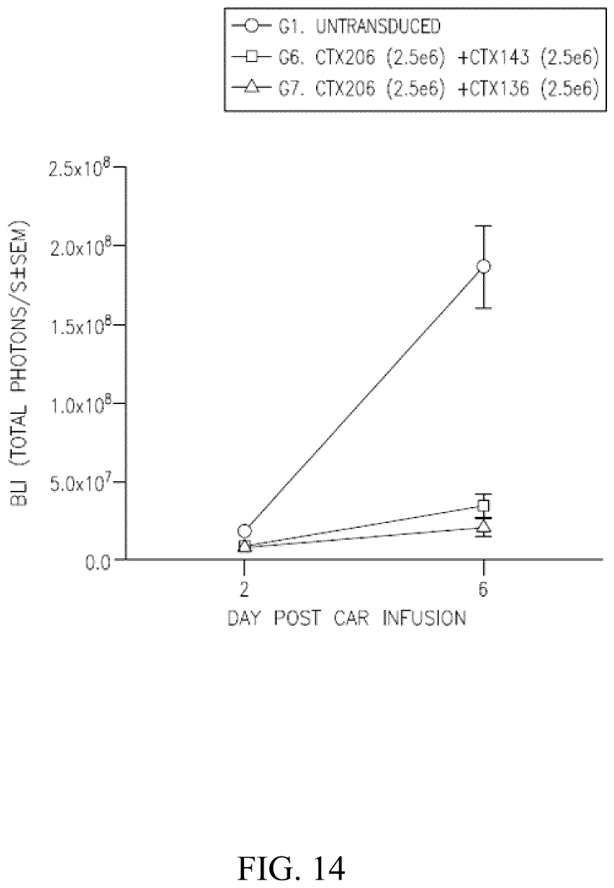

CTX208 and a CER, CTX137 were co-transduced in T cells and transduction efficacy was evaluated. As shown in FIG. 14A, T cells were transduced with either CTX137 alone, CTX208 alone, or equivalent amounts of CTX137 and CTX208. At 6 d post transduction, expression of the tEGFR (CTX137) and tCD34 (CTX208) tags were measured on transduced cells by flow cytometry. To assess cytotoxic capacity, T cells transduced with CTX208 or co-transduced with CTX137 and CTX208 were incubated at a 1:1 ratio (FIG. 14B) or varying ratios (FIG. 14C) with 25,000 Jeko-1 cells engineered to express mCherry. Cells were incubated for 4.5 d in an Incucyte live-cell imaging system for the assay.

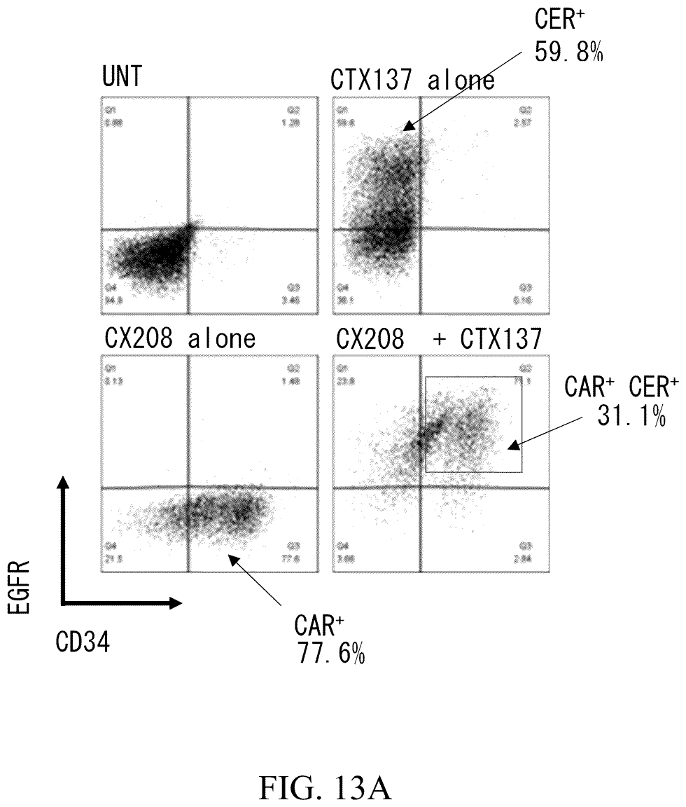

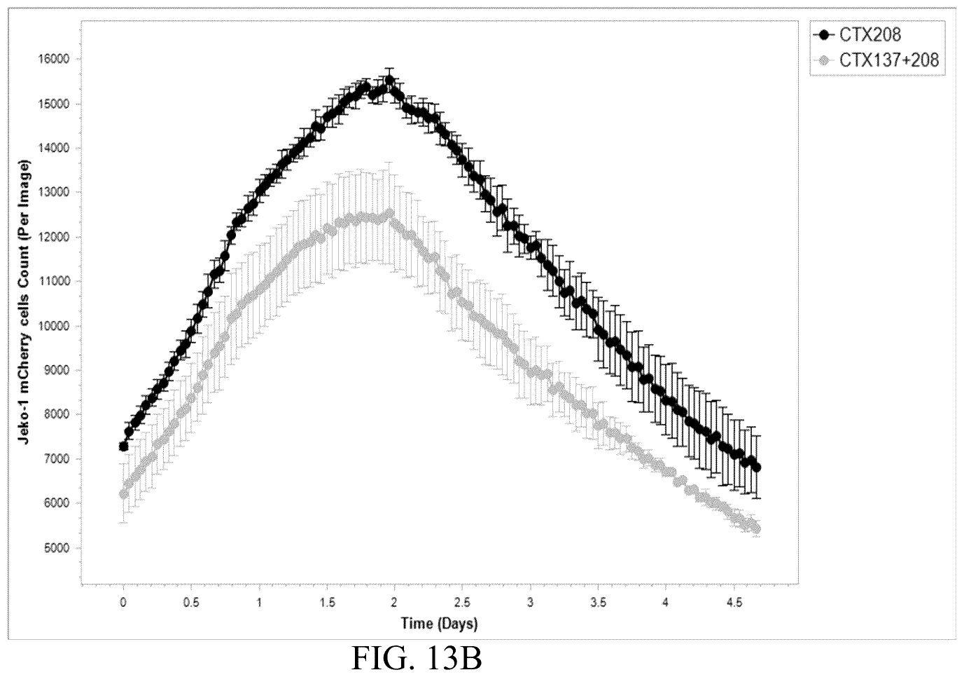

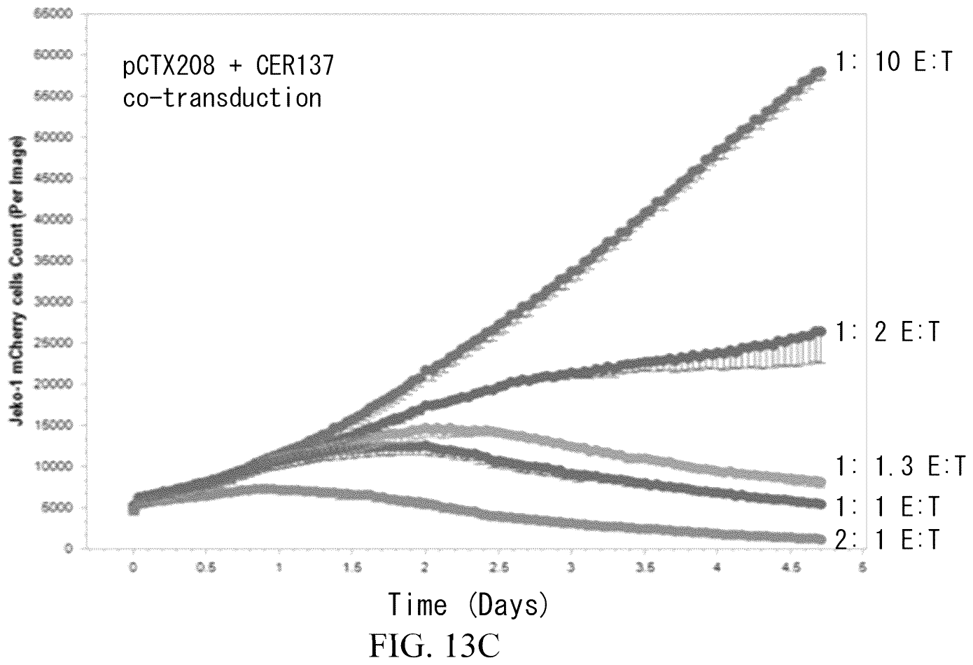

A murine model was used to evaluate in vivo tumor control by the co-infusion of CTX206 CAR-T cells and T cells expressing CERs, CTX143 or CTX136. As seen in FIG. 13, cohorts were inoculated with 1e6 JeKo-1 ffluc via tail injection and 2.5e6 CTX206 CAR-T cells 7 days after tumor inoculation. After 72 hours, animals were infused with a second infusion of 2.5e6 T cells expressing CTX136 or CTX143. Serial bioluminescence imaging and quantification are illustrated through day +6 post CAR infusion (day +3 post CER infusion).

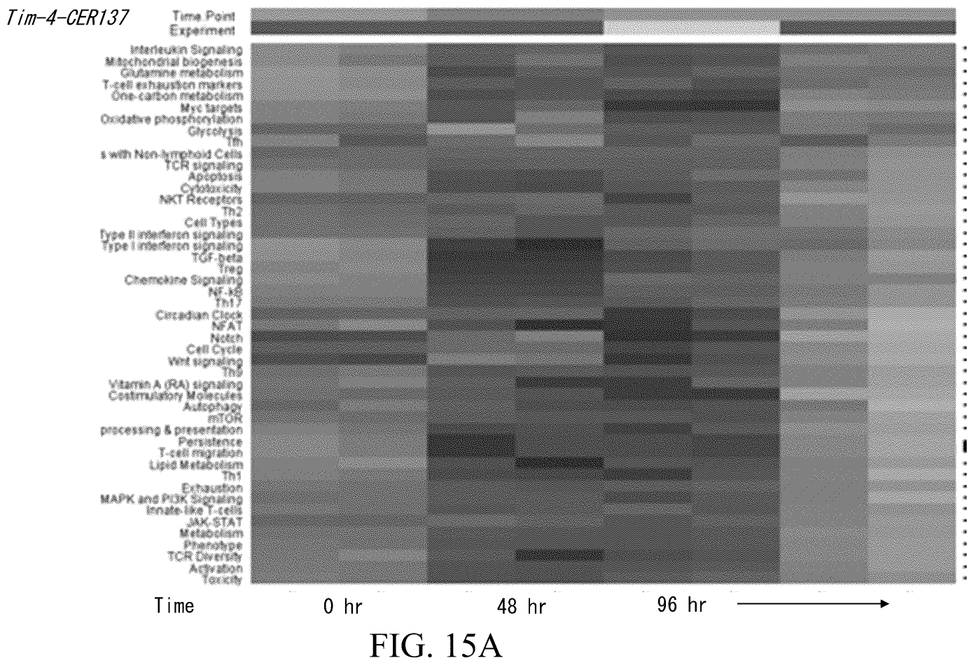

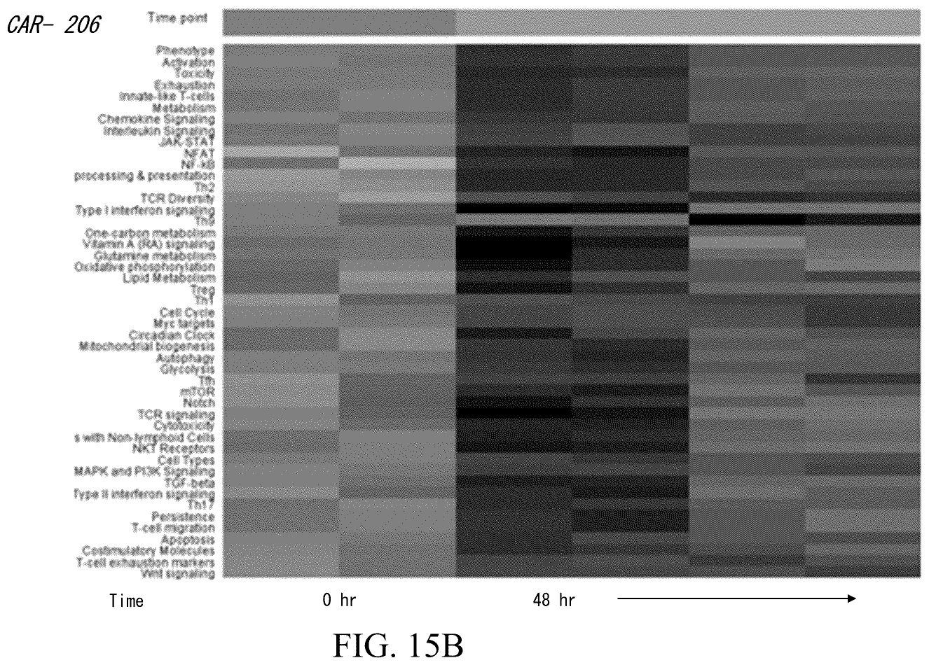

Differentially expressed RNA transcripts from sorted chimeric TIM-4 receptor T cells (FIG. 15A) and CAR-T cells (FIG. 15B) after antigen encounter were measured. Gene ontology analysis was performed to assess the presence of divergent transcriptional programs. Cells were cultured similarly through day 8 post-activation and transduction. CAR and CER-T cells were sorted after 48 and 96 hrs after co-culture with Ag+ cells (JeKo-1). Heat maps show progression of transcriptional programs over time in the culture relative to baseline.

### Example 3

### Materials & Methods

### T Cell Transduction

CD4 and CD8 cells were isolated from frozen healthy donor PBMC using the StemCell EasySep CD4 or CD8 isolation kits (Stemcell). CD4 and CD8 cells were mixed at a 1:1 ratio and incubated overnight in a 24-well gRex tissue culture plate (Wilson Wolf) in the presence of TransACT (Miltenyi Biotec). 24 h post stimulation, cells were transduced with 5MOI of pCTX206, pCTX207, or pCTX208.

### Measurement of T Cell Transduction

Transduced cells were stained with anti-CD34 (Clone 561, Biolegend). Cells were analyzed by flow cytometry on a CytoFLEX FACs machine (Beckman Coulter). Flow data was analyzed on FlowJo (FlowJo LLC.).

### Measurement of Fold Expansion

T cells were measured immediately prior to transduction. After 5 d of culture at 37° C., cells were resuspended and counted using a Vi-Cell cell counter (Beckman Coulter). Fold expansion was calculated by dividing number of cells at d5 by number of cells at do.

### CAR T Cell Killing of Lymphoid Targets

A 96 well flat-bottomed plate was coated overnight at 4° C. with 1 ug/cm² fibronectin (Sigma Aldrich). 25,000 Jeko-1 mantle cell lymphoma cells engineered to overexpress mCherry were incubated with CTX206, CTX207, or CTX208-transduced T cells at a 2:1, 1:1, or 0.5:1 Effector: Target ratio. CellEvent Caspase3/7 Green detection reagent (Thermofisher Scientific) was added to each well. Cells were incubated for 4 d at 37° C. in an Incucyte live-cell imaging and analysis machine (Essen Bioscience). Wells were imaged every 2 h at 4× using phase-contrast, green, and red fluorescence imaging. Absolute number of mCherry+ objects in the field of view was counted for each well.

### Example 4

### Phosphatidylserine Induction

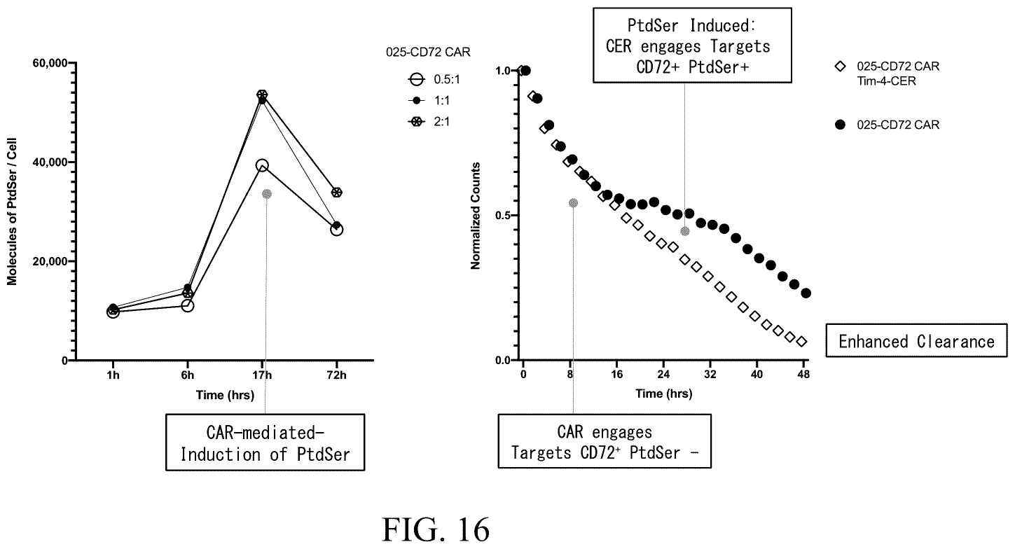

CD72 specific CAR engagement primes target cells for clearance by CERs. As shown in FIG. 16 (left panel), treatment of CD72⁺ target cells with a CD72 specific CAR (having “025” scFv comprising VH of SEQ ID NO:356 and VL of SEQ ID NO:357) induces phosphatidylserine on target cell surface. Upon induction of phosphatidylserine, a CER with a Tim4 binding domain engages CD72+/PtdSer+ target cells and enhances clearance (FIG. 16, right panel).

### Example 5

### Improving Potency of Dual Targeting Cer-Car-Engineered Cells

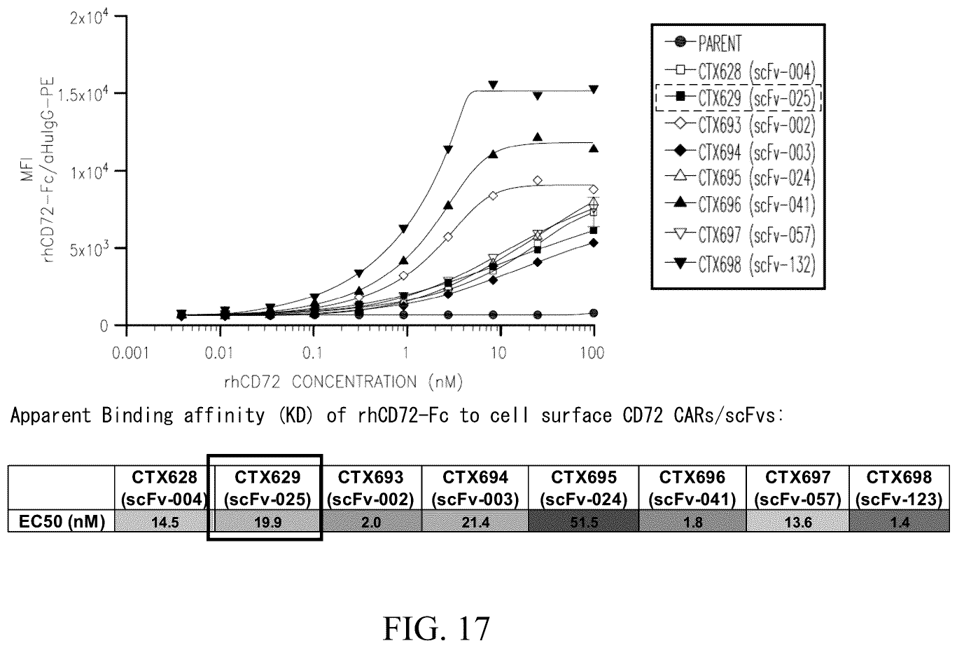

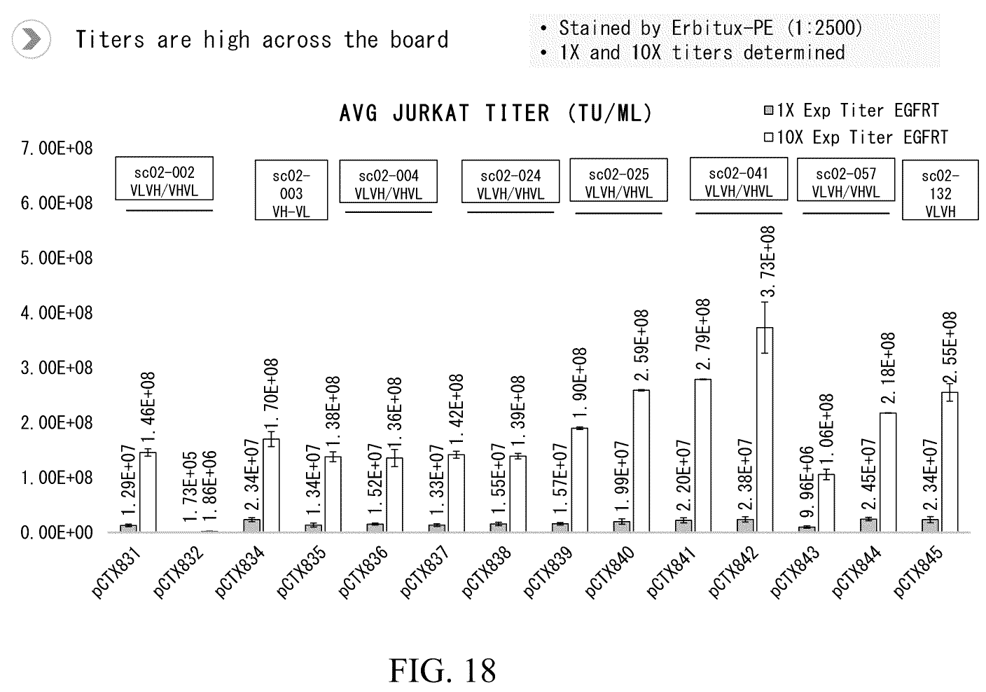

Alternative scFvs may have different binding affinities or stabilities, which may affect T cell activation kinetics, PtdSer induction, and CER-CAR T function. To improve performance of CER-CAR T cells, a search for a superior scFv was conducted. FIG. 17 shows screening of various CD72 specific scFvs (all having same linker) for binding affinity. FIG. 18 shows expression titers of various scFvs and variable region orientations based on detection of co-expression of EGFRt using anti-EGFRt antibody staining. FIG. 18 shows expression titers of various CD72 specific CARs having different scFvs; the expression titers were found to be high across the board.

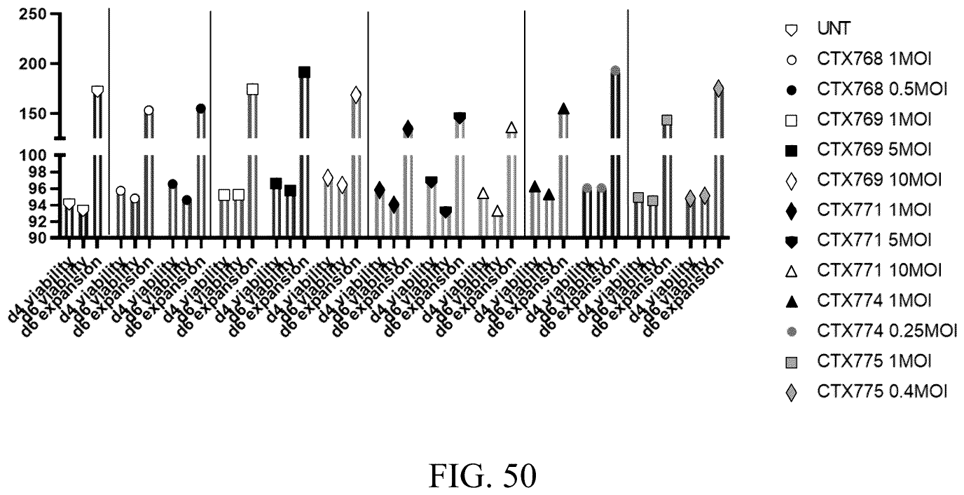

FIG. 50 shows for multiple CD72 CARs, high viability and fold expansion observed throughout cell production. CD4 and CD8 cells were mixed at a 1:1 ratio in the presence of TransACT. 24 h post stimulation, cells were transduced with a range of lentiviruses encoding CER constructs across different MOIs. Cells were expanded in the presence of IL-2, IL-7, and IL-15 in G-Rex vessels. Viabilities and fold expansion were quantified day +4 and day +6 post-transduction.

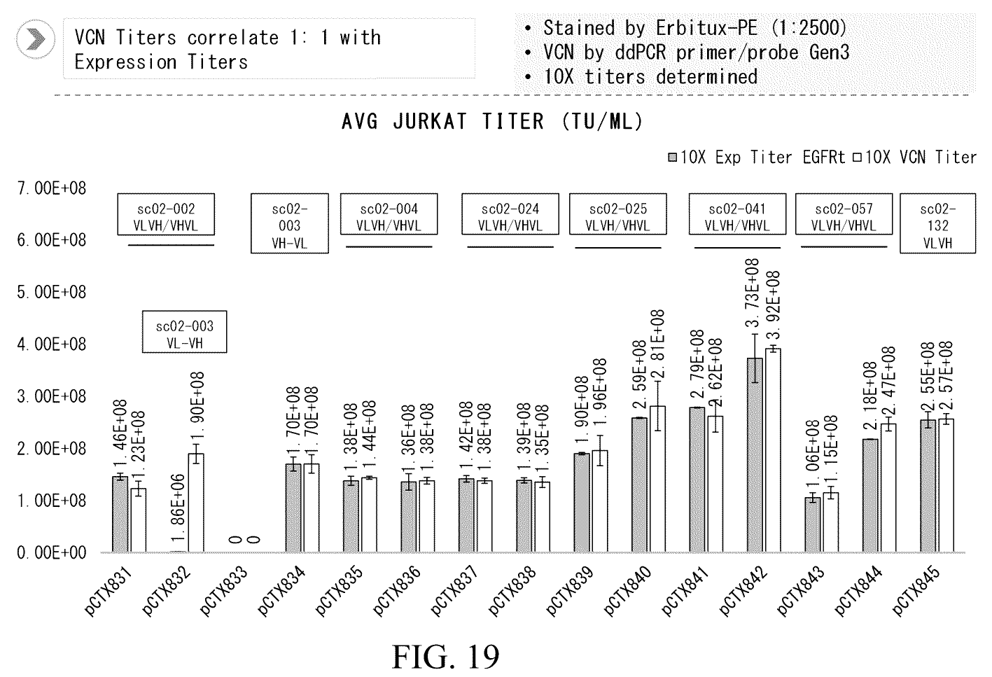

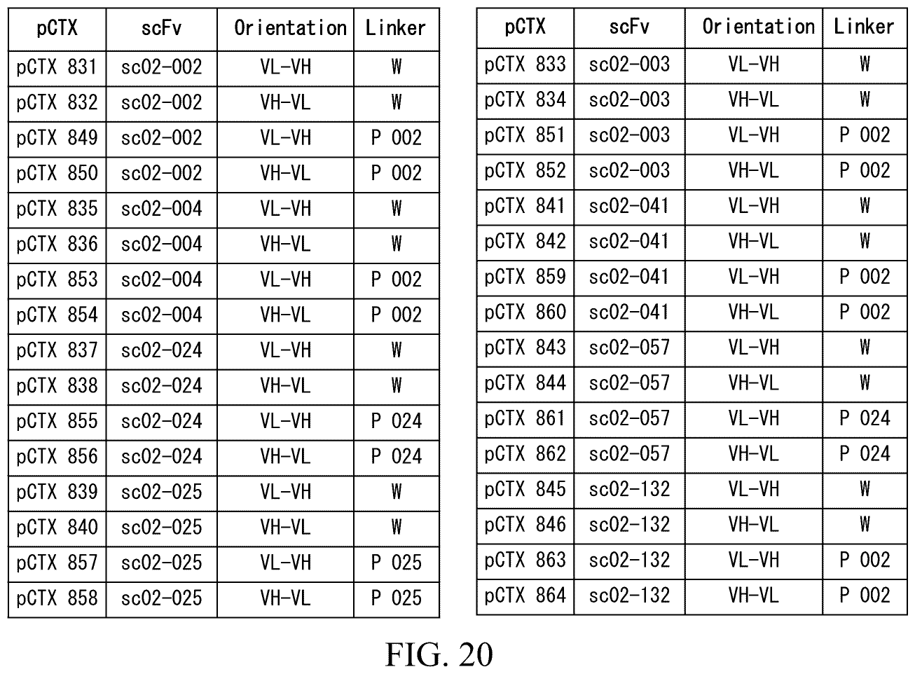

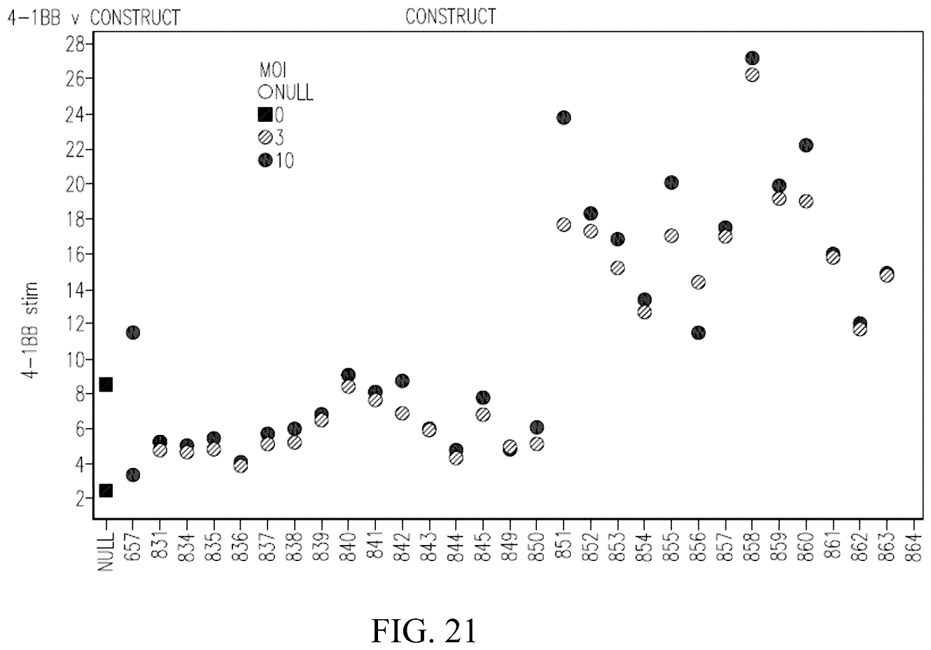

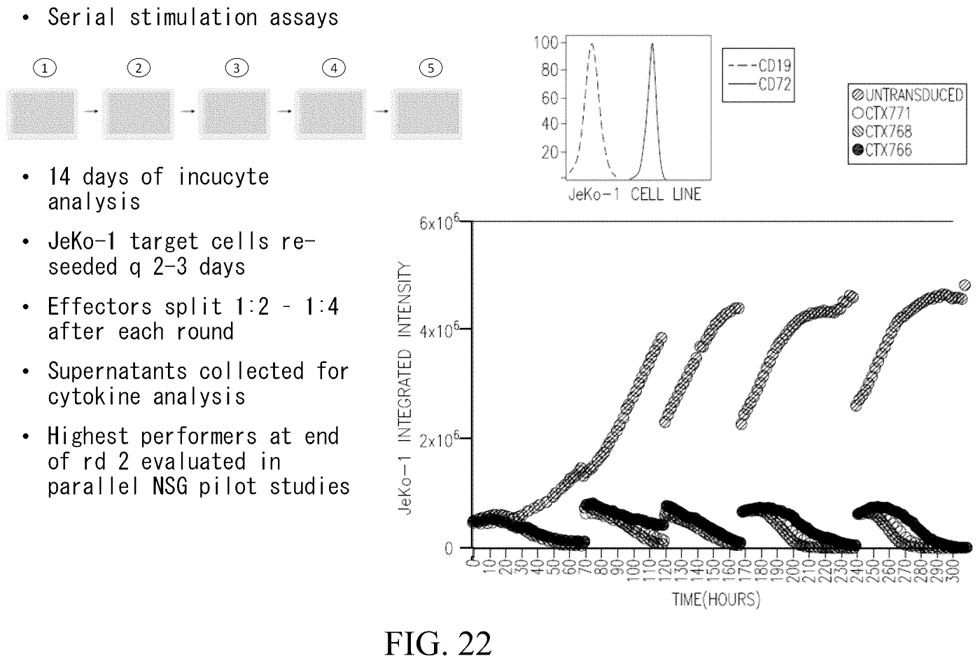

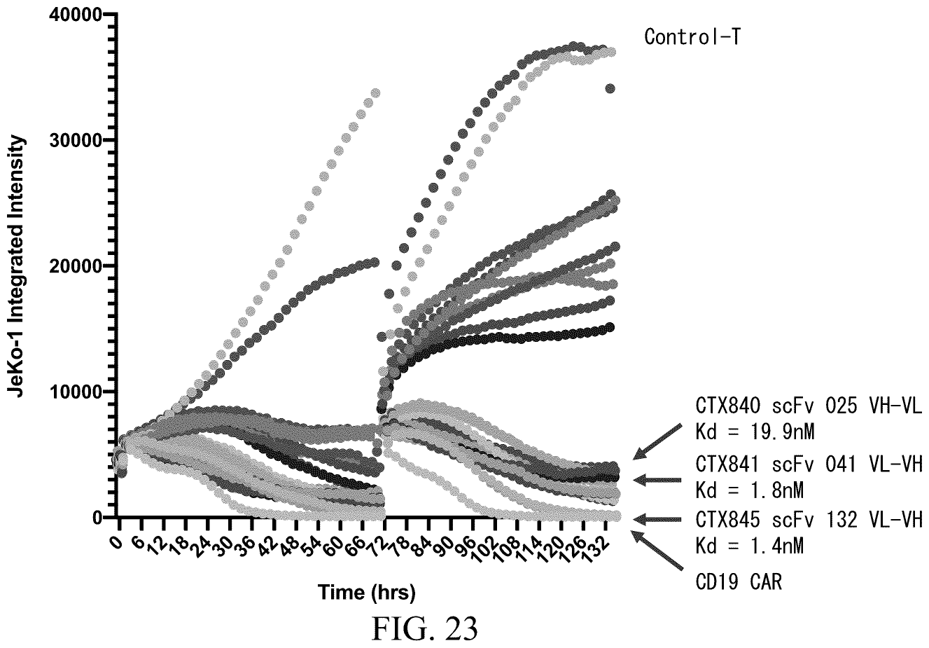

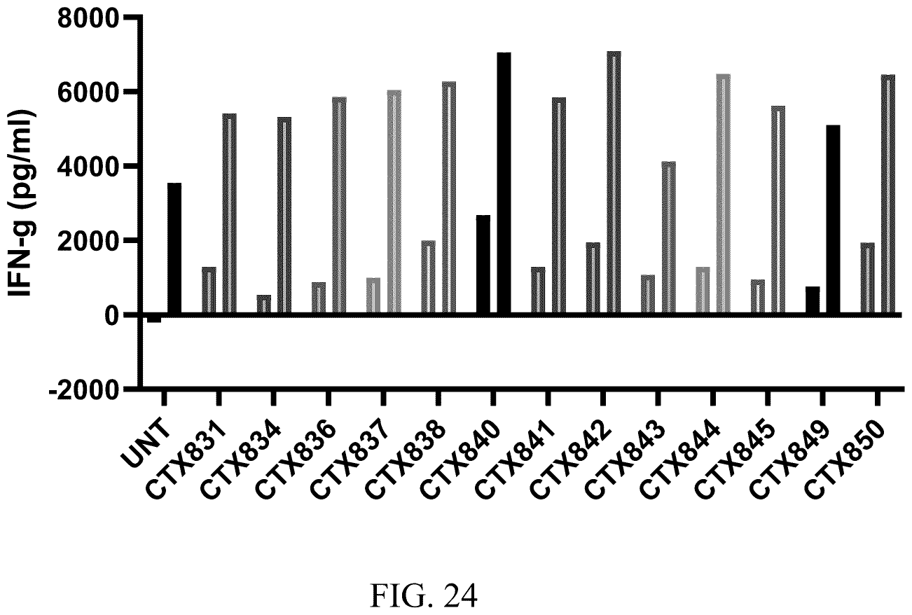

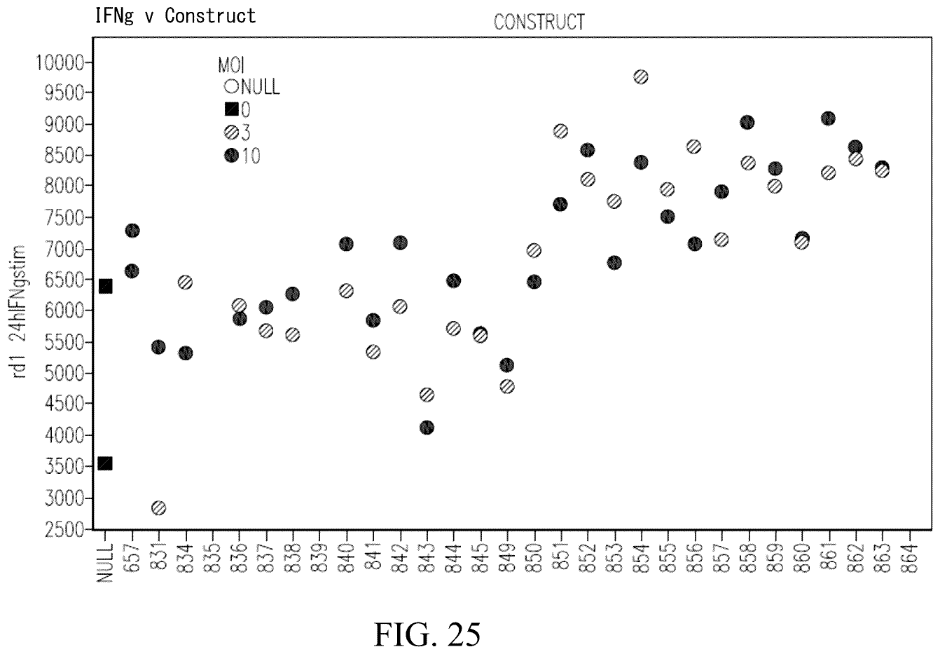

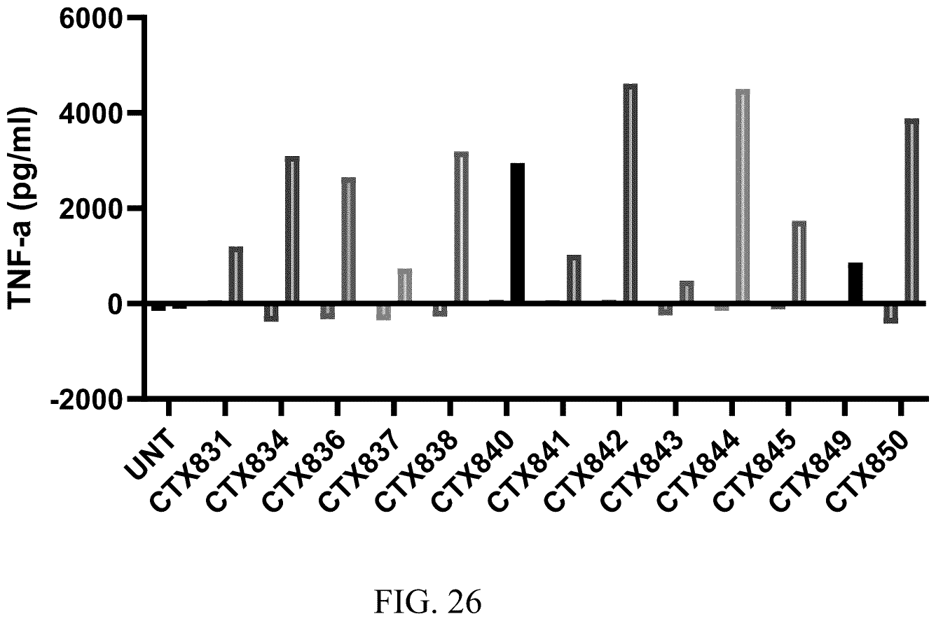

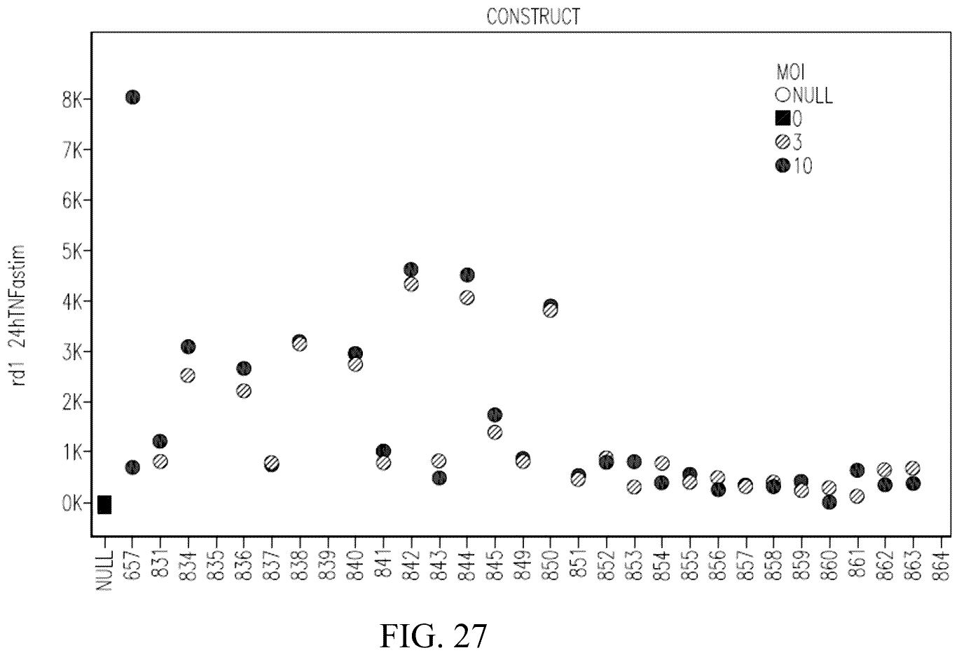

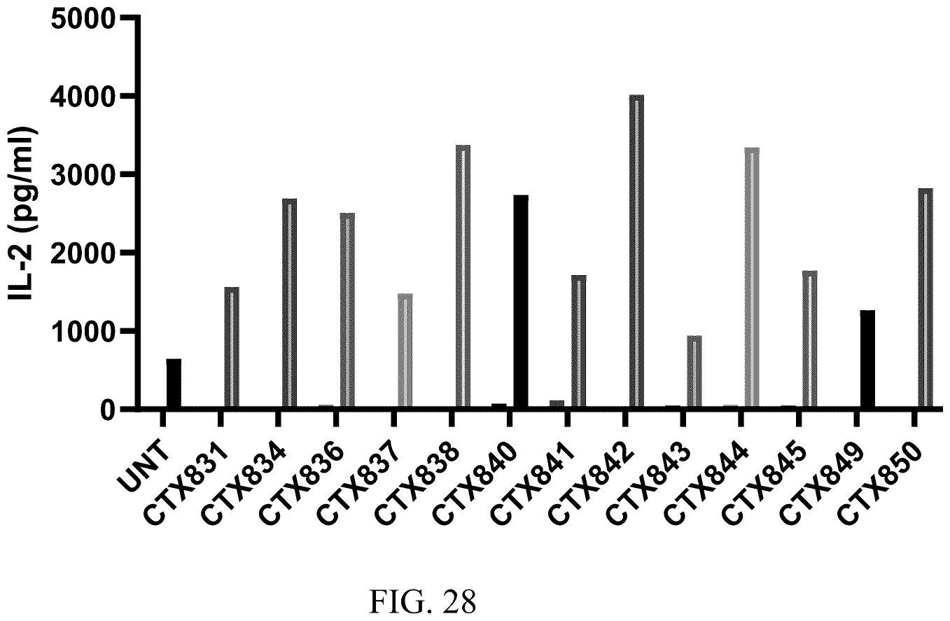

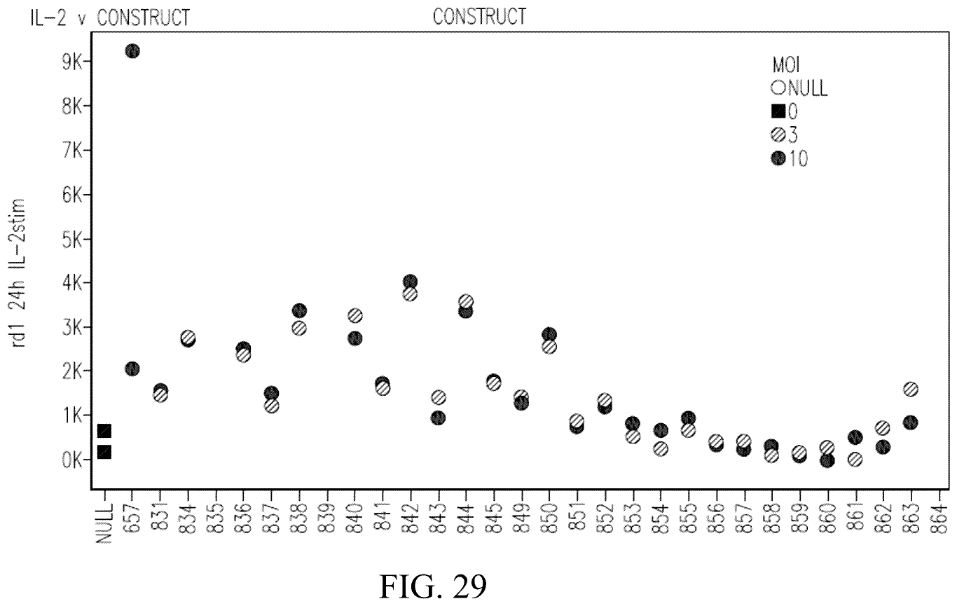

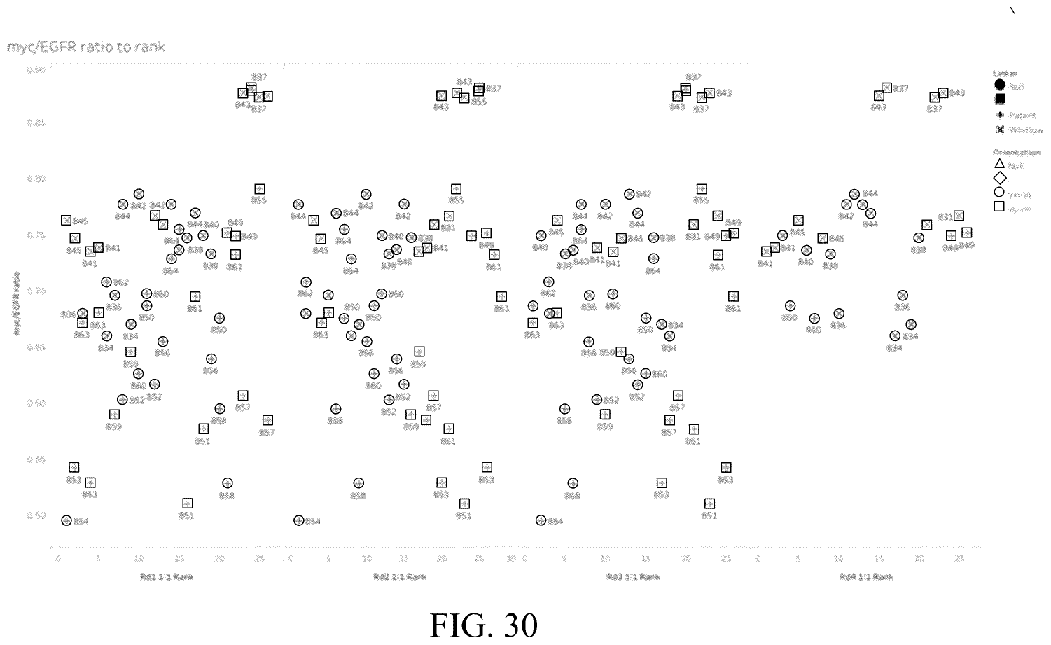

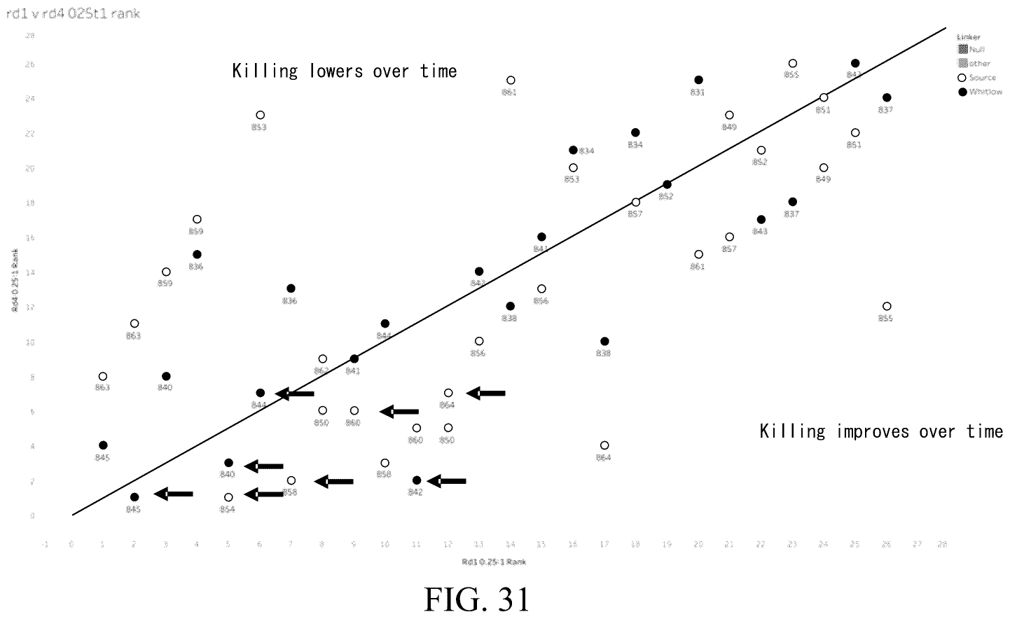

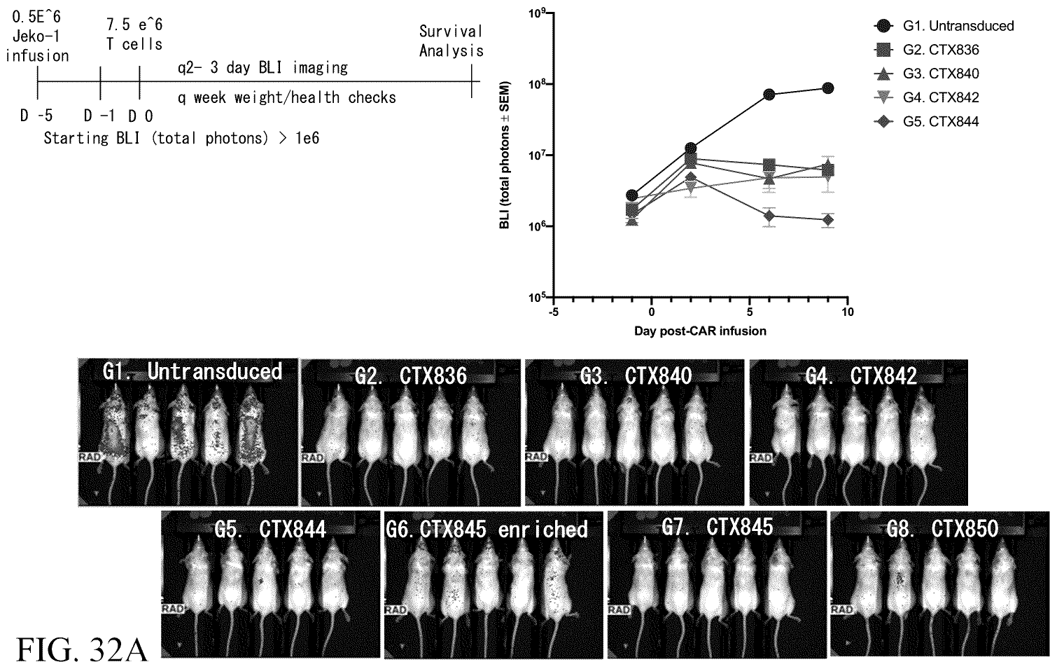

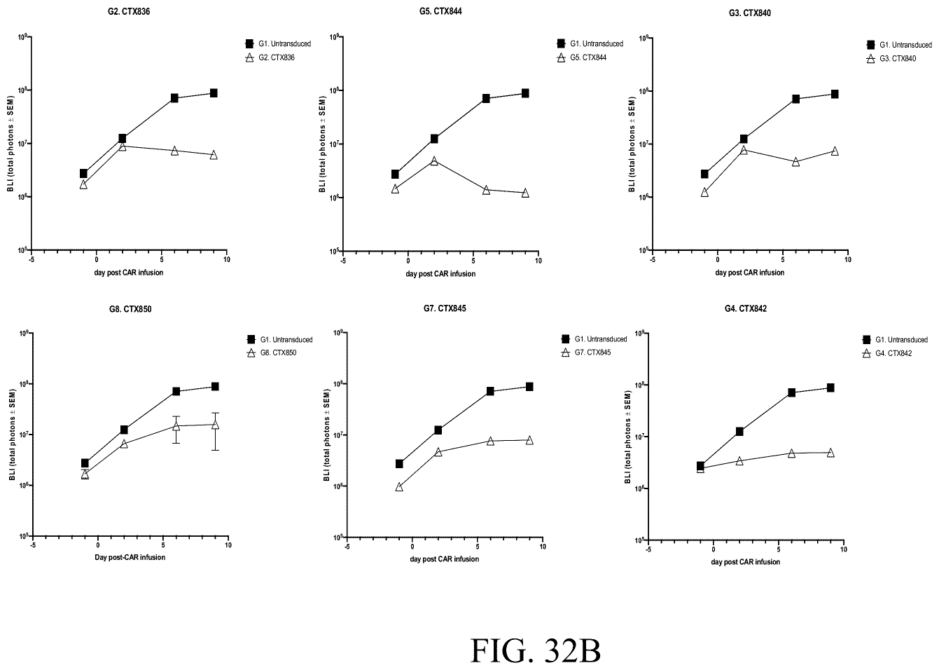

FIG. 19 shows that vector copy number (VCN) titers correlate with CAR expression titers. FIG. 20 shows CD72 specific scFv variable region orientation and linker selection for various CAR (IgG4 hinge, CD28 transmembrane domain, 4-1BB signaling domain, and CD3ζ signaling domain) constructs. FIG. 21 shows that evaluation of 4-1BB in various CD72 specific CAR T cells shows minimal auto-activation. Some variation observed between cell preparations. All CD72 specific scFvs have relatively low baseline activation (˜2 fold over UNT). FIG. 22 shows set up for serial stimulation assays. pCTX768 CER/CD72-CAR single cells elicit durable anti-lymphoma responses in vitro. FIG. 23 is a graph showing that higher affinity scFvs show greater potency in initial stimulation rounds. Cytotoxicity evaluated by incucyte over time comparing CD72 CAR scFvs and linker orientations (VH-VL or VL-VH) across 32 constructs at MOIs 3 and 10. Shown are 0.25 Effector: Target ratios. Targets re-seeded every 72 hrs. Function observed throughout 4 rounds of recursive killing. High stringency applied to assay to differentiate CARs. Evaluated at low E:T (0.1, 0.25, and 1:1), normalized using % CAR+. Between rounds, 75% of T cells removed and reseeded with Jeko-1 targets. FIG. 24 shows that high IFNγ secretion observed after co-culture with target cells in all CARs. CARs show some baseline secretion of IFN-γ (left bars). Upon stimulation with CD72+Jeko-1 cells, all CARs have strong IFN-γ response (Right bars). FIG. 25 shows that IFNγ secretion is similar between constructs and transduction rounds. FIG. 26 shows that high TNFα was observed from CARs with high in vitro cytotoxicity. All CARs have negligible baseline secretion of TNF-α (left bars). Upon stimulation, variable TNF-α secretion is observed (right bars). CARs with best in vitro cytotoxicity have higher TNF-α. FIG. 27 shows that TNF-α secretion can be more variable, but high secretion may be correlated with cytotoxicity. FIG. 28 shows that high IL-2 secretion was observed from CARs with high in vitro cytotoxicity. All CARs have negligible baseline IL-2 secretion (left bars). All CARs secrete IL-2 in response to targets. Magnitude of response correlates to strong in vitro function. FIG. 29 shows that IL-2 secretion can be more variable, but high secretion may be correlated with cytotoxicity. FIG. 30 shows EGFR: CAR surface expression (myc) ratio across constructs. CARs ranked from best to worst after each serial killing round. Highest % JeKo-1 elimination ranked #1. High concordance not necessarily associated with good function. FIG. 31 shows that a subset of CARs improve over time in serial stimulation studies. Low E:T cytotoxicity predicts longer term function in vitro. FIG. 32A shows evaluation of CD72 specific CARs (CTX836, CTX840, CTX842, CTX844, CTX845, and CTX850) in JeKo-1 Mantle cell lymphoma (MCL) model in NSG mice as measured by bioluminescence (BLI) imaging at day 9 post T-cell infusion. FIG. 32B shows bioluminescence measurement by individual CD72 specific CAR (CTX836, CTX840, CTX842, CTX844, CTX845, and CTX850). n=7-10 per group.

TNF-α death ligand produced by stimulated CAR T cells, can augment PtdSer exposure on ibrutinib treated Jeko-1 cells. Given CAR T cells produce varying levels of TNF-α, the magnitude and kinetics of PtdSer induction are measured to identify enhanced CAR formats.

32 scFv formats under analysis. Multiple alternative scFvs have promising function, with potent multi-round killing observed in vitro; high induction of IFN-γ, TNF-α, and IL-2; and low baseline activation/tonicity.

### Example 6

### Dual Targeting Cer-Cd72 Specific Car Functional Assays

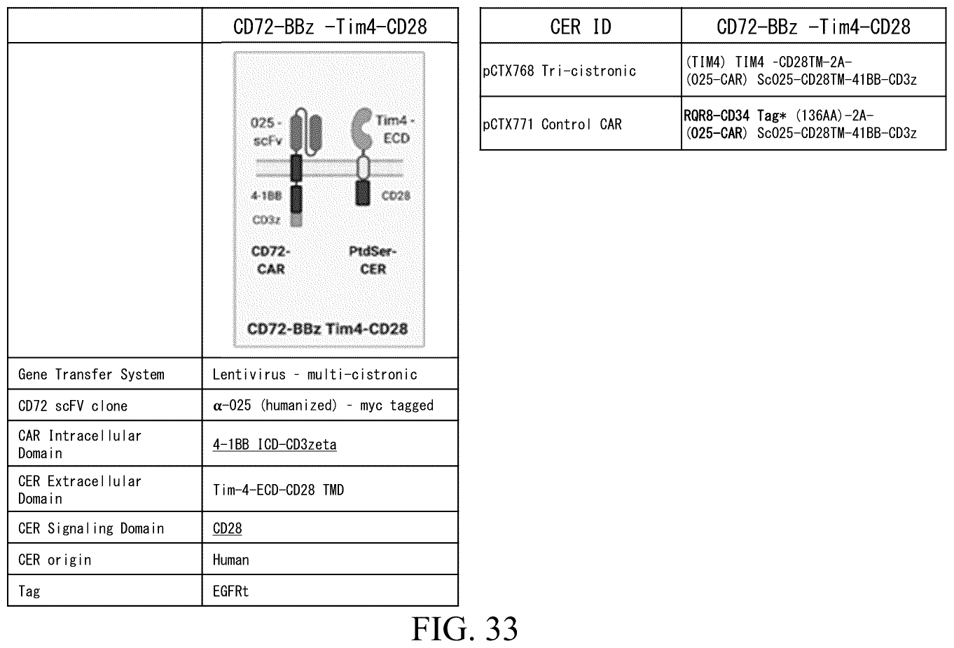

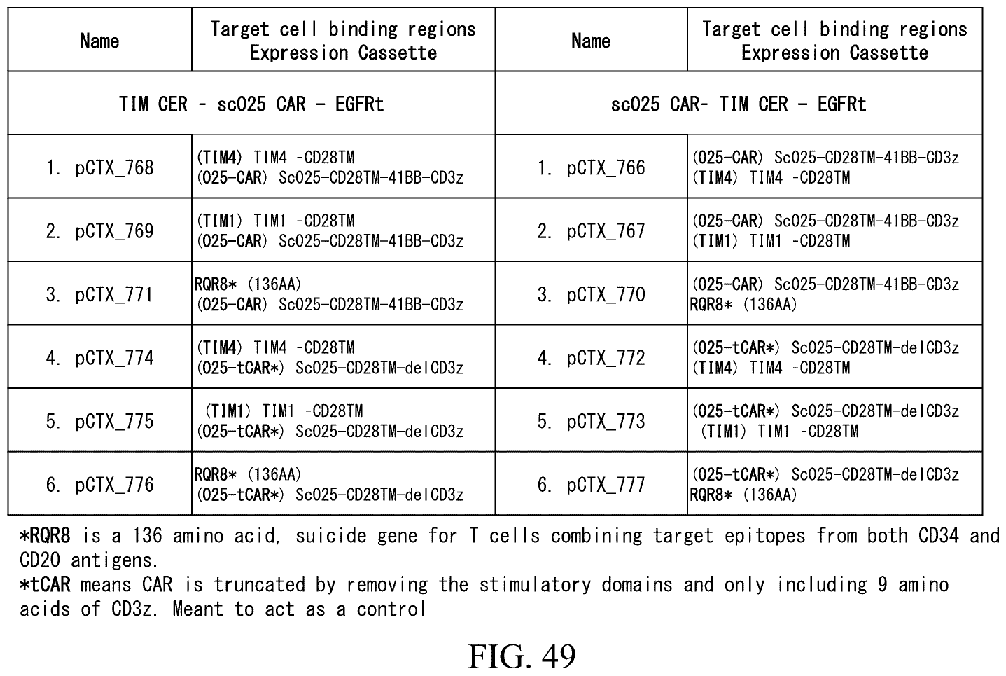

T cells expression CER and CD72 specific CAR were assayed for cytotoxicity, cytokine response, and proliferation. FIG. 33 shows exemplary schematic and design for dual targeting CER-CAR T cell, and FIG. 49 shows exemplary tricistronic CER/CAR-EGFRt Constructs.

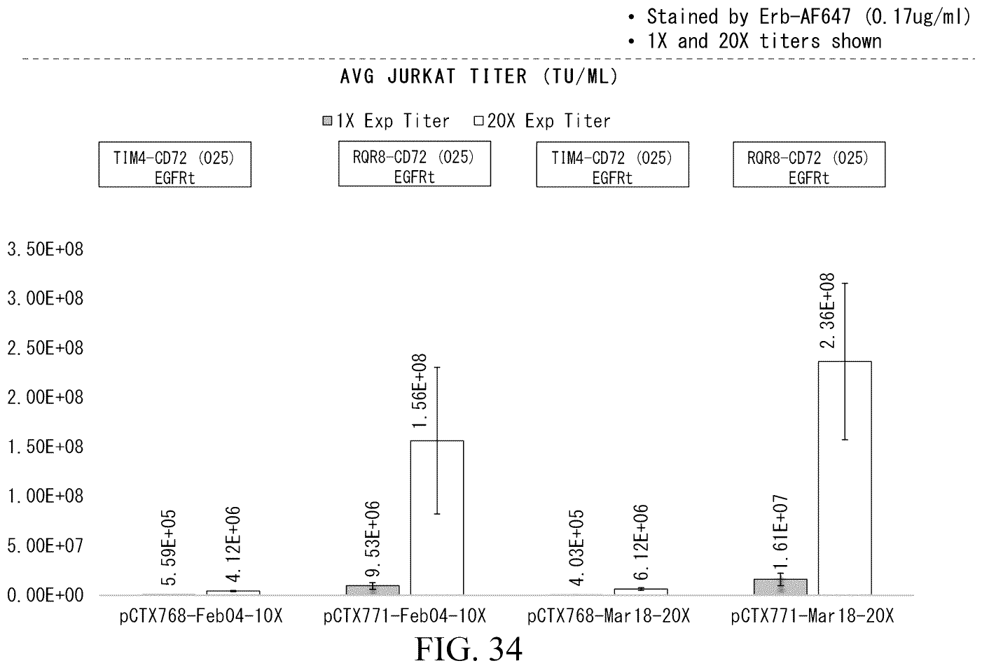

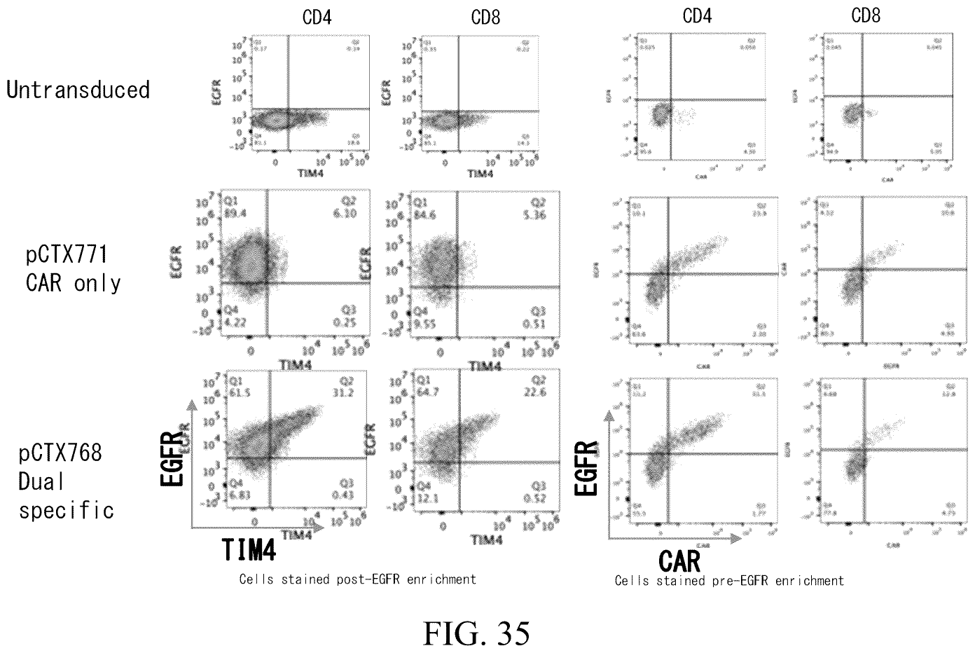

FIG. 34 shows expression titers for tricistronic vectors pCTX768 and pCTX771 at 1× and 20× titers. FIG. 35 shows PtdSer-CER (anti-Tim4) and CD72-CAR staining (anti-myc) of transduced T cells. Single cells express both a CAR and a CER. FACS plots shown are at day +10 post-transduction.

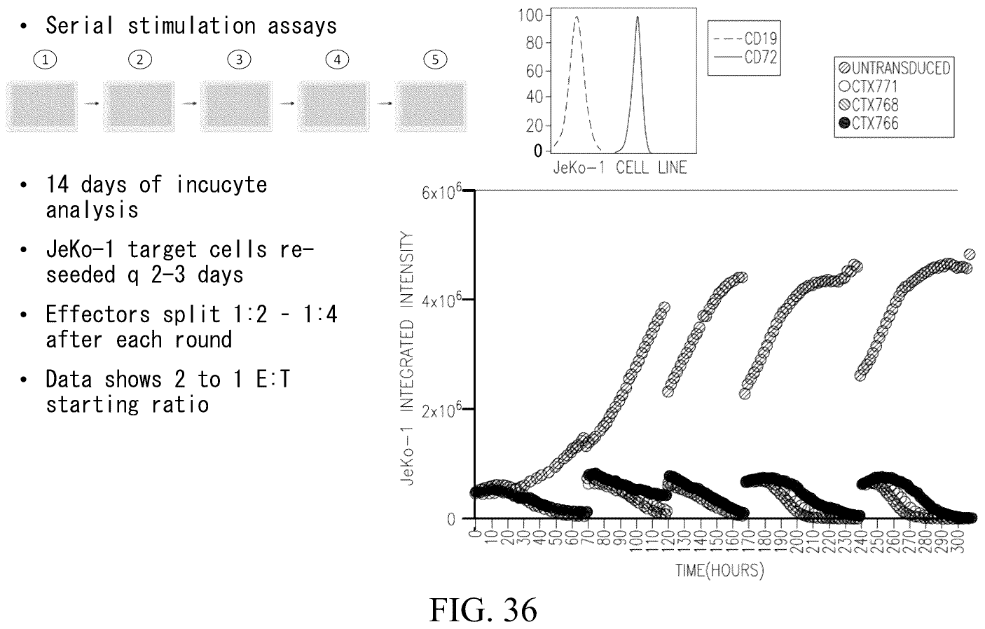

FIG. 36 shows that pCTX768 CER/CD72-CAR single cells elicit durable anti-lymphoma responses in vitro in serial stimulation assay.

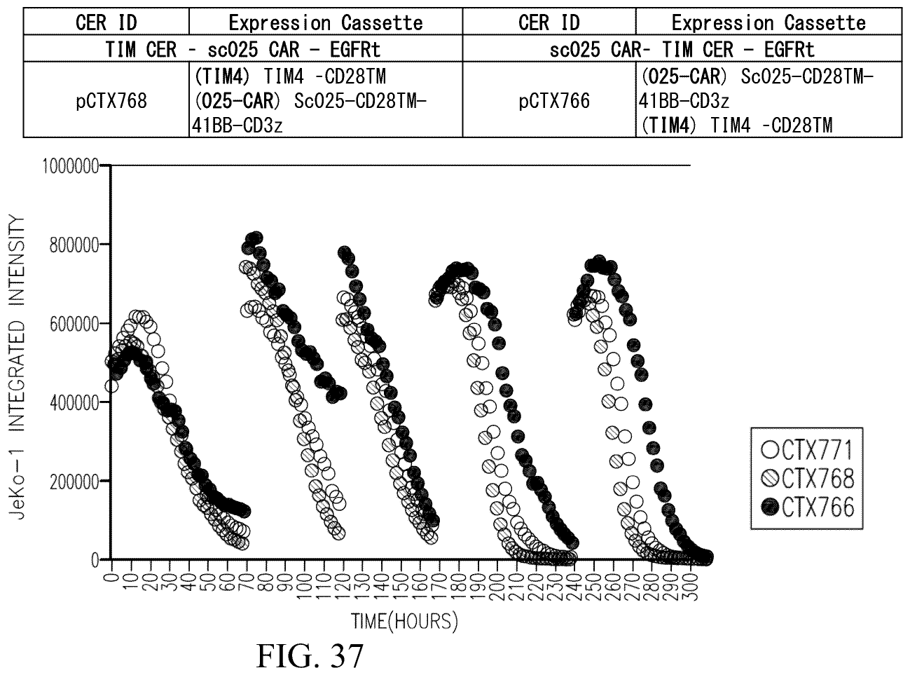

FIG. 37 shows improvement of function by tailoring CER-T2A-CAR sequence order in a multi-cistronic vector. CTX768-CER-T2A-CAR shows superior cytotoxicity to CAR-alone in 4 rounds of serial killing. Order of placement within the multi-cistronic vector favors encoding the CER 1st, followed by the CAR.

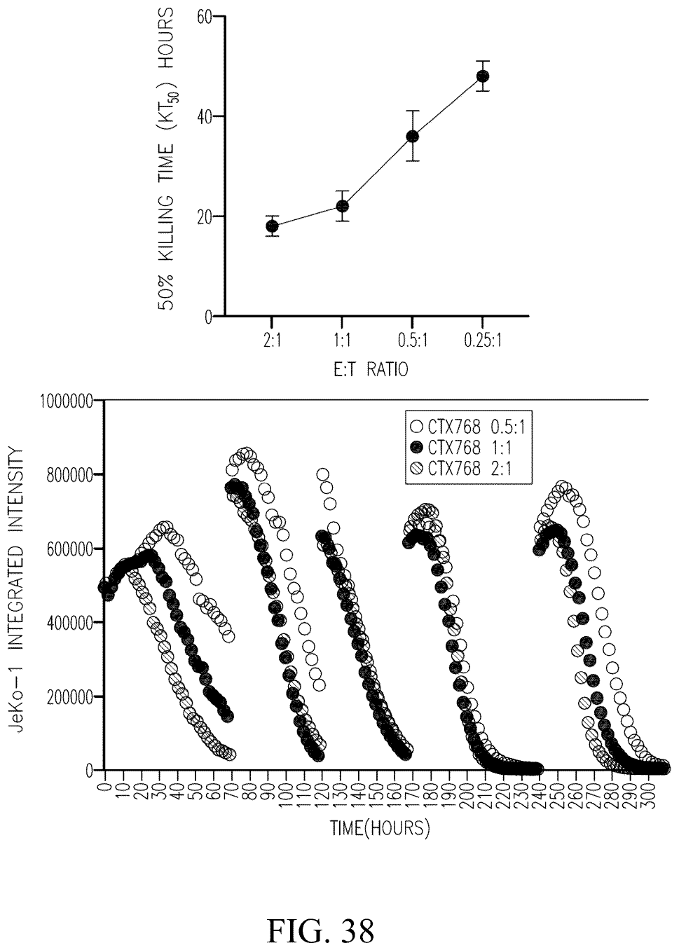

FIG. 38 shows that dual specific CER/CD72 CAR T cell has sustained anti-tumor responses at low effector: target ratios. Cytotoxicity evaluated by incucyte over time at various effector:target ratios (right). 50% killing time (the amount of time it takes to kill 50% of target tumor cells) are calculated from the various conditions (left). CTX768-CER-T2A-CAR shows clearance of targets across a range of effector: target ratios in serial killing assays. 50% Killing Time (KT50) Rd 2 shows effect of T cell dilution.

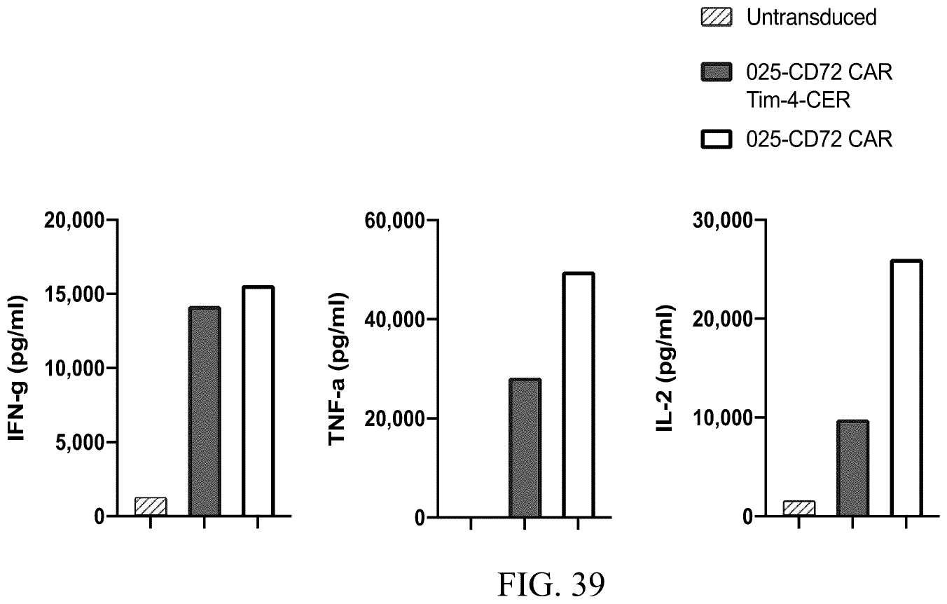

FIG. 39 shows differential cytokine production observed between pCTX768 and pCTX771. CTX768-CER-T2A-CAR has equivalent IFN-γ secretion as CTX771 CAR alone, and lower TNF-α and IL-2 secretion despite superior killing.

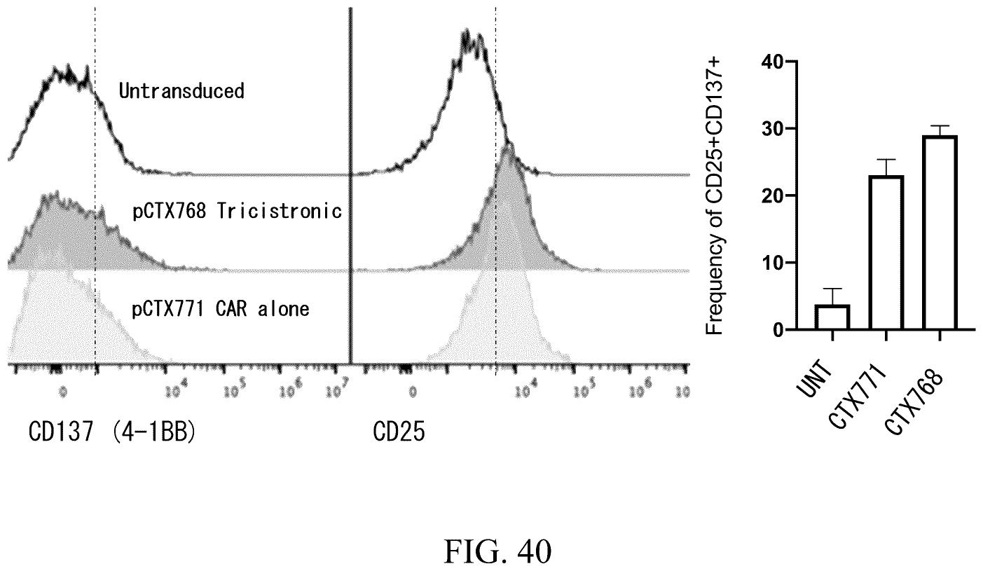

FIG. 40 shows that CER co-expression with CAR does not alter antigen dependent activation. 4-1BB and CD25 are upregulated on T cells after activation. Both CTX768-CER-T2A-CAR and CTX771 CAR alone have comparable expression of activation markers, suggesting CER co-expression does not lead to overactivation

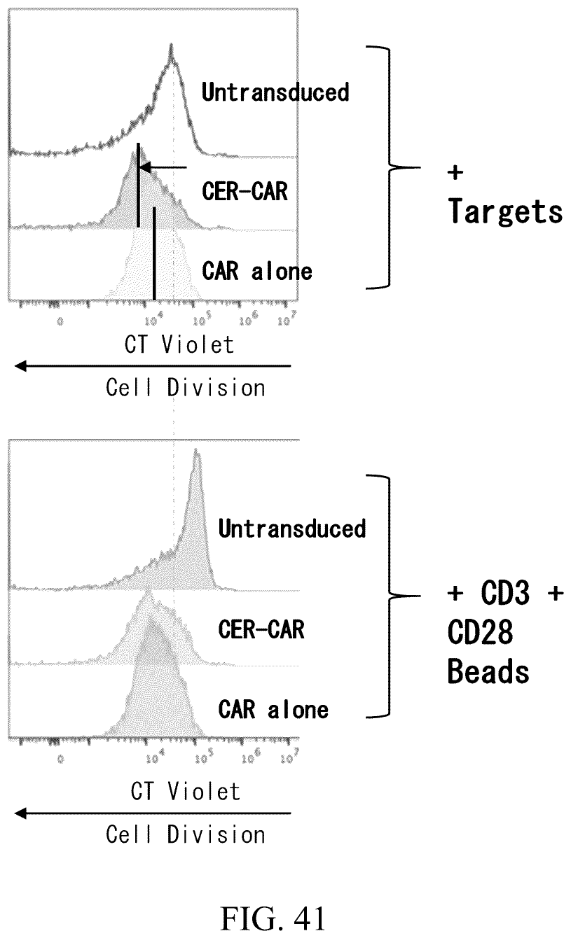

FIG. 41 shows dye dilution assay to monitor cell proliferation of pcTX768 and pCTX771 T cells. T cells labeled with CFSE dye before stimulation. After stimulation, dye dilutes with each cell division, leading to lower MFI. Extent of T cell proliferation in response to antigen is dependent on strength of signal. Enhanced proliferative capacity of CER-CAR (pCTX768) was observed compared to CAR-alone (pCTX771). Both CTX768-CER-T2A-CAR and CTX771 CAR alone proliferate in response to CD72+ target cells. CTX768-CER-T2A-CAR has slightly increased proliferation

### Example 7

### Dual Targeting Cer-Cd72 Specific Car: Tumor Uptake

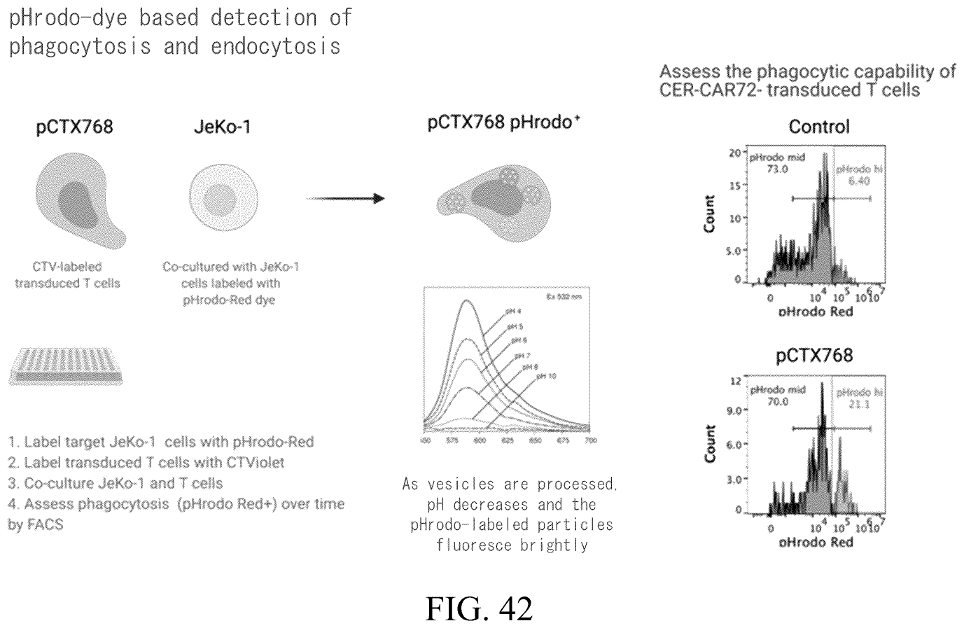

When co-expressed with a CAR, CER receptors can mediate target cell phagocytosis and subsequent lysosome acidification. FIG. 42 shows a schematic for assessing phagocytic and endocytic capabilities in engineered T cells.

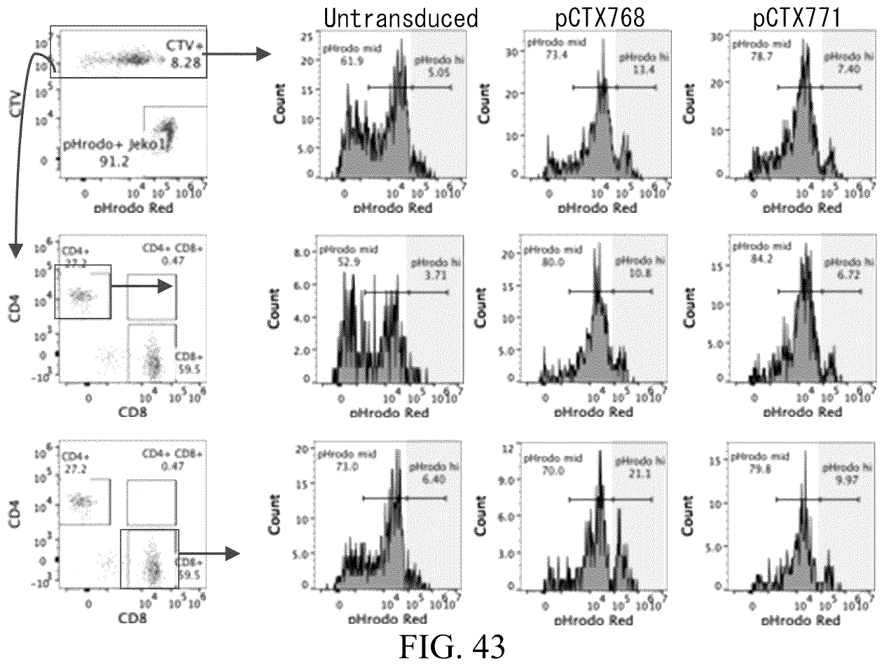

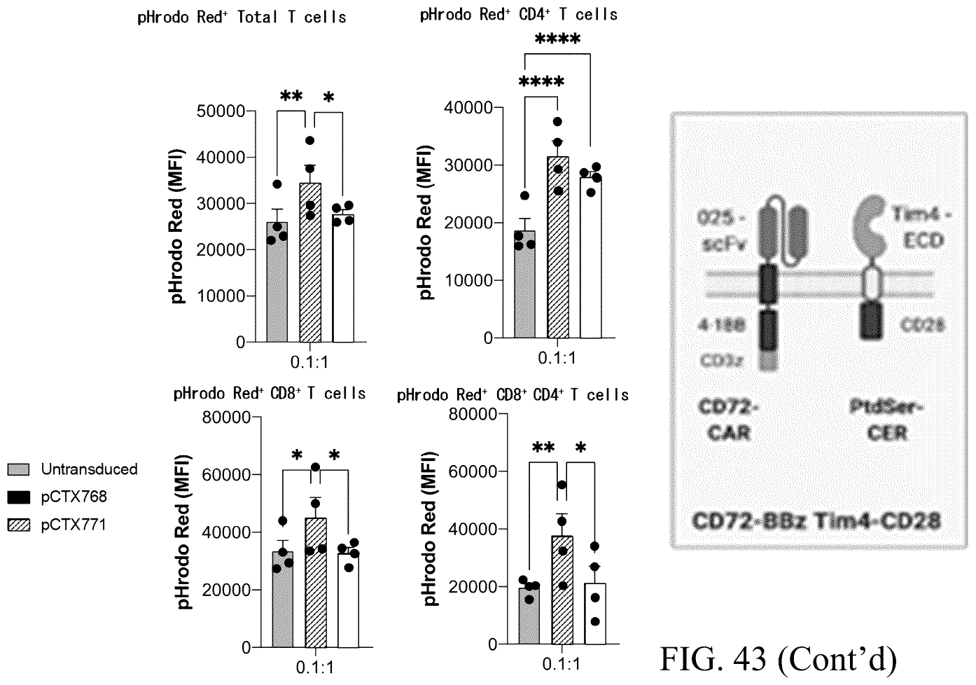

FIG. 43 shows a chimeric Tim-4 protein in combination with a CD72 CAR (pCTX768) enhances tumor uptake in CD8⁺ and CD4⁺CD8⁺ T cells over CD72 CAR alone (pCTX771).

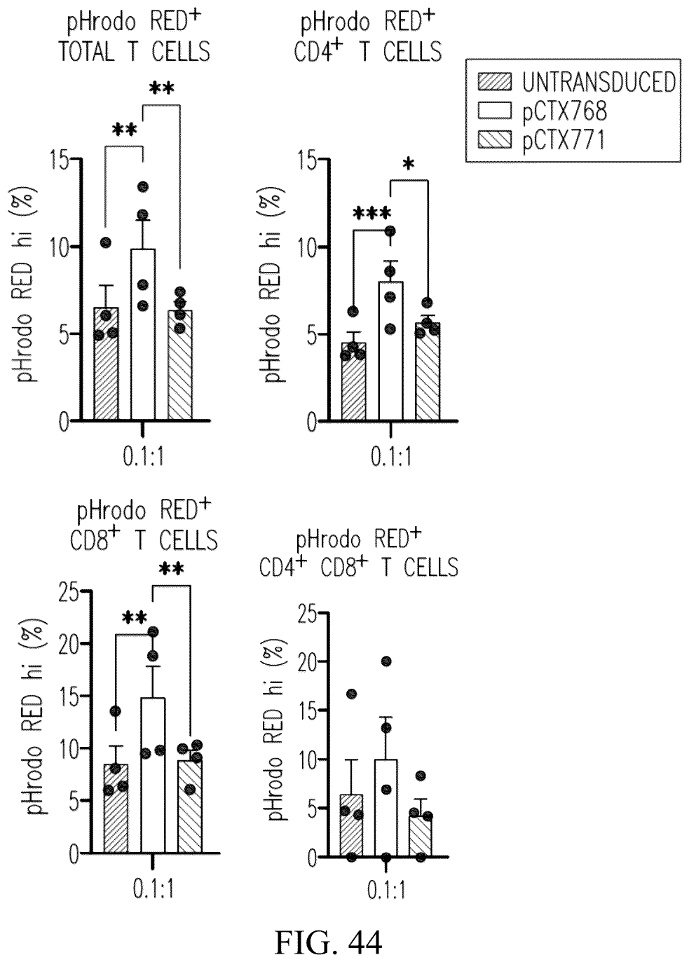

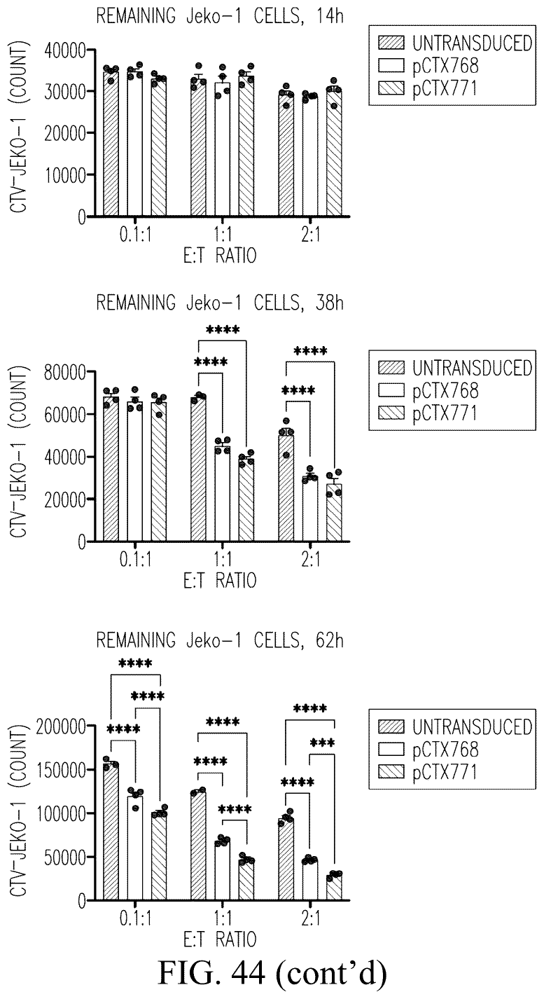

FIG. 44 shows that CER-CAR (pCTX768) T cells both engulf and kill JeKo-1 tumor targets. The frequency of pHrodo+/CT-Violet+ cells is shown across various T cell subsets after 14 hr of co-culture (left). Remaining JeKo-1-GFP+ cells quantified over time, 14 hr, 38 hr and 62 hr (right). CTX768 has capabilities to both engulf and kill JeKo-1 tumor targets.

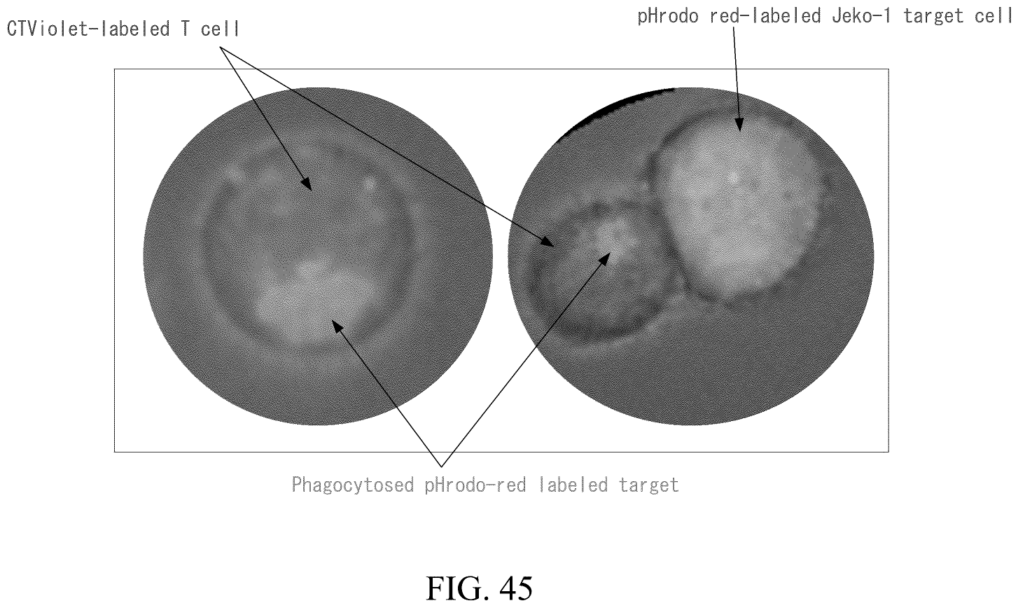

FIG. 45 shows microscopy image of CER-CAR (pCTX768) engulfing pHrodo-labeled Jeko-1 targets.

CARs are shown to be potent inducers of PtdSer. A multi-cistronic vector was engineered to express both a CER and a CD72 CAR and can detect expression of both receptors on transduced cells. CER-CAR T cells show potent cooperative killing of CD72⁺Jeko-1 cells in vitro through multiple rounds of stimulation, and improved killing compared to CAR alone. CER-CAR T cells also have favorable proliferative, activation, and cytokine secretion profiles.

### Example 8

### Dual Targeting Cer-Cd72 Specific Car Functional Assays: Memory Immunophenotyping

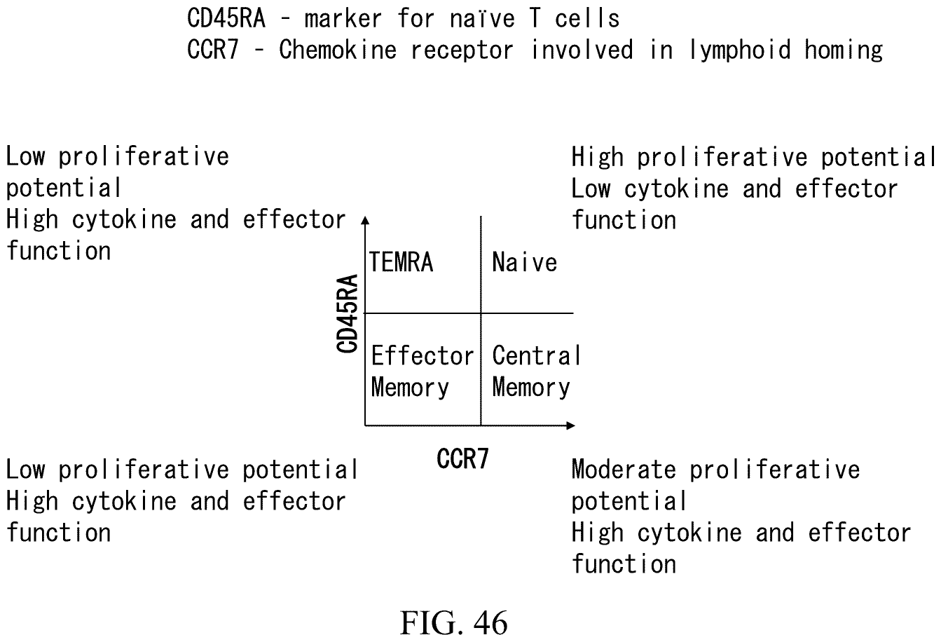

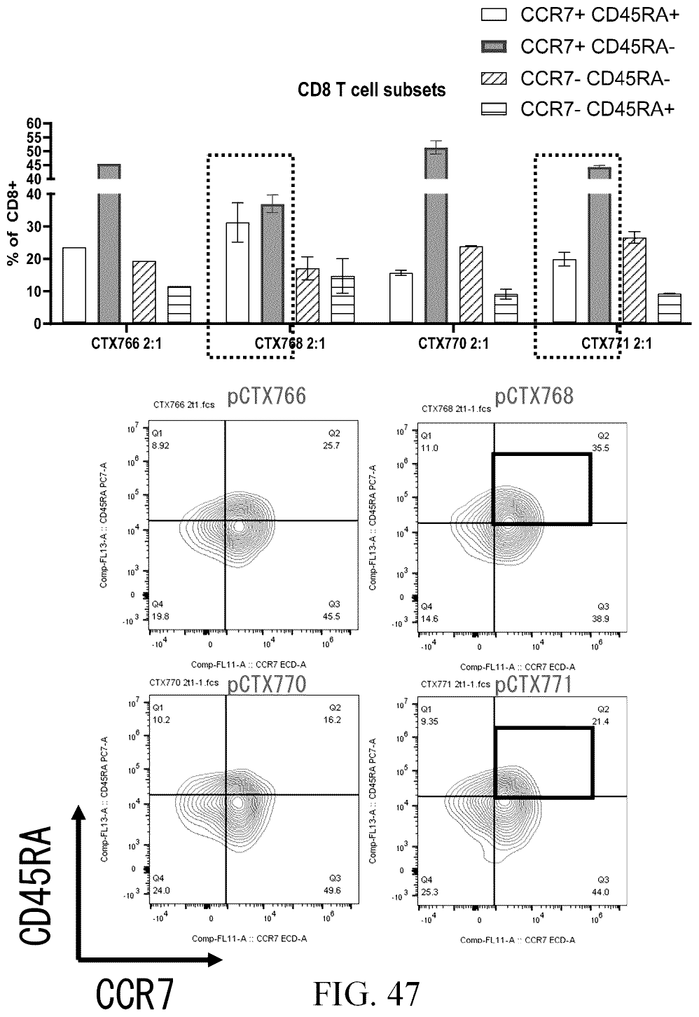

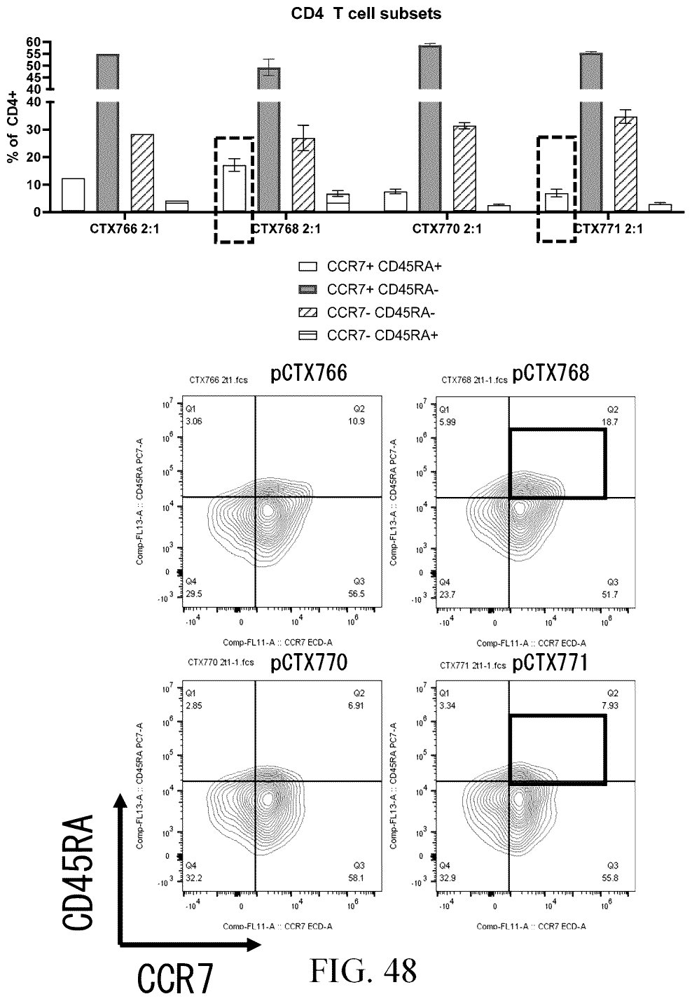

Assays were performed to immunophenotype CER/CD72 CAR transduced T cells. FIG. 46 is schematic describing role of T cell maturation markers on function. FIG. 47 shows dual-targeting CAR/CER CD8+ T cells have reduced effector memory and increased CCR7+ populations, including naïve and central memory phenotypes. FIG. 48 shows dual-targeting CAR/CER CD4+ T cells have less effector memory phenotype and increased CD45RA+CCR7+naïve T cells.

CER co-expression with CAR affects phenotype of both CD4 and CD8 cells. In CD8+ T cells, a marked increase in more persistent naïve and central memory T cells is observed, and a reduction in more differentiated effector memory T cells. In CD4+ T cells, CER co-expression promotes a larger naïve population

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to, U.S. Provisional Patent Application No. 63/066,149, filed on Aug. 14, 2020, U.S. Provisional Patent Application No. 63/172,057 filed on Apr. 7, 2021, and U.S. Provisional Patent Application No. 63/226,736, filed on Jul. 28, 2021, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A chimeric antigen receptor comprising:
(a) an extracellular domain comprising a binding domain that specifically binds to CD72, wherein the binding domain comprises:
(i) a heavy chain variable (VH) region, wherein the VH region comprises a heavy chain complementarity determining region 1 (HCDR-1) comprising the amino acid sequence set forth in SEQ ID NO:1; a heavy chain complementarity determining region 2 (HCDR-2) comprising the amino acid sequence set forth in SEQ ID NO:2; and a heavy chain complementarity determining region 3 (HCDR-3) comprising the amino acid sequence set forth in SEQ ID NO:3; and a light chain variable (VL) region, wherein the VL region comprises a light chain complementarity determining region 1 (LCDR-1) comprising the amino acid sequence set forth in SEQ ID NO:4; a light chain complementarity determining region 2 (LCDR-2) comprising the amino acid sequence set forth in SEQ ID NO:5; and a light chain complementarity determining region 3 (LCDR-3) comprising the amino acid sequence set forth in SEQ ID NO:6;
(ii) a heavy chain variable (VH) region, wherein the VH region comprises a heavy chain complementarity determining region 1 (HCDR-1) comprising the amino acid sequence set forth in SEQ ID NO:7; a heavy chain complementarity determining region 2 (HCDR-2) comprising the amino acid sequence set forth in SEQ ID NO:8; and a heavy chain complementarity determining region 3 (HCDR-3) comprising the amino acid sequence set forth in SEQ ID NO:9; and a light chain variable (VL) region, wherein the VL region comprises a light chain complementarity determining region 1 (LCDR-1) comprising the amino acid sequence set forth in SEQ ID NO:10; a light chain complementarity determining region 2 (LCDR-2) comprising the amino acid sequence set forth in SEQ ID NO: 11; and a light chain complementarity determining region 3 (LCDR-3) comprising the amino acid sequence set forth in SEQ ID NO:12;
(iii) a heavy chain variable (VH) region, wherein the VH region comprises a heavy chain complementarity determining region 1 (HCDR-1) comprising the amino acid sequence set forth in SEQ ID NO:302; a heavy chain complementarity determining region 2 (HCDR-2) comprising the amino acid sequence set forth in SEQ ID NO:303; and a heavy chain complementarity determining region 3 (HCDR-3) comprising the amino acid sequence set forth in SEQ ID NO:304; and a light chain variable (VL) region, wherein the VL region comprises a light chain complementarity determining region 1 (LCDR-1) comprising the amino acid sequence set forth in SEQ ID NO:305; a light chain complementarity determining region 2 (LCDR-2) comprising the amino acid sequence set forth in SEQ ID NO:306; and a light chain complementarity determining region 3 (LCDR-3) comprising the amino acid sequence set forth in SEQ ID NO:307;
(iv) a heavy chain variable (VH) region, wherein the VH region comprises a heavy chain complementarity determining region 1 (HCDR-1) comprising the amino acid sequence set forth in SEQ ID NO:308; a heavy chain complementarity determining region 2 (HCDR-2) comprising the amino acid sequence set forth in SEQ ID NO:309; and a heavy chain complementarity determining region 3 (HCDR-3) comprising the amino acid sequence set forth in SEQ ID NO:310; and a light chain variable (VL) region, wherein the VL region comprises a light chain complementarity determining region 1 (LCDR-1) comprising the amino acid sequence set forth in SEQ ID NO:311; a light chain complementarity determining region 2 (LCDR-2) comprising the amino acid sequence set forth in SEQ ID NO:312; and a light chain complementarity determining region 3 (LCDR-3) comprising the amino acid sequence set forth in SEQ ID NO:313;
(v) a heavy chain variable (VH) region, wherein the VH region comprises a heavy chain complementarity determining region 1 (HCDR-1) comprising the amino acid sequence set forth in SEQ ID NO:314; a heavy chain complementarity determining region 2 (HCDR-2) comprising the amino acid sequence set forth in SEQ ID NO:315; and a heavy chain complementarity determining region 3 (HCDR-3) comprising the amino acid sequence set forth in SEQ ID NO:316; and a light chain variable (VL) region, wherein the VL region comprises a light chain complementarity determining region 1 (LCDR-1) comprising the amino acid sequence set forth in SEQ ID NO:317; a light chain complementarity determining region 2 (LCDR-2) comprising the amino acid sequence set forth in SEQ ID NO:318; and a light chain complementarity determining region 3 (LCDR-3) comprising the amino acid sequence set forth in SEQ ID NO:319;
(vi) a heavy chain variable (VH) region, wherein the VH region comprises a heavy chain complementarity determining region 1 (HCDR-1) comprising the amino acid sequence set forth in SEQ ID NO:320; a heavy chain complementarity determining region 2 (HCDR-2) comprising the amino acid sequence set forth in SEQ ID NO:321; and a heavy chain complementarity determining region 3 (HCDR-3) comprising the amino acid sequence set forth in SEQ ID NO:322; and a light chain variable (VL) region, wherein the VL region comprises a light chain complementarity determining region 1 (LCDR-1) comprising the amino acid sequence set forth in SEQ ID NO:323; a light chain complementarity determining region 2 (LCDR-2) comprising the amino acid sequence set forth in SEQ ID NO:324; and a light chain complementarity determining region 3 (LCDR-3) comprising the amino acid sequence set forth in SEQ ID NO:325;
(vii) a heavy chain variable (VH) region, wherein the VH region comprises a heavy chain complementarity determining region 1 (HCDR-1) comprising the amino acid sequence set forth in SEQ ID NO:326; a heavy chain complementarity determining region 2 (HCDR-2) comprising the amino acid sequence set forth in SEQ ID NO:327; and a heavy chain complementarity determining region 3 (HCDR-3) comprising the amino acid sequence set forth in SEQ ID NO:328; and a light chain variable (VL) region, wherein the VL region comprises a light chain complementarity determining region 1 (LCDR-1) comprising the amino acid sequence set forth in SEQ ID NO:329; a light chain complementarity determining region 2 (LCDR-2) comprising the amino acid sequence set forth in SEQ ID NO:330; and a light chain complementarity determining region 3 (LCDR-3) comprising the amino acid sequence set forth in SEQ ID NO:331;
(viii) a heavy chain variable (VH) region, wherein the VH region comprises a heavy chain complementarity determining region 1 (HCDR-1) comprising the amino acid sequence set forth in SEQ ID NO:332; a heavy chain complementarity determining region 2 (HCDR-2) comprising the amino acid sequence set forth in SEQ ID NO:333; and a heavy chain complementarity determining region 3 (HCDR-3) comprising the amino acid sequence set forth in SEQ ID NO:334; and a light chain variable (VL) region, wherein the VL region comprises a light chain complementarity determining region 1 (LCDR-1) comprising the amino acid sequence set forth in SEQ ID NO:335; a light chain complementarity determining region 2 (LCDR-2) comprising the amino acid sequence set forth in SEQ ID NO:336; and a light chain complementarity determining region 3 (LCDR-3) comprising the amino acid sequence set forth in SEQ ID NO:337;
(ix) a heavy chain variable (VH) region, wherein the VH region comprises a heavy chain complementarity determining region 1 (HCDR-1) comprising the amino acid sequence set forth in SEQ ID NO:338; a heavy chain complementarity determining region 2 (HCDR-2) comprising the amino acid sequence set forth in SEQ ID NO:339; and a heavy chain complementarity determining region 3 (HCDR-3) comprising the amino acid sequence set forth in SEQ ID NO:340; and a light chain variable (VL) region, wherein the VL region comprises a light chain complementarity determining region 1 (LCDR-1) comprising the amino acid sequence set forth in SEQ ID NO:341; a light chain complementarity determining region 2 (LCDR-2) comprising the amino acid sequence set forth in SEQ ID NO:342; and a light chain complementarity determining region 3 (LCDR-3) comprising the amino acid sequence set forth in SEQ ID NO:343; or
(x) a heavy chain variable (VH) region, wherein the VH region comprises a heavy chain complementarity determining region 1 (HCDR-1) comprising the amino acid sequence set forth in SEQ ID NO:344; a heavy chain complementarity determining region 2 (HCDR-2) comprising the amino acid sequence set forth in SEQ ID NO:345; and a heavy chain complementarity determining region 3 (HCDR-3) comprising the amino acid sequence set forth in SEQ ID NO:346; and a light chain variable (VL) region, wherein the VL region comprises a light chain complementarity determining region 1 (LCDR-1) comprising the amino acid sequence set forth in SEQ ID NO:347; a light chain complementarity determining region 2 (LCDR-2) comprising the amino acid sequence set forth in SEQ ID NO:348; and a light chain complementarity determining region 3 (LCDR-3) comprising the amino acid sequence set forth in SEQ ID NO:349;
(b) an intracellular signaling domain, wherein the intracellular signaling domain comprises an immunoreceptor tyrosine-based activation motif (ITAM); and
(c) a transmembrane domain connecting the extracellular domain and intracellular signaling domain.

2. The chimeric antigen receptor of claim 1, wherein the binding domain comprises:
(i) a VH region comprising the amino acid sequence set forth in SEQ ID NO:13 or a sequence having at least 90% identity to SEQ ID NO:13, and a VL region comprising the amino acid sequence set forth in SEQ ID NO: 14 or a sequence having at least 90% identity to SEQ ID NO:14;
(ii) a VH region comprising the amino acid sequence set forth in SEQ ID NO:15 or a sequence having at least 90% identity to SEQ ID NO:15, and a VL region comprising the amino acid sequence set forth in SEQ ID NO:16 or a sequence having at least 90% identity to SEQ ID NO:16;
(iii) a VH region comprising the amino acid sequence set forth in SEQ ID NO:350 or a sequence having at least 90% identity to SEQ ID NO:350, and a VL region comprising the amino acid sequence set forth in SEQ ID NO:351 or a sequence having at least 90% identity to SEQ ID NO:351;
(iv) a VH region comprising the amino acid sequence set forth in SEQ ID NO:352 or a sequence having at least 90% identity to SEQ ID NO:352, and a VL region comprising the amino acid sequence set forth in SEQ ID NO:353 or a sequence having at least 90% identity to SEQ ID NO:353;
(v) a VH region comprising the amino acid sequence set forth in SEQ ID NO:354 or a sequence having at least 90% identity to SEQ ID NO:354, and a VL region comprising the amino acid sequence set forth in SEQ ID NO:355 or a sequence having at least 90% identity to SEQ ID NO:355;
(vi) a VH region comprising the amino acid sequence set forth in SEQ ID NO:356 or a sequence having at least 90% sequence identity to SEQ ID NO:356, and a VL region comprising the amino acid sequence set forth in SEQ ID NO:357 or a sequence having at least 90% sequence identity to SEQ ID NO:357;
(vii) a VH region comprising the amino acid sequence set forth in SEQ ID NO:358 or a sequence having at least 90% identity to SEQ ID NO:358, and a VL region comprising the amino acid sequence set forth in SEQ ID NO:359 or a sequence having at least 90% identity to SEQ ID NO:359;
(viii) a VH region comprising the amino acid sequence set forth in SEQ ID NO:360 or a sequence having at least 90% identity to SEQ ID NO:360, and a VL region comprising the amino acid sequence set forth in SEQ ID NO:361 or a sequence having at least 90% identity to SEQ ID NO:361; or
(ix) a VH region comprising the amino acid sequence set forth in SEQ ID NO:362 or a sequence having at least 90% identity to SEQ ID NO:362, and a VL region comprising the amino acid sequence set forth in SEQ ID NO:363 or a sequence having at least 90% identity to SEQ ID NO:363.

3. The chimeric antigen receptor of claim 1 or 2, wherein the binding domain comprises a VH region and a VL region that are joined by a flexible linker.

4. (canceled)

5. The chimeric antigen receptor of claim 3, wherein the flexible linker comprises the amino acid sequence set forth in SEQ ID NO:19, SEQ ID NO:20, SEQ ID NO:396, SEQ ID NO:397, SEQ ID NO:398, or SEQ ID NO:399.

6. The chimeric antigen receptor of claim 1, wherein the binding domain comprises the amino acid sequence set forth in any one of SEQ ID NOS: 17, 18, and 364-395.

7. The chimeric antigen receptor of claim 1, wherein the extracellular domain further comprises an extracellular spacer domain between the binding domain and the transmembrane domain.

8.-12. (canceled)

13. The chimeric antigen receptor of claim 1, wherein the transmembrane domain comprises a CD2, CD4, CD8a, CD28, CD5, CD3ε, CD3δ, CD3ζ, CD9, CD16, CD22, CD25, CD27, CD33, CD37, CD40, CD45, CD64, CD79A, CD79B, CD80, CD86, CD95 (Fas), CD134 (OX40), CD137 (4-1BB), CD150 (SLAMF1), CD152 (CTLA4), CD154 (CD40L), CD200R, CD223 (LAG3), CD270 (HVEM), CD272 (BTLA), CD273 (PD-L2), CD274 (PD-L1), CD278 (ICOS), CD279 (PD-1), CD300, CD357 (GITR), A2aR, DAP10, FcRα, FcRβ, FcRγ, Fyn, GAL9, KIR, Lck, LAT, LRP, NKG2D, NOTCH1, NOTCH2, NOTCH3, NOTCH4, PTCH2, ROR2, Ryk, Slp76, SIRPα, pTα, TCRα, TCRβ, TIM3, TRIM, LPA5, and Zap70 transmembrane domain.

14. The chimeric antigen receptor of claim 13, wherein the CD8a transmembrane domain comprises the amino acid sequence set forth in SEQ ID NO:24 or the CD28 transmembrane domain comprises the amino acid sequence set forth in SEQ ID NO:25.

15. The chimeric antigen receptor of claim 1, wherein the intracellular signaling domain comprises a CD3γ, CD3δ, CD3ε, CD3ζ, CD5, CD22, CD79a, CD278 (ICOS), DAP10, DAP12, or CD66d signaling domain.

16. The chimeric antigen receptor of claim 15, wherein the CD3ζ signaling domain comprises the amino acid sequence set forth in SEQ ID NO:26 or 27.

17. The chimeric antigen receptor of claim 1, wherein the intracellular signaling domain further comprises a costimulatory signaling domain.

18. The chimeric antigen receptor of claim 17, wherein the costimulatory signaling domain comprises a CD27, CD28, CD40L, GITR, NKG2C, CARD1, CD2, CD7, CD27, CD30, CD40, CD54 (ICAM), CD83, CD134 (OX-40), CD137 (4-1BB), CD150 (SLAMF1), CD152 (CTLA4), CD223 (LAG3), CD226, CD270 (HVEM), CD273 (PD-L2), CD274 (PD-L1), CD278 (ICOS), DAP10, LAT, LFA-1, LIGHT, NKG2C, SLP76, TRIM, ZAP70 costimulatory signaling domain, or any combination thereof.

19. The chimeric antigen receptor of claim 18, wherein the CD28 costimulatory signaling domain comprises the amino acid sequence set forth in SEQ ID NO:28 or 29 or the 4-1BB costimulatory signaling domain comprises the amino acid sequence set forth in SEQ ID NO:30.

20. The chimeric antigen receptor of claim 1, comprising the amino acid sequence set forth in any one of SEQ ID NOS:31-46 and 400-436, or the amino acid sequence set forth in any one of SEQ ID NOS:31-46 and 400-404 absent the signal peptide.

21. A polynucleotide encoding the chimeric antigen receptor of claim 1.

22. A vector comprising the polynucleotide of claim 21.

23. An engineered cell comprising the polynucleotide of claim 21.

24.-26. (canceled)

27. The engineered cell of claim 23, further comprising a polynucleotide encoding a chimeric engulfment receptor.

28. (canceled)

29. A pharmaceutical composition comprising the chimeric antigen receptor of any one of claims 1-20, the polynucleotide of claim 21, or the engineered cell of any one of claims 23-28 and a pharmaceutically acceptable excipient.

30. (canceled)

31. A method of treating a disease associated with CD72 in a subject comprising administering an effective amount of the engineered cell of claim 23 to the subject.

32. The method of claim 31, further comprising administering an engineered cell expressing a chimeric engulfment receptor to the subject.

33.-36. (canceled)

37. The method of claim 31, wherein the disease associated with CD72 is a B cell malignancy.

38.-41. (canceled)

42. The method of claim 41, wherein the additional therapeutic agent comprises radiation, cellular immunotherapy, antibody, immune checkpoint molecule inhibitor, chemotherapy, hormone therapy, peptide, antibiotic, anti-viral agent, anti-fungal agent, anti-inflammatory agent, UV light therapy, electric pulse therapy, high intensity focused ultrasound therapy, oncolytic virus therapy, a small molecule therapy, a molecularly targeted therapy, or any combination thereof.

43.-55. (canceled)
